(19) United States
(12) Patent Application Publication
(10) Pub. No.: US 2009/0326008 A1
(43) Pub. Date: Dec. 31, 2009

(54) NF-kappa B Inhibitor

(76) Inventors: Hironobu Ishiyama, Tokushima (JP); Kazuhide Ohta, Tokushima (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/309,417

(22) PCT Filed: Jul. 18, 2007
(86) PCT No.: PCT/JP2007/064613

§ 371 Date: Jan. 16, 2009

(30) Foreign Application Priority Data
Jul. 20, 2006 (JP) 2006-198116
Oct. 19, 2006 (JP) 2006-285169

(51) Int. Cl. A61K 31/4709; A61P 35/04; C07D 415/00

(52) U.S. Cl. 514/312; 546/157

(57) ABSTRACT

The present invention provides an NF-κB inhibitor. The NF-κB inhibitor of the present invention contains a carbostyril compound represented by General Formula (1)

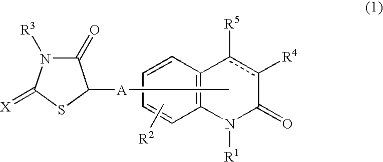

or a salt thereof, wherein A is a direct bond, a lower alkylene group, or a lower alkylidene group; X is an oxygen atom or a sulfur atom; R⁴ and R⁵ each represent a hydrogen atom; the bond between the 3 and 4 positions of the carbostyril skeleton is a single bond or a double bond; R¹ is a hydrogen atom, etc; R² is a hydrogen atom, etc; and R³ is a hydrogen atom, etc.

### TECHNICAL FIELD

The present invention relates to a NF-κB inhibitor.

### BACKGROUND ART

NF-κB, a substance known to be one of such transcriptional regulatory factors, is a heterodimer of p65 and p50 proteins. In the cytoplasm, NF-κB is usually present as substance binding with IκB, an inhibition factor, and thereby prevented from migrating into the nucleus. However, when a cell is stimulated by cytokines, ischemia, or reperfusion for whatever reason, IκB is phosphorylated and decomposed so that the NF-κB is activated and penetrates into the nucleus. NF-κB attaches itself to the NF-κB binding site of the chromosome and then promotes transcription of the gene located at downstreams. The gene controlled by NF-κB includes cytokines such as TNF-α, IL-2, IL-1, IL-6, IL-8, etc. and adhesion factors such as VCAM-1, ICAM-1, etc. . . . .

Two isomers, COX-1 and COX-2, are known as enzyme cyclooxygenase (COX). COX-1 is known to mainly be involved in gastrointestinal membrane protection, etc., whereas COX-2 is known to be involved in inflammation and pain. Therefore, pharmaceutical agents having COX-2 inhibitory activity are known to exhibit anti-inflammatory, anti-pyrogenic, and analgesic effects.

It is also known that compounds having COX-2 inhibitory activity exhibit radiosensitivity-enhancing effects in radiotherapy (Cancer Research 60, 1326-1331, Mar. 1, 2000; Clinical Cancer Research, Vol. 7, 2998-3005, October 2001; etc.), and tumor-induced angiogenesis inhibitory effects (BMC Cancer, 2006, January 12, 6:9; Breast Cancer Reseach, Vol. 7 No. 4, R422-35; etc.).

A carbostyril compound capable of inducing the production of TFF is also known (WO2006/035954). It is also known that the carbostyril compound that has the action of inducing the production of TFF is useful in preventing or treating eye diseases (such as dry eye, corneal ulcer, corneal infiltration, corneal perforation, keratitis, superficial punctate keratopathy, corneal epithelial defects, corneal erosion, recurrent corneal erosion, persistent corneal epithelial defects, corneal opacity, conjunctivitis, conjunctival epithelial defects, keratoconjunctivitis sicca, superior limbic keratoconjunctivitis, and filamentary keratitis).

However, it is not known whether such a carboxtyril compound has NF-κB inhibitory activity and/or COX-2 inhibitory activity.

### DISCLOSURE OF INVENTION

An object of the present invention is to provide a preventive or therapeutic agent for NF-κB-associated disease.

The present inventors carried out extensive research on carbostyril compounds represented by General Formula (1) shown below, and found that the carbostyril compounds exhibit NF-κB inhibitory activity, and/or COX-2 inhibitory activity, especially COX-2 production-inhibitory activity. The present invention has been accomplished based on this finding.

The present invention provides pharmaceutical compositions, methods, and use described in items 1 to 34 below.

1. A preventive or therapeutic agent for an NF-κB-associated disease comprising as an active ingredient a carbostyril compound represented by General Formula (1)

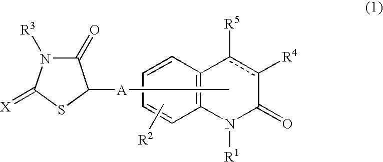

or a salt thereof,

wherein A is a direct bond, a lower alkylene group, or a lower alkylidene group;

X is an oxygen atom or a sulfur atom;

the bond between the 3 and 4 positions of the carbostyril skeleton is a single bond or a double bond;

R⁴ and R⁵ each represent a hydrogen atom, with the proviso that when the bond between the 3 and 4 positions of the carbostyril skeleton is a double bond, R⁴ and R⁵ instead may be linked together in the form of a —CH═CH—CH═CH— group;

R¹ is one of the following (1-1) to (1-30):

(1-1) a hydrogen atom,
(1-2) a lower alkyl group,
(1-3) a phenyl lower alkyl group optionally substituted on the phenyl ring with one or more members selected from the group consisting of a phenyl group, lower alkyl groups, lower alkoxy groups, halogen atoms, —(B)₁NR⁶R⁷ groups, a nitro group, a carboxy group, lower alkoxycarbonyl groups, a cyano group, phenyl lower alkoxy groups, a phenoxy group, a piperidinyl lower alkoxycarbonyl groups, amino lower alkoxycarbonyl groups optionally substituted with one or more cycloalkyl groups, 2-imidazolinylcarbonyl groups optionally substituted on the 2-imidazoline ring with one or more lower alkylthio groups, 3-pyrrolinylcarbonyl groups optionally substituted on the 3-pyrroline ring with one or more lower alkyl groups, thiazolidinylcarbonyl groups optionally substituted on the thiazolidine ring with a phenyl group, 3-azabicyclo[3.2.2]nonylcarbonyl groups, piperidinyl lower alkyl groups, anilino lower alkyl groups optionally substituted on the amino group with one or more lower alkyl groups, phenylthio lower alkyl groups, indolinyl lower alkyl groups, and piperidinylcarbonyl groups optionally substituted on the piperidine ring with one or more lower alkyl groups,
(1-4) a cycloalkyl lower alkyl group,
(1-5) a phenoxy lower alkyl group,
(1-6) a naphthyl lower alkyl group,
(1-7) a lower alkoxy lower alkyl group,
(1-8) a carboxy lower alkyl group,
(1-9) a lower alkoxycarbonyl lower alkyl group,
(1-10) a pyridyl lower alkyl group optionally substituted on the pyridine ring with one or more members selected from the group consisting of halogen atoms; piperidinyl groups; a morpholino group; piperazinyl groups optionally substituted on the piperazine ring with one or more members selected from the group consisting of a phenyl group and lower alkyl group; thienyl groups; a phenyl group; pyridyl groups; piperidinyl lower alkyl groups; phenylthio lower alkyl groups; biphenyl groups; lower alkyl groups optionally substituted with one or more halogen atoms; pyridylamino groups; pyridylcarbonylamino groups; lower alkoxy groups; anilino lower alkyl groups optionally substituted on the amino group with one or more lower alkyl groups; and anilino groups optionally substituted on the amino group with one or more lower alkyl groups,
(1-11) a cyano lower alkyl group,
(1-12) an -A₁-CONR⁸R⁹ group,
(1-13) a group of the following formula

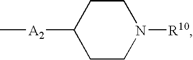

(1-14) a phenyl group,
(1-15) a quinolyl lower alkyl group,
(1-16) a lower alkoxy lower alkoxy-substituted lower alkyl group,
(1-17) a hydroxy-substituted lower alkyl group,
(1-18) a thiazolyl lower alkyl group optionally substituted on the thiazole ring with one or more members selected from the group consisting of halogen atoms, a phenyl group, thienyl groups, and pyridyl groups,
(1-19) a lower alkyl group optionally substituted with one or more halogen atoms,
(1-20) a lower alkylsilyloxy lower alkyl group,
(1-21) a phenoxy lower alkyl group optionally substituted on the phenyl ring with one or more members selected from the group consisting of lower alkyl groups optionally substituted with one or more halogen atoms; lower alkoxy groups; halogen atoms; lower alkenyl groups; cycloalkyl groups; a nitro group; and a phenyl group,
(1-22) a phenylthio lower alkyl group optionally substituted on the phenyl ring with one or more halogen atoms,
(1-23) a piperidinyl lower alkyl groups optionally substituted on the piperidine ring with one or more members selected from the group consisting of phenyl lower alkyl groups and a phenyl group,
(1-24) a piperazinyl lower alkyl group optionally substituted on the piperazine ring with one or more phenyl groups,
(1-25) a 1,2,3,4-tetrahydroisoquinolyl lower alkyl group,
(1-26) a naphthyloxy lower alkyl group,
(1-27) a benzothiazolyloxy lower alkyl group optionally substituted on the benzothiazole ring with one or more alkyl groups,
(1-28) a lower alkyl group substituted with one or more members selected from the group consisting of quinolyloxy groups and isoquinolyloxy groups,
(1-29) a pyridyloxy lower alkyl group optionally substituted on the pyridine ring with one or more lower alkyl groups,
(1-30) a lower alkenyl;

R² is one of the following (2-1) to (2-33):

(2-1) a hydrogen atom,
(2-2) a lower alkoxy group,
(2-3) a lower alkyl group,
(2-4) a carboxy lower alkoxy group,
(2-5) a lower alkoxycarbonyl lower alkoxy group,
(2-6) a hydroxy group,
(2-7) a phenyl lower alkoxy group optionally substituted on the phenyl ring with one or more members selected from the group consisting of halogen atoms; lower alkyl groups optionally substituted with one or more halogen atoms; lower alkylthio groups optionally substituted with one or more halogen atoms; lower alkoxy groups; a nitro group; lower alkylsulfonyl groups; lower alkoxycarbonyl groups; phenyl lower alkenyl groups; lower alkanoyloxy groups; and 1,2,3-thiadiazolyl groups,
(2-8) a piperidinyl lower alkoxy group optionally substituted on the piperidine ring with one or more lower alkyl groups,
(2-9) an amino-substituted lower alkoxy group optionally substituted with one or more lower alkyl groups,
(2-10) a lower alkenyloxy group,
(2-11) a pyridyl lower alkoxy group optionally substituted on the pyridine ring with one or more lower alkyl groups, each lower alkyl substituent optionally being substituted with one or more halogen atoms,
(2-12) a lower alkynyloxy group,
(2-13) a phenyl lower alkynyloxy group,
(2-14) a phenyl lower alkenyloxy group,
(2-15) a furyl lower alkoxy group optionally substituted on the furan ring with one or more lower alkoxycarbonyl groups,
(2-16) a tetrazolyl lower alkoxy group optionally substituted on the tetrazole ring with one member selected from the group consisting of a phenyl group, phenyl lower alkyl groups, and cycloalkyl lower alkyl groups,
(2-17) a 1,2,4-oxadiazolyl lower alkoxy group optionally substituted on the 1,2,4-oxadiazole ring with a phenyl group, the phenyl substituent optionally being substituted on the phenyl ring with one or more lower alkyl groups,
(2-18) an isoxazolyl lower alkoxy group optionally substituted on the isoxazole ring with one or more lower alkyl groups,
(2-19) a 1,3,4-oxadiazolyl lower alkoxy group optionally substituted on the 1,3,4-oxadiazole ring with a phenyl group, the phenyl substituent optionally being substituted on the phenyl ring with one or more lower alkyl groups,
(2-20) a lower alkanoyl lower alkoxy group,
(2-21) a thiazolyl lower alkoxy group optionally substituted on the thiazole ring with one or more members selected from the group consisting of lower alkyl groups and a phenyl group, each phenyl substituent optionally being substituted on the phenyl ring with one or more halogen atoms,
(2-22) a piperidinyloxy group optionally substituted on the piperidine ring with one or more benzoyl groups, each benzoyl substituent optionally being substituted on the phenyl ring with one or more halogen atoms,
(2-23) a thienyl lower alkoxy group,
(2-24) a phenylthio lower alkoxy group,
(2-25) a carbamoyl-substituted lower alkoxy group optionally substituted with one or more lower alkyl groups,
(2-26) a benzoyl lower alkoxy group,
(2-27) a pyridylcarbonyl lower alkoxy group,
(2-28) an imidazolyl lower alkoxy group optionally substituted on the imidazole ring with one or more phenyl lower alkyl groups,
(2-29) a phenoxy lower alkoxy group,
(2-30) a phenyl lower alkoxy-substituted lower alkoxy group,
(2-31) a 2,3-dihydro-1H-indenyloxy group,
(2-32) an isoindolinyl lower alkoxy group optionally substituted on the isoindoline ring with one or more oxo groups,
(2-33) a phenyl group;

R³ is one of the following (3-1) to (3-19):

(3-1) a hydrogen atom,
(3-2) a lower alkyl group,
(3-3) a hydroxy-substituted lower alkyl group,
(3-4) a cycloalkyl lower alkyl group,
(3-5) a carboxy lower alkyl group,
(3-6) a lower alkoxycarbonyl lower alkyl group,
(3-7) a phenyl lower alkyl group optionally substituted on the phenyl ring with one or more members selected from the group consisting of halogen atoms; lower alkyl groups optionally substituted with one or more halogen atoms; lower alkoxy groups optionally substituted with one or more halogen atoms; a phenyl group; lower alkoxycarbonyl groups; a phenoxy group; lower alkylthio groups; lower alkylsulfonyl groups; phenyl lower alkoxy groups; and amino groups optionally substituted with one or more lower alkanoyl groups,
(3-8) a naphthyl lower alkyl group,
(3-9) a furyl lower alkyl group optionally substituted on the furan ring with one or more lower alkoxycarbonyl groups,
(3-10) a thiazolyl lower alkyl group optionally substituted on the thiazole ring with one or more members selected from the group consisting of lower alkyl groups and a phenyl group, each phenyl substituent optionally being substituted on the phenyl ring with one or more optionally halogen-substituted lower alkyl groups,
(3-11) a tetrazolyl lower alkyl group optionally substituted on the tetrazole ring with one or more lower alkyl groups,
(3-12) a benzothienyl lower alkyl group optionally substituted on the benzothiophene ring with one or more halogen atoms,
(3-13) a lower alkynyl group,
(3-14) a lower alkenyl group,
(3-15) a phenyl lower alkenyl group,
(3-16) a benzoimidazolyl lower alkyl group,
(3-17) a pyridyl lower alkyl group,
(3-18) an imidazolyl lower alkyl group optionally substituted on the imidazole ring with one or more phenyl lower alkyl groups,
(3-19) a quinolyl lower alkyl group;

B is a carbonyl group or an —NHCO— group;

l is 0 or 1;

R⁶ and R⁷ each independently represent one of the following (4-1) to (4-79):

(4-1) a hydrogen atom,
(4-2) a lower alkyl group,
(4-3) a lower alkanoyl group,
(4-4) a lower alkylsulfonyl group optionally substituted with one or more halogen atoms,
(4-5) an alkoxycarbonyl group optionally substituted with one or more halogen atoms,
(4-6) a hydroxy-substituted lower alkyl group,
(4-7) a pyridylcarbonyl group optionally substituted on the pyridine ring with one or more members selected from the group consisting of pyrrolyl groups and halogen atoms,
(4-8) a pyridyl group optionally substituted on the pyridine ring with one or more members selected from the group consisting of lower alkyl groups and lower alkoxy groups,
(4-9) a pyridyl lower alkyl group,
(4-10) a phenyl group optionally substituted on the phenyl ring with one or more members selected from the group consisting of halogen atoms; lower alkyl groups optionally substituted with one or more halogen atoms; a phenoxy group; lower alkoxy groups optionally substituted with one or more halogen atoms; lower alkylthio groups; lower alkylsulfonyl groups; amino groups optionally substituted with one or more members selected from the group consisting of lower alkyl groups and lower alkanoyl groups; pyrrolidinyl groups optionally substituted on the pyrrolidine ring with one or more oxo groups; piperidinyl groups optionally substituted on the piperidine ring with one or more lower alkyl groups; lower alkenyl groups; an aminosulfonyl group; a hydroxy group; carbamoyl groups optionally substituted with one or more lower alkyl groups; phenyl lower alkoxy groups; and a cyano group,
(4-11) a cycloalkyl group optionally substituted on the cycloalkyl ring with one or more lower alkyl groups,
(4-12) a benzoyl group optionally substituted on the phenyl ring with one or more members selected from the group consisting of halogen atoms; a phenoxy group; a phenyl group; lower alkyl groups optionally substituted with one or more halogen atoms; lower alkoxy groups; lower alkanoyl groups; a nitro group; a cyano group; amino groups optionally substituted with one or more members selected from the group consisting of a phenyl group and lower alkyl groups; pyrrolidinyl groups optionally substituted on the pyrrolidine ring with one or more oxo groups; pyrrolyl groups; pyrazolyl groups; 1,2,4-triazolyl groups; and imidazolyl groups,
(4-13) a benzoyl group substituted on the phenyl ring with one or more lower alkylenedioxy groups,
(4-14) a cycloalkylcarbonyl group,
(4-15) a furylcarbonyl group,
(4-16) a naphthylcarbonyl group,
(4-17) a phenoxycarbonyl group optionally substituted on the phenyl ring with one or more members selected from the group consisting of lower alkoxy groups, lower alkyl groups, halogen atoms, and a nitro group,
(4-18) a phenyl lower alkoxycarbonyl group optionally substituted on the phenyl ring with one or more members selected from the group consisting of halogen atoms and a nitro group,
(4-19) a piperidinyl group optionally substituted on the piperidine ring with one or more members selected from the group consisting of lower alkyl groups; lower alkanoyl groups; benzoyl groups optionally substituted on the phenyl ring with one or more halogen atoms; and phenyl groups optionally substituted on the phenyl ring with one or more halogen atoms,
(4-20) a tetrahydropyranyl lower alkyl group,
(4-21) a cycloalkyl lower alkyl group,
(4-22) a lower alkenyl group,
(4-23) a phenyl lower alkyl group optionally substituted on the alkyl group with one or more lower alkoxycarbonyl groups; and optionally substituted on the phenyl ring with one or more members selected from the group consisting of halogen atoms, lower alkyl groups optionally substituted with one or more halogen atoms, lower alkoxy groups optionally substituted with one or more halogen atoms, and a hydroxy group,
(4-24) a lower alkylenedioxy-substituted phenyl lower alkyl group,
(4-25) a furyl lower alkyl group,
(4-26) a carbamoyl lower alkyl group optionally substituted with one or more members selected from lower alkyl groups and a phenyl group, each phenyl substituent optionally being substituted on the phenyl ring with one or more lower alkyl groups,
(4-27) a lower alkoxy lower alkyl group,
(4-28) an imidazolyl lower alkyl group optionally substituted on the lower alkyl group with one or more members selected from the group consisting of a carbamoyl group and lower alkoxycarbonyl groups,
(4-29) an amino-substituted lower alkyl group optionally substituted with one or more lower alkyl groups,
(4-30) a 2,3,4,5-tetrahydrofuryl group optionally substituted on the 2,3,4,5-tetrahydrofuran ring with one or more oxo groups,
(4-31) a lower alkoxycarbonyl lower alkyl group,
(4-32) a pyrrolidinyl lower alkyl group optionally substituted on the pyrrolidine ring with one or more lower alkyl groups,
(4-33) a phenoxy lower alkanoyl group,
(4-34) a morpholino lower alkyl group,
(4-35) a indolyl group,
(4-36) a thiazolyl group,
(4-37) a 1,2,4-triazolyl group,
(4-38) a pyridyl lower alkanoyl group,
(4-39) a thienylcarbonyl group,
(4-40) a thienyl lower alkanoyl group,
(4-41) a cycloalkyl lower alkanoyl group,
(4-42) an isoxazolylcarbonyl group optionally substituted on the isoxazole ring with one or more lower alkyl groups,
(4-43) a pyrazylcarbonyl group,
(4-44) a piperidinylcarbonyl group optionally substituted on the piperidine ring with one or more members selected from a benzoyl group and lower alkanoyl groups,
(4-45) a chromanylcarbonyl group,
(4-46) an isoindolinyl lower alkanoyl group optionally substituted on the isoindoline ring with one or more oxo groups,
(4-47) a thiazolidinyl lower alkanoyl group optionally substituted on the thiazolidine ring with one or more members selected from an oxo group and a thioxo group,
(4-48) a piperidinyl lower alkanoyl group,
(4-49) a phenyl lower alkenylcarbonyl group optionally substituted on the phenyl ring with one or more halogen atoms,
(4-50) a phenyl lower alkenylcarbonyl group substituted on the phenyl ring with one or more alkylenedioxy groups,
(4-51) a pyridyl lower alkenyl carbonyl group,
(4-52) a pyridylthio lower alkanoyl group,
(4-53) an indolylcarbonyl group,
(4-54) a pyrrolylcarbonyl group,
(4-55) a pyrrolidinylcarbonyl group optionally substituted on the pyrrolidine ring with one or more oxo groups,
(4-56) a benzofurylcarbonyl group,
(4-57) an indolyl lower alkanoyl group,
(4-58) a benzothienylcarbonyl group,
(4-59) a phenyl lower alkanoyl group optionally substituted on the phenyl ring with one or more halogen atoms,
(4-60) a phenylsulfonyl group optionally substituted on the phenyl ring with one or more members selected from the group consisting of lower alkoxycarbonyl groups; a cyano group; a nitro group; amino groups optionally substituted with one or more alkanoyl groups; a hydroxy group; a carboxyl group; lower alkoxycarbonyl lower alkyl groups; halogen atoms; lower alkyl groups optionally substituted with one or more halogen atoms; and lower alkoxy groups optionally substituted with one or more halogen atoms,
(4-61) a thienylsulfonyl group optionally substituted on the thiophene ring with one or more members selected from the group consisting of halogen atoms and lower alkoxycarbonyl groups,
(4-62) a quinolylsulfonyl group,
(4-63) an imidazolylsulfonyl group optionally substituted on the imidazole ring with one or more lower alkyl groups,
(4-64) a phenylsulfonyl group optionally substituted on the phenyl ring with one or more lower alkylenedioxy groups,
(4-65) a lower alkenylsulfonyl group,
(4-66) a cycloalkyl lower alkylsulfonyl group,
(4-67) a 3,4-dihydro-2H-1,4-benzoxazinylsulfonyl group optionally substituted on the 3,4-dihydro-2H-1,4-benzoxazine ring with one or more lower alkyl groups,
(4-68) a pyrazolylsulfonyl group optionally substituted on the pyrazole ring with one or more members selected from halogen atoms and lower alkyl groups,
(4-69) an isoxazolylsulfonyl group optionally substituted on the isoxazole ring with one or more lower alkyl groups,
(4-70) a thiazolylsulfonyl group optionally substituted on the thiazole ring with one or more members selected from the group consisting of lower alkyl groups and an amino group, each amino substituent optionally being substituted with one or more alkanoyl groups,
(4-71) a phenyl lower alkylsulfonyl group,
(4-72) a phenyl lower alkenylsulfonyl group,
(4-73) a naphthyloxycarbonyl group,
(4-74) a lower alkynyloxycarbonyl group,
(4-75) a lower alkenyloxycarbonyl group,
(4-76) a phenyl lower alkoxy-substituted lower alkoxycarbonyl group,
(4-77) a cycloalkyloxycarbonyl group optionally substituted on the cycloalkyl ring with one or more lower alkyl groups,
(4-78) a tetrazolyl group,
(4-79) an isoxazolyl group optionally substituted on the isoxazole ring with one or more lower alkyl groups; or instead,

R⁶ and R⁷ may be linked together to form, together with the nitrogen atom to which they are bound, a 1,2,3,4-tetrahydroisoquinolyl group, an isoindolinyl group, or a 5- to 7-membered saturated heterocyclic group, the heterocyclic group optionally containing one or more additional heteroatoms and optionally being substituted with one to three members from the following (5-1) to (5-28):

(5-1) lower alkyl groups,
(5-2) lower alkoxy groups,
(5-3) an oxo group,
(5-4) a hydroxy group,
(5-5) pyridyl lower alkyl groups,
(5-6) phenyl groups optionally substituted on the phenyl ring with one or more members selected from the group consisting of halogen atoms; lower alkoxy groups optionally substituted with one or more halogen atoms; lower alkyl groups optionally substituted with one or more halogen atoms; a cyano group; and a hydroxy group,
(5-7) lower alkylenedioxy-substituted phenyl lower alkyl groups,
(5-8) phenyl lower alkyl groups optionally substituted on the phenyl ring with one or more halogen atoms,
(5-9) pyrimidyl groups,
(5-10) pyrazyl groups,
(5-11) cycloalkyl groups,
(5-12) phenyl lower alkoxy groups optionally substituted on the phenyl ring with one or more halogen atoms,
(5-13) benzoyl groups optionally substituted on the phenyl ring with one or more halogen atoms,
(5-14) benzoyl groups substituted on the phenyl ring with one or more lower alkylenedioxy groups,
(5-15) carbamoyl lower alkyl groups optionally substituted with one or more members selected from the group consisting of a phenyl group and lower alkyl groups,
(5-16) benzoxazolyl groups,
(5-17) lower alkoxycarbonyl groups,
(5-18) a carbamoyl group,
(5-19) phenyl lower alkylidene groups optionally substituted on the phenyl ring with one or more halogen atoms,
(5-20) phenyl lower alkoxycarbonyl groups,
(5-21) pyridyl groups optionally substituted on the pyridine ring with one or more members selected from the group consisting of a cyano group and lower alkyl groups,
(5-22) furyl lower alkyl groups,
(5-23) tetrahydropyranyl groups,
(5-24) imidazolyl lower alkyl groups,
(5-25) naphthyl groups,
(5-26) 2,3-dihydro-1H-indenyl groups,
(5-27) 1,3-dioxolanyl lower alkyl groups,
(5-28) -(A₃)ₘNR¹¹R¹² groups;

A¹ is a lower alkylene group;

R⁸ and R⁹ each independently represent one of the following (6-1) to (6-25):

(6-1) a hydrogen atom,
(6-2) a lower alkyl group,
(6-3) a phenyl group optionally substituted on the phenyl ring with one or more members selected from the group consisting of lower alkyl groups optionally substituted with one or more halogen atoms; lower alkylthio groups; lower alkoxy groups optionally substituted with one or more halogen atoms; halogen atoms; a phenyl group; lower alkylamino groups; a cyano group; a phenoxy group; cycloalkyl groups; pyrrolidinyl groups optionally substituted with one or more oxo groups; 1,2,3,4-tetrahydroisoquinolylcarbonyl groups; 1,2,3,4-tetrahydroquinolylcarbonyl groups optionally substituted with one or more lower alkyl groups; 1,2,3,4-tetrahydroquinoxalinylcarbonyl groups optionally substituted with one or more lower alkyl groups; thiazolyl groups optionally substituted with one or more phenyl groups; a carbamoyl group; phenyl lower alkoxy groups; lower alkylsulfonylamino groups; anilino groups optionally substituted with one or more halogen atoms; phenyl lower alkyl groups; and hydroxy-substituted lower alkyl groups,
(6-4) a cycloalkyl group,
(6-5) a cycloakyl lower alkyl group,
(6-6) a carbamoyl lower alkyl group,
(6-7) a phenyl lower alkyl group optionally substituted on the phenyl ring with one or more members selected from the group consisting of lower alkyl groups optionally substituted with one or more halogen atoms; lower alkoxy groups optionally substituted with one or more halogen atoms; halogen atoms; and a phenyl group,
(6-8) lower alkyl-substituted amino lower alkyl group,
(6-9) a naphthyl group,
(6-10) a naphthyl lower alkyl group,
(6-11) a tetrahydronaphthyl lower alkyl group,
(6-12) a fluorenyl group,
(6-13) a pyridyl group,
(6-14) a pyridyl lower alkyl group,
(6-15) a pyrimidinyl group,
(6-16) a pyrazinyl lower alkyl group optionally substituted on the pyrazine ring with one or more lower alkyl groups,
(6-17) a thiazolyl group,
(6-18) a pyrazolyl lower alkyl group optionally substituted on the pyrazole ring with one or more lower alkyl groups,
(6-19) a thienyl lower alkyl group
(6-20) a piperidinyl group optionally substituted on the piperidine ring with one or more members selected from the group consisting of lower alkyl groups; a benzoyl group; and phenyl lower alkyl groups optionally substituted on the phenyl ring with one or more members selected from the group consisting of halogen atoms and lower alkyl groups,
(6-21) an indolyl group,
(6-22) an indazolyl group,
(6-23) a 3,4-dihydrocarbostyril optionally substituted with one or more lower alkyl groups,
(6-24) a quinolyl group optionally substituted with one or more lower alkyl groups,
(6-25) a carbazolyl group optionally substituted with one or more lower alkyl groups; or

R⁸ and R⁹ may be linked together to form, together with the nitrogen atom to which they are bound, a 5- to 8-membered saturated heterocyclic group optionally containing one or more additional heteroatoms and optionally substituted on the heterocyclic ring with one or more members selected from the group consisting of the following (6-28-1) to (6-28-24):

(6-28-1) lower alkyl groups,
(6-28-2) phenyl lower alkyl groups optionally substituted on the phenyl ring with one or more members selected from halogen atoms and lower alkoxy groups optionally substituted with one or more halogen atoms,
(6-28-3) naphthyl lower alkyl groups,
(6-28-4) phenyl lower alkylcarbamoyl lower alkyl groups,
(6-28-5) phenylcarbamoyl lower alkyl groups,
(6-28-6) phenyl lower alkoxycarbonyl groups,
(6-28-7) phenoxy lower alkyl groups optionally substituted on the phenyl ring with one or more members selected from the group consisting of halogen atoms and lower alkyl groups optionally substituted with one or more halogen atoms,
(6-28-8) biphenyl groups,
(6-28-9) phenyl groups optionally substituted on the phenyl ring with one or more halogen atoms,
(6-28-10) 2,3-dihydroindenyl groups optionally substituted with one or more halogen atoms,
(6-28-11) benzothiazolyl groups optionally substituted with one or more halogen atoms,
(6-28-12) pyridyl groups optionally substituted with one or more halogen atoms,
(6-28-13) benzothienyl groups,
(6-28-14) benzoisothiazolyl groups,
(6-28-15) thienopyridyl groups,
(6-28-16) a carbamoyl group,
(6-28-17) phenyl lower alkoxy groups optionally substituted on the phenyl ring with one or more halogen atoms,
(6-28-18) phenoxy groups optionally substituted with one or more halogen atoms,
(6-28-19) benzoyl groups optionally substituted on the phenyl ring with one or more members selected from halogen atoms and lower alkoxy groups,
(6-28-20) anilino groups optionally substituted on the phenyl ring with one or more lower alkyl groups, each lower alkyl substituent optionally being substituted with one or more halogen atoms,
(6-28-21) anilino groups substituted on the amino group with one or more lower alkyl groups, and optionally further substituted on the phenyl ring with one or more halogen atoms,
(6-28-22) benzofuryl groups,
(6-28-23) naphthyl groups,
(6-28-24) an oxo group; or

R⁸ and R⁹ may be linked together to form, together with the nitrogen atom to which they are bound, a 5- or 6-membered unsaturated heterocyclic group, the unsaturated heterocyclic group optionally being substituted on the heterocyclic ring with one or more members selected from the group consisting of the following (6-29-1) to (6-29-3):

(6-29-1) phenyl groups optionally substituted with one or more halogen atoms,
(6-29-2) 2,3-dihydroindenyl groups,
(6-29-3) benzothienyl groups; or instead,

R⁸ and R⁹ may be linked together to form, together with the nitrogen atom to which they are bound, a 1,2,3,4-tetrahydroquinolyl group; a 1,2,3,4-tetrahydroisoquinolyl group, a 1,3-dihydroisoindolyl group; an octahydropyrrolo[1,2-a]pyrazinyl group optionally substituted on the pyrazine ring with one or more lower alkyl groups; or an 8-azabicyclo[3.2.1]octyl group optionally substituted on the 8-azabicyclo[3.2.1]octyl group with one or more phenoxy groups, each phenoxy substituent optionally being substituted on the phenyl ring with one or more halogen atoms;

A₂ is a lower alkylene group;

R¹⁰ is one of the following (7-1) to (7-44):

(7-1) a hydrogen atom,
(7-2) a lower alkyl group,
(7-3) an alkoxycarbonyl group optionally substituted with one or more halogen atoms,
(7-4) a benzoyl group optionally substituted on the phenyl ring with one or more members selected from the group consisting of lower alkyl groups optionally substituted with one or more halogen atoms; a phenyl group; halogen atoms; a cyano group; a phenoxy group; lower alkoxycarbonyl groups; pyrazolyl groups; and lower alkoxy groups optionally substituted with one or more halogen atoms,
(7-5) an alkanoyl group,
(7-6) a phenyl lower alkanoyl group optionally substituted on the phenyl ring with one or more members selected from the group consisting of halogen atoms and lower alkyl groups,
(7-7) a cycloalkyl lower alkanoyl group,
(7-8) a phenyl group optionally substituted on the phenyl ring with one or more lower alkyl groups,
(7-9) a phenoxy lower alkanoyl group optionally substituted on the phenyl ring with one or more halogen atoms,
(7-10) a phenyl lower alkenylcarbonyl group,
(7-11) a pyridylcarbonyl group optionally substituted on the pyridine ring with one or more members selected from the group consisting of halogen atoms and lower alkyl groups, each lower alkyl substituent optionally being substituted with one or more halogen atoms,
(7-12) a furylcarbonyl group,
(7-13) a thienylcarbonyl group,
(7-14) a piperidinylcarbonyl group optionally substituted on the piperidine ring with one or more lower alkanoyl groups,
(7-15) a pyrrolidinylcarbonyl group optionally substituted on the pyrrolidine ring with one or more oxo groups,
(7-16) a tetrahydropyranylcarbonyl group,
(7-17) a naphthylcarbonyl group,
(7-18) an indolylcarbonyl group,
(7-19) a benzofurylcarbonyl group,
(7-20) a benzothienylcarbonyl group optionally substituted on the benzothiophene ring with one or more halogen atoms,
(7-21) a furyl lower alkyl group,
(7-22) a pyridyl lower alkyl group optionally substituted on the pyridine ring with one or more members selected from the group consisting of halogen atoms and lower alkyl groups, each lower alkyl substituent optionally being substituted with one or more halogen atoms,
(7-23) a thienyl lower alkyl group optionally substituted on the thiophene ring with one or more halogen atoms,
(7-24) a phenyl lower alkyl group optionally substituted on the phenyl ring with one or more members selected from the group consisting of lower alkoxy groups optionally substituted with one or more halogen atoms; a cyano group; lower alkyl groups optionally substituted with one or more halogen atoms; amino groups optionally substituted with one or more members selected from the group consisting of lower alkyl groups and lower alkanoyl groups; halogen atoms; lower alkoxycarbonyl groups; lower alkanoyloxy groups; lower alkylsulfonyl groups; lower alkylthio groups; and pyrrolidinyl groups,
(7-25) a thiazolyl lower alkyl group,
(7-26) an imidazolyl lower alkyl group optionally substituted on the imidazole ring with one or more lower alkyl groups,
(7-27) a pyrrolyl lower alkyl group optionally substituted on the pyrrole ring with one or more lower alkyl groups,
(7-28) a cycloalkyl lower alkyl group,
(7-29) a lower alkylthio lower alkyl group,
(7-30) a phenoxycarbonyl group optionally substituted on the phenyl ring with one or more members selected from the group consisting of halogen atoms, lower alkyl groups, and lower alkoxy groups,
(7-31) a phenyl lower alkoxycarbonyl group optionally substituted on the phenyl ring with one or more halogen atoms,
(7-32) a naphthyloxycarbonyl group,
(7-33) a lower alkynyloxycarbonyl group,
(7-34) a cycloalkylcarbonyl group,
(7-35) a quinoxalinylcarbonyl group,
(7-36) a —CO—NR¹³R¹⁴ group,
(7-37) a piperidinyl group optionally substituted on the piperidine ring with one or more lower alkyl groups,
(7-38) a cycloalkyl group,
(7-39) a tetrahydropyranyl group,
(7-40) a lower alkoxy lower alkyl group,
(7-41) a tetrahydro-2H-thiopyranyl group,
(7-42) a naphthyl group,
(7-43) a biphenyl group,
(7-44) a lower alkylsilyl lower alkoxycarbonyl group;

A³ is a lower alkylene group;

m is 0 or 1;

R¹¹ and R¹² each independently represent one of the following (8-1) to (8-5):

(8-1) a hydrogen atom,
(8-2) a lower alkyl group,
(8-3) a lower alkanoyl group,
(8-4) a phenyl lower alkanoyl group,
(8-5) a phenyl group optionally substituted on the phenyl ring with one or more halogen atoms; or instead,

R¹¹ and R¹² may be linked together to form, together with the nitrogen atom to which they are bound, a 5- or 6-membered saturated heterocyclic group which optionally contains one or more additional heteroatoms, the heterocyclic group optionally being substituted with one to three members selected from the group consisting of the following (9-1) and (9-2):

(9-1) lower alkyl groups,
(9-2) a phenyl group; and

R¹³ and R¹⁴ each independently represent one of the following (10-1) to (10-3):

(10-1) a hydrogen atom,
(10-2) a lower alkyl group,
(10-3) a phenyl group, or instead

R¹³ and R¹⁴ may be linked together to form, together with the nitrogen atom to which they are bound, a 5- or 6-membered saturated heterocyclic group which optionally contains one or more additional heteroatoms.

2. The agent according to item 1 wherein the NF-κB-associated disease is selected from the group consisting of reperfusion disorders in ischemic diseases, aggravation of the prognosis of organ transplantation or organ surgery, post-PTCA restenosis, cancer metastasis and invasion, and cachexia.

3. The agent according to item 1, which is for regulating transcription of cytokines and/or adhesion factors.

4. The agent according to item 3, which is for inhibiting activity of COX-2, TNF-α, and/or IL-8.

5. A preventive or therapeutic agent for a COX-2-associated disease or disorder comprising the carbostyril compound of item 1 or a salt thereof as an active ingredient.

6. An anticancer-effect reinforcer for use in radiotherapy or chemotherapy comprising the carbostyril compound of item 1 or a salt thereof as an active ingredient.

7. A radiosensitivity enhancer for use in radiotherapy comprising the carbostyril compound of item 1 or a salt thereof as an active ingredient.

8. A tumor-induced angiogenesis inhibitor comprising the carbostyril compound of item 1 or a salt thereof as an active ingredient.

9. A method for reinforcing anticancer effects in radiotherapy or chemotherapy, comprising administering an effective amount of the carbostyril compound of item 1 or a salt thereof to a patient.

10. Use of the carbostyril compound of item 1 or a salt thereof to produce an anticancer-effect reinforcer for use in radiotherapy or chemotherapy.

11. The agent according to item 1, wherein the carbostyril compound is represented by General Formula (1) in which R⁴ and R⁵ each represent a hydrogen atom.

12. The agent according to item 11, wherein the carbostyril compound is represented by General Formula (1) in which a group of the formula

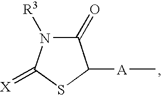

in which R³, A and X are as defined in item 1 above, is bound to the 3, 4, 5, 6, 7 or 8 position of the carbostyril skeleton.

13. The agent according to item 12, wherein the carbostyril compound is represented by General Formula (1) in which the bond between the 3 and 4 positions of the carbostyril skeleton is a single bond, and the group of the formula,

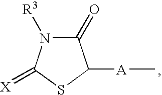

in which R³, A and X are as defined in item 1 above, is bound to the 5 or 6 position of the carbostyril skeleton.

14. The agent according to item 12 or 13, wherein the carbostyril compound is represented by General Formula (1) in which A is a lower alkylene group or a lower alkylidene group.

15. The agent according to item 14, wherein the carbostyril compound is represented by General Formula (1) in which R¹ is one of (1-2), (1-3), (1-4), (1-6), (1-10), (1-12), (1-13), (1-18) and (1-21) as defined in item 1 above.

16. The agent according to item 15, wherein the carbostyril compound is represented by General Formula (1) in which the group of the formula

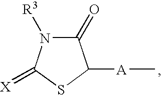

in which R³, A and X are as defined in item 1 above, is bound to the 5 position of the carbostyril skeleton.

17. The agent according to item 16, wherein the carbostyril compound is represented by General Formula (1) in which R¹ is a phenyl lower alkyl group optionally substituted on the phenyl ring with one or more members selected from the group consisting of a phenyl ring, halogen atoms, —(B)ₗNR⁶R⁷ groups wherein B, l, R⁶ and R⁷ are as defined in item 1, lower alkoxycarbonyl groups, and phenyl lower alkoxy groups.

18. The agent according to item 17, wherein the carbostyril compound is represented by General Formula (1) in which A is a lower alkylene group, R² is a hydrogen atom or a lower alkoxy group, R³ is a hydrogen atom, and X is an oxygen atom or a sulfur atom.

19. The agent according to item 16, wherein the carbostyril compound is represented by General Formula (1) in which A is a lower alkylene group, R¹ is a lower alkyl group, R² is a hydrogen atom or a lower alkoxy group, R³ is a hydrogen atom, and X is an oxygen atom or a sulfur atom.

20. The agent according to item 16, wherein the carbostyril compound is represented by General Formula (1) in which A is a lower alkylene group, R¹ is a naphthyl lower alkyl group, R² is a hydrogen atom or a lower alkoxy group, R³ is a hydrogen atom, and X is an oxygen atom or a sulfur atom.

21. The agent according to item 16, wherein the carbostyril compound is represented by General Formula (1) in which A is a lower alkylene group, R¹ is a group of the formula

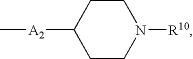

in which R¹⁰ and A₂ are as defined in item 1 above, R² is a hydrogen atom or a lower alkoxy group, R³ is a hydrogen atom, and X is an oxygen atom or a sulfur atom.

22. The agent according to item 12, wherein the carbostyril compound is represented by General Formula (1) in which the bond between the 3 and 4 positions of the carbostyril skeleton is a double bond, and a group of the formula

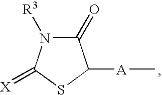

in which R³, A and X are as defined in item 1 above, is bound to the 3, 4 or 5 position of the carbostyril skeleton.

23. The agent according to item 22, wherein the carbostyril compound is represented by General Formula (1) in which R¹ is one of (1-2) and (1-3) as defined in item 1.

24. The agent according to item 23, wherein the carbostyril compound is represented by General Formula (1) in which A is a lower alkylene group or a lower alkylidene group, and R² is a hydrogen atom or a lower alkoxy group.

25. The agent according to item 1, wherein the carbostyril compound is represented by General Formula (1) in which the bond between the 3 and 4 positions of the carbostyril skeleton is a double bond, and R⁴ and R⁵ are linked together in the form of a —CH═CH—CH═CH— group.

26. The agent according to item 25, wherein the carbostyril compound is represented by General Formula (1) in which a group of the formula

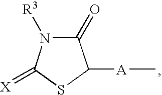

in which R³, A and X are as defined in item 1 above, is bound to the 7 position of the carbostyril skeleton.

27. The agent according to item 26, wherein the carbostyril compound is represented by General Formula (1) in which R¹ is one of (1-2) and (1-3) as defined in item 1 above.

28. The agent according to item 27, wherein the carbostyril compound is represented by General Formula (1) in which A is a lower alkylene group or a lower alkylidene group, R² and R³ are both hydrogen atoms, and X is an oxygen atom or a sulfur atom.

29. The agent according to item 1, wherein the carbostyril compound is represented by General Formula (1) in which A is a direct bond.

30. The agent according to item 1, wherein the carbostyril compound is represented by General Formula (1) in which A is a lower alkylene group.

31. The agent according to item 1, wherein the carbostyril compound is represented by General Formula (1) in which A is a lower alkylidene group.

32. The agent according to any one of items 29 to 31, wherein the carbostyril compound is represented by General Formula (1) in which the bond between the 3 and 4 positions of the carbostyril skeleton is a single bond or a double bond, and R⁴ and R⁵ each represent a hydrogen atom.

33. The agent according to any one of items 29 to 31, wherein the carbostyril compound is represented by General Formula (1) in which the bond between the 3 and 4 positions of the carbostyril skeleton is a double-bond, and R⁴ and R⁵ are linked together in the form of a —CH═CH—CH═CH— group.

34. The agent according to item 1, wherein the carbostyril compound is selected from the group consisting of the following compounds:
5-[1-(biphenyl-4-ylmethyl)-8-methoxy-2-oxo-1,2,3,4-tetrahydroquinolin-5-ylmethyl]thiazolidine-2,4-dione,
5-[1-(4-chlorobenzyl)-8-methoxy-2-oxo-1,2,3,4-tetrahydroquinolin-5-ylmethyl]thiazolidine-2,4-dione,
5-[1-(4-bromobenzyl)-8-methoxy-2-oxo-1,2,3,4-tetrahydroquinolin-5-ylmethyl]thiazolidine-2,4-dione,
5-[1-(2-naphthylmethyl)-8-methoxy-2-oxo-1,2,3,4-tetrahydroquinolin-5-ylmethyl]thiazolidine-2,4-dione,
5-{1-[4-(heptyloxycarbonylamino)benzyl]-8-methoxy-2-oxo-1,2,3,4-tetrahydroquinolin-5-ylmethyl}thiazolidine-2,4-dione,
5-[1-(1-biphenyl-4-ylpiperidin-4-ylmethyl)-2-oxo-1,2,3,4-tetrahydroquinolin-5-ylmethyl]thiazolidine-2,4-dione,
5-{1-[1-(4-methylphenyl)piperidin-4-ylmethyl]-2-oxo-1,2,3,4-tetrahydroquinolin-5-ylmethyl}thiazolidine-2,4-dione,
5-{1-[4-(2-chlorobenzyloxycarbonylamino)benzyl]-8-methoxy-2-oxo-1,2,3,4-tetrahydroquinolin-5-ylmethyl}thiazolidine-2,4-dione,
1-(biphenyl-4-ylmethyl)-8-methoxy-5-(4-oxo-2-thioxothiazolidin-5-ylmethyl)-3,4-dihydro-1H-quinolin-2-one,
8-methoxy-1-methyl-5-(4-oxo-2-thioxothiazolidin-5-ylmethyl)-3,4-dihydro-1H-quinolin-2-one,
8-methoxy-1-(3-methylbutyl)-5-(4-oxo-2-thioxothiazolidin-5-ylmethyl)-3,4-dihydro-1H-quinolin-2-one,
1-propyl-8-methoxy-5-(4-oxo-2-thioxothiazolidin-5-ylmethyl)-3,4-dihydro-1H-quinolin-2-one,
1-isobutyl-8-methoxy-5-(4-oxo-2-thioxothiazolidin-5-ylmethyl)-3,4-dihydro-1H-quinolin-2-one,
8-methoxy-1-phenethyl-5-(4-oxo-2-thioxothiazolidin-5-ylmethyl)-3,4-dihydro-1H-quinolin-2-one, and
1-(4-phenylthiomethyl)benzyl-5-(4-oxo-2-thioxothiazolidin-5-ylmethyl)-3,4-dihydro-1H-quinolin-2-one; or a salt thereof.

### Carbostyril Compounds

Carbostyril compounds represented by General Formula (1) used in the present invention are known compounds, and disclosed, for example, in WO2006/035954.

Specific examples of groups in the above General Formula (1) are as follows.

Examples of lower alkylene groups include straight and branched C₁₋₆ alkylene groups, such as methylene, ethylene, trimethylene, 2-methyltrimethylene, 2,2-dimethylethylene, 2,2-dimethyltrimethylene, 1-methyltrimethylene, methylmethylene, ethylmethylene, tetramethylene, pentamethylene, and hexamethylene.

Examples of lower alkylidene groups include straight and branched C₁₋₆ alkylidene groups, such as methylidene, ethylidene, propylidene, butylidene, pentylidene, and hexylidene.

Examples of lower alkyl groups include straight and branched C₁₋₆ alkyl groups, such as methyl, ethyl, n-propyl, isopropyl, n-butyl, isobutyl, tert-butyl, sec-butyl, n-pentyl, isopentyl, neopentyl, n-hexyl, isohexyl, and 3-methylpentyl.

Examples of lower alkoxy groups include straight and branched C₁₋₆ alkoxy groups, such as methoxy, ethoxy, n-propoxy, isopropoxy, n-butoxy, isobutoxy, tert-butoxy, sec-butoxy, n-pentyloxy, isopentyloxy, neopentyloxy, n-hexyloxy, isohexyloxy, and 3-methylpentyloxy.

Examples halogen atoms include fluorine, chlorine, bromine, and iodine.

Examples of lower alkoxycarbonyl groups include alkoxycarbonyl groups wherein the alkoxy moiety is a straight or branched C₁₋₆ alkoxy group, such as methoxycarbonyl, ethoxycarbonyl, n-propoxycarbonyl, isopropoxycarbonyl, n-butoxycarbonyl, isobutoxycarbonyl, tert-butoxycarbonyl, sec-butoxycarbonyl, n-pentyloxycarbonyl, neopentyloxycarbonyl, n-hexyloxycarbonyl, isohexyloxycarbonyl, and 3-methylpentyloxycarbonyl.

Examples of phenyl lower alkoxy groups include phenylalkoxy groups wherein the alkoxy moiety is a straight or branched C₁₋₆ alkoxy group, such as benzyloxy, 2-phenylethoxy, 1-phenylethoxy, 3-phenylpropoxy, 4-phenylbutoxy, 5-phenylpentyloxy, 6-phenylhexyloxy, 1,1-dimethyl-2-phenylethoxy, and 2-methyl-3-phenylpropoxy.

Examples of piperidinyl lower alkoxycarbonyl groups include piperidinylalkoxycarbonyl groups wherein the alkoxy moiety is a straight or branched C₁₋₆alkoxy group, such as [(1-, 2-, 3-, or 4-)piperidinyl]methoxycarbonyl, 2-[(1-, 2-, 3-, or 4-)piperidinyl]ethoxycarbonyl, 1-[(1-, 2-, 3-, or 4-)piperidinyl]ethoxycarbonyl, 3-[(1-, 2-, 3-, or 4-)piperidinyl]propoxycarbonyl, 4-[(1-, 2-, 3-, or 4-)piperidinyl]butoxycarbonyl, 5-[(1-, 2-, 3-, or 4-)piperidinyl]pentyloxycarbonyl, 6-[(1-, 2-, 3-, or 4-)piperidinyl]hexyloxycarbonyl, 1,1-dimethyl-2-[(1-, 2-, 3-, or 4-)piperidinyl]ethoxycarbonyl, and 2-methyl-3-[(1-, 2-, 3-, or 4-)piperidinyl]propoxy carbonyl.

Examples of cycloalkyl groups include C₃₋₈ cycloalkyl groups, such as cyclopropyl, cyclobutyl, cyclopentyl, cyclohexyl, cycloheptyl, and cyclooctyl.

Examples of amino lower alkoxycarbonyl groups optionally substituted with one or more cycloalkyl groups include:

amino-substituted alkoxycarbonyl groups wherein the alkoxy moiety is a straight or branched C₁₋₆ alkoxy group, optionally substituted with one or two C₃₋₈ cycloalkyl groups;

such as aminomethoxycarbonyl, 2-aminoethoxycarbonyl, cyclopropylaminomethoxycarbonyl, 2-cyclohexylaminoethoxycarbonyl, 1-cyclobutylaminoethoxycarbonyl, 3-cyclopentylaminopropoxycarbonyl, 4-cycloheptylaminobutoxycarbonyl, 5-cyclooctylaminopentyloxycarbonyl, 6-cyclohexylaminohexyloxycarbonyl, 1,1-dimethyl-2-cyclohexylaminoethoxycarbonyl, 2-methyl-3-cyclopropylaminopropoxycarbonyl, and 2-(N-cyclopropyl-N-cyclohexylamino)ethoxycarbonyl.

Examples of lower alkylthio groups include straight and branched C₁₋₆ alkylthio groups such as methylthio, ethylthio, n-propylthio, isopropylthio, n-butylthio, tert-butylthio, n-pentylthio, and n-hexylthio.

Examples of 2-imidazolinylcarbonyl groups optionally substituted on the 2-imidazoline ring with one or more alkylthio groups include 2-imidazolinylcarbonyl groups optionally substituted on the 2-imidazoline ring with one to three lower alkylthio groups, such as (1-, 2-, 4-, or 5-)2-imidazolinylcarbonyl, 2-methylthio-(1-, 4-, or 5-)2-imidazolinylcarbonyl, 2-ethylthio-(1-, 4-, or 5-)2-imidazolinylcarbonyl, 4-propylthio-(1-, 2-, or 5-)2-imidazolinylcarbonyl, 5-isopropylthio-(1-, 2-, or 4-)2-imidazolinylcarbonyl, 2-n-butylthio-(1-, 4-, or 5-)2-imidazolinylcarbonyl, 2-n-pentylthio-(1-, 4-, or 5-)2-imidazolinylcarbonyl, 2-n-hexylthio-(1-, 4-, or 5-)2-imidazolinylcarbonyl, 2,4-dimethylthio-(1- or 5-)2-imidazolinylcarbonyl, and 2,4,5-trimethylthio-(1-)2-imidazolinylcarbonyl.

Examples of 3-pyrrolinylcarbonyl groups optionally substituted on the 3-pyrroline ring with one or more lower alkyl groups include 3-pyrrolinylcarbonyl groups optionally substituted on the 3-pyrroline ring with one to three lower alkyl groups, such as (1-, 2-, or 3-)3-pyrrolinylcarbonyl, 2-methyl-(1-, 2-, 3-, 4-, or 5-)3-pyrrolinylcarbonyl, 2-ethyl-(1-, 2-, 3-, 4-, or 5-)3-pyrrolinylcarbonyl, 3-propyl-(1-, 2-, 4-, or 5-)3-pyrrolinylcarbonyl, 4-isopropyl-(1-, 2-, 3-, or 5-)3-pyrrolinylcarbonyl, 5-n-butyl-(1-, 2-, 3-, 4-, or 5-)3-pyrrolinylcarbonyl, 2-n-pentyl-(1-, 2-, 3-, 4-, or 5-)3-pyrrolinylcarbonyl, 2-n-hexyl-(1-, 2-, 3-, 4-, or 5-)3-pyrrolinylcarbonyl, 2,5-dimethyl-(1-, 2-, 3-, 4-, or 5-)3-pyrrolinylcarbonyl, 2,4-dimethyl-(1-, 2-, 3-, or 5-)3-pyrrolinylcarbonyl, 2,3-dimethyl-(1-, 2-, 4-, or 5-)3-pyrrolinylcarbonyl, and 2,4,5-trimethylthio-(1-, 2-, 3-, or 5-)3-pyrrolinylcarbonyl.

Examples of thiazolidinylcarbonyl groups optionally substituted on the thiazolidine ring with a phenyl group includes (2-, 3-, 4-, or 5-)thiazolidinylcarbonyl, 2-phenyl-(3-, 4-, or 5-)thiazolidinylcarbonyl, 3-phenyl-(2-, 4-, or 5-) thiazolidinylcarbonyl, 4-phenyl-(2-, 3-, or 5-) thiazolidinylcarbonyl, and 5-phenyl-(2-, 3-, or 4-) thiazolidinylcarbonyl.

Examples of piperidinyl lower alkyl groups include piperidinylalkyl groups wherein the alkyl moiety is a straight or branched C₁₋₆ alkyl group, such as [(1-, 2-, 3-, or 4-) piperidinyl]methyl, 2-[(1-, 2-, 3-, or 4-)piperidinyl]ethyl, 1-[(1-, 2-, 3-, or 4-)piperidinyl]ethyl, 3-[(1-, 2-, 3-, or 4-) piperidinyl]propyl, 4-[(1-, 2-, 3-, or 4-)piperidinyl]butyl, 5-[(1-, 2-, 3-, or 4-)piperidinyl]pentyl, 6-[(1-, 2-, 3-, or 4-) piperidinyl]hexyl, 1,1-dimethyl-2-[(1-, 2-, 3-, or 4-) piperidinyl]ethyl, and 2-methyl-3-[(1-, 2-, 3-, or 4-) piperidinyl]propyl.

Examples of anilino lower alkyl groups optionally substituted on the amino group with one or more lower alkyl groups include anilinoalkyl groups optionally substituted on the amino group with one or more straight and/or branched C₁₋₆ alkyl groups, such as anilinomethyl, N-methylanilinomethyl, N-ethylanilinomethyl, N-n-propylanilinomethyl, N-isopropylanilinomethyl, N-n-butylanilinomethyl, N-sec-butylanilinomethyl, N-tert-butylanilinomethyl, N-n-pentylanilinomethyl, N-n-hexylanilinomethyl, 2-anilinoethyl, 2-(N-methylanilino)ethyl, 2-(N-ethylanilino)ethyl, 2-(N-n-propylanilino)ethyl, 2-(N-isopropylanilino)ethyl, 2-(N-n-butylanilino)ethyl, 2-(N-sec-butylanilino)ethyl, 2-(N-tert-butylanilino)ethyl, 2-(N-n-pentylanilino)ethyl, 2-(N-n-hexylanilino)ethyl, 3-anilinopropyl, 3-(N-methylanilino)propyl, 4-(N-ethylanilino)butyl, 4-(N-n-propylanilino)butyl, 5-(N-isopropylanilino)pentyl, 5-(N-n-butylanilino)pentyl, 6-(N-sec-butylanilino)hexyl, 6-(N-tert-butylanilino)hexyl, 6-(N-n-pentylanilino)hexyl, and 6-(N-n-hexylanilino)hexyl.

Examples of phenylthio lower alkyl groups include phenylthioalkyl groups wherein the alkyl moiety is a straight or branched C₁₋₆ alkyl group, such as phenylthiomethyl, 2-phenylthioethyl, 1-phenylthioethyl, 3-phenylthiopropyl, 4-phenylthiobutyl, 5-phenylthiopentyl, 6-phenylthiohexyl, 1,1-dimethyl-2-phenylthioethyl, and 2-methyl-3-phenylthiopropyl.

Examples of indolinyl lower alkyl groups include indolinylalkyl groups wherein the alkyl moiety is a straight or branched C₁₋₆ alkyl group, such as [(1-, 2-, 3-, 4-, 5-, 6-, or 7-)indolinyl]methyl, 2-[(1-, 2-, 3-, 4-, or 5-)indolinyl]ethyl, 1-[(1-, 2-, 3-, 4-, 5-, 6-, or 7)indolinyl]ethyl, 3-[(1-, 2-, 3-, 4-, 5-, 6-, or 7)indolinyl]propyl, 4-[(1-, 2-, 3-, 4-, 5-, 6-, or 7)indolinyl]butyl, 5-[(1-, 2-, 3-, 4-, 5-, 6-, or 7)indolinyl]pentyl, 6-[(1-, 2-, 3-, 4-, 5-, 6-, or 7)indolinyl]hexyl, 1,1-dimethyl-2-[(1-, 2-, 3-, 4-, 5-, 6-, or 7)indolinyl]ethyl, and 2-methyl-3-[(1-, 2-, 3-, 4-, 5-, 6-, or 7)indolinyl]propyl.

Examples of piperidinylcarbonyl groups optionally substituted on the piperidine ring with one or more lower alkyl groups include piperidinylcarbonyl groups optionally substituted on the piperidine ring with one to three straight and/or branched C₁₋₆alkyl groups, such as (1-, 2-, 3-, or 4-)piperidinylcarbonyl, 1-methyl-(2-, 3-, or 4-)piperidinylcarbonyl, 1-ethyl-(2-, 3-, or 4-)piperidinylcarbonyl, 1-n-propyl-(2-, 3-, or 4-)piperidinylcarbonyl, 1-n-butyl-(2-, 3-, or 4-)piperidinylcarbonyl, 1-n-pentyl-(2-, 3-, or 4-)piperidinylcarbonyl, 1-n-hexyl-(2-, 3-, or 4-)piperidinylcarbonyl, 1,2-dimethyl-(3-, 4-, 5-, or 6-)piperidinylcarbonyl, 1,2,3-trimethyl-(4-, 5-, or 6-)piperidinylcarbonyl, 2-n-propyl-(1-, 3-, 4-, 5-, or 6-)piperidinylcarbonyl, 3-ethyl-(1-, 2-, 4-, 5-, or 6-) piperidinylcarbonyl, and 2-methyl-4-isopropyl-(1-, 3-, 5-, or 6-)piperidinylcarbonyl.

Examples of phenyl lower alkyl groups optionally substituted on the phenyl ring with one or more members selected from the group consisting of a phenyl group; lower alkyl groups; lower alkoxy groups; halogen atoms; —(B)ₗNR⁶R⁷ groups; a nitro group; a carboxy group; lower alkoxycarbonyl groups; a cyano group; phenyl lower alkoxy groups; a phenoxy group; piperidinyl lower alkoxycarbonyl groups; amino lower alkoxycarbonyl groups optionally substituted with one or more cycloalkyl groups; 2-imidazolinylcarbonyl groups optionally substituted on the 2-imidazoline ring with one or more lower alkylthio groups; 3-pyrrolinylcarbonyl groups optionally substituted on the pyrroline ring with one or more lower alkyl groups; a thiazolidinylcarbonyl groups optionally substituted on the thiazolidine ring with a phenyl group; 3-azabicyclo[3.2.2] nonylcarbonyl groups; piperidinyl lower alkyl groups; anilino lower alkyl groups optionally substituted on the amino group with one or more lower alkyl groups; phenylthio lower alkyl groups; indolinyl lower alkyl groups; and piperidinylcarbonyl groups optionally substituted on the piperidine ring with one or more lower alkyl groups include:

mono- and di-phenylalkyl groups wherein the alkyl moiety is a straight or branched C₁₋₆ alkyl group, optionally substituted on the phenyl ring with one to three members selected from the group consisting of a phenyl group; the above-described straight and branched C₁₋₆ alkyl groups; the above-described straight and branched C₁₋₆ alkoxy groups; halogen atoms; the below-described —(B)ₗNR⁶R⁷ groups; a nitro group; a carboxyl group; the above-described straight and branched C₁₋₆ alkoxycarbonyl groups; a cyano group; the above-described phenylalkoxy groups wherein the alkoxy moiety is a straight or branched C₁₋₆ alkoxy group; a phenoxy group; the above-described piperidinylalkoxycarbonyl groups wherein the alkoxy moiety is a straight or branched C₁₋₆ alkoxy group; the above-described aminoalkoxycarbonyl groups wherein the alkoxy moiety is a straight or branched C₁₋₆ alkoxy group, optionally substituted with one or two C₃₋₈ cycloalkyl groups; the above-described 2-imidazolinylcarbonyl groups optionally substituted on the 2-imidazoline ring with one to three straight and/or branched C₁₋₆ alkylthio groups; the above-described 3-pyrrolinylcarbonyl groups optionally substituted on the 3-pyrroline ring with one to three straight and/or branched C₁₋₆ alkyl groups; thiazolidinylcarbonyl groups optionally substituted on the thiazolidine ring with a phenyl group; 3-azabicyclo[3.2.2]nonylcarbonyl groups; piperidinylalkyl groups wherein the alkyl moiety is a straight or branched C₁₋₆ alkyl group; anilinoalkyl groups wherein the alkyl moiety is a straight or branched C₁₋₆ alkyl group, optionally substituted on the amino group with one or two straight and/or branched C₁₋₆ alkyl groups; phenylthioalkyl groups wherein the alkyl moiety is a straight or branched C₁₋₆ alkyl group; indolinylalkyl groups wherein the alkyl moiety is a straight or branched C₁₋₆ alkyl group; and the above-described piperidinylcarbonyl groups optionally substituted on the piperidine ring with one to three straight and/or branched C₁₋₆ alkyl groups;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-diphenylmethyl, 2,2-diphenylethyl, 3,3-diphenylpropyl, 1,2-diphenylethyl, 4-[N-(3-pyridyl)aminocarbonyl]benzyl, 4-[N-(2-methoxyphenyl)aminocarbonyl]benzyl, 4-[2-(2-piperidinyl)ethoxycarbonyl]benzyl, 4-[2-(cyclohexylamino) ethoxycarbonyl]benzyl, 4-[4-(3-pyridylmethyl)-1-piperazinylcarbonyl]benzyl, 4-[4-(4-pyridylmethyl)-1-piperazinylcarbonyl]benzyl, 4-[4-(2-pyridylmethyl)-1-piperazinylcarbonyl]benzyl, 4-[4-(2-pyridyl)-1-piperazinylcarbonyl]benzyl, 4-[4-(3-chlorophenyl)-1-piperazinylcarbonyl]benzyl, 4-[4-(2-fluorophenyl)-1-piperazinylcarbonyl]benzyl, 4-[4-(2-pyrimidyl)-1-piperazinylcarbonyl]benzyl, 4-(4-cyclopentyl-1-piperazinylcarbonyl)benzyl, 4-[4-(2-methoxyphenyl)-1-piperazinylcarbonyl]benzyl, 4-[4-(4-fluorophenyl)-1-piperazinylcarbonyl]benzyl, 4-[4-(3,4-methylenedioxybenzyl)-1-piperazinylcarbonyl]benzyl, 4-(N-cyclohexyl-N-methylaminocarbonyl)benzyl, 4-(N,N-di-n-butylaminocarbonyl)benzyl, 4-[4-(1-piperidinyl)-1-piperidinylcarbonyl]benzyl, 4-(1-homopiperidinylcarbonyl)benzyl, 4-[2-methylthio-1-(2-imidazolinyl)carbonyl]benzyl, 4-{N-[2-(2-pyridyl)ethyl]-N-methylaminocarbonyl}benzyl, 4-[N-(1-methyl-4-piperidinyl)-N-methylaminocarbonyl]benzyl, 4-(N,N-diisobutylaminocarbonyl)benzyl, 4-[N-(2-tetrahydropyranyl)methyl-N-ethylaminocarbonyl]benzyl, 4-(4-thiomorpholinocarbonyl)benzyl, 4-[2,5-dimethyl-1-(3-pyronyl)carbonyl]benzyl, 4-(3-thiazolidinylcarbonyl)benzyl, 4-(N-cyclopropylmethyl-N-n-propylaminocarbonyl)benzyl, 4-[1-(3-azabicyclo[3.2.2]nonylcarbonyl)benzyl, 4-(N-cyclopentyl-N-allylaminocarbonyl)benzyl, 4-[4-(4-pyridyl)-1-piperazinylcarbonyl]benzyl, 4-[4-(4-trifluoromethylphenyl)-1-piperazinylcarbonyl]benzyl, 4-[4-(2-phenylethyl)-1-piperazinylcarbonyl]benzyl, 4-[4-(2-pyrazyl)-1-piperazinylcarbonyl]benzyl, 4-(N-n-butylaminocarbonyl)benzyl, 4-(N-cyclopropylaminocarbonyl)benzyl, 4-[N-(1-methyl-1-phenylethyl)aminocarbonyl]benzyl, 4-(N-benzylaminocarbonyl)benzyl, 4-[N-(2-chlorobenzyl)aminocarbonyl]benzyl, 4-[N-(3-chlorobenzyl)aminocarbonyl]benzyl, 4-[N-(4-chlorobenzyl)aminocarbonyl]benzyl, 4-[N-(2-pyridyl)methylaminocarbonyl]benzyl, 4-[N-(3-pyridyl)methylaminocarbonyl]benzyl, 4-[(4-pyridyl)methylaminocarbonyl] benzyl, 4-[3,5-dimethyl-1-piperidinylcarbonyl]benzyl, 4-[N-(2-furyl)methylaminocarbonyl]benzyl, 4-[4-(2-fluorobenzyloxy)-1-piperidinylcarbonyl]benzyl, 4-{4-[N-(2-phenylacetyl)-N-methylamino]-1-piperidinylcarbonyl}benzyl, 4-[(4-methoxy-1-piperidinyl)carbonyl]benzyl, 4-{[4-(3,4-dimethyl-1-piperazinyl)-1-piperidinyl]carbonyl}benzyl, 4-{[4-(4-chlorobenzoyl)-1-piperidinyl]carbonyl}benzyl, 4-{[4-(4-chlorobenzyl)-1-piperidinyl]carbonyl}benzyl, 4-[(4-ethylcarbamoylmethyl-1-piperidinyl)carbonyl]benzyl, 4-[(4-cyclohexyl-1-piperidinyl)carbonyl]benzyl, 4-{[4-(4-methoxyphenyl)-1-piperidinyl]carbonyl}benzyl, 4-{[4-(2-benzoxazolyl)-1-piperazinyl]carbonyl}benzyl, 4-[(4-anilinocarbonylmethyl-1-piperazinyl)carbonyl]benzyl, 4-[(4-methyl-2-benzyl-1-piperazinyl)carbonyl]benzyl, 4-[(4-phenyl-3-oxo-1-piperazinyl)carbonyl]benzyl, 4-[(4-tert-butyl-3-oxo-1-piperazinyl)carbonyl]benzyl, 4-[N-(1-benzoyl-4-piperidinyl)-N-methylaminocarbonyl]benzyl, 4-[N-(1-acetyl-4-piperidinyl)-N-methylaminocarbonyl]benzyl, 4-{[4-(4-cyanophenyl)-1-piperazinyl]carbonyl}benzyl, 4-[N-methylcarbamoylmethyl-N-benzylaminocarbonyl]benzyl, 4-[N-benzyl-N-cyclohexylaminocarbonyl]benzyl, 4-[2-(N-methyl-N-phenylcarbamoyl)ethyl-N-methylaminocarbonyl]benzyl, 4-{[4-(3-phenyl-1-pyrrolidinyl)-1-piperidinyl]carbonyl}benzyl, 4-[(1,2,3,4-tetrahydroisoquinoline-2-yl)carbonyl]benzyl, 4-[(4-benzyl-1-piperidinyl)carbonyl]benzyl, 4-{[4-(3,4-methylenedioxybenzoyl)-1-piperazinyl]carbonyl}benzyl, 4-[N-methyl-N-(4-methylbenzyl)aminocarbonyl]benzyl, 4-[N-methyl-N-(3,4-methylenedioxybenzyl)aminocarbonyl]benzyl, 4-[N-methyl-N-(2-methoxybenzyl)aminocarbonyl]benzyl, 4-[(4-phenyl-1-piperazinyl) carbonyl]benzyl, 4-[(4-phenyl-4-hydroxy-1-piperidinyl)carbonyl] benzyl, 4-(N-isopropyl-N-benzylaminocarbonyl)benzyl, 4-(N-ethyl-N-cyclohexylaminocarbonyl)benzyl, 4-[N-ethyl-N-(4-pyridyl)methylaminocarbonyl]benzyl, 4-(N-n-propylaminocarbonyl)benzyl, 4-[N-ethyl-N-(4-ethoxybenzyl)aminocarbonyl]benzyl, 4-(N-ethyl-N-cyclohexylmethylaminocarbonyl)benzyl, 4-[N-(2-ethoxyethyl)aminocarbonyl]benzyl, 4-[N-(1,1-dimethyl-2-phenylethyl)aminocarbonyl]benzyl, 4-[{4-[N-methyl-N-(4-chlorophenyl)amino]-1-piperidinyl}carbonyl]benzyl, 4-[N-(1-methyl-1-cyclopentyl)aminocarbonyl]benzyl, 4-[N-(1-methyl-1-cyclohexyl)aminocarbonyl] benzyl, 4-{N-[2-(3-methoxyphenyl)ethyl]aminocarbonyl}benzyl, 4-[N-(4-trifluoromethoxybenzyl)aminocarbonyl]benzyl, 4-{N-[2-(4-chlorophenyl)ethyl]aminocarbonyl}benzyl, 4-[N-(3,4-methylenedioxybenzyl)aminocarbonyl]benzyl, 4-(N-cyclohexylmethylaminocarbonyl)benzyl, 4-[N-(4-fluorobenzyl)aminocarbonyl]benzyl, 4-[N-(1-phenylethyl)aminocarbonyl]benzyl, 4-[N-(3-phenylpropyl)aminocarbonyl]benzyl, 4-{N-[3-(1-imidazolyl) propyl]aminocarbonyl}benzyl, 4-[N-(2-phenylethyl)aminocarbonyl] benzyl, 4-[2-(N,N-diisopropylamino)ethylaminocarbonyl]benzyl, 4-{N-[1-methoxycarbonyl-2-(4-hydroxyphenyl)ethyl]aminocarbonyl} benzyl, 4-[N-(carbamoylmethyl)aminocarbonyl]benzyl, 4-{N-[1-carbamoyl-2-(5-imidazolyl)ethyl]aminocarbonyl}benzyl, 4-{N-[1-methoxycarbonyl-2-(5-imidazolyl)ethyl]amino carbonyl}benzyl, 4-[N-(2-oxo-2,3,4,5-tetrahydrofuran-3-yl)aminocarbonyl]benzyl, 4-[(2-ethoxycarbonyl-1-piperidinyl)carbonyl]benzyl, 4-(N-methoxycarbonylmethyl-N-methylaminocarbonyl)benzyl, 4-[(2-carbamoyl-1-pyrrolidinyl)carbonyl]benzyl, 4-{[N-(2,6-dimethylbenzyl)-N-ethyl]aminocarbonyl}benzyl, 4-{N-[(4-methylphenyl)carbamoylmethyl]-N-methylaminocarbonyl}benzyl, 4-[N-(4-chlorobenzyl)-N-ethylaminocarbonyl]benzyl, 4-[N-(4-trifluoromethylbenzyl)-N-ethylaminocarbonyl]benzyl, 4-[N-(3-bromobenzyl)-N-ethylaminocarbonyl]benzyl, 4-{[4-(2-chlorobenzyl)-1-piperidinyl]carbonyl}benzyl, 4-{[4-(3-chlorobenzyl)-1-piperidinyl]carbonyl}benzyl, 4-{[4-(2-chlorobenzylidene)-1-piperidinyl]carbonyl}benzyl, 4-[N-(2-methoxybenzyl)aminocarbonyl] benzyl, 4-{N-[2-(2-fluorophenyl)ethyl]aminocarbonyl}benzyl, 4-{N-[2-(3-fluorophenyl)ethyl]aminocarbonyl}benzyl, 4-[(4-benzyloxycarbonyl-1-piperazinyl)carbonyl]benzyl, 4-{[4-(3-cyano-2-pyridyl)-1-piperazinyl]carbonyl}benzyl, 4-[(4-phenyl-1-piperidinyl)carbonyl]benzyl, 4-[{4-[(3-furyl)methyl]-1-piperazinyl}carbonyl]benzyl, 4-{[4-(3-pyridyl)-1-piperazinyl]carbonyl}benzyl, 4-{[4-(4-tetrahydropyranyl)-1-piperazinyl]carbonyl}benzyl, 4-{[4-(2-fluorobenzyl)-1-piperidinyl]carbonyl} benzyl, 4-{[4-(4-morpholino)-1-piperidinyl]carbonyl}benzyl, 4-{4-[2-(1,3-dioxolane-2-yl)ethyl]-1-piperazinyl}carbonyl]benzyl, 4-phenylbenzyl, 2-phenylbenzyl, 3-phenylbenzyl, 4-tert-butylbenzyl, 4-aminobenzyl, 4-nitrobenzyl, 4-methoxycarbonylbenzyl, 4-carboxybenzyl, 3-methoxy-4-chlorobenzyl, 4-methoxybenzyl, 2,4,6-trimethoxybenzyl, 3,4-dichlorobenzyl, 4-chlorobenzyl, 4-bromobenzyl, 2,4,6-trifluorobenzyl, 4-fluorobenzyl, 4-cyanobenzyl, 4-piperidinylcarbonylbenzyl, 4-anilinocarbonylbenzyl, 4-(N-cyclohexylaminocarbonyl)benzyl, 4-(N-benzoylamino)benzyl, 4-(N-cyclohexylamino)benzyl, 4-phenylcarbamoylaminobenzyl, 4-methylbenzyl, 3,4-dimethylbenzyl, 3,4,5-trimethylbenzyl, 4-benzyloxybenzyl, 4-ethylcarbamoylaminobenzyl, 4-ethylaminocarbonylbenzyl, 4-isopropylaminocarbonylbenzyl, 4-[N-(2-hydroxyethyl)aminocarbonyl]benzyl, 4-[N-(3-pyridyl)aminocarbonyl]benzyl, 4-[N-(4-chlorophenyl)aminocarbonyl]benzyl, 4-[N-(4-isopropylphenyl)aminocarbonyl]benzyl, 4-[N-(4-phenoxyphenyl)aminocarbonyl]benzyl, 4-[N-(3-phenoxyphenyl)aminocarbonyl]benzyl, 4-[N-(3-phenoxybenzoyl)amino]benzyl, 4-[N-(4-phenoxybenzoyl)amino]benzyl, 4-[N-(4-chlorobenzoyl)amino] benzyl, 4-[N-(2-chlorobenzoyl)amino]benzyl, 4-[N-(2,6-dichlorobenzoyl)amino]benzyl, 4-[N-(4-methoxyphenyl)aminocarbonyl]benzyl, 4-[N-(2-furylcarbonyl)amino]benzyl, 4-[N-(4-methoxybenzoyl)amino]benzyl, 4-[N-(3-methoxybenzoyl)amino] benzyl, 4-[N-(2-methoxybenzoyl)amino]benzyl, 4-phenoxybenzyl, 4-n-pentyloxycarbonylaminobenzyl, 4-[N-(4-methoxyphenoxycarbonyl)amino]benzyl, 4-[N-(4-methylphenoxycarbonyl)amino]benzyl, 4-benzyloxycarbonylaminobenzyl, 4-ethanoylaminobenzyl, 4-(N-acetylamino)benzyl, 4-methylsulfonylaminobenzyl, methoxycarbonylaminobenzyl, 4-[N-(4-isopropylphenyl)aminocarbonyl]benzyl, 4-[4-{2-[(1-, 2-, or 3-)imidazolyl]ethyl}-1-piperazinylcarbonyl]benzyl, 4-{4-[3-methyl-(2-, 3-, or 4-) pyridyl]-1-piperazinyl carbonyl}benzyl, 4-{4-[4-methyl-(2-, 3-, or 4-)pyridyl]-1-piperazinylcarbonyl}benzyl, 4-[4-{2-[(2-, 3-, or 4-)pyridyl]ethyl}-1-piperazinylcarbonyl]benzyl, 4-{4-4-[(1- or 2-)naphthyl]-(1-, 2-, or 3-)piperazinylcarbonyl} benzyl, 4-[(1-, 2-, 3-, or 4-piperazinylcarbonyl)] benzyl, 4-[2-methyl-(1-, 3-, 4-, 5-, or 6-)piperidinylcarbonyl]benzyl, 4-[3-ethoxycarbonyl-(1-, 2-, 4-, 5-, or 6-)piperidinyl]benzyl, 4-[4-(3-hydroxyphenyl)-(1-, 2-, 4-, 5-, or 6-)piperidinyl]benzyl, 4-[4-hydroxy-4-benzyl-(1-, 2-, or 3-)piperidinylcarbonyl]benzyl, 4-[3-acetylamino-(1-, 2-, 4-, or 5-)pyrrolidinylcarbonyl]benzyl, 4-[N-{2-[1-ethyl-(2- or 3-)pyrrolidinyl]ethyl}aminocarbonyl]benzyl, 4-[N-{2-[(2- or 3-)pyrrolidinyl]ethyl}aminocarbonyl]benzyl, 4-[N-{2-([2-, 3-, or 4-]morpholino)ethyl}aminocarbonyl]benzyl, 4-[N-{3-([2-, 3-, or 4-]morpholino)propyl}aminocarbonyl]benzyl, 4-[2,6-dimethyl-(3-, 4-, or 5-)morpholinocarbonyl]benzyl, 4-[4-(4-trifluoromethylanilino)-(1-, 2-, or 3-)piperazinylcarbonyl]benzyl, 4-{2-[(1-, 2-, 3-, or 4-) piperidinylmethyl]-(3-, 4-, 5- or 6-) morpholinocarbonyl}benzyl, 4-(N-methyl-N-n-pentylaminocarbonyl)benzyl, 4-{4-[(1-, 2-, 4-, or 5-)2,3-dihydro-1H-indenyl]-(1-, 2-, or 3-)piperidinylcarbonyl}benzyl, 4-[N-(2-methylcyclohexyl)aminocarbonyl]benzyl, 4-isoindolinylcarbonylbenzyl, 4-[2-phenyl-(1-, 3-, 4- or 5-) pyrrolidinylcarbonyl]benzyl, 4-{2-[(1-, 2-, 3-, or 4-morpholinomethyl)-(1-, 3-, 4-, or 5-)pyrrolidinylcarbonyl]benzyl, 4-[2-dimethylaminomethyl-(1-, 3-, 4-, or 5-) pyrrolidinylcarbonyl]benzyl, 4-{N-[1-(4-fluorobenzoyl)-(2-, 3-, or 4-)piperidinyl]-N-methylaminocarbonyl}benzyl, 4-[2-phenyl-(3-, 4-, or 5-)thiazolidinylcarbonyl]benzyl, 4-[N-methyl-(2-methoxyanilino)carbonyl]benzyl, 4-(3-methylthioanilinocarbonyl)benzyl, 4-(2-methylthioanilinocarbonyl)benzyl, 4-(3,4-dichloroanilinocarbonyl)benzyl, 4-(4-trifluoromethoxy-4-anilinocarbonyl)benzyl, 4-anilinocarbonylbenzyl, 4-(4-chloroanilinocarbonyl)benzyl, 4-(4-methoxyanilinocarbonyl)benzyl, 4-(3-methoxyanilinocarbonyl)benzyl, 4-(2-chloroanilinocarbonyl)benzyl, 4-(4-methylanilinocarbonyl)benzyl, 4-(2,4-dimethoxyanilinocarbonyl)benzyl, 4-(4-methoxy-5-chloroanilinocarbonyl)benzyl, 4-(2-methoxy-5-acetylaminoanilinocarbonyl)benzyl, 4-(3,4-dimethoxyanilinocarbonyl)benzyl, 4-[2-(1-methylallyl) anilinocarbonyl]benzyl, 4-(3-trifluoromethoxyanilinocarbonyl)benzyl, 4-(2-methylanilinocarbonyl)benzyl, 4-(2-fluoroanilinocarbonyl)benzyl, 4-(3-fluoroanilinocarbonyl)benzyl, 4-(4-fluoroanilinocarbonyl)benzyl, 4-(3-dimethylaminoanilinocarbonyl)benzyl, 4-(4-ethoxyanilinocarbonyl)benzyl, 4-(3-trifluoromethylanilinocarbonyl)benzyl, 4-(4-trifluoromethylanilinocarbonyl)benzyl, 4-(3-acetylaminoanilinocarbonyl)benzyl, 4-(4-acetylaminoanilinocarbonyl)benzyl, 4-[(2-, 3-, or 4-pyridylaminocarbonyl)benzyl, 4-[N-methyl-(3-methylanilino)carbonyl]benzyl, 4-[3-methoxy-(2-, 4-, 5-, or 6-)pyridylaminocarbonyl]benzyl, 4-(2-phenoxyanilinocarbonyl)benzyl, 4-(3-phenoxyanilinocarbonyl)benzyl, 4-(4-phenoxyanilinocarbonyl)benzyl, 4-(3,5-dichloroanilinocarbonyl)benzyl, 4-(2,3-dimethylanilinocarbonyl)benzyl, 4-(2,4-dimethylanilinocarbonyl)benzyl, 4-(3,5-dimethylanilinocarbonyl)benzyl, 4-(3,5-difluoroanilinocarbonyl)benzyl, 4-[(1-, 2-, 3-, 4-, 5-, 6-, or 7-)indolylaminocarbonyl]benzyl, 4-(3-fluoro-4-methoxyanilinocarbonyl)benzyl, 4-(4-aminosulfonylanilinocarbonyl)benzyl, 4-(4-methyl-3-methoxyanilinocarbonyl)benzyl, 4-(3-chloro-4-methoxyanilinocarbonyl)benzyl, 4-(3-chloro-4-methylanilinocarbonyl)benzyl, 4-(3-methoxy-5-trifluoromethylanilinocarbonyl)benzyl, 4-(3-chloro-4-fluoroanilinocarbonyl)benzyl, 4-[3-methyl-(2-, 4-, 5- or 6-)pyridylaminocarbonyl]benzyl, 4-[(2-, 4- or 5-thiazolylaminocarbonyl)benzyl, 4-(3-chloro-4-hydroxyanilinocarbonyl)benzyl, 4-(2-chloro-5-acetylaminoanilinocarbonyl)benzyl, 4-(4-methylthioanilinocarbonyl)benzyl, 4-(4-isopropylanilinocarbonyl)benzyl, 4-(4-tert-butylanilinocarbonyl)benzyl, 4-[(2- or 4-)1,2,4-triazolylaminocarbonyl]benzyl, 4-{4-[2-oxo-(1-, 3-, 4-, or 5-) pyrrolidinyl]anilinocarbonyl}benzyl, 4-(4-methylsulfonylamino) benzyl, 4-(4-methylcarbamoylanilinocarbonyl)benzyl, anilinocarbonylbenzyl, 4-(2-benzyloxyanilinocarbonyl)benzyl, 4-(4-vinylanilinocarbonyl)benzyl, 4-(4-acetylaminoanilinocarbonyl)benzyl, 4-(3-acetylaminoanilinocarbonyl)benzyl, 4-(4-trifluoromethylanilinocarbonyl)benzyl, 4-{3-[(2-, 3-, or 4-) pyridyl]propionylamino}benzyl, 4-(3-phenoxypropionylamino)benzyl, 4-[(2-, 3- or 4-) pyridylcarbonylamino]benzyl, 4-{2-[(2-, 3-, or 4-) pyridyl]acetylamino}benzyl, 4-[(2- or 3-)furylcarbonylamino] benzyl, 4-[(2- or 3-)thienylcarbonylamino]benzyl, 4-{2-[(2- or 3-)thienyl]acetylamino}benzyl, 4-{2-[(1-, 2-, or 3-) pyrrolyl]-(3-, 4-, 5-, or 6-)pyridyl carbonylamino}benzyl, 4-cyclopentylcarbonylaminobenzyl, 4-cyclohexylcarbonylaminobenzyl, 4-(2-cyclopentylacetylamino)benzyl, 4-(2-cyclohexylcarbonylamino) benzyl, 4-[1-benzoyl-(2-, 3-, or 4-)piperidinylcarbonylamino] benzyl, 4-[1-acetyl-(2-, 3-, or 4-)piperidinylcarbonylamino] benzyl, 4-[(2-, 3-, 4-, 5-, 6-, 7-, or 8-)chromanyl]benzyl, 4-(2-nitrobenzoylamino)benzyl, 4-(3-nitrobenzoylamino)benzyl, 4-(4-nitrobenzoylamino)benzyl, 4-(2-phenylbenzoylamino)benzyl, 4-(2-dimethylaminobenzoylamino)benzyl, 4-(2-anilinobenzoylamino)benzyl, 4-(2,6-dichlorobenzoylamino)benzyl, 4-(2-cyanobenzoylamino)benzyl, 4-(3-phenoxybenzoylamino)benzyl, 4-(2-phenoxybenzoylamino)benzyl, 4-(4-phenoxybenzoylamino)benzyl, 4-[(1- or 2-) naphthylcarbonylamino]benzyl, 4-(2-methyl-3-fluorobenzoylamino) benzyl, 4-(3,4-methylenedioxybenzoylamino)benzyl, 4-{2-[1,3-dioxo-(2-, 4-, or 5-)isoindolinyl]acetylamino}benzyl, 4-{2-[2-thioxo-4-oxothiazolidinyl]acetylamino}benzyl, 4-{3-[(1-, 2-, 3-, or 4-)piperidinyl]propionylamino}benzyl, 4-(4-acetylbenzoylamino)benzyl, 4-(2-trifluoromethylbenzoylamino) benzyl, 4-(3-trifluoromethylbenzoylamino)benzyl, 4-(4-trifluoromethylbenzoylamino)benzyl, 4-[2-(2-chlorophenyl)acetylamino]benzyl, 4-(2-chloro-4-fluorobenzoylamino)benzyl, 4-(2-chlorocinnamoylamino)benzyl, 4-(3,4-methylenedioxycinnamoylamino)benzyl, 4-[3-(2-, 3-, or 4-) pyridylvinylcarbonylamino]benzyl, 4-[2-chloro-(3-, 4-, 5-, or 6-) pyridylcarbonylamino]benzyl, 4-{2-[(2-, 3-, or 4-)pyridylthio]acetylamino}benzyl, 4-[(2-, 3-, 4-, 5-, 6-, or 7-) indolylcarbonylamino]benzyl, 4-[(1-, 2-, or 3-) pyrrolylcarbonylamino]benzyl, 4-[2-oxo-(1-, 3-, 4-, or 5-) pyrrolidinylcarbonylamino]benzyl, 4-[(2-, 3-, 4-, 5-, 6-, or 7-) benzofurylcarbonylamino]benzyl, 4-[2,6-dichloro-(3-, 4-, or 5-) pyridylcarbonylamino]benzyl, 4-{2-[(1-, 2-, 3-, 4-, 5-, 6-, or 7-)indolyl]acetylamino}benzyl, 4-[(2-, 3-, 4-, 5-, 6-, or 7-)benzothienylcarbonylamino]benzyl, 4-{4-[2-oxo-(1-, 3-, 4-, or 5-)pyrrolidinyl]benzoylamino}benzyl, 4-{4-[(1-, 2-, or 3-)pyrrolyl]benzoylamino}benzyl, 4-{4-[(1-, 3-, 4-, or 5-) pyrazolyl]benzoylamino}benzyl, 4-{4-[(1-, 3-, or 5-)1,2,4-triazolyl]benzoylamino}benzyl, 4-{4-[(1-, 2-, 4-, or 5-)imidazolyl]benzoylamino}benzyl, 4-[4-(3,5-dimethyl-4-isoxazolyl)benzoylamino]benzyl, 4-[(2- or 3-) pyrazylcarbonylamino]benzyl, 4-(2-methoxybenzoylamino)benzyl, 4-(2-methoxy-5-chlorobenzoylamino)benzyl, 4-(4-chlorobenzoylamino) benzyl, 4-(2-phenoxyacetylamino)benzyl, 4-(3-phenylpropionyl)benzyl, 4-[(2-, 3-, or 4-)pyridylcarbonylamino]benzyl, 4-benzoylaminobenzyl, 4-cinnamoylaminobenzyl, 4-(4-methoxyphenylsulfonylamino)benzyl, 4-(3-methoxyphenylsulfonylamino)benzyl, 4-(2-methoxyphenylsulfonylamino)benzyl, 4-(4-chlorophenylsulfonylamino)benzyl, 4-(3-chlorophenylsulfonylamino)benzyl, 4-(2-chlorophenylsulfonylamino)benzyl, 4-(2-methylphenylsulfonylamino)benzyl, 4-(3-methylphenylsulfonylamino)benzyl, 4-(4-methylphenylsulfonylamino)benzyl, 4-(4-fluorophenylsulfonylamino)benzyl, 4-(3-fluorophenylsulfonylamino)benzyl, 4-(2-fluorophenylsulfonylamino)benzyl, 4-(2-methoxy-5-chlorophenylsulfonylamino)benzyl, 4-(2-trifluoromethylphenylsulfonylamino)benzyl, 4-(3-trifluoromethylphenylsulfonylamino)benzyl, 4-(4-trifluoromethylphenylsulfonylamino)benzyl, 4-[(2- or 3-)thienylsulfonylamino]benzyl, 4-(2-chlorophenylsulfonylamino)benzyl, 4-(2-trifluoromethoxyphenylsulfonylamino)benzyl, 4-(3-trifluoromethoxyphenylsulfonylamino)benzyl, 4-(4-trifluoromethoxyphenylsulfonylamino)benzyl, 4-(2-methoxycarbonylphenylsulfonylamino)benzyl, 4-(2-cyanophenylsulfonylamino)benzyl, 4-(3-cyanophenylsulfonylamino)benzyl, 4-(4-cyanophenylsulfonylamino)benzyl, 4-(3,4-dimethoxyphenylsulfonylamino)benzyl, 4-(2,5-dimethoxyphenylsulfonylamino)benzyl, 4-(2-nitrophenylsulfonylamino)benzyl, 4-(3-nitrophenylsulfonylamino)benzyl, 4-(4-nitrophenylsulfonylamino)benzyl, 4-(4-bromophenylsulfonylamino)benzyl, 4-(3-bromophenylsulfonylamino)benzyl, 4-(2-bromophenylsulfonylamino)benzyl, 4-(4-n-butylphenylsulfonylamino)benzyl, 4-(2-methoxy-5-chlorophenylsulfonylamino)benzyl, 4-(2,6-dichlorophenylsulfonylamino)benzyl, 4-[(1-, 2-, 3-, 4-, 5-, 6-, 7-, or 8-)quinolylsulfonylamino] benzyl, 4-[1-methyl-(2-, 4-, or 5-)imidazolylsulfonylamino]benzyl, 4-(2,3-dichlorophenylsulfonylamino)benzyl, 4-(2,5-dichlorophenylsulfonylamino)benzyl, 4-(2,4-dichlorophenylsulfonylamino)benzyl, 4-(3-nitro-4-methylphenylsulfonylamino)benzyl, 4-(2-chloro-4-fluorophenylsulfonylamino)benzyl, 4-(2,4-dichloro-5-methylphenylsulfonylamino)benzyl, 4-(2-methyl-5-nitrophenylsulfonylamino)benzyl, 4-(2-chloro-5-nitrophenylsulfonylamino)benzyl, 4-(2-chloro-4-cyanophenylsulfonylamino)benzyl, 4-(2,4,6-trimethylphenylsulfonylamino)benzyl, 4-(4-acetylaminophenylsulfonylamino)benzyl, 4-(3,5-dichloro-2-hydroxyphenylsulfonylamino)benzyl, 4-(4-methoxy-2-nitrophenylsulfonylamino)benzyl, 4-(3,4-dichlorophenylsulfonylamino)benzyl, 4-(4-tert-butylphenylsulfonylamino)benzyl, 4-(4-carboxyphenylsulfonylamino)benzyl, 4-(2-bromo-5-chlorophenylsulfonylamino)benzyl, 4-(4-ethylphenylsulfonylamino)benzyl, 4-(2,5-dimethylsulfonylamino)benzyl, 4-(4-n-butoxyphenylsulfonylamino)benzyl, 4-(2,5-difluorophenylsulfonylamino)benzyl, 4-(2-chloro-4-acetylaminophenylsulfonylamino)benzyl, 4-(2,4-difluorophenylsulfonylamino)benzyl, 4-(2-methoxy-4-methylphenylsulfonylamino)benzyl, 4-(2-methyl-3-chlorophenylsulfonylamino)benzyl, 4-(2,6-difluorophenylsulfonylamino)benzyl, 4-(3,4-difluorophenylsulfonylamino)benzyl, 4-(2-methyl-5-fluorophenylsulfonylamino)benzyl, 4-(3-methyl-4-chlorophenylsulfonylamino)benzyl, 4-(2-methyl-6-chlorophenylsulfonylamino)benzyl, 4-(4-isopropylphenylsulfonylamino)benzyl, 4-(3,4-dichlorophenylsulfonylamino)benzyl, 4-(2-fluoro-4-bromophenylsulfonylamino)benzyl, 4-(4-methyl-3-chlorophenylsulfonylamino)benzyl, 4-vinylsulfonylaminobenzyl, 4-(3-chloropropylphenylsulfonylamino) benzyl, 4-cyclohexylmethylsulfonylaminobenzyl, 4-[2-chloro-(3-, 4-, or 5-) thienylsulfonylamino]benzyl, 4-(3,5-dichlorophenylsulfonylamino) benzyl, 4-{4-[2-(4-methoxycarbonyl)ethyl]phenylsulfonylamino} benzyl, 4-[4-methyl-(2-, 3-, 4-, 5-, 6-, 7-, or 8-)3,4-dihydro-2H-1,4-dihydro-2H-1,4-benzoxazinylsulfonylamino]benzyl, 4-(2,2,2-trifluoroethylsulfonylamino)benzyl, 4-(2,3,5-trimethyl-4-methoxyphenylsulfonylamino)benzyl, 4-[(1,3-dimethyl-5-chloro-4-pyrazolyl)sulfonylamino]benzyl, 4-[(3,5-dimethyl-4-isoxazolyl) sulfonylamino]benzyl, 4-(3-carboxy-4-hydroxyphenylsulfonylamino) benzyl, 4-{[2,3-dichloro-(4- or 5-)thienyl]sulfonylamino}benzyl, 4-{[2,5-dichloro-(3- or 4-)thienyl]sulfonylamino}benzyl, 4-{[2-bromo-(3-, 4-, or 5-)thienyl]sulfonylamino}benzyl, 4-(4-carboxyphenylsulfonylamino)benzyl, 4-(2-acetylamino-4-methyl-5-thiazolylsulfonylamino)benzyl, 4-{[2-methoxycarbonyl-(3-, 4-, or 5-)thienyl]sulfonylamino}benzyl, 4-benzylsulfonylaminobenzyl, 4-styrylsulfonylaminobenzyl, 4-(2,4,5-trifluorophenylsulfonylamino)benzyl, 4-phenylsulfonylaminobenzyl, 4-phenoxycarbonylaminobenzyl, 4-[(4-chlorophenoxy)carbonylamino] benzyl, 4-[(4-bromophenoxy)carbonylamino]benzyl, 4-benzyloxycarbonylaminobenzyl, 4-methoxycarbonylaminobenzyl, 4-n-butoxycarbonylaminobenzyl, 4-[(4-methoxyphenoxy)carbonylamino] benzyl, 4-[(3-methoxyphenoxy)carbonylamino]benzyl, 4-[(2-methoxyphenoxy)carbonylamino]benzyl, 4-[(1- or 2-) naphthyloxycarbonylamino]benzyl, 4-[(4-fluorophenoxy) carbonylamino]benzyl, 4-[(4-methylphenoxy)carbonylamino]benzyl, 4-[(2-chlorobenzyloxy)carbonylamino]benzyl, 4-[2-propynyloxycarbonylamino]benzyl, 4-[(4-nitrophenoxy) carbonylamino]benzyl, 4-(2-fluoroethoxycarbonylamino)benzyl, 4-(3-butenyloxycarbonylamino)benzyl, 4-(4-chlorobutoxycarbonylamino)benzyl, 4-(2-chloroethoxycarbonylamino) benzyl, 4-[2-(benzyloxy)ethoxycarbonylamino]benzyl, 4-propoxycarbonylaminobenzyl, 4-n-butoxycarbonylaminobenzyl, 4-(2-isopropyl-5-methylcyclohexyloxycarbonylamino)benzyl, 4-[(4-nitrobenzyloxy)carbonylamino]benzyl, 4-(2-ethylhexyloxycarbonylamino)benzyl, 4-[N-methyl-(4-chloroanilino)carbonyl]benzyl, 4-[(2-chloroanilino)carbonyl] benzyl, 4-[(3-cyanoanilino)carbonyl]benzyl, 4-[(4-cyanoanilino) carbonyl]benzyl, 4-[(2-cyanoanilino)carbonyl]benzyl, 4-[(2-chloro-4-fluoroanilino)carbonyl]benzyl, 4-[(1- or 5-) tetrazolylaminocarbonyl]benzyl, 4-[5-methyl-(3- or 4-) isoxazolylaminocarbonyl]benzyl, 4-{4-[4-methyl-(1-, 2-, 3-, or 4-)piperazinyl]anilinocarbonyl}benzyl, (2-, 3-, or 4-)(1-piperidinylmethyl)benzyl, (2-, 3-, or 4-)(N-methylanilinomethyl)benzyl, (2-, 3-, or 4-)(phenylthiomethyl)benzyl, and (2-, 3-, or 4-)(1-indolylmethyl)benzyl.

Examples of cycloalkyl lower alkyl groups include C₃₋₈ cycloalkylalkyl groups wherein the alkyl moiety is a straight or branched C₁₋₆ alkyl group, such as cyclopropylmethyl, cyclohexylmethyl, 2-cyclopropylethyl, 1-cyclobutylethyl, cyclopentylmethyl, 3-cyclopentylpropyl, 4-cyclohexylbutyl, 5-cycloheptylpentyl, 6-cyclooctylhexyl, 1,1-dimethyl-2-cyclohexylethyl, and 2-methyl-3-cyclopropylpropyl.

Examples of phenoxy lower alkyl groups include phenoxy alkyl groups wherein the alkyl moiety is a straight or branched C₁₋₆ alkyl group, such as phenoxymethyl, 2-phenoxyethyl, 1-phenoxyethyl, 3-phenoxypropyl, 4-phenoxybutyl, 1,1-dimethyl-2-phenoxyethyl, 5-phenoxypentyl, 6-phenoxyhexyl, 1-phenoxyisopropyl, and 2-methyl-3-phenoxypropyl.

Examples of naphthyl lower alkyl groups include naphthylalkyl groups wherein the alkyl moiety is a straight or branched C₁₋₆alkyl group, such as (1- or 2-)naphthylmethyl, 2-[(1- or 2-)naphthyl]ethyl, 1-[(1- or 2-)naphthyl]ethyl, 3-[(1- or 2-)naphthyl]propyl, 4-[(1- or 2-)naphthyl]butyl, 5-[(1- or 2-)naphthyl]pentyl, 6-[(1- or 2-)naphthyl]hexyl, 1,1-dimethyl-2-[(1- or 2-)naphthyl]ethyl, and 2-methyl-3-[(1- or 2-) naphthyl]propyl.

Examples of lower alkoxy lower alkyl groups include alkoxyalkyl groups wherein the alkyl moiety is a straight or branched C₁₋₆ alkyl group and the alkoxy moiety is a straight or branched C₁₋₆ alkoxy group, such as methoxymethyl, 2-methoxyethyl, 1-ethoxyethyl, 2-ethoxyethyl, 3-n-butoxypropyl, 4-n-propoxybutyl, 1-methyl-3-isobutoxypropyl, 1,1-dimethyl-2-n-pentyloxyethyl, 5-n-hexyloxypentyl, 6-methoxyhexyl, 1-ethoxyisopropyl, and 2-methyl-3-methoxypropyl.

Examples of carboxy lower alkyl groups include carboxyalkyl groups wherein the alkyl moiety is a straight or branched C₁₋₆ alkyl group, such as carboxymethyl, 2-carboxyethyl, 1-carboxyethyl, 3-carboxypropyl, 4-carboxybutyl, 5-carboxypentyl, 6-carboxyhexyl, 1,1-dimethyl-2-carboxyethyl, and 2-methyl-3-carboxypropyl.

Examples of lower alkoxycarbonyl lower alkyl groups include alkoxycarbonylalkyl groups wherein the alkoxy moiety is a straight or branched C₁₋₆ alkoxy group and the alkyl moiety is a straight or branched C₁₋₆ alkyl group, such as methoxycarbonylmethyl, ethoxycarbonylmethyl, 2-methoxycarbonylethyl, 2-ethoxycarbonylethyl, 1-ethoxycarbonylethyl, 3-methoxycarbonylpropyl, 3-ethoxycarbonylpropyl, 4-ethoxycarbonylbutyl, 5-isopropoxycarbonylpentyl, 6-n-propoxycarbonylhexyl, 1,1-dimethyl-2-n-butoxycarbonylethyl, 2-methyl-3-tert-butoxycarbonylpropyl, 2-n-pentyloxycarbonylethyl, and n-hexyloxycarbonyl methyl.

Examples of piperazinyl groups optionally substituted on the piperazine ring with one or more members selected from the group consisting of a phenyl group and lower alkyl groups include:

piperazinyl groups optionally substituted on the piperazine ring with one to three members selected from the group consisting of a phenyl group and straight and branched C₁₋₆ alkyl groups;

such as (1- or 2-)piperazinyl, 4-methyl-(1-, 2-, or 3-) piperazinyl, 4-ethyl-(1-, 2-, or 3-)piperazinyl, 4-n-propyl-1-, 2-, or 3-)piperazinyl, 4-tert-butyl-(1-, 2-, or 3-) piperazinyl, 4-sec-butyl-(1-, 2-, or 3-) piperazinyl, 4-n-butyl-(1-, 2-, or 3-)piperazinyl, 4-n-pentyl-(1-, 2-, or 3-)piperazinyl, 4-n-hexyl-(1-, 2-, or 3-)piperazinyl, 3,4-dimethyl-(1-, 2-, 5-, or 6-)piperazinyl, 3,4,5-trimethyl-(1- or 2-) piperazinyl, 4-phenyl-(1-, 2-, or 3-)piperazinyl, 2,4-diphenyl-(1-, 3-, 5-, or 6-)piperazinyl, 2,3,4-triphenyl-(1-, 5-, or 6-) piperazinyl, and 4-phenyl-2-methyl-(1-, 3-, 5-, or 6-)piperazinyl.

Examples of pyridylamino groups include (2-, 3-, or 4-) pyridylamino.

Examples of pyridylcarbonylamino groups include (2-, 3-, or 4-)pyridylcarbonylamino.

Examples of anilino groups optionally substituted on the amino group with one or more lower alkyl groups include anilino groups optionally substituted on the amino group with one or more straight and/or branched C₁₋₆ alkyl groups, such as anilino, N-methylanilino, N-ethylanilino, N-n-propylanilino, N-isopropylanilino, N-n-butylanilino, N-sec-butylanilino, N-tert-butylanilino, N-n-pentylanilino, and N-n-hexylanilino.

Examples of pyridyl lower alkyl groups optionally substituted on the pyridine ring with one or more members selected from the group consisting of halogen atoms; piperidinyl groups; a morpholino group; piperazinyl groups optionally substituted on the piperizine ring with one or more members selected from the group consisting of a phenyl group and lower alkyl groups; thienyl groups; a phenyl group; pyridyl groups; piperidinyl lower alkyl groups; phenylthio lower alkyl groups; biphenyl groups; lower alkyl groups optionally substituted with one or more halogen atoms; pyridylamino groups; pyridylcarbonylamino groups; lower alkoxy groups; anilino lower alkyl groups optionally substituted on the amino group with one or more lower alkyl groups; and anilino groups optionally substituted on the amino group with one or more lower alkyl groups include:

pyridyl alkyl groups wherein the alkyl moiety is a C₁₋₆ straight or branched alkyl group, optionally substituted on the pyridine ring with one to three members selected from the group consisting of the above-described halogen atoms; piperidinyl groups; a morpholino group; the above-described piperazinyl groups optionally substituted on the piperazine ring with one to three members selected from the group consisting of a phenyl group and straight and branched C₁₋₆ alkyl groups; thienyl groups; a phenyl group; pyridyl groups; piperidinylalkyl groups wherein the alkyl moiety is a straight or branched C₁₋₆ alkyl group; phenylthioalkyl groups wherein the alkyl moiety is a straight or branched C₁₋₆ alkyl group; biphenyl groups; lower alkyl groups wherein the alkyl moiety is a straight or branched C₁₋₆ alkyl group, optionally substituted with one to three halogen atoms; pyridylamino groups; pyridylcarbonylamino groups; straight and branched C₁₋₆ alkoxy groups; anilinoalkyl groups wherein the alkyl moiety is a straight or branched C₁₋₆ alkyl group, optionally substituted on the amino group with one or two straight and/or branched C₁₋₆ alkyl groups; and the above-described anilino groups optionally substituted on the amino group with one or more straight and/or branched C₁₋₆ alkyl groups;

such as (2-, 3-, or 4-)pyridylmethyl, 2-[(2-, 3-, or 4-)pyridyl]ethyl, 1-[(2-, 3-, or 4-) pyridyl]ethyl, 3-[(2-, 3-, or 4-)pyridyl]propyl, 4-[(2-, 3-, or 4-)pyridyl]butyl, 1,1-dimethyl-2-[(2-, 3-, or 4-)pyridyl]ethyl, 5-[(2-, 3-, or 4-) pyridyl]pentyl, 6-[(2-, 3-, or 4-)pyridyl]hexyl, 1-[(2-, 3-, or 4-)pyridyl]isopropyl, 2-methyl-3-[(2-, 3-, or 4-) pyridyl]propyl, (2-chloro-3-pyridyl)methyl, [2-chloro-(3-, 4-, 5-, or 6-)pyridyl]methyl, [2,3-dichloro-(4-, 5-, or 6-) pyridyl]methyl, [2-bromo-(3-, 4-, 5-, or 6-)pyridyl]methyl, [2,4,6-trifluoro-(3-, 5-, or 6-)pyridyl]methyl, [2-(1-piperidinyl)-(3-, 4-, 5-, or 6-)pyridyl]methyl, [2-(4-morpholino)-(3-, 4-, 5-, or 6-)pyridyl]methyl, [2-(4-methyl-1-piperazinyl)-(3-, 4-, 5-, or 6-)pyridyl]methyl, 2-[2-(4-ethyl-1-piperazinyl)-(3-, 4-, 5-, or 6-)pyridyl]ethyl, 3-[2-(4-isopropyl-1-piperazinyl)-(3-, 4-, 5-, or 6-) pyridyl]propyl, 4-[2-(4-sec-butyl-1-piperazinyl)-(3-, 4-, 5-, or 6-)pyridyl]butyl, 5-[2-(4-n-pentyl-1-piperazinyl)-(3-, 4-, 5-, or 6-)pyridyl]pentyl, 6-[2-(4-n-hexyl-1-piperazinyl)-(3-, 4-, 5-, or 6-)pyridyl]hexyl, [2-(4-phenyl-2-methyl-1-piperazinyl)-(3-, 4-, 5-, or 6-)pyridyl]methyl, [2-(4-phenyl-1-piperazinyl)-(3-, 4-, 5-, or 6-)pyridyl]methyl, [2-(3-thienyl)-(3-, 4-, 5-, or 6-)pyridyl]methyl, [2-phenyl-(3-, 4-, 5-, or 6-)pyridyl]methyl, 2-[2,4-diphenyl-(3-, 5-, or 6-)pyridyl]ethyl, 3-[2-(2-pyridyl)-6-(3-thienyl)-(3-, 4-, or 5-)pyridyl]propyl, 4-(3-anilino-(2-, 4-, 5-, or 6-) pyridylbutyl, 5-[2-(4-morpholino)-(3-, 4-, 5-, or 6-)pyridyl] pentyl, 6-[2-(1-piperidinyl)-(3-, 4-, 5-, or 6-)pyridyl]hexyl, [2-(2-pyridyl)-(3-, 4-, 5-, or 6-)pyridyl]methyl, (3-, 4-, 5-, or 6-)(1-piperidinylmethyl)-2-pyridylmethyl, (3-, 4-, 5-, or 6-)phenylthiomethyl-2-pyridylmethyl, (4-, 5-, or 6-)biphenyl-3-pyridylmethyl, (4-, 5-, or 6-) trifluoromethyl-3-pyridylmethyl, (4-, 5-, or 6-)(2-pyridylamino)-3-pyridylmethyl, (4-, 5-, or 6-)[(2- or 3-)pyridylcarbonylamino]-3-pyridylmethyl, 3,5-dimethyl-4-methoxy-2-pyridylmethyl, (3-, 4-, 5-, or 6-)(N-methylanilinomethyl)-2-pyridylmethyl, [2-(N-methylanilino)-(3-, 4-, 5-, or 6-)pyridyl]methyl, 2-[2-(N-ethylanilino)-(3-, 4-, 5-, or 6-)pyridyl]ethyl, 3-[2-(N-n-propylanilino)-(3-, 4-, 5-, or 6-)pyridyl]propyl, 4-[2-(N-n-butylanilino)-(3-, 4-, 5-, or 6-)pyridyl]ethyl, 5-[2-(N-n-pentylanilino)-(3-, 4-, 5-, or 6-)pyridyl]pentyl, and 6-[2-(N-n-hexylanilino)-(3-, 4-, 5-, or 6-)pyridyl]hexyl.

Examples of cyano lower alkyl groups include cyanoalkyl groups wherein the alkyl moiety is a straight or branched C₁₋₆ alkyl group, such as cyanomethyl, 2-cyanoethyl, 1-cyanoethyl, 3-cyanopropyl, 4-cyanobutyl, 1,1-dimethyl-2-cyanoethyl, 5-cyanopentyl, 6-cyanohexyl, 1-cyanoisopropyl, and 2-methyl-3-cyanopropyl.

Examples of quinolyl lower alkyl groups include quinolylalkyl groups wherein the alkyl moiety is a straight or branched C₁₋₆alkyl group, such as [(2-, 3-, 4-, 5-, 6-, 7-, or 8-) quinolyl]methyl, 2-[(2-, 3-, 4-, 5-, 6-, 7-, or 8-)quinolyl]ethyl, 1-[(2-, 3-, 4-, 5-, 6-, 7-, or 8-)quinolyl]ethyl, 3-[(2-, 3-, 4-, 5-, 6-, 7-, or 8-)quinolyl]propyl, 4-[(2-, 3-, 4-, 5-, 6-, 7-, or 8-)quinolyl]butyl, 1,1-dimethyl-2-[(2-, 3-, 4-, 5-, 6-, 7-, or 8-)quinolyl]ethyl, 5-[(2-, 3-, 4-, 5-, 6-, 7-, or 8-)quinolyl]pentyl, 6-[(2-, 3-, 4-, 5-, 6-, 7-, or 8-)quinolyl]hexyl, 1-[(2-, 3-, 4-, 5-, 6-, 7-, or 8-)quinolyl]isopropyl, and 2-methyl-3-[(2-, 3-, 4-, 5-, 6-, 7-, or -8)quinolyl]propyl.

Examples of lower alkoxy lower alkoxy-substituted lower alkyl groups include alkoxyalkoxy-substituted alkyl groups wherein each of the two alkoxy moieties is a straight or branched C₁₋₆alkoxy group and the alkyl moiety is a straight or branched C₁₋₆alkyl group, such as methoxymethoxymethyl, 2-(methoxymethoxy)ethyl, 1-(ethoxymethoxy)ethyl, 3-(2-n-butoxyethoxy)propyl, 4-(3-n-propoxypropoxy)butyl, 1,1-dimethyl-2-(4-n-pentyloxybutoxy)ethyl, 5-(5-n-hexyloxypentyloxy)pentyl, 6-(6-methoxyhexyloxy)hexyl, 1-ethoxymethoxyisopropyl, 2-methyl-3-(2-methoxyethoxy)propyl, and 3,3-dimethyl-3-(methoxymethoxy) propyl.

Examples of hydroxy-substituted lower alkyl groups include straight and branched C₁₋₆alkyl groups substituted with one to three hydroxy groups, such as hydroxymethyl, 2-hydroxyethyl, 1-hydroxyethyl, 3-hydroxypropyl, 2,3-dihydroxypropyl, 4-hydroxybutyl, 3,4-dihydroxybutyl, 1,1-dimethyl-2-hydroxyethyl, 5-hydroxypentyl, 6-hydroxyhexyl, 3,3-dimethyl-3-hydroxypropyl, 2-methyl-3-hydroxypropyl, and 2,3,4-trihydroxybutyl.

Examples of thiazolyl lower alkyl groups optionally substituted on the thiazole ring with one or more members selected from the group consisting of halogen atoms, a phenyl group, thienyl groups, and pyridyl groups include:

thiazolylalkyl groups wherein the alkyl moiety is a straight or branched C₁₋₆ alkyl group, optionally substituted on the thiazole ring with one to three members selected from the group consisting of halogen atoms, a phenyl group, thienyl groups, and pyridyl groups;

such as [(2-, 4-, or 5-)thiazolyl]methyl, 2-[(2-, 4-, or 5-)thiazolyl]ethyl, 1-[(2-, 4-, or 5-)thiazolyl]ethyl, 3-[(2-, 4-, or 5-)thiazolyl]propyl, 4-[(2-, 4-, or 5-) thiazolyl]butyl, 5-[(2-, 4-, or 5-)thiazolyl]pentyl, 6-[(2-, 4-, or 5-)thiazolyl]hexyl, 1,1-dimethyl-2-[(2-, 4-, or 5-) thiazolyl]ethyl, [2-methyl-3-[(2-, 4-, or 5-)thiazolyl]propyl, [2-chloro-(4- or 5-)thiazolyl]methyl, 2-[2-chloro-(4- or 5-) thiazolyl]ethyl, 1-[2-fluoro-(4- or 5-)thiazolyl]ethyl, 3-[2-bromo-(4- or 5-)thiazolyl]propyl, 4-[2-iodo-(4- or 5-) thiazolyl]butyl, [2-phenyl-(4- or 5-)thiazolyl]methyl, 2-[2-phenyl-(4- or 5-)thiazolyl]ethyl, 1-[2-phenyl-(4- or 5-) thiazolyl]ethyl, 3-[2-phenyl-(4- or 5-)thiazolyl]propyl, 4-[2-phenyl-(4- or 5-)thiazolyl]butyl, 5-[2-phenyl-(4- or 5-) thiazolyl]pentyl, 6-[2-phenyl-(4- or 5-)thiazolyl]hexyl, 1,1-dimethyl-2-[2-phenyl-(4- or 5-)thiazolyl]ethyl, [2-methyl-3-[2-phenyl-(4- or 5-)thiazolyl]propyl, [2-(2- or 3-)thienyl-(4- or 5-)thiazolyl]methyl, 2-[2-(2- or 3-)thienyl-(4- or 5-) thiazolyl]ethyl, 1-[2-(2- or 3-)thienyl-(4- or 5-)thiazolyl]ethyl, 3-[2-(2- or 3-)thienyl-(4- or 5-)thiazolyl]propyl, 4-[2-(2- or 3-)thienyl-(4- or 5-)thiazolyl]butyl, 5-[2-(2- or 3-) thienyl-(4- or 5-)thiazolyl]pentyl, 6-[2-(2- or 3-)thienyl-(4- or 5-)thiazolyl]hexyl, 1,1-dimethyl-2-[2-(2- or 3-)thienyl-(4- or 5-)thiazolyl]ethyl, [2-methyl-3-[2-(2- or 3-)thienyl-(4- or 5-) thiazolyl]propyl, [2-(2-, 3-, or 4-)pyridyl-(4- or 5-) thiazolyl]methyl, 2-[2-(2-, 3-, or 4-)pyridyl-(4- or 5-) thiazolyl]ethyl, 1-[2-(2-, 3-, or 4-)pyridyl-(4- or 5-)thiazolyl] ethyl, 3-[2-(2-, 3-, or 4-)pyridyl-(4- or 5-)thiazolyl]propyl, 4-[2-(2-, 3-, or 4-) pyridyl-(4- or 5-)thiazolyl]butyl, 5-[2-(2-, 3-, or 4-)pyridyl-(4- or 5-)thiazolyl]pentyl, 6-[2-(2-, 3-, or 4-)pyridyl-(4- or 5-)thiazolyl]hexyl, 1,1-dimethyl-2-[2-(2-, 3-, or 4-)pyridyl-(4- or 5-)thiazolyl]ethyl, and [2-methyl-3-[2-(2-, 3-, or 4-)pyridyl-(4- or 5-)thiazolyl]propyl.

Examples of lower alkylsilyloxy lower alkyl groups include alkylsilyloxyalkyl groups wherein each of the two alkyl moieties is a straight or branched C₁₋₆ alkyl group, such as trimethylsilyloxymethyl, (1- or 2-)(triethylsilyloxy)ethyl, 3-(trimethylsilyloxy)propyl, dimethyl-tert-butylsilyloxymethyl, 2-(dimethyl-tert-butylsilyloxy)ethyl, 3-(dimethyl-tert-butylsilyloxy)propyl, 4-(dimethyl-tert-butylsilyloxy)butyl, 5-(dimethyl-tert-butylsilyloxy)pentyl, and 6-(dimethyl-tert-butylsilyloxy)hexyl.

Examples of phenoxy lower alkyl groups optionally substituted on the phenyl ring with one or more members selected from the group consisting of lower alkyl groups optionally substituted with one or more halogen atoms; lower alkoxy groups; halogen atoms; lower alkenyl groups, cycloalkyl groups, a nitro group; and a phenyl group include:

phenoxy alkyl groups wherein the alkyl moiety is a straight or branched C₁₋₆ alkyl group, optionally substituted on the phenyl ring with one to three members selected from the group consisting of straight and branched C₁₋₆ alkyl groups optionally substituted with one to three halogen atoms; straight and branched C₁₋₆ alkoxy groups; halogen atoms; straight and branched C₂₋₆ alkenyl groups; C₃₋₈ cycloalkyl groups; a nitro group; and a phenyl group;

such as 3-[(2-, 3-, or 4-)methylphenoxy]propyl, 3-[(2-, 3-, or 4-)propylphenoxy]propyl, 3-[(2-, 3-, or 4-) methoxyphenoxy]propyl, 3-[(2,3- or 3,4-)dichlorophenoxy]propyl, 3-[(2,3- or 3,4-)difluorophenoxy]propyl, 3-[3-fluoro-4-chlorophenoxy]propyl, 3-[(2-, 3-, or 4-)trifluoromethylphenoxy] propyl, 3-[2-methoxy-4-propenylphenoxy]propyl, 3-[2-chloro-4-methoxyphenoxy]propyl, (2-, 3-, or 4-)cyclopentylphenoxypropyl, 3-[(2-, 3-, or 4-)nitrophenoxy]propyl, 3-[(2,3- or 3,4-) dimethylphenoxy]propyl, and 3-[(2-, 3-, or 4-)phenylphenoxy] propyl.

Examples of phenylthio lower alkyl groups optionally substituted on the phenyl ring with one or more halogen atoms include:

phenylthioalkyl groups wherein the alkyl moiety is a straight or branched C₁₋₆ alkyl group, optionally substituted on the phenyl ring with one to three halogen atoms;

such as phenylthiomethyl, 2-phenylthioethyl, 1-phenylthioethyl, 3-phenylthiopropyl, 4-phenylthiobutyl, 5-phenylthiopentyl, 6-phenylthiohexyl, 1,1-dimethyl-2-phenylthioethyl, 2-methyl-3-phenylthiopropyl, (2-, 3-, or 4-) chlorophenylthiomethyl, 2-[(2-, 3-, or 4-)chlorophenylthio]ethyl, 3-[(2-, 3-, or 4-)chlorophenylthio]propyl, 4-[(2-, 3-, or 4-) fluorophenylthio]butyl, 5-[(2-, 3-, or 4-)bromophenylthio]pentyl, and 6-[(2-, 3-, or 4-)iodophenylthio]hexyl.

Examples of piperidinyl lower alkyl groups optionally substituted on the piperidine ring with one or more members selected from the group consisting of a phenyl group and phenyl lower alkyl groups include:

piperidinylalkyl groups wherein the alkyl moiety is a straight or branched C₁₋₆ alkyl group, optionally substituted on the piperidine ring with one to three members selected from the group consisting of a phenyl group and phenylalkyl groups wherein the alkyl moiety is a straight or branched C₁₋₆ alkyl group;

such as [(1-, 2-, 3-, or 4-)piperidinyl]methyl, 2-[(1-, 2-, 3-, or 4-)piperidinyl]ethyl, 1-[(1-, 2-, 3-, or 4-) piperidinyl]ethyl, 3-[(1-, 2-, 3-, or 4-)piperidinyl]propyl, 4-[(1-, 2-, 3-, or 4-) piperidinyl]butyl, 5-[(1-, 2-, 3-, or 4-) piperidinyl]pentyl, 6-[(1-, 2-, 3-, or 4-)piperidinyl]hexyl, 1,1-dimethyl-2-[(1-, 2-, 3-, or 4-)piperidinyl]ethyl, 2-methyl-3-[(1-, 2-, 3-, or 4-)piperidinyl]propyl, [4-phenyl-1-piperidinyl]methyl, 3-[4-phenyl-1-piperidinyl]propyl, [4-phenylmethyl-1-piperidinyl]methyl, 3-[4-phenylmethyl-1-piperidinyl]propyl, 2-[4-phenyl-(1-, 2-, or 3-)piperidinyl]ethyl, 3-[4-phenylmethyl-(1-, 2-, or 3-)piperidinyl]propyl, 4-[4-phenylethyl-(1-, 2-, or 3-)piperidinyl]butyl, 5-[4-phenyl-(1-, 2-, or 3-) piperidinyl]pentyl, and 6-[4-phenyl-(1-, 2-, or 3-)piperidinyl] hexyl.

Examples of piperazinyl lower alkyl groups optionally substituted on the piperazine ring with one or more phenyl groups include:

piperazinylalkyl groups wherein the alkyl moiety is a straight or branched C₁₋₆ alkyl group, optionally substituted on the piperazine ring with one to three phenyl groups;

such as (1- or 2-)piperazinylmethyl, 2-[(1- or 2-) piperazinyl]ethyl, [4-phenyl-(1-, 2-, or 3-)piperazinyl]methyl, 2-[4-phenyl-(1-, 2-, or 3-)piperazinyl]ethyl, 3-[4-phenyl-(1-, 2-, or 3-)piperazinyl]propyl, 4-[4-phenyl-(1-, 2-, or 3-) piperazinyl]butyl, 5-[4-phenyl-(1-, 2-, or 3-)piperazinyl]pentyl, and 6-[4-phenyl-(1-, 2-, or 3-)piperazinyl]hexyl.

Examples of 1,2,3,4-tetrahydroisoquinolyl lower alkyl groups include 1,2,3,4-tetrahydroisoquinolylalkyl groups wherein the alkyl moiety is a straight or branched C₁₋₆ alkyl group, such as (1,2,3,4-tetrahydroisoquinolin-2-yl)methyl, 2-(1,2,3,4-tetrahydroisoquinolin-2-yl)ethyl, 3-(1,2,3,4-tetrahydroisoquinolin-2-yl)propyl, 4-(1,2,3,4-tetrahydroisoquinolin-2-yl)butyl, 5-(1,2,3,4-tetrahydroisoquinolin-2-yl)pentyl, and 6-(1,2,3,4-tetrahydroisoquinolin-2-yl)hexyl.

Examples of naphthyloxy lower alkyl groups include naphthyloxyalkyl groups wherein the alkyl moiety is a straight or branched C₁₋₆ alkyl group, such as 1-naphthyloxymethyl, 2-(2-naphthyloxy)ethyl, 3-(1-naphthyloxy)propyl, 3-(2-naphthyloxy)propyl, 4-(1-naphthyloxy)butyl, 5-(2-naphthyloxy)pentyl and 6-(1-naphthyloxy)hexyl.

Examples of benzothiazolyloxy lower alkyl group optionally substituted on the benzothiazole ring with one or more alkyl groups include:

benzothiazolyloxyalkyl groups wherein the alkyl moiety is a straight or branched C₁₋₆ alkyl group, optionally substituted on the benzothiazoline ring with one to three straight and/or branched C₁₋₆ alkyl groups;

such as 1-[benzothiazol-(2-, 4-, 5-, 6- or 7-)yloxy]methyl, 2-[benzothiazol-(2-, 4-, 5-, 6- or 7-)yloxy]ethyl, 3-[benzothiazol-(2-, 4-, 5-, 6- or 7-)yloxy]propyl, 3-[benzothiazol-(2-, 4-, 5-, 6- or 7-)yloxy]propyl, 4-[benzothiazol-(2-, 4-, 5-, 6- or 7-)yloxy]butyl, 5-[benzothiazol-(2-, 4-, 5-, 6- or 7-)yloxy]pentyl, 6-[benzothiazol-(2-, 4-, 5-, 6- or 7-)yloxy]hexyl, 2-methylbenzothiazol-5-yloxymethyl, 2-(2-methylbenzothiazol-5-yloxy)ethyl, 3-(2-methylbenzothiazol-5-yloxy)propyl, 4-(2-ethylbenzothiazol-5-yloxy)butyl, 5-(2-ethylbenzothiazol-5-yloxy)pentyl, and 6-(2-ethylbenzothiazol-5-yloxy)hexyl.

Examples of lower alkyl groups substituted with one or more members selected from the group consisting of quinolyloxy groups and isoquinolyloxy groups include:

alkyl groups wherein the alkyl moiety is a straight or branched C₁₋₆ alkyl group, substituted with one to three members selected from the group consisting of quinolyloxy groups and isoquinolyloxy groups;

such as (5-quinolyloxy)methyl, 2-(5-quinolyloxy)ethyl, 3-(5-quinolyloxy)propyl, 4-(5-quinolyloxy)butyl, 5-(5-quinolyloxy)pentyl, 6-(5-quinolyloxy)hexyl, (5-isoquinolyloxy)methyl, 2-(5-isoquinolyloxy)ethyl, 3-(5-isoquinolyloxy)propyl, 4-(5-isoquinolyloxy)butyl, 5-(5-isoquinolyloxy)pentyl, and 6-(5-isoquinolyloxy)hexyl.

Examples of pyridyloxy lower alkyl groups optionally substituted on the pyridine ring with one or more lower alkyl groups include:

pyridyloxyalkyl groups wherein the alkyl moiety is a straight or branched C₁₋₆ alkyl group, optionally substituted on the pyridine ring with one to three straight and/or branched C₁₋₆ alkyl groups;

such as (2-, 3-, or 4-)pyridyloxymethyl, 2-[(2-, 3-, or 4-)pyridyloxy]ethyl, 1-[(2-, 3-, or 4-)pyridyloxy]ethyl, 3-[(2-, 3-, or 4-)pyridyloxy]propyl, 4-[(2-, 3-, or 4-) pyridyloxy]butyl, 1,1-dimethyl-2-[(2-, 3-, or 4-)pyridyloxy]ethyl, 5-[(2-, 3-, or 4-)pyridyloxy]pentyl, 6-[(2-, 3-, or 4-) pyridyloxy]hexyl, [6-methyl-(2-, 3-, 4-, or 5-)pyridyloxy]methyl, 2-[6-ethyl-(2-, 3-, 4-, or 5-)pyridyloxy]ethyl, 3-[6-methyl-(2-, 3-, 4-, or 5-)pyridyloxy]propyl, 4-[6-methyl-(2-, 3-, 4-, or 5-)pyridyloxy]butyl, 5-[6-methyl-(2-, 3-, 4-, or 5-)pyridyloxy]pentyl, and 6-[6-methyl-(2-, 3-, 4-, or 5-)pyridyloxy]hexyl.

Examples of carboxy lower alkoxy groups include carboxyalkoxy groups wherein the alkoxy moiety is a straight or branched C₁₋₆alkoxy group, such as carboxymethoxy, 2-carboxyethoxy, 1-carboxyethoxy, 3-carboxypropoxy, 4-carboxybutoxy, 5-carboxypentyloxy, 6-carboxyhexyloxy, 1,1-dimethyl-2-carboxyethoxy, and 2-methyl-3-carboxypropoxy.

Examples of lower alkoxycarbonyl lower alkoxy groups include alkoxycarbonylalkoxy groups wherein each of the two alkoxy moieties is a straight or branched C₁₋₆ alkoxy group, such as methoxycarbonylmethoxy, ethoxycarbonylmethoxy, 2-methoxycarbonylethoxy, 2-ethoxycarbonylethoxy, 1-ethoxycarbonylethoxy, 3-methoxycarbonylpropoxy, 3-ethoxycarbonylpropoxy, 4-ethoxycarbonylbutoxy, 5-isopropoxycarbonylpentyloxy, 6-n-propoxycarbonylhexyloxy, 1,1-dimethyl-2-n-butoxycarbonylethoxy, 2-methyl-3-tert-butoxycarbonylpropoxy, 2-n-pentyloxycarbonylethoxy, and n-hexyloxycarbonylmethoxy.

Examples of lower alkyl groups optionally substituted with one or more halogen atoms include straight and branched C₁₋₆ alkyl groups optionally substituted with one to three halogen atoms, such as, in addition to the above-described lower alkyl groups, trifluoromethyl, trichloromethyl, chloromethyl, bromomethyl, fluoromethyl, iodomethyl, difluoromethyl, dibromomethyl, 2-chloroethyl, 2,2,2-trifluoroethyl, 2,2,2-trichloroethyl, 3-chloropropyl, 2,3-dichloropropyl, 4,4,4-trichlorobutyl, 4-fluorobutyl, 4,4,4-trifluorobutyl, 5-chloropentyl, 3-chloro-2-methylpropyl, 5-bromohexyl, and 5,6-dibromhexyl.

Examples of lower alkylthio groups optionally substituted with one or more halogen atoms include straight and branched C₁₋₆ alkylthio groups optionally substituted with one to three halogen atoms, such as, in addition to the above-described lower alkylthio groups, trifluoromethylthio, trichloromethylthio, chloromethylthio, bromomethylthio, fluoromethylthio, iodomethylthio, difluoromethylthio, dibromomethylthio, 2-chloroethylthio, 2,2,2-trifluoroethylthio, 2,2,2-trichloroethylthio, 3-chloropropylthio, 2,3-dichloropropylthio, 4,4,4-trichlorobutylthio, 4-fluorobutylthio, 4,4,4-trifluorobutylthio, 5-chloropentylthio, 3-chloro-2-methylpropylthio, 5-bromohexylthio, and 5,6-dibromohexylthio.

Examples of lower alkylsulfonyl groups include straight and branched C₁₋₆ alkyl sulfonyl groups optionally substituted with one to three halogen atoms, such as methylsulfonyl, ethylsulfonyl, n-propylsulfonyl, isopropylsulfonyl, n-butylsulfonyl, isobutylsulfonyl, tert-butylsulfonyl, sec-butylsulfonyl, n-pentylsulfonyl, isopentylsulfonyl, neopentylsulfonyl, n-hexylsulfonyl, isohexylsulfonyl, and 3-methylpentylsulfonyl.

Examples of phenyl lower alkenyl groups include phenylalkenyl groups containing one to three double bonds wherein the alkenyl moiety is a straight or branched C₂₋₆ alkenyl group, such as styryl, 3-phenyl-2-propenyl (trivial name: cinnamyl), 4-phenyl-2-butenyl, 4-phenyl-3-butenyl, 5-phenyl-4-pentenyl, 5-phenyl-3-pentenyl, 6-phenyl-5-hexenyl, 6-phenyl-4-hexenyl, 6-phenyl-3-hexenyl, 4-phenyl-1,3-butadienyl, and 6-phenyl-1,3,5-hexatrienyl.

Examples of lower alkanoyloxy groups include straight and branched C₂₋₆ alkanoyloxy groups such as acetyloxy, propionyloxy, butyryloxy, isobutyryloxy, pentanoyloxy, tert-butylcarbonyloxy, and hexanoyloxy.

Examples of phenyl lower alkoxy groups optionally substituted on the phenyl ring with one or more members selected from the group consisting of halogen atoms; lower alkyl groups optionally substituted with one or more halogen atoms; lower alkylthio groups optionally substituted with one or more halogen atoms; lower alkoxy groups; a nitro group; lower alkylsulfonyl groups; lower alkoxycarbonyl groups; phenyl lower alkenyl groups; lower alkanoyloxy groups; and 1,2,3-thiadiazolyl groups include:

phenylalkoxy groups wherein the alkoxy moiety is a straight or branched C₁₋₆ alkoxy group, optionally substituted on the phenyl ring with one to three members selected from the group consisting of the above-described halogen atoms; the above-described straight and branched C₁₋₆ alkyl groups optionally substituted with one to three halogen atoms; the above-described straight and branched C₁₋₆ alkylthio groups optionally substituted with one to three halogen atoms; the above-described straight and branched C₁₋₆ alkoxy groups; a nitro group; the above-described straight and branched C₁₋₆ alkylsulfonyl groups; the above-described straight and branched C₁₋₆ alkoxycarbonyl groups; the above-described phenylalkenyl groups containing one to three double bonds wherein the alkenyl moiety is a straight or branched C₂₋₆ alkenyl group; the above-described straight and branched C₁₋₆ alkanoyloxy groups; and 1,2,3-thiadiazolyl groups;

such as benzyloxy, 2-phenylethoxy, 1-phenylethoxy, 3-phenylpropoxy, 4-phenylbutoxy, 5-phenylpentyloxy, 6-phenylhexyloxy, 1,1-dimethyl-2-phenylethoxy, 2-methyl-3-phenylpropoxy, 4-chlorobenzyloxy, 2-chlorobenzyloxy, 3-chlorobenzyloxy, 3-fluorobenzyloxy, 4-fluorobenzyloxy, 2,4-dibromobenzyloxy, 2,4,6-trifluorobenzyloxy, 3-trifluoromethylbenzyloxy, 4-trifluoromethylbenzyloxy, 4-methylbenzyloxy, 3-methylbenzyloxy, 2,4-dimethylbenzyloxy, 2,4,6-trimethylbenzyloxy, 4-methoxycarbonylbenzyloxy, 3-methoxybenzyloxy, 2-methoxybenzyloxy, 3-methoxycarbonylbenzyloxy, 2,3-dimethoxybenzyloxy, 2,4,5-trimethoxybenzyloxy, 3-nitrobenzyloxy, 2-(2,3-dinitrophenyl)ethoxy, 3-(2,4,6-trinitrophenyl)ethoxy, 2-nitro-4-methylbenzyloxy, 4-methylsulfonylbenzyloxy, 4-(4-ethylsulfonylphenyl)butoxy, 5-(4-propylsulfonylphenyl)pentyloxy, 4-acetyloxybenzyloxy, 6-(4-propionyloxyphenyl)hexyloxy, 4-styrylbenzyloxy, 4-(1,2,3-thiadiazol-4-yl)benzyloxy, 4-trifluoromethylthiobenzyloxy, 3-methylthiobenzyloxy, 2,4-dimethylthiobenzyloxy, and 2,4,6-trimethylthiobenzyloxy.

Examples of piperidinyl lower alkoxy groups optionally substituted on the piperidine ring with one or more lower alkyl groups include:

piperidinylalkoxy groups wherein the alkoxy moiety is a straight or branched C₁₋₆ alkoxy group, optionally substituted on the piperidine ring with one to three straight and/or branched C₁₋₆ alkyl groups;

such as [(1-, 2-, 3-, or 4-) piperidinyl]methoxy, 2-[(1-, 2-, 3-, or 4-)piperidinyl]ethoxy, 1-[(1-, 2-, 3-, or 4-) piperidinyl]ethoxy, 3-[(1-, 2-, 3-, or 4-)piperidinyl]propoxy, 4-[(1-, 2-, 3-, or 4-)piperidinyl]butoxy, 5-[(1-, 2-, 3-, or 4-) piperidinyl]pentyloxy, 6-[(1-, 2-, 3-, or 4-)piperidinyl]hexyloxy, 1,1-dimethyl-2-[(1-, 2-, 3-, or 4-)piperidinyl]ethoxy, 2-methyl-3-[(1-, 2-, 3-, or 4-)piperidinyl]propoxy, [1-methyl-(2-, 3-, or 4-)piperidinyl]methoxy, 2-[1-ethyl-(2-, 3-, or 4-) piperidinyl]ethoxy, 3-[1-n-propyl-(2-, 3-, or 4-)piperidinyl] propoxy, 4-[1-n-butyl-(2-, 3-, or 4-piperidinyl)butoxy, 5-[1-n-pentyl-(2-, 3-, or 4-)piperidinyl]pentyloxy, 6-[1-n-hexyl-(2-, 3-, or 4-)piperidinyl]hexyloxy, [1,2-dimethyl-(3-, 4-, 5-, or 6-) piperidinyl]methoxy, [1,2,3-trimethyl-(4-, 5-, or 6-)piperidinyl]methoxy, 2-[2-n-propyl-(3-, 4-, 5-, or 6-)piperidinyl]ethoxy, 2-[3-ethyl-(2-, 4-, 5-, or 6-)piperidinyl]ethoxy, and [2-methyl-4-isopropyl-(3-, 5-, or 6-piperidinyl)methoxy.

Examples of amino-substituted lower alkoxy groups optionally substituted on each amino group with one or more lower alkyl groups include amino-substituted straight and branched C₁₋₆ alkoxy groups optionally substituted on the amino group with one or two straight and/or branched C₁₋₆alkyl groups, such as aminomethoxy, 2-aminomethoxy, 1-aminoethoxy, 3-aminopropoxy, 4-aminobutoxy, 5-aminopentyloxy, 6-aminohexyloxy, 1,1-dimethyl-2-aminoethoxy, 2-methyl-3-aminopropoxy, methylaminomethoxy, 1-ethylaminoethoxy, 2-n-propylaminoethoxy, 3-isopropylaminopropoxy, 4-n-butylaminobutoxy, 5-n-pentylaminopentyloxy, 6-n-hexylaminohexyloxy, dimethylaminomethoxy, 3-dimethylaminopropoxy, 2-diisopropylaminoethoxy, (N-ethyl-N-n-propylamino)methoxy, and 2-(N-methyl-N-n-hexylamino)ethoxy.

Examples of lower alkenyloxy groups include straight and branched C₂₋₆ alkenyloxy groups containing one to three double bonds, such as vinyloxy, 1-propenyloxy, 1-methyl-1-propenyloxy, 2-methyl-1-propenyloxy, 2-propenyloxy, 2-butenyloxy, 1-butenyloxy, 3-butenyloxy, 2-pentenyloxy, 1-pentenyloxy, 3-pentenyloxy, 4-pentenyloxy, 1,3-butadienyloxy, 1,3-pentadienyloxy, 2-penten-4-yloxy, 2-hexenyloxy, 1-hexenyloxy, 5-hexenyloxy, 3-hexenyloxy, 4-hexenyloxy, 3,3-dimethyl-1-propenyloxy, 2-ethyl-1-propenyloxy, 1,3,5-hexatrienyloxy, 1,3-hexadienyloxy, and 1,4-hexadienyloxy.

Examples of pyridyl lower alkoxy groups optionally substituted on the pyridine ring with one or more lower alkyl groups, each lower alkyl substituent optionally being substituted with one or more halogen atoms include:

pyridylalkoxy groups wherein the alkoxy moiety is a straight or branched C₁₋₆ alkoxy group, optionally substituted on the pyridine ring with one to three above-described straight and/or branched C₁₋₆ alkyl groups, each alkyl substituent optionally being substituted with one to three halogen atoms;

such as [(2-, 3-, or 4-)pyridyl]methoxy, 2-[(2-, 3-, or 4-)pyridyl]ethoxy, 1-[(2-, 3-, or 4-) pyridyl]ethoxy, 3-[(2-, 3-, or 4-)pyridyl]propoxy, 4-[(2-, 3-, or 4-)pyridyl] butoxy, 5-[(2-, 3-, or 4-) pyridyl]pentyloxy, 6-[(2-, 3-, or 4-) pyridyl]hexyloxy, 1,1-dimethyl-2-[(2-, 3-, or 4-)pyridyl]ethoxy, 2-methyl-3-[(2-, 3-, or 4-)pyridyl]propoxy, [2-trifluoromethyl-(3-, 4-, 5-, or 6-)pyridyl]methoxy, [2-methyl-(3-, 4-, 5-, or 6-) pyridyl]methoxy, [2,4-dimethyl-(3-, 5-, or 6-)pyridyl]methoxy, [2,4,6-trimethyl-(3- or 5-)pyridyl]methoxy), [2-trifluoromethyl-4-methyl-(3-, 5-, or 6-)pyridyl]methoxy, 2-[3-ethyl-(2-, 4-, 5-, or 6-) pyridyl]ethoxy, 3-[4-n-propyl-(2- or 3-)pyridyl]propoxy, 4-[3-n-butyl-(2-, 4-, 5-, or 6-) pyridyl]butyl, 5-[3-trifluoromethyl-(2-, 4-, 5-, or 6-)pyridyl] pentyloxy, 6-[2-n-pentyl-(3-, 4-, 5-, or 6-)pyridyl]hexyloxy, and [2-n-hexyl-(3-, 4-, 5-, or 6-)pyridyl]methoxy.

Examples of lower alkynyloxy groups include straight and branched C₂₋₆ alkynyloxy groups, such as ethynyloxy, 2-propynyloxy, 2-butynyloxy, 3-butynyloxy, 1-methyl-2-propynyloxy, 2-pentynyloxy, and 2-hexynyloxy.

Examples of phenyl lower alkynyloxy groups include phenylalkynyloxy groups wherein the alkynyloxy moiety is a straight or branched C₂₋₆ alkynyloxy group, such as 2-phenylethynyloxy, 3-phenyl-2-propynyloxy, 4-phenyl-2-butynyloxy, 4-phenyl-3-butynyloxy, 3-phenyl-1-methyl-2-propynyloxy, 5-phenyl-2-pentynyloxy, and 6-phenyl-2-hexynyloxy.

Examples of phenyl lower alkenyloxy groups include phenylalkenyloxy groups containing one to three double bonds wherein the alkenyloxy moiety is a straight or branched C₂₋₆ alkenyloxy group, such as styryloxy, 3-phenyl-1-propenyloxy, 3-phenyl-1-methyl-1-propenyloxy, 3-phenyl-2-methyl-1-propenyloxy, 3-phenyl-2-propenyloxy, 4-phenyl-2-butenyloxy, 4-phenyl-1-butenyloxy, 4-phenyl-3-butenyloxy, 4-phenyl-2-pentenyloxy, 5-phenyl-1-pentenyloxy, 5-phenyl-3-pentenyloxy, 5-phenyl-4-pentenyloxy, 4-phenyl-1,3-butadienyloxy, 5-phenyl-1,3-pentadienyloxy, 5-phenyl-2-penten-4-yloxy, 6-phenyl-2-hexenyloxy, 6-phenyl-1-hexenyloxy, 6-phenyl-5-hexenyloxy, 6-phenyl-3-hexenyloxy, 6-phenyl-4-hexenyloxy, 3-phenyl-3,3-dimethyl-1-propenyloxy, 3-phenyl-2-ethyl-1-propenyloxy, 6-phenyl-1,3,5-hexatrienyloxy, 6-phenyl-1,3-hexadienyloxy, and 6-phenyl-1,4-hexadienyloxy.

Examples of furyl lower alkoxy groups optionally substituted on the furan ring with one or more lower alkoxycarbonyl groups include:

furylalkoxy groups wherein the alkoxy moiety is a straight or branched C₁₋₆ alkoxy group, optionally substituted on the furan ring with one to three above-described alkoxycarbonyl groups wherein the alkoxy moiety is a straight or branched C₁₋₆ alkoxy group;

such as [(2- or 3-)furyl]methoxy, 2-[(2- or 3-)furyl]ethoxy, 1-[(2- or 3-)furyl]ethoxy, 3-[(2- or 3-)furyl]propoxy, 4-[(2- or 3-)furyl]butoxy, 5-[(2- or 3-)furyl]pentyloxy, 6-[(2- or 3-)furyl]hexyloxy, 1,1-dimethyl-2-[(2- or 3-)furyl]ethoxy, 2-methyl-3-[(2- or 3-)furyl]propoxy, [2-ethoxycarbonyl-(3-, 4-, or 5-)furyl]methoxy, [2-methoxycarbonyl-(3-, 4-, or 5-) furyl]methoxy, [3-n-propoxycarbonyl-(2-, 4-, or 5-)furyl]methoxy, [2-n-butoxycarbonyl-(3-, 4-, or 5-)furyl]methoxy, [3-n-pentyloxycarbonyl-(2-, 4-, or 5-)furyl]methoxy, [2-n-hexyloxycarbonyl-(3-, 4-, or 5-)furyl]methoxy, [2,3-diethoxycarbonyl-(4- or 5-)furyl]methoxy, 2,3,4-trimethoxycarbonyl-5-furyl)methoxy, 2-[3-n-propoxycarbonyl-(2-, 4-, or 5-)furyl]ethoxy, 3-[2-n-butoxycarbonyl-(3-, 4-, or 5-)furyl]propoxy, 4-[3-n-pentyloxycarbonyl-(2-, 4-, or 5-)furyl]butoxy, 5-[2-n-hexyloxycarbonyl-(3-, 4-, or 5-)furyl]pentyloxy, and 6-[2-n-hexyloxycarbonyl-(3-, 4-, or 5-)furyl]hexyloxy.

Examples of tetrazolyl lower alkoxy groups optionally substituted on the tetrazole ring with one member selected from the group consisting of a phenyl group, phenyl lower alkyl groups, and cycloalkyl lower alkyl groups include:

tetrazolylalkoxy groups wherein the alkoxy moiety is a straight or branched C₁₋₆ alkoxy group, optionally substituted on the tetrazole ring with one member selected from the group consisting of a phenyl group, the above-described phenylalkyl groups wherein the alkyl moiety is a straight or branched C₁₋₆ alky group, and the above-descried C₃₋₈ cycloalkyl alkylgroups wherein the alkyl moiety is a straight or branched C₁₋₆ alkyl group;

such as [(1- or 5-)tetrazolyl]methoxy, 2-[(1- or 5-) tetrazolyl]ethoxy, 1-[(1- or 5-)tetrazolyl]ethoxy, 3-[(1- or 5-)tetrazolyl]propoxy, 4-[(1- or 5-)tetrazolyl] butoxy, 5-[(1- or 5-)tetrazolyl]pentyloxy, 6-[(1- or 5-) tetrazolyl]hexyloxy, 1,1-dimethyl-2-[(1- or 5-)tetrazolyl]ethoxy, 2-methyl-3-[(1- or 5-) tetrazolyl]propoxy, (1-benzyl-5-tetrazolyl)methoxy, (1-phenyl-5-tetrazolyl)methoxy, (1-cyclohexylmethyl-5-tetrazolyl)methoxy, [5-(2-phenylethyl)-1-tetrazolyl]methoxy, [1-(1-phenylethyl)-5-tetrazolyl]methoxy, [1-(3-phenylpropyl)-5-tetrazolyl]methoxy, [5-(4-phenylbutyl)-1-tetrazolyl]methoxy, [1-(5-phenylpentyl)-5-tetrazolyl]methoxy, [1-(6-phenylhexyl)-5-tetrazolyl]methoxy, [5-(2-cyclohexylethyl)-1-tetrazolyl]methoxy, [1-(1-cyclopropylethyl)-5-tetrazolyl]methoxy, [1-(3-cyclobutylpropyl)-5-tetrazolyl]methoxy, [5-(4-cyclopentylbutyl)-1-tetrazolyl]methoxy, [1-(5-cycloheptylpentyl)-5-tetrazolyl]methoxy, [1-(6-cyclooctylhexyl)-5-tetrazolyl]methoxy, 2-(1-phenyl-5-tetrazolyl)ethoxy, 3-(1-cyclohexylmethyl-5-tetrazolyl)propoxy, 4-[5-(2-phenylethyl)-1-tetrazolyl]butoxy, 5-(1-benzyl-5-tetrazolyl)pentyloxy, 6-(1-phenyl-5-tetrazolyl)hexyloxy, and 1-(1-cyclohexylmethyl-5-tetrazolyl)ethoxy.

Examples of phenyl groups optionally substituted on the phenyl ring with one or more lower alkyl groups include phenyl groups optionally substituted on the phenyl ring with one to three straight and/or branched C₁₋₆alkyl groups, such as phenyl, 2-methylphenyl, 3-methylphenyl, 4-methylphenyl, 2-ethylphenyl, 3-ethylphenyl, 4-ethylphenyl, 4-isopropylphenyl, 3-n-butylphenyl, 4-n-pentylphenyl, 4-n-hexylphenyl, 3,4-dimethylphenyl, 3,4-diethylphenyl, 2,4-dimethylphenyl, 2,5-dimethylphenyl, 2,6-dimethylphenyl, and 3,4,5-trimethylphenyl.

Examples of 1,2,4-oxadiazolyl lower alkoxy groups optionally substituted on the 1,2,4-oxadiazole ring with a phenyl group, the phenyl substituent optionally being substituted on the phenyl ring with one or more lower alkyl groups, include:

1,2,4-oxadiazolylalkoxy groups wherein the alkoxy moiety is a straight or branched C₁₋₆ alkoxy group, optionally substituted on the 1,2,4-oxadiazole ring with one of the above-described phenyl groups optionally substituted on the phenyl ring with one to three straight and/or branched C₁₋₆ alkyl groups;

such as [(3- or 5-)1,2,4-oxadiazolyl]methoxy, 2-[(3- or 5-)1,2,4-oxadiazolyl]ethoxy, 1-[(3- or 5-) 1,2,4-oxadiazolyl]ethoxy, 3-[(3- or 5-)1,2,4-oxadiazolyl] propoxy, 4-[(3- or 5-)1,2,4-oxadiazolyl]butoxy, 5-[(3- or 5-) 1,2,4-oxadiazolyl]pentyloxy, 6-[(3- or 5-)1,2,4-oxadiazolyl] hexyloxy, 1,1-dimethyl-2-[(3- or 5-)1,2,4-oxadiazolyl]ethoxy, 2-methyl-3-[(3- or 5-)1,2,4-oxadiazolyl]propoxy, [3-(4-tert-butylphenyl)-5-1,2,4-oxadiazolyl]methoxy, [3-(3-methylphenyl)-5-1,2,4-oxadiazolyl]methoxy, [5-(2-ethylphenyl)-3-1,2,4-oxadiazolyl]methoxy, [3-(4-n-propylphenyl)-5-1,2,4-oxadiazolyl]methoxy, [5-(3-n-pentylphenyl)-3-1,2,4-oxadiazolyl]methoxy, [3-(2-n-hexylphenyl)-5-1,2,4-oxadiazolyl]methoxy, [3-(2,4-dimethylphenyl)-5-1,2,4-oxadiazolyl]methoxy, [3-(2,3,5-trimethylphenyl)-5-1,2,4-oxadiazolyl]methoxy, 2-[3-(4-tert-butylphenyl)-5-1,2,4-oxadiazolyl]ethoxy, 1-[3-(3-methylphenyl)-5-1,2,4-oxadiazolyl]ethoxy, 3-[5-(2-ethylphenyl)-3-1,2,4-oxadiazolyl]propoxy, 4-[3-(4-n-propylphenyl)-5-1,2,4-oxadiazolyl] butoxy, 5-[5-(3-n-pentylphenyl)-3-1,2,4-oxadiazolyl]pentyloxy, 6-[3-(2-n-hexylphenyl)-5-1,2,4-oxadiazolyl]hexyloxy, 2-[3-(2,4-dimethylphenyl)-5-1,2,4-oxadiazolyl]ethoxy and 1-[3-(2,3,5-trimethylphenyl)-5-1,2,4-oxadiazolyl]ethoxy.

Examples of isoxazolyl lower alkoxy groups optionally substituted on the isoxazole ring with one or more lower alkyl groups include:

isoxazolylalkoxy groups wherein the alkoxy moiety is a straight or branched C₁₋₆ alkoxy group, optionally substituted on the isoxazole ring with one or two above-described straight and/or branched C₁₋₆ alkyl groups;

such as [(3-, 4-, or 5-)isoxazolyl]methoxy, 2-[(3-, 4-, or 5-)isoxazolyl]ethoxy, 1-[(3-, 4-, or 5-) isoxazolyl]ethoxy, 3-[(3-, 4-, or 5-)isoxazolyl]propoxy, 4-[(3-, 4-, or 5-)isoxazolyl]butoxy, 5-[(3-, 4-, or 5-)isoxazolyl] pentyloxy, 6-[(3-, 4-, or 5-)isoxazolyl]hexyloxy, 1,1-dimethyl-2-[(3-, 4-, or 5-)isoxazolyl]ethoxy, 2-methyl-3-[(3-, 4-, or 5-) isoxazolyl]propoxy, (3,5-dimethyl-4-isoxazolyl)methoxy, [3-methyl-(4- or 5-) isoxazolyl]methoxy, [3-ethyl-(4- or 5-) isoxazolyl]methoxy, [4-n-propyl-(3- or 5-) isoxazolyl]methoxy, [5-n-butyl-(3- or 4-)isoxazolyl]methoxy, [3-n-pentyl-(4- or 5-) isoxazolyl]methoxy, [4-n-hexyl-(3- or 5-)isoxazolyl]methoxy, 2-[3-methyl-(4- or 5-)isoxazolyl]ethoxy, 1-[3-ethyl-(4- or 5-) isoxazolyl]ethoxy, 3-[4-n-propyl-(3- or 5-) isoxazolyl]propoxy, 4-[5-n-butyl-(3- or 4-)isoxazolyl]butoxy, 5-[3-n-pentyl-(4- or 5-)isoxazolyl]pentyloxy, and 6-[4-n-hexyl-(3- or 5-) isoxazolyl]hexyloxy.

Examples of 1,3,4-oxadiazolyl lower alkoxy groups optionally substituted on the 1,3,4-oxadiazole ring with a phenyl group, the phenyl substituent optionally being substituted on the phenyl ring with one or more lower alkyl groups include:

1,3,4-oxadiazolylalkoxy groups wherein the alkoxy moiety is a straight or branched C₁₋₆ alkoxy group, optionally substituted on the 1,3,4-oxadiazole ring with one of the above-described phenyl groups optionally substituted on the phenyl ring with one to three straight and/or branched C₁₋₆ alkyl groups;

such as [(2- or 5-)1,3,4-oxadiazolyl]methoxy, 2-[(2- or 5-)1,3,4-oxadiazolyl]ethoxy, 1-[(2- or 5-)1,3,4-oxadiazolyl]ethoxy, 3-[(2- or 5-) 1,3,4-oxadiazolyl]propoxy, 4-[(2- or 5-)1,3,4-oxadiazolyl]butoxy, 5-[(2- or 5-)1,3,4-oxadiazolyl]pentyloxy, 6-[(2- or 5-) 1,3,4-oxadiazolyl]hexyloxy, 1,1-dimethyl-2-[(2- or 5-)1,3,4-oxadiazolyl]ethoxy, 2-methyl-3-[(2- or 5-)1,3,4-oxadiazolyl]propoxy, [2-(4-tert-butylphenyl)-5-1,3,4-oxadiazolyl]methoxy, [2-(4-methylphenyl)-5-1,3,4-oxadiazolyl]methoxy, [5-(2-ethylphenyl)-2-1,3,4-oxadiazolyl]methoxy, [2-(4-n-propylphenyl)-5-1,3,4-oxadiazolyl]methoxy, [5-(3-n-pentylphenyl)-2-1,3,4-oxadiazolyl]methoxy, [2-(2-n-hexylphenyl)-5-1,3,4-oxadiazolyl]methoxy, [2-(2,4-dimethylphenyl)-5-1,3,4-oxadiazolyl]methoxy, [2-(2,3,5-trimethylphenyl)-5-1,3,4-oxadiazolyl]methoxy, 2-[2-(4-tert-butylphenyl)-5-1,3,4-oxadiazolyl]ethoxy, 1-[2-(3-methylphenyl)-5-1,3,4-oxadiazolyl]ethoxy, 3-[5-(2-ethylphenyl)-2-1,3,4-oxadiazolyl]propoxy, 4-[2-(4-n-propylphenyl)-5-1,3,4-oxadiazolyl] butoxy, 5-[5-(3-n-pentylphenyl)-2-1,3,4-oxadiazolyl]pentyloxy, 6-[2-(2-n-hexylphenyl)-5-1,3,4-oxadiazolyl]hexyloxy, 2-[2-(2,4-dimethylphenyl)-5-1,3,4-oxadiazolyl]ethoxy, and 1-[2-(2,3,5-trimethylphenyl)-5-1,3,4-oxadiazolyl]ethoxy.

Examples of lower alkanoyl lower alkoxy groups include alkanoylalkoxy groups wherein the alkanoyl moiety is a straight or branched C₂₋₆ alkanoyl group and the alkoxy moiety is a straight or branched C₁₋₆ alkoxy group, such as acetylmethoxy, propionylmethoxy, 2-acetylethoxy, 2-propionylethoxy, 1-acetylethoxy, 3-acetylpropoxy, 3-propionylpropoxy, 4-acetylbutoxy, 5-butyrylpentyloxy, 6-pentanoylhexyloxy, 1,1-dimethyl-2-hexanoylethoxy, 2-methyl-3-acetylpropoxy, 2-pentanoylethoxy, and hexanoylmethoxy.

Examples of phenyl groups optionally substituted on the phenyl ring with one or more halogen atoms include phenyl groups optionally substituted on the phenyl ring with one to three halogen atoms, such as phenyl, 4-fluorophenyl, 2,5-difluorophenyl, 2,4-difluorophenyl, 3,4-difluorophenyl, 3,5-difluorophenyl, 2,6-difluorophenyl, 2-chlorophenyl, 3-chlorophenyl, 4-chlorophenyl, 2,3-dichlorophenyl, 2,4-dichlorophenyl, 2,5-dichlorophenyl, 3,4-dichlorophenyl, 2,6-dichlorophenyl, 3-fluorophenyl, 2-fluorophenyl, 3-bromophenyl, 4-iodophenyl, 2-bromophenyl, 4-bromophenyl, 3,5-dichlorophenyl, 2,4,6-trifluorophenyl, 3,4-difluorophenyl, 2-iodophenyl, 3-iodophenyl, 4-iodophenyl, 2,3-dibromophenyl, 2,4-diiodophenyl, and 2,4,6-trichlorophenyl.

Examples of thiazolyl lower alkoxy groups optionally substituted on the thiazole ring with one or more members selected from the group consisting of lower alkyl groups and a phenyl group, each phenyl substituent optionally being substituted on the phenyl ring with one or more halogen atoms, include:

thiazolylalkoxy groups wherein the alkoxy moiety is a straight or branched C₁₋₆ alkoxy group, optionally substituted on the thiazole ring with one or two members selected from the group consisting of the above-described straight and branched C₁₋₆ alkyl groups and phenyl groups optionally substituted on the phenyl ring with one to three halogen atoms;

such as [(2-, 4-, or 5-)thiazolyl]methoxy, 2-[(2-, 4-, or 5-)thiazolyl]ethoxy, 1-[(2-, 4-, or 5-)thiazolyl]ethoxy, 3-[(2-, 4-, or 5-)thiazolyl]propoxy, 4-[(2-, 4-, or 5-) thiazolyl]butoxy, 5-[(2-, 4-, or 5-) thiazolyl]pentyloxy, 6-[(2-, 4-, or 5-)thiazolyl]hexyloxy, 1,1-dimethyl-2-[(2-, 4-, or 5-)thiazolyl]ethoxy, 2-methyl-3-[(2-, 4-, or 5-) thiazolyl]propoxy, [2-phenyl-(4- or 5-)thiazolyl]methoxy, [2-(4-chlorophenyl)-4-methyl-5-thiazolyl]methoxy, [2-(3-bromophenyl)-(4- or 5-)thiazolyl]methoxy, [2-(2-fluorophenyl)-(4- or 5-) thiazolyl]methoxy, [2-(3,4-dichlorophenyl)-(4- or 5-)thiazolyl]methoxy, [2-(2,4,6-trifluorophenyl)-(4- or 5-)thiazolyl]methoxy, [2-methyl-(4- or 5-)thiazolyl]methoxy, 2-[2-ethyl-(4- or 5-) thiazolyl]methoxy, 2-[4-phenyl-(2- or 5-)thiazolyl]ethoxy, 3-[5-n-propyl-(2- or 4-)thiazolyl]propoxy, 4-[4-n-butyl-(2- or 5-)thiazolyl]butoxy, 5-[2-n-pentyl-(4- or 5-)thiazolyl] pentyloxy, 6-[5-n-hexyl-(2- or 4-)thiazolyl]hexyloxy, [2,4-dimethyl-5-thiazolyl]methoxy, and [2,4-diphenyl-5-thiazolyl]methoxy.

Examples of benzoyl groups optionally substituted on the phenyl ring with one or more halogen atoms include benzoyl groups optionally substituted on the phenyl ring with one to three halogen atoms, such as benzoyl, 4-fluorobenzoyl, 2,5-difluorobenzoyl, 2,4-difluorobenzoyl, 3,4-difluorobenzoyl, 3,5-difluorobenzoyl, 2,6-difluorobenzoyl, 2-chlorobenzoyl, 3-chlorobenzoyl, 4-chlorobenzoyl, 2,3-dichlorobenzoyl, 2,4-dichlorobenzoyl, 2,5-dichlorobenzoyl, 3,4-dichlorobenzoyl, 2,6-dichlorobenzoyl, 3-fluorobenzoyl, 2-fluorobenzoyl, 3-bromobenzoyl, 4-iodobenzoyl, 2-bromobenzoyl, 4-bromobenzoyl, 3,5-dichlorobenzoyl, 2,4,6-trifluorobenzoyl, 2-iodobenzoyl, 3-iodobenzoyl, 4-iodobenzoyl, 2,3-dibromobenzoyl, 2,4-diiodobenzoyl, and 2,4,6-trichlorobenzoyl.

Examples of piperidinyloxy groups optionally substituted on the piperidine ring with one or more benzoyl groups, each benzoyl substituent optionally being substituted on the phenyl ring with one or more halogen atoms, include:

piperidinyloxy groups optionally substituted on the piperidine ring with one to three above-described benzoyl groups, each benzoyl substituent optionally being substituted on the phenyl ring with one to three halogen atoms;

such as (1-, 2-, 3-, or 4-)piperidinyloxy, 1-(4-chlorobenzoyl)-(2-, 3-, or 4-piperidinyloxy, 1-(3-bromobenzoyl)-(2-, 3-, or 4-)piperidinyloxy, 1-benzoyl-(2-, 3-, or 4-) piperidinyloxy, 1-(2-fluorobenzoyl)-(2-, 3-, or 4-)piperidinyloxy, 1-(2,4-dichlorobenzoyl)-(2-, 3-, or 4-)piperidinyloxy, 1-(2,4,6-trifluorobenzoyl)-(2-, 3-, or 4-)piperidinyloxy, 2-(3-chlorobenzoyl)-(1-, 3-, or 4-)piperidinyloxy, 3-(2-chlorobenzoyl)-(1-, 2-, or 4-)piperidinyloxy, 4-(2,3-dibromobenzoyl)-(1-, 2-, or 3-)piperidinyloxy, 1,2-dibenzoyl-(3- or 4-)piperidinyloxy, and 1,2,4-tribenzoyl-3-piperidinyloxy.

Examples of thienyl lower alkoxy groups include thienylalkoxy groups wherein the alkoxy moiety is a straight or branched C₁₋₆alkoxy group, such as [(2- or 3-)thienyl]methoxy, 2-[(2- or 3-)thienyl]ethoxy, 1-[(2- or 3-)thienyl]ethoxy, 3-[(2- or 3-)thienyl]propoxy, 4-[(2- or 3-)thienyl]butoxy, 5-[(2- or 3-) thienyl]pentyloxy, 6-[(2- or 3-)thienyl]hexyloxy, 1,1-dimethyl-2-[(2- or 3-)thienyl]ethoxy, and 2-methyl-3-[(2- or 3-)thienyl]propoxy.

Examples of phenylthio lower alkoxy groups include phenylthioalkoxy groups wherein the alkoxy moiety is a straight or branched C₁₋₆alkoxy group, such as phenylthiomethoxy, 2-phenylthioethoxy, 1-phenylthioethoxy, 3-phenylthiopropoxy, 4-phenylthiobutoxy, 5-phenylthiopentyloxy, 6-phenylthiohexyloxy, 1,1-dimethyl-2-phenylthioethoxy, and 2-methyl-3-phenylthiopropoxy.

Examples of carbamoyl-substituted lower alkoxy groups optionally substituted with one or more lower alkyl groups include:

carbamoyl-substituted straight and branched C₁₋₆alkoxy groups optionally substituted on the carbamoyl group with one or two straight and/or branched C₁₋₆ alkyl groups;

such as carbamoylmethoxy, 2-carbamoylethoxy, 1-carbamoylethoxy, 3-carbamoylpropoxy, 4-carbamoylbutoxy, 5-carbamoylpentyloxy, 6-carbamoylhexyloxy, 1,1-dimethyl-2-carbamoylethoxy, 2-methyl-3-carbamoylpropoxy, methylcarbamoylmethoxy, 1-ethylcarbamoylethoxy, 2-n-propylcarbamoylethoxy, 3-isopropylcarbamoylpropoxy, 4-n-butylcarbamoylbutoxy, 5-n-pentylcarbamoylpentyloxy, 6-n-hexylcarbamoylhexyloxy, dimethylcarbamoylmethoxy, 3-dimethylcarbamoylpropoxy, 2-diisopropylcarbamoylethoxy, (N-ethyl-N-n-propylcarbamoyl)methoxy, and 2-(N-methyl-N-n-hexylcarbamoyl)ethoxy.

Examples of benzoyl lower alkoxy groups include benzoylalkoxy groups wherein the alkoxy moiety is a straight or branched C₁₋₆ alkoxy group, such as benzoylmethoxy, 2-benzoylethoxy, 1-benzoylethoxy, 3-benzoylpropoxy, 4-benzoylbutoxy, 5-benzoylpentyloxy, 6-benzoylhexyloxy, 1,1-dimethyl-2-benzoylethoxy, and 2-methyl-3-benzoylpropoxy.

Examples of pyridylcarbonyl lower alkoxy groups include pyridylcarbonylalkoxy groups wherein the alkoxy moiety is a straight or branched C₁₋₆ alkoxy group, such as [(2-, 3-, or 4-)pyridylcarbonyl]methoxy, 2-[(2-, 3-, or 4-) pyridylcarbonyl]ethoxy, 1-[(2-, 3-, or 4-)pyridylcarbonyl]ethoxy, 3-[(2-, 3-, or 4-)pyridylcarbonyl]propoxy, 4-[(2-, 3-, or 4-)pyridylcarbonyl]butoxy, 5-[(2-, 3-, or 4-) pyridylcarbonyl]pentyloxy, 6-[(2-, 3-, or 4-)pyridylcarbonyl] hexyloxy, 1,1-dimethyl-2-[(2-, 3-, or 4-)pyridylcarbonyl]ethoxy, and 2-methyl-3-[(2-, 3-, or 4-)] propoxy.

Examples of imidazolyl lower alkoxy groups optionally substituted on the imidazole ring with one or more phenyl lower alkyl groups include:

imidazolylalkoxy groups wherein the alkoxy moiety is a straight or branched C₁₋₆ alkoxy group, optionally substituted on the imidazole ring with one to three phenylalkyl groups wherein the alkyl moiety is a straight or branched C₁₋₆ alkyl group;

such as [(1-, 2-, 4-, or 5-)imidazolyl]methoxy, 2-[(1-, 2-, 4-, or 5-)imidazolyl]ethoxy, 1-[(1-, 2-, 4-, or 5-) imidazolyl]ethoxy, 3-[(1-, 2-, 4-, or 5-)imidazolyl]propoxy, 4-[(1-, 2-, 4-, or 5-)imidazolyl]butoxy, 5-[(1-, 2-, 4-, or 5-) imidazolyl]pentyloxy, 6-[(1-, 2-, 4-, or 5-)imidazolyl] hexyloxy, 1,1-dimethyl-2-[(1-, 2-, 4-, or 5-)imidazolyl]ethoxy, 2-methyl-3-[(1-, 2-, 4-, or 5-)imidazolyl]propoxy, [1-benzyl-(2-, 4-, or 5-)imidazolyl]methoxy, [1-(2-phenylethyl)-(2-, 4-, or 5-)imidazolyl]methoxy, 2-[2-(3-phenylpropyl)-(1-, 4-, or 5-)imidazolyl]ethoxy, 3-[4-(4-phenylbutyl)-(1-, 2-, or 5-)imidazolyl]propoxy, 5-[4-(5-phenylpentyl)-(1-, 2-, or 4-)imidazolyl]pentyloxy, 6-[1-(6-phenylhexyloxy)-(2-, 4-, or 5-)imidazolyl]hexyloxy, [1,2-dibenzyl-(4- or 5-) imidazolyl]methoxy, and [1,2,4-tribenzyl-5-imidazolyl]methoxy.

Examples of phenoxy lower alkoxy groups include phenoxyalkoxy groups wherein the alkoxy moiety is a straight or branched C₁₋₆ alkoxy group, such as phenoxymethoxy, 2-phenoxyethoxy, 1-phenoxyethoxy, 3-phenoxypropoxy, 4-phenoxybutoxy, 5-phenoxypentyloxy, 6-phenoxyhexyloxy, 1,1-dimethyl-2-phenoxyethoxy, and 2-methyl-3-phenoxypropoxy.

Examples of phenyl lower alkoxy-substituted lower alkoxy groups include phenylalkoxy-substituted alkoxy groups wherein each of the two alkoxy moieties is a straight or branched C₁₋₆alkoxy group, such as phenylmethoxymethoxy, 2-(phenylmethoxy)ethoxy, 1-(phenylmethoxy)ethoxy, 3-(phenylmethoxy) propoxy, 4-(phenylmethoxy)butoxy, 5-(phenylmethoxy)pentyloxy, 6-(phenylmethoxy)hexyloxy, 1,1-dimethyl-2-(phenylmethoxy)ethoxy, 2-methyl-3-(phenylmethoxy) propoxy, 1-(2-phenylethoxy)ethoxy, 2-(1-phenylethoxy)ethoxy, 3-(3-phenylpropoxy)propoxy, 4-(4-phenylbutoxy)butoxy, 5-(5-phenylpentyloxy)pentyloxy, 6-(6-phenylhexyloxy)hexyloxy, (1,1-dimethyl-2-phenylethoxy)methoxy, and 3-(2-methyl-3-phenylpropoxy)propoxy.

Examples of isoindolinyl lower alkoxy groups optionally substituted on the isoindoline ring with one or more oxo groups include:

isoindolinylalkoxy groups wherein the alkoxy moiety is a straight or branched C₁₋₆ alkoxy group, optionally substituted on the isoindoline ring with one or two oxo groups;

such as [(1-, 2-, 4-, or 5-)isoindolinyl]methoxy, 2-[(1-, 2-, 4-, or 5-)isoindolinyl]ethoxy, 1-[(1-, 2-, 4-, or 5-) isoindolinyl]ethoxy, 3-[(1-, 2-, 4-, or 5-)isoindolinyl]propoxy, 4-[(1-, 2-, 4-, or 5-) isoindolinyl]butoxy, 5-[(1-, 2-, 4-, or 5-)isoindolinyl]pentyloxy, 6-[(1-, 2-, 4-, or 5-)isoindolinyl]hexyloxy, 1,1-dimethyl-2-[(1-, 2-, 4-, or 5-)isoindolinyl]ethoxy, 2-methyl-3-[(1-, 2-, 4-, or 5-)isoindolinyl]propoxy, 3-[1,3-dioxo-(2-, 4-, or 5-)isoindolinyl]propoxy, [1-oxo-(2-, 3-, 4-, 5-, 6-, or 7-)isoindolinyl]methoxy, 2-[1,3-dioxo-(1-, 4-, or 5-)isoindolinyl]ethoxy, 4-[1-oxo-(2-, 3-, 4-, 5-, 6-, or 7-)isoindolinyl]butoxy, 5-[1,3-dioxo-(1-, 4-, or 5-)isoindolinyl]pentyloxy, and 6-[1-oxo-(2-, 3-, 4-, 5-, 6-, or 7-)isoindolinyl]hexyloxy.

Examples of lower alkoxy groups optionally substituted with one or more halogen atoms include straight and branched C₁₋₆ alkoxy groups optionally substituted with one to three halogen atoms, such as, in addition to the above-described lower alkoxy groups, trifluoromethoxy, trichloromethoxy, chloromethoxy, bromomethoxy, fluoromethoxy, iodomethoxy, difluoromethoxy, dibromomethoxy, 2-chloroethoxy, 2,2,2-trifluoroethoxy, 2,2,2-trichloroethoxy, 3-chloropropoxy, 2,3-dichloropropoxy, 4,4,4-trichlorobutoxy, 4-fluorobutoxy, 5-chloropentyloxy, 3-chloro-2-methylpropoxy, 5-bromohexyloxy, and 5,6-dibromohexyloxy.

Examples of lower alkanoyl groups include straight and branched C₁₋₆ alkanoyl groups, such as formyl, acetyl, propionyl, butyryl, isobutyryl, pentanoyl, tert-butylcarbonyl, and hexanoyl.

Examples of amino groups optionally substituted with one or more lower alkanoyl groups include amino groups optionally substituted with one or two straight and/or branched C₁₋₆ alkanoyl groups, such as amino, formylamino, acetylamino, propionylamino, butyrylamino, isobutyrylamino, pentanoylamino, tert-butylcarbonylamino, hexanoylamino, N,N-diacetylamino, and N-acetyl-N-propionylamino.

Examples of phenyl lower alkyl groups optionally substituted on the phenyl ring with one or more members selected from the group consisting of halogen atoms; lower alkyl groups optionally substituted with one or more halogen atoms; lower alkoxy groups optionally substituted with one or more halogen atoms; a phenyl group; lower alkoxycarbonyl groups; a phenoxy group; lower alkylthio groups; lower alkylsulfonyl groups; phenyl lower alkoxy groups; and amino groups optionally substituted with one or more lower alkanoyl groups include:

mono- and di-phenylalkyl groups wherein the alkyl moiety is a straight or branched C₁₋₆ alkyl group, optionally substituted on the phenyl ring with one to three members selected from the group consisting of the above-described halogen atoms; the above-described straight and branched C₁₋₆ alkyl groups optionally substituted with one to three halogen atoms; the above-described straight and branched C₁₋₆ alkoxy groups optionally substituted with one to three halogen atoms; a phenyl group; the above-described alkoxycarbonyl groups wherein the alkoxy moiety is a straight or branched C₁₋₆ alkoxy group; a phenoxy group, the above-described straight and branched C₁₋₆ alkylthio groups; the above-described straight and branched C₁₋₆ alkylsulfonyl groups; the above-described phenylalkoxy groups wherein the alkoxy moiety is a straight or branched C₁₋₆ alkoxy group; and the above-described amino groups optionally substituted with one or two straight and/or branched C₁₋₆ alkanoyl groups;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-diphenylmethyl, 2,2-diphenylethyl, 3,3-diphenylpropyl, 1,2-diphenylethyl, 4-chlorobenzyl, 2-chlorobenzyl, 3-chlorobenzyl, 3-fluorobenzyl, 4-fluorobenzyl, 2,3-dichlorobenzyl, 2,4,6-trifluorobenzyl, 3-trifluoromethylbenzyl, 4-trifluoromethylbenzyl, 2-methylbenzyl, 3-methylbenzyl, 4-methylbenzyl, 4-tert-butylbenzyl, 2,4-dimethylbenzyl, 2,4,6-trimethylbenzyl, 2-phenylbenzyl, 4-phenylbenzyl, 2,4-diphenylbenzyl, 2,4,6-triphenylbenzyl, 2-trifluoromethoxybenzyl, 3-trifluoromethoxybenzyl, 4-trifluoromethoxybenzyl, 2-methoxybenzyl, 3-methoxybenzyl, 4-methoxybenzyl, 3,4-dimethoxybenzyl, 3,4,5-trimethoxybenzyl, 4-methoxycarbonylbenzyl, 3-ethoxycarbonylbenzyl, 2-n-propoxycarbonylbenzyl, 2,4-dimethoxycarbonylbenzyl, 2,4,6-trimethoxycarbonylbenzyl, 4-tert-butoxycarbonylbenzyl, 3-phenoxybenzyl, 2-phenoxybenzyl, 4-phenoxybenzyl, 3,4-diphenoxybenzyl, 3,4,5-triphenoxybenzyl, 4-methylthiobenzyl, 3-methylthiobenzyl, 2-methylthiobenzyl, 2,4-dimethylthiobenzyl, 2,4,6-trimethylthiobenzyl, 4-methylsulfonylbenzyl, 3-methylsulfonylbenzyl, 2-methylsulfonylbenzyl, 3,4-dimethylsulfonylbenzyl, 3,4,5-trimethylsulfonylbenzyl, 4-benzyloxybenzyl, 3-benzyloxybenzyl, 2-benzyloxybenzyl, 2,4-dibenzyloxybenzyl, 2,4,6-tribenzyloxybenzyl, 4-methoxy-3-chlorobenzyl, 4-(N-acetylamino)benzyl, 3-aminobenzyl, 2-aminobenzyl, 4-aminobenzyl, 2,3-diaminobenzyl, 3,4,5-triaminobenzyl, and 4-methyl-3-fluorobenzyl.

Examples of naphthyl lower alkyl groups include naphthylalkyl groups wherein the alkyl moiety is a straight or branched C₁₋₆alkyl group, such as [(1- or 2-)naphthyl]methyl, 1-[(1- or 2-)naphthyl]ethyl, 2-[(1- or 2-)naphthyl]ethyl, 3-[(1- or 2-)naphthyl]propyl, 2-[(1- or 2-)naphthyl]propyl, 4-[(1- or 2-)naphthyl]butyl, 5-[(1- or 2-)naphthyl]pentyl, 4-[(1- or 2-)naphthyl]pentyl, 6-[(1- or 2-)naphthyl]hexyl, 2-methyl-3-[(1- or 2-)naphthyl]propyl, and 1,1-dimethyl-2-[(1- or 2-)naphthyl]ethyl.

Examples of furyl lower alkyl groups optionally substituted on the furan ring with one or more lower alkoxycarbonyl groups include:

furylalkyl groups wherein the alkyl moiety is a straight or branched C₁₋₆ alkyl group, optionally substituted on the furan ring with one to three alkoxycarbonyl groups wherein the alkoxy moiety is a straight or branched C₁₋₆ alkoxy group;

such as [(2- or 3-)furyl]methyl, 2-[(2- or 3-)furyl]ethyl, 1-[(2- or 3-)furyl]ethyl, 3-[(2- or 3-)furyl]propyl, 4-[(2- or 3-)furyl]butyl, 5-[(2- or 3-)furyl]pentyl, 6-[(2- or 3-) furyl]hexyl, 1,1-dimethyl-2-[(2- or 3-)furyl]ethyl, 2-methyl-3-[(2- or 3-)furyl]propyl, [5-ethoxycarbonyl-(2-, 3-, or 4-)furyl]methyl, [5-methoxycarbonyl-(2-, 3-, or 4-)furyl]methyl, [2-n-propoxycarbonyl-(3-, 4-, or 5-)furyl]methyl, [3-tert-butoxycarbonyl-(2-, 4-, or 5-)furyl]methyl, [4-n-pentyloxycarbonyl-(2-, 3-, or 5-)furyl]methyl, [2-n-hexyloxycarbonyl-(3-, 4-, or 5-)furyl]methyl, [2,5-diethoxycarbonyl-(3- or 4-)furyl]methyl, and [2,4,5-triethoxycarbonyl-3-furyl]methyl.

Examples of phenyl groups optionally substituted on the phenyl ring with one or more lower alkyl groups, each lower alkyl substituent optionally being substituted with one or more halogen atoms, include:

phenyl groups optionally substituted on the phenyl ring with one to three straight and/or branched C₁₋₆ alkyl groups, each alkyl substituent optionally being substituted with one to three above-described halogen atoms;

such as phenyl, 2-methylphenyl, 3-methylphenyl, 4-methylphenyl, 2-ethylphenyl, 3-ethylphenyl, 4-ethylphenyl, 4-isopropylphenyl, 3-n-butylphenyl, 4-n-pentylphenyl, 4-n-hexylphenyl, 3,4-dimethylphenyl, 3,4-diethylphenyl, 2,4-dimethylphenyl, 2,5-dimethylphenyl, 2,6-dimethylphenyl, 3,4,5-trimethylphenyl, 2-trifluoromethylphenyl, 3-trifluoromethylphenyl, 4-trifluoromethylphenyl, 3,5-difluoromethylphenyl, 2,4,6-tri (trifluoromethyl)phenyl, and 2-methyl-4-trifluoromethylphenyl.

Examples of thiazolyl lower alkyl groups optionally substituted on the thiazole ring with one or more members selected from the group consisting of lower alkyl groups and a phenyl group, each phenyl substituent optionally being substituted with one or more optionally halogen-substituted lower alkyl groups, include thiazolylalkyl groups wherein the alkyl moiety is a straight or branched C₁₋₆alkyl group. Such thiazoylalkyl groups include those optionally substituted on the thiazole ring with one or two members selected from the above-described straight and branched C₁₋₆ alkyl groups and the above-described phenyl groups optionally substituted on the phenyl ring with one to three straight and/or branched C₁₋₆ alkyl groups, each alkyl substituent on the phenyl substituent optionally further being substituted with one to three halogen atoms. More specific examples of the thiazolyl lower alkyl groups are [(2-, 4-, or 5-) thiazolyl]methyl, 2-[(2-, 4-, or 5-) thiazolyl]ethyl, 1-[(2-, 4-, or 5-)thiazolyl]ethyl, 3-[(2-, 4-, or 5-)thiazolyl] propyl, 4-[(2-, 4-, or 5-)thiazolyl]butyl, 5-[(2-, 4-, or 5-) thiazolyl]pentyl, 6-[(2-, 4-, or 5-)thiazolyl]hexyl, 1,1-dimethyl-2-[(2-, 4-, or 5-)thiazolyl]ethyl, [2-methyl-(4- or 5-) thiazolyl]methyl, [2-(4-trifluoromethylphenyl)-[(4- or 5-) thiazolyl]methyl, 2-[4-ethyl-(2- or 5-)thiazolyl]ethyl, 1-[5-(3-methylphenyl)-(2- or 4-)thiazolyl]ethyl, 3-[5-isopropyl-(2- or 4-)thiazolyl]propyl, 4-[2-(2,4-dimethylphenyl)-(4- or 5-) thiazolyl]butyl, 5-[2-n-butyl-(4- or 5-)thiazolyl]pentyl, 6-[4-(2,4,6-trimethylphenyl)-(2- or 5-)thiazolyl]hexyl, (2,4-dimethyl-5-thiazolyl)methyl, [2-(4-trifluoromethylphenyl)-4-phenyl-5-thiazolyl]methyl, and (2-phenyl-4-thiazolyl)methyl.

Examples of tetrazolyl lower alkyl groups optionally substituted on the tetrazole ring with one or more lower alkyl groups include:

tetrazolylalkyl groups wherein the alkyl moiety is a straight or branched C₁₋₆ alkyl group, optionally substituted on the tetrazole ring with one or more straight and/or branched C₁₋₆ alkyl groups,

such as [(1- or 5-)tetrazolyl]methyl, 2-[(1- or 5-) tetrazolyl]ethyl, 1-[(1- or 5-)tetrazolyl]ethyl, 3-[(1- or 5-) tetrazolyl]propyl, 4-[(1- or 5-)tetrazolyl]butyl, 5-[(1- or 5-) tetrazolyl]pentyl, 6-[(1- or 5-)tetrazolyl]butyl, 5-(1-methyl-5-tetrazolyl)pentyl, 6-(1-methyl-5-tetrazolyl)hexyl, (5-methyl-1-tetrazolyl)methyl, 2-(5-ethyl-1-tetrazolyl)hexyl, 1,1-dimethyl-2-[(1- or 5-) tetrazolyl]ethyl, 2-methyl-3-[(1- or 5-)tetrazolyl] propyl, (1-methyl-5-tetrazolyl)methyl, (1-ethyl-5-tetrazolyl)methyl, 2-(1-n-propyl-5-tetrazolyl)ethyl, 1-(1-n-butyl-5-tetrazolyl)ethyl, 3-(1-n-pentyl-5-tetrazolyl)propyl, 4-(1-n-hexyl-4-tetrazolyl)butyl, 3-(5-isopropyl-1-tetrazolyl)propyl, 4-(5-sec-butyl-1-tetrazolyl)butyl, 5-(5-isopentyl-1-tetrazolyl) pentyl, and 6-(5-n-hexyl-1-tetrazolyl)hexyl.

Examples of benzothienyl lower alkyl groups optionally substituted on the benzothiophene ring with one or more halogen atoms include:

benzothienylalkyl groups wherein the alkyl moiety is a straight and branched C₁₋₆ alkyl group, optionally substituted on the benzothiophene ring with one to three halogen atoms;

such as [(2-, 3-, 4-, 5-, 6-, or 7-)benzothienyl]methyl, 2-[(2-, 3-, 4-, 5-, 6-, or 7-)benzothienyl]ethyl, 1-[(2-, 3-, 4-, 5-, 6-, or 7-)benzothienyl]ethyl, 3-[(2-, 3-, 4-, 5-, 6-, or 7-)benzothienyl]propyl, 4-[(2-, 3-, 4-, 5-, 6-, or 7-)benzothienyl]butyl, 5-[(2-, 3-, 4-, 5-, 6-, or 7-)benzothienyl]pentyl, 6-[(2-, 3-, 4-, 5-, 6-, or 7-)benzothienyl]hexyl, 1,1-dimethyl-2-[(2-, 3-, 4-, 5-, 6-, or 7-)benzothienyl]ethyl, 2-methyl-3-[(2-, 3-, 4-, 5-, 6-, or 7-)benzothienyl]propyl, [5-chloro-(2-, 3-, 4-, 6-, or 7-)benzothienyl]methyl, [4-bromo-(2-, 3-, 5-, 6-, or 7-)benzothienyl]methyl, [6-fluoro-(2-, 3-, 4-, 5-, or 7-)benzothienyl]methyl, [7-iodo-(2-, 3-, 4-, 5-, or 6-)benzothienyl]methyl, [2-chloro-(3-, 4-, 5-, 6-, or 7-)benzothienyl]methyl, [4,5-dichloro-(2-, 3-, 6-, or 7-)benzothienyl]methyl, [2,4,5-chloro-(3-, 6- or 7-)benzothienyl]methyl, 2-[6-fluoro-(2-, 3-, 4-, 5-, or 7-)benzothienyl]ethyl, 1-[7-iodo-(2-, 3-, 4-, 5-, or 6-)benzothienyl]ethyl, 3-[2-chloro-(3-, 4-, 5-, 6-, or 7-)benzothienyl]propyl, 4-[4,5-dichloro-(2-, 3-, 6-, or 7-)benzothienyl]butyl, 5-[2,4,5-trichloro-(3-, 6- or 7-)benzothienyl]pentyl, and 6-[5-chloro-(2-, 3-, 4-, 6-, or 7-)benzothienyl]hexyl.

Examples of lower alkynyl groups include C₂₋₆ straight and branched alkynyl groups, such as ethynyl, 2-propynyl, 2-butynyl, 3-butynyl, 1-methyl-2-propynyl, 2-pentynyl, and 2-hexynyl.

Examples of lower alkenyl groups include straight and branched C₂₋₆ alkenyl groups containing one to three double bonds, such as vinyl, 1-propenyl, 1-methyl-1-propenyl, 2-methyl-1-propenyl, 2-propenyl, 2-butenyl, 1-butenyl, 3-butenyl, 2-penthenyl, 1-penthenyl, 3-penthenyl, 4-penthenyl, 1,3-butadienyl, 1,3-pentadienyl, 2-penten-4-yl, 2-hexenyl, 1-hexenyl, 5-hexenyl, 3-hexenyl, 4-hexenyl, 3,3-dimethyl-1-propenyl, 2-ethyl-1-propenyl, 1,3,5-hexatrienyl, 1,3-hexadienyl, and 1,4-hexadienyl.

Examples of benzoimidazolyl lower alkyl groups include benzoimidazolylalkyl groups wherein the alkyl moiety is a straight or branched C₁₋₆alkyl group, such as [(1-, 2-, 4-, or 5-) benzoimidazolyl]methyl, 2-[(1-, 2-, 4-, or 5-)benzoimidazolyl]ethyl, 1-[(1-, 2-, 4-, or 5-)benzoimidazolyl]ethyl, 3-[(1-, 2-, 4-, or 5-)benzoimidazolyl]propyl, 4-[(1-, 2-, 4-, or 5-)benzoimidazolyl]butyl, 5-[(1-, 2-, 4-, or 5-) benzoimidazolyl]pentyl, 6-[(1-, 2-, 4-, or 5-)benzoimidazolyl] hexyl, 1,1-dimethyl-2-[(1-, 2-, 4-, or 5-)benzoimidazolyl]ethyl, and 2-methyl-3-[(1-, 2-, 4-, or 5-)benzoimidazolyl]propyl.

Examples of pyridyl lower alkyl groups include pyridylalkyl groups wherein the alkyl moiety is a straight or branched C₁₋₆ alkyl group, such as [(2-, 3-, or 4-)pyridyl]methyl, 2-[(2-, 3-, or 4-)pyridyl]ethyl, 1-[(2-, 3-, or 4-)pyridyl]ethyl, 3-[(2-, 3-, or 4-)pyridyl]propyl, 4-[(2-, 3-, or 4-)pyridyl]butyl, 1,1-dimethyl-2-[(2-, 3-, or 4-)pyridyl]ethyl, 5-[(2-, 3-, or 4-) pyridyl]pentyl, 6-[(2-, 3-, or 4-)pyridyl]hexyl, 1-[(2-, 3-, or 4-)pyridyl]isopropyl, and 2-methyl-3-[(2-, 3-, or 4-)pyridyl]propyl.

Examples of imidazolyl lower alkyl groups optionally substituted on the imidazole ring with one or more phenyl lower alkyl groups include:

imidazolylalkyl groups wherein the alkyl moiety is a straight or branched C₁₋₆ alkyl group, optionally substituted on the imidazole ring with one to three above-described phenylalkyl groups wherein the alkyl moiety is a straight or branched C₁₋₆ alkyl group;

such as [(1-, 2-, 4-, or 5-)imidazolyl]methyl, 2-[(1-, 2-, 4-, or 5-)imidazolyl]ethyl, 1-[(1-, 2-, 4-, or 5-) imidazolyl]ethyl, 3-[(1-, 2-, 4-, or 5-)imidazolyl]propyl, 4-[(1-, 2-, 4-, or 5-)imidazolyl]butyl, 1,1-dimethyl-2-[(1-, 2-, 4-, or 5-)imidazolyl]ethyl, 5-[(1-, 2-, 4-, or 5-) imidazolyl]pentyl, 6-[(1-, 2-, 4-, or 5-)imidazolyl]hexyl, 1-[(1-, 2-, 4-, or 5-)imidazolyl]isopropyl, 2-methyl-3-[(1-, 2-, 4-, or 5-)imidazolyl]propyl, [1-benzyl-(2-, 4-, or 5-) imidazolyl]methyl, [1-(2-phenylethyl)-(2-, 4-, or 5-)imidazolyl]methyl, [1-(1-phenylethyl)-(2-, 4-, or 5-)imidazolyl]methyl, [1-(3-phenylpropyl)-(2-, 4-, or 5-)imidazolyl]methyl, [1-(4-phenylbutyl)-(2-, 4-, or 5-)imidazolyl]methyl, [1-(5-phenylpentyl)-(2-, 4-, or 5-)imidazolyl]methyl, [1-(6-phenylhexyl)-(2-, 4-, or 5-)imidazolyl]methyl, 2-[2-benzyl-(1-, 4-, or 5-)imidazolyl]ethyl, 1-[4-(4-phenylethyl)-(1- or 2-)imidazolyl]ethyl, 3-[2-(2-phenylethyl)-(1-, 4-, or 5-)imidazolyl]methyl, 4-[1-(3-phenylpropyl)-(2-, 4-, or 5-)imidazolyl]butyl, 5-[1-(4-phenylbutyl)-(2-, 4-, or 5-)imidazolyl]pentyl, 6-[1-(5-phenylpentyl)-(2-, 4-, or 5-)imidazolyl]hexyl, [1,2-dibenzyl-(4- or 5-)imidazolyl]methyl, and (1,2,4-tribenzyl-5-imidazolyl)methyl.

Examples of lower alkylsulfonyl groups optionally substituted with one or more halogen atoms include straight and branched C₁₋₆ alkylsulfonyl groups optionally substituted with one to three halogen atoms, such as, in addition to the above-described lower alkylsulfonyl groups, trifluoromethylsulfonyl, trichloromethylsulfonyl, chloromethylsulfonyl, bromomethylsulfonyl, fluoromethylsulfonyl, iodomethylsulfonyl, difluoromethylsulfonyl, dibromomethylsulfonyl, 2-chloroethylsulfonyl, 2,2,2-trifluoroethylsulfonyl, 2,2,2-trichloroethylsulfonyl, 3-chloropropylsulfonyl, 2,3-dichloropropylsulfonyl, 4,4,4-trichlorobutylsulfonyl, 4-fluorobutylsulfonyl, 5-chloropentylsulfonyl, 3-chloro-2-methylpropylsulfonyl, 5-bromohexylsulfonyl, and 5,6-dibromohexylsulfonyl.

Examples of alkoxycarbonyl groups optionally substituted with one or more halogen atoms include:

alkoxycarbonyl groups wherein the alkoxy moiety is a straight or branched C₁₋₁₀ alkoxy group, optionally substituted with one to three halogen atoms;

such as, in addition to the above-described lower alkoxycarbonyl groups, n-heptyloxycarbonyl, n-octyloxycarbonyl, n-nonyloxycarbonyl, n-decyloxycarbonyl, 2-ethylhexyloxycarbonyl, trifluoromethoxycarbonyl, trichloromethoxycarbonyl, chloromethoxycarbonyl, bromomethoxycarbonyl, fluoromethoxycarbonyl, iodomethoxycarbonyl, difluoromethoxycarbonyl, dibromomethoxycarbonyl, 2-chloroethoxycarbonyl, 2-fluoroethoxycarbonyl, 2,2,2-trifluoroethoxycarbonyl, 2,2,2-trichloroethoxycarbonyl, 3-chloropropoxycarbonyl, 2,3-dichloropropoxycarbonyl, 4,4,4-trichlorobutoxycarbonyl, 4-fluorobutoxycarbonyl, 4-chlorobutoxycarbonyl, 5-chloropentyloxycarbonyl, 3-chloro-2-methylpropoxycarbonyl, 5-bromohexyloxycarbonyl, 5,6-dibromohexyloxycarbonyl, 7,7,6-trichloroheptyloxycarbonyl, 8-bromooctyloxycarbonyl, 9,9,9-trifluorononyloxycarbonyl, and 10,10,10-trichlorodecyloxycarbonyl.

Examples of pyridylcarbonyl groups optionally substituted on the pyridine ring with one or more members selected from the group consisting of pyrrolyl groups and halogen atoms include:

pyridylcarbonyl groups optionally substituted on the pyridine ring with one to three members selected from the group consisting of pyrrolyl groups and halogen atoms;

such as (2-, 3-, or 4-)pyridylcarbonyl, 2-chloro-(3-, 4-, 5-, or 6-)pyridylcarbonyl, 2,6-dichloro-(3-, 4-, or 5-) pyridylcarbonyl, 2-(1-pyrrolyl)-(3-, 4-, 5-, or 6-) pyridylcarbonyl, 2-bromo-(3-, 4-, 5-, or 6-)pyridylcarbonyl, 2,6-difluoro-(3-, 4-, or 5-)pyridylcarbonyl, 4-(1-pyrrolyl)-(2- or 3-)pyridylcarbonyl, 3-chloro-(2-, 4-, 5-, or 6-) pyridylcarbonyl, 2,5-dibromo-(3-, 4-, or 6-)pyridylcarbonyl, 2-(1-pyrrolyl)-4-chloro-(3-, 5-, or 6-)pyridylcarbonyl, 2,4,6-trifluoro-(3- or 5-)pyridylcarbonyl, and 2,4-di(1-pyrrolyl)-(3-, 5-, or 6-)pyridylcarbonyl.

Examples of pyridyl groups optionally substituted on the pyridine ring with one or more members selected from the group consisting of lower alkyl groups and lower alkoxy groups include:

pyridyl groups optionally substituted on the pyridine ring with one to three members selected from the group consisting of the above-described straight and branched C₁₋₆ alkyl groups and the above-described straight and branched C₁₋₆ alkoxy groups;

such as (2-, 3-, or 4-)pyridyl, 2-methyl-(3-, 4-, 5-, or 6-)pyridyl, 3-methyl-(2-, 4-, 5-, or 6-)pyridyl, 2-methoxy-(3-, 4-, 5-, or 6-)pyridyl, 4-ethyl-(2- or 3-)pyridyl, 3-n-propyl-(2-, 4-, 5-, or 6-)pyridyl, 2-tert-butyl-(3-, 4-, 5-, or 6-)pyridyl, 2-n-pentyl-(3-, 4-, 5-, or 6-)pyridyl, 3-n-hexyl-(2-, 4-, 5-, or 6-)pyridyl, 2,4-dimethyl-(3-, 5-, or 6-)pyridyl, 2,4,6-trimethyl-(3- or 5-)pyridyl, 3-ethoxy-(2-, 4-, 5-, or 6-)pyridyl, 2-isopropoxy-(3-, 4-, 5-, or 6-)pyridyl, 2-n-butoxy-(3-, 4-, 5-, or 6-)pyridyl, 4-n-pentyloxy-(2- or 3-)pyridyl, 2-n-hexyloxy-(3-, 4-, 5-, or 6-) pyridyl, 2,3-dimethoxy-(4-, 5-, or 6-)pyridyl, 3-methyl-(2-, 4-, 5-, or 6-)pyridyl, 3,4,5-trimethoxy-(2- or 6-)pyridyl, and 2-methyl-3-methoxy-(4-, 5-, or 6-)pyridyl.

Examples of amino groups optionally substituted with one or more members selected from the group consisting of lower alkyl groups and lower alkanoyl groups:

include amino groups optionally substituted with one or two members selected from the group consisting of straight and branched C₁₋₆ alkyl groups and straight and branched C₁₋₆ alkanoyl groups;

such as amino, methylamino, ethylamino, n-propylamino, isopropylamino, n-butylamino, tert-butylamino, n-pentylamino, n-hexylamino, dimethylamino, diethylamino, di-n-propylamino, di-n-butylamino, di-n-pentylamino, di-n-hexylamino, N-methyl-N-ethylamino, N-ethyl-N-n-propylamino, N-methyl-N-n-butylamino, N-methyl-N-n-hexylamino, formylamino, acetylamino, propionylamino, butyrylamino, isobutyrylamino, pentanoylamino, tert-butylcarbonylamino, hexanoylamino, N,N-diacetylamino, N-acetyl-N-propionylamino, N-methyl-N-acetylamino, and N-ethyl-N-propionylamino.

Examples of pyrrolidinyl groups optionally substituted on the pyrrolidine ring with one or more oxo groups include pyrrolidinyl groups optionally substituted with one or two oxo groups, such as (1-, 2-, or 3-)pyrrolidinyl, 2-oxo-(1-, 3-, 4-, or 5-)pyrrolidinyl, and 2,5-dioxo-(1- or 3-)pyrrolidinyl.

Examples of piperidinyl groups optionally substituted on the piperidine ring with one or more lower alkyl groups include piperidinyl groups optionally substituted on the piperidine ring with one to three straight and/or branched C₁₋₆ alkyl groups, such as (1-, 2-, 3-, or 4-)piperidinyl, 1-methyl-(2-, 3-, or 4-)piperidinyl, 1-ethyl-(2-, 3-, or 4-)piperidinyl, 1-n-propyl-(2-, 3-, or 4-)piperidinyl, 1-isopropyl-(2-, 3-, or 4-)piperidinyl, 1-n-butyl-(2-, 3-, or 4-)piperidinyl, 1-n-pentyl-(2-, 3-, or 4-)piperidinyl, 1-n-hexyl-(2-, 3-, or 4-) piperidinyl, 1,2-dimethyl-(3-, 4-, 5-, or 6-)piperidinyl, 1,2,3-trimethyl-(4-, 5-, or 6-)piperidinyl, 2-n-propyl-(1-, 3-, 4-, 5- or 6-)piperidinyl, 3-ethyl-(1-, 2-, 4-, 5-, or 6-)piperidinyl, and 2-methyl-4-isopropyl-(1-, 3-, 5-, or 6-)piperidinyl.

Examples of carbamoyl groups optionally substituted with one or more lower alkyl groups include carbamoyl groups optionally substituted with one or two straight and/or branched C₁₋₆alkyl groups, such as carbamoyl, methylcarbamoyl, ethylcarbamoyl, n-propylcarbamoyl, isopropylcarbamoyl, n-butylcarbamoyl, tert-butylcarbamoyl, n-pentylcarbamoyl, n-hexylcarbamoyl, dimethylcarbamoyl, diethylcarbamoyl, di-n-propyl carbamoyl, di-n-butylcarbamoyl, di-n-pentylcarbamoyl, di-n-hexylcarbamoyl, N-methyl-N-ethylcarbamoyl, N-ethyl-N-n-propylcarbamoyl, N-methyl-N-n-butylcarbamoyl, and N-methyl-N-n-hexylcarbamoyl.

Examples of phenyl groups optionally substituted with on the phenyl ring one or more members selected from the group consisting of halogen atoms; lower alkyl groups optionally substituted with one or more halogen atoms; a phenoxy group; lower alkoxy groups optionally substituted with one or more halogen atoms; lower alkylthio groups; lower alkylsulfonyl groups; amino groups optionally substituted with one or more members selected from the group consisting of lower alkyl groups and lower alkanoyl groups; pyrrolidinyl groups optionally substituted on the pyrrolidine ring with one or more oxo groups; piperidinyl groups optionally substituted on the piperidine ring with one or more lower alkyl groups; lower alkenyl groups; an aminosulfonyl group; a hydroxy group; carbamoyl groups optionally substituted with one or more lower alkyl groups; phenyl lower alkoxy groups; and a cyano group include:

phenyl groups optionally substituted on the phenyl ring with one to three members selected from the group consisting of the above-described halogen atoms; the above-described straight and branched C₁₋₆ alkyl groups optionally substituted with one to three halogen atoms; a phenoxy group; the above-described straight and branched C₁₋₆ alkoxy groups optionally substituted with one to three halogen atoms; the above-described straight and branched C₁₋₆ alkylthio groups; the above-described straight and branched C₁₋₆ alkylsulfonyl groups; the above-described amino groups optionally substituted with one or two members selected from the group consisting of straight and branched C₁₋₆ alkyl groups and straight and branched C₁₋₆ alkanoyl groups; the above-described pyrrolidinyl groups optionally substituted on the pyrrolidine ring with one or two oxo groups; the above-described piperidinyl groups optionally substituted on the piperidine ring with one to three straight and/or branched C₁₋₆ alkyl groups; the above-described straight and branched C₂₋₆ alkenyl groups containing one to three double bonds; an aminosulfonyl group; a hydroxy group; the above-described carbamoyl groups optionally substituted with one or two straight and/or branched C₁₋₆ alkyl groups; the above-described phenylalkoxy groups wherein the alkoxy moiety is a straight or branched C₁₋₆ alkoxy group; and a cyano group;

such as phenyl, 4-phenoxyphenyl, 3-phenoxyphenyl, 2-phenoxyphenyl, 4-isopropylphenyl, 3-isopropylphenyl, 2-isopropylphenyl, 4-tert-butylphenyl, 4-methylphenyl, 3-methylphenyl, 2-methylphenyl, 2,3-dimethylphenyl, 2,4-dimethylphenyl, 3,5-dimethylphenyl, 2,4,6-trimethylphenyl, 4-methyl-3-methoxyphenyl, 4-trifluoromethylphenyl, 3-trifluoromethylphenyl, 2-trifluoromethylphenyl, 4-methyl-3-chlorophenyl, 4-chlorophenyl, 3-chlorophenyl, 2-chlorophenyl, 2-fluorophenyl, 3-fluorophenyl, 4-fluorophenyl, 3-bromophenyl, 3,4-dichlorophenyl, 3,5-dichlorophenyl, 3,4,5-trichlorophenyl, 2,4,6-trifluorophenyl, 3,5-difluorophenyl, 3-chloro-4-fluorophenyl, 2-chloro-5-fluorophenyl, 3-fluoro-4-methoxyphenyl, 3-chloro-4-methoxyphenyl, 3-chloro-4-hydroxyphenyl, 4-methoxyphenyl, 3-methoxyphenyl, 2-methoxyphenyl, 2,4-dimethoxyphenyl, 3,4-dimethoxyphenyl, 2,4,6-trimethoxyphenyl, 2-methoxy-5-chlorophenyl, 2-methoxy-5-acetylaminophenyl, 2-chloro-5-acetylaminophenyl, 4-ethoxyphenyl, 4-trifluoromethoxyphenyl, 3-trifluoromethoxyphenyl, 2-trifluoromethoxyphenyl, 3-methoxy-5-trifluoromethylphenyl, 4-methylthiophenyl, 3-methylthiophenyl, 2-methylthiophenyl, 2-(1-methyl-1-vinyl)phenyl, 4-vinylphenyl, 3-dimethylaminophenyl, 4-methylaminophenyl, 2-(N-methyl-N-acetylamino)phenyl, 3-acetylaminophenyl, 4-propionylaminophenyl, 4-acetylaminophenyl, 2-acetylaminophenyl, 4-aminosulfonylphenyl, 3-aminosulfonylphenyl, 2-aminosulfonylphenyl, 4-methylthiophenyl, 3-methylthiophenyl, 2-methylthiophenyl, 4-methylsulfonylphenyl, 3-methylsulfonylphenyl, 2-methylsulfonylphenyl, 4-methylcarbamoylphenyl, 3-carbamoylphenyl, 2-ethylcarbamoylphenyl, 2-benzyloxyphenyl, 3-benzyloxyphenyl, 4-benzyloxyphenyl, 2-phenylphenyl, 3-phenylphenyl, 4-phenylphenyl, 2-cyanophenyl, 3-cyanophenyl, 4-cyanophenyl, 4-[2-oxo-(1-, 3-, 4-, or 5-)pyrrolidinyl]phenyl, 3-[2,5-dioxo-(1- or 3-)pyrrolidinyl]phenyl, 4-[4-methyl-(1-, 2-, or 3-)piperazinyl]phenyl, 3-[4-ethyl-(1-, 2-, or 3-) piperazinyl]phenyl, and 2-[4-isopropyl-(1-, 2-, or 3-) piperazinyl]phenyl.

Examples of cycloalkyl groups optionally substituted on the cycloalkyl ring with one or more lower alkyl groups include C₃₋₈ cycloalkyl groups optionally substituted on the cycloalkyl ring with one to three straight and/or branched C₁₋₆ alkyl groups, such as, in addition to the above-described cycloalkyl groups, 1-methylcyclopropyl, 1-methylcyclopentyl, 1-methylcyclohexyl, 2-methylcyclohexyl, 1-methylcyclobutyl, 1-ethylcyclooctyl, 1-n-propylcycloheptyl, 1,2-dimethylcyclohexyl, 1,4,5-trimethylcyclooctyl, 1-n-butylcyclopropyl, 1-n-pentylcyclopentyl, and 1-n-hexylcyclohexyl.

Examples of amino groups optionally substituted with one or more members selected from the group consisting of a phenyl group and lower alkyl groups include:

amino groups optionally substituted with one or two members selected from the group consisting of a phenyl group and straight and branched C₁₋₆ alkyl groups;

such as amino, methylamino, ethylamino, n-propylamino, isopropylamino, n-butylamino, tert-butylamino, n-pentylamino, n-hexylamino, dimethylamino, diethylamino, di-n-propylamino, di-n-butylamino, di-n-pentylamino, di-n-hexylamino, N-methyl-N-ethylamino, N-ethyl-N-n-propylamino, N-methyl-N-n-butylamino, N-methyl-N-n-hexylamino, phenylamino, N,N-diphenylamino, N-methyl-N-phenylamino, N-ethyl-N-phenylamino, and N-n-propyl-N-phenylamino.

Examples of benzoyl groups optionally substituted on the phenyl ring with one or more members selected from the group consisting of halogen atoms; a phenoxy group; a phenyl group; lower alkyl groups optionally substituted with one or more halogen atoms; lower alkoxy groups; lower alkanoyl groups; a nitro group; a cyano group; amino groups optionally substituted with one or more members selected from the group consisting of a phenyl group and lower alkyl groups; pyrrolidinyl groups optionally substituted on the pyrrolidine ring with one or more oxo groups; pyrrolyl groups; pyrazolyl groups; 1,2,4-triazolyl groups; and imidazolyl groups include:

benzoyl groups optionally substituted on the phenyl ring with one to three members selected from the group consisting of halogen atoms; a phenoxy group; a phenyl group; the above-described straight and branched C₁₋₆ alkyl groups optionally substituted with one to three halogen atoms; the above-described straight and branched C₁₋₆ alkoxy groups; the above-described straight and branched C₁₋₆ alkanoyl groups; a nitro group; a cyano group; the above-described amino groups optionally substituted with one or two members selected from the group consisting of a phenyl group and straight and branched C₁₋₆ alkyl groups; the above-described pyrrolidinyl groups optionally substituted on the pyrrolidine ring with one or two oxo groups; pyrrolyl groups; pyrazolyl groups; 1,2,4-triazolyl groups; and imidazolyl groups;

such as benzoyl, 4-methoxybenzoyl, 3-methoxybenzoyl, 2-methoxybenzoyl, 2,4-dimethoxybenzoyl, 3,4,5-trimethoxybenzoyl, 2-methoxy-5-chlorobenzoyl, 4-phenoxybenzoyl, 2-phenoxybenzoyl, 3-phenoxybenzoyl, 4-chlorobenzoyl, 3-chlorobenzoyl, 2-chlorobenzoyl, 2,6-dichlorobenzoyl, 2-chloro-4-fluorobenzoyl, 2,4,6-trifluorobenzoyl, 4-bromobenzoyl, 3-fluorobenzoyl, 4-trifluoromethylbenzoyl, 3-trifluoromethylbenzoyl, 2-trifluoromethylbenzoyl, 3-fluoro-2-methylbenzoyl, 4-methylbenzoyl, 3-methylbenzoyl, 2-methylbenzoyl, 3,4-dimethylbenzoyl, 2,4,5-trimethylbenzoyl, 2-phenylbenzoyl, 3-phenylbenzoyl, 4-phenylbenzoyl, 4-nitrobenzoyl, 3-nitrobenzoyl, 2-nitrobenzoyl, 2-dimethylaminobenzoyl, 3-methylaminobenzoyl, 4-(N-methylanilino)benzoyl, 2-anilinobenzoyl, 3-cyanobenzoyl, 4-cyanobenzoyl, 2-cyanobenzoyl, 4-acetylbenzoyl, 2-propionylbenzoyl, 3-butyrylbenzoyl, 4-[(1-, 2-, or 3-)pyrrolyl]benzoyl, 4-[(1-, 3-, 4-, or 5-)pyrazolyl]benzoyl, 4-[(1-, 3- or 5-)1,2,4-triazolyl]benzoyl, 4-[(1-, 2-, 4-, or 5-)imidazolyl]benzoyl, and 4-[2-oxo-(1-, 3-, 4-, or 5-)pyrrolidinyl]benzoyl.

Examples of lower alkylenedioxy groups include straight and branched C₁₋₄ alkylene groups, such as methylenedioxy, ethylenedioxy, trimethylenedioxy, and tetramethylenedioxy.

Examples of benzoyl groups substituted on the phenyl ring with one or more lower alkylenedioxy groups include:

benzoyl groups substituted on the phenyl ring with one or more of the above-described straight and branched C₁₋₄ alkylenedioxy groups;

such as 3,4-methylenedioxybenzoyl, 2,3-ethylenedioxybenzoyl, 3,4-trimethylenedioxybenzoyl, and 2,3-tetramethylenedioxybenzoyl.

Examples of cycloalkylcarbonyl groups include cycloalkylcarbonyl groups wherein the cycloalkyl moiety is a C₃₋₈ cycloalkyl group, such as cyclopropylcarbonyl, cyclobutylcarbonyl, cyclopentylcarbonyl, cyclohexylcarbonyl, cycloheptylcarbonyl, and cyclooctylcarbonyl.

Examples of furylcarbonyl groups include (2- or 3-) furylcarbonyl.

Examples of naphthylcarbonyl groups include (1- or 2-) naphthylcarbonyl.

Examples of phenoxycarbonyl groups optionally substituted on the phenyl ring with one or more members selected from the group consisting of lower alkoxy groups, lower alkyl groups, halogen atoms, and a nitro group include:

phenoxycarbonyl groups optionally substituted on the phenyl ring with one to three members selected from the group consisting of the above-described straight and branched C₁₋₆ alkoxy groups, the above-described straight and branched C₁₋₆ alkyl groups, halogen atoms, and a nitro group;

such as phenoxycarbonyl, 4-chlorophenoxycarbonyl, 3-chlorophenoxycarbonyl, 2-chlorophenoxycarbonyl, 3,4-dichlorophenoxycarbonyl, 2,4,6-trichlorophenoxycarbonyl, 4-fluorophenoxycarbonyl, 3-fluorophenoxycarbonyl, 2-fluorophenoxycarbonyl, 2,4-difluorophenoxycarbonyl, 3,4,5-trifluorophenoxycarbonyl, 4-bromophenoxycarbonyl, 2-chloro-4-methoxyphenoxycarbonyl, 3-fluoro-5-methylphenoxycarbonyl, 4-methoxyphenoxycarbonyl, 3-methoxyphenoxycarbonyl, 2-methoxyphenoxycarbonyl, 3,4-dimethoxyphenoxycarbonyl, 2,4,5-trimethoxyphenoxycarbonyl, 4-methylphenoxycarbonyl, 3-methylphenoxycarbonyl, 2-methylphenoxycarbonyl, 2,5-dimethylphenoxycarbonyl, 2,3,4-trimethylphenoxycarbonyl, 4-nitrophenoxycarbonyl, 3-nitrophenoxycarbonyl, 2-nitrophenoxycarbonyl, 2,4-dinitrophenoxycarbonyl, and 2,4,6-trinitrophenoxycarbonyl.

Examples of phenyl lower alkoxycarbonyl groups optionally substituted on the phenyl ring with one or more members selected from the group consisting of halogen atoms and a nitro group include:

phenylalkoxycarbonyl groups wherein the alkoxy moiety is a straight or branched C₁₋₆ alkoxy group, optionally substituted on the phenyl ring with one to three members selected from the group consisting of halogen atoms and a nitro group;

such as benzyloxycarbonyl, 2-phenylethoxycarbonyl, 1-phenylethoxycarbonyl, 3-phenylpropoxycarbonyl, 4-phenylbutoxycarbonyl, 5-phenylpentyloxycarbonyl, 6-phenylhexyloxycarbonyl, 1,1-dimethyl-2-phenylethoxycarbonyl, 2-methyl-3-phenylpropoxycarbonyl, 4-chlorobenzyloxycarbonyl, 3-chlorobenzyloxycarbonyl, 2-chlorobenzyloxycarbonyl, 3,4-dichlorobenzyloxycarbonyl, 2,4,6-trichlorobenzyloxycarbonyl, 4-fluorobenzyloxycarbonyl, 3-fluorobenzyloxycarbonyl, 2-fluorobenzyloxycarbonyl, 2,4-difluorobenzyloxycarbonyl, 3,4,5-trifluorobenzyloxycarbonyl, 4-bromobenzyloxycarbonyl, 4-nitrobenzyloxycarbonyl, 3-nitrobenzyloxycarbonyl, 2-nitrobenzyloxycarbonyl, 2,4-dinitrobenzyloxycarbonyl, 2,4,6-trinitrobenzyloxycarbonyl, and 2-nitro-4-chlorobenzyloxycarbonyl.

Examples of piperidinyl groups optionally substituted on the piperidine ring with one or more members selected from the group consisting of lower alkyl groups; lower alkanoyl groups; benzoyl groups optionally substituted on the phenyl ring with one or more halogen atoms; and phenyl groups optionally substituted on the phenyl ring with one or more halogen atoms include:

piperidinyl groups optionally substituted on the piperidine ring with one to three members selected from the group consisting of the above-described straight and branched C₁₋₆ alkyl groups; the above-described straight and branched C₁₋₆ alkanoyl groups; the above-described benzoyl groups optionally substituted on the phenyl ring with one to three halogen atoms; and the above-described phenyl groups optionally substituted on the phenyl ring with one to three halogen atoms;

such as (1-, 2-, 3-, or 4-)piperidinyl, 1-methyl-(2-, 3-, or 4-)piperidinyl, 1-acetyl-(2-, 3-, or 4-)piperidinyl, 1-benzoyl-(2-, 3-, or 4-)piperidinyl, 1-(4-chlorobenzoyl)-(2-, 3-, or 4-)piperidinyl, 1-(3-bromobenzoyl)-(2-, 3-, or 4-) piperidinyl, 1-benzoyl-(2-, 3-, or 4-)piperidinyl, 1-(4-fluorobenzoyl)-(2-, 3-, or 4-)piperidinyl, 1-(2,4-dichloro benzoyl)-(2-, 3-, or 4-)piperidinyl, 1-(2,4,6-trifluorobenzoyl)-(2-, 3-, or 4-)piperidinyl, 2-(3-chlorobenzoyl)-(1-, 3-, or 4-) piperidinyl, 3-(2-chlorobenzoyl)-(1-, 2-, or 4-)piperidinyl, 4-(2,3-dibromobenzoyl)-(1-, 2-, or 3-)piperidinyl, 1,2-dibenzoyl-(3- or 4-)piperidinyl, 1,2,4-tribenzoyl-3-piperidinyl, 1,4-dimethyl-(2-, 3-, 5-, or 6-)piperidinyl, 1,2,4-trimethyl-(3-, 5-, or 6-)piperidinyl, 1-benzoyl-2-methyl-(3-, 4-, 5-, or 6-)piperidinyl, 1-phenyl-2-methyl-(3-, 4-, 5-, or 6-)piperidinyl, 1-acetyl-3-methyl-(2-, 4-, 5-, or 6-)piperidinyl, 1-phenyl-(2-, 3-, or 4-) piperidinyl, 1-(4-chlorophenyl)-(2-, 3-, or 4-)piperidinyl, 1-(3-bromophenyl)-(2-, 3-, or 4-)piperidinyl, 1-(4-iodophenyl)-(2-, 3-, or 4-)piperidinyl, 1-(4-fluorophenyl)-(2-, 3-, or 4-) piperidinyl, 1-(2,4-dichlorophenyl)-(2-, 3-, or 4-)piperidinyl, 1-(2,4,6-trifluorophenyl)-(2-, 3-, or 4-)piperidinyl, 2-(3-chlorophenyl)-(1-, 3-, 4-, 5-, or 6-)piperidinyl, 3-(2-chlorophenyl)-(1-, 2-, 4-, 5-, or 6-)piperidinyl, 4-(2,3-dibromophenyl)-(1-, 2-, or 3-)piperidinyl, 1,2-diphenyl-(3-, 4-, 5- or 6-)piperidinyl, and 1,2,4-triphenyl-(3-, 5-, or 6-) piperidinyl.

Examples of tetrahydropyranyl lower alkyl groups include tetrahydropyranylalkyl groups wherein the alkyl moiety is a straight or branched C₁₋₆ alkyl group, such as [(2-, 3-, or 4-)tetrahydropyranyl]methyl, 2-[(2-, 3-, or 4-) tetrahydropyranyl]ethyl, 1-[(2-, 3-, or 4-)tetrahydropyranyl]ethyl, 3-[(2-, 3-, or 4-)tetrahydropyranyl]propyl, 4-[(2-, 3-, or 4-)tetrahydropyranyl]butyl, 1,1-dimethyl-2-[(2-, 3-, or 4-)tetrahydropyranyl]ethyl, 5-[(2-, 3-, or 4-) tetrahydropyranyl]pentyl, 6-[(2-, 3-, or 4-)tetrahydropyranyl] hexyl, 1-[(2-, 3- or 4-)tetrahydropyranyl]isopropyl, and 2-methyl-3-[(2-, 3-, or 4-)tetrahydropyranyl]propyl.

Examples of phenyl lower alkyl groups optionally substituted on the alkyl group with one or more lower alkoxycarbonyl groups; and optionally further substituted on the phenyl ring with one or more members selected from the group consisting of halogen atoms, lower alkyl groups optionally substituted with one or more halogen atoms, lower alkoxy groups optionally substituted with one or more halogen atoms, and a hydroxy group include:

mono- and di-phenylalkyl groups wherein the alkyl moiety is a straight or branched C₁₋₆ alkyl group, optionally substituted on the alkyl group with one or more lower alkoxycarbonyl groups wherein the alkoxy moiety is a straight or branched C₁₋₆ alkoxy group; and optionally further substituted on the phenyl group with one to three members selected from the group consisting of halogen atoms, the above-described straight and branched C₁₋₆ alkyl groups optionally substituted with one to three halogen atoms, the above-described straight and branched C₁₋₆ alkoxy groups optionally substituted with one to three halogen atoms, and a hydroxy group;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-dimethyl-1-phenylmethyl, 1,1-diphenylmethyl, 2,2-diphenylethyl, 3,3-diphenylpropyl, 1,2-diphenylethyl, 4-chlorobenzyl, 2-chlorobenzyl, 3-chlorobenzyl, 3-fluorobenzyl, 4-fluorobenzyl, 3-bromobenzyl, 2,3-dichlorobenzyl, 2,6-dichlorobenzyl, 2,4,6-trifluorobenzyl, 2-(4-chlorophenyl)ethyl, 2-(2-fluorophenyl)ethyl, 2-(3-fluorophenyl)ethyl, 3-trifluoromethylbenzyl, 4-trifluoromethylbenzyl, 2-methylbenzyl, 3-methylbenzyl, 4-methylbenzyl, 4-tert-butylbenzyl, 2,4-dimethylbenzyl, 2,4,6-trimethylbenzyl, 2-trifluoromethoxybenzyl, 3-trifluoromethoxybenzyl, 4-trifluoromethoxybenzyl, 2-methoxybenzyl, 3-methoxybenzyl, 4-methoxybenzyl, 4-ethoxybenzy, 2-(3-methoxyphenyl)ethyl, 3,4-dimethoxybenzyl, 3,4,5-trimethoxybenzyl, 4-hydroxybenzyl, 3-hydroxybenzyl, 2-hydroxybenzyl, 2,4-dihydroxybenzyl, 3,4,5-trihydroxybenzyl, 2-methoxy-4-chlorobenzyl, 3-methyl-5-fluorobenzyl, 2-(4-hydroxyphenyl)-1-methoxycarbonylethyl, and 2-(4-chlorophenyl)-1-ethoxycarbonylethyl.

Examples of lower alkylenedioxy-substituted phenyl lower alkyl groups include:

alkylenedioxy-substituted phenylalkyl groups wherein the alkyl moiety is a straight or branched C₁₋₆ alkyl group, substituted on the phenyl ring with one or more of the above-described straight and branched C₁₋₄ alkylenedioxy groups;

such as 3,4-methylenedioxybenzyl, 3,4-trimethylenedioxybenzyl, 2-(2,3-ethylenedioxyphenyl)ethyl, 1-(3,4-trimethylenedioxyphenyl)ethyl, 3-(2,3-tetramethylenedioxyphenyl)propyl, 4-(3,4-methylenedioxyphenyl) butyl, 5-(2,3-ethylenedioxyphenyl)pentyl, 6-(3,4-trimethylenedioxyphenyl)hexyl, 1,1-dimethyl-2-(2,3-methylenedioxyphenyl)ethyl, and 2-methyl-3-(3,4-ethylenedioxyphenyl)propyl.

Examples of furyl lower alkyl groups include furylalkyl groups wherein the alkyl moiety is a straight or branched C₁₋₆ alkyl group, such as [(2- or 3-)furyl]methyl, 2-[(2- or 3-)furyl]ethyl, 1-[(2- or 3-)furyl]ethyl, 3-[(2- or 3-)furyl]propyl, 4-[(2- or 3-)furyl]butyl, 5-[(2- or 3-)furyl]pentyl, 6-[(2- or 3-) furyl]hexyl, 1,1-dimethyl-2-[(2- or 3-)furyl]ethyl, and 2-methyl-3-[(2- or 3-)furyl]propyl.

Examples of carbamoyl lower alkyl groups optionally substituted with one or more members selected from the group consisting of lower alkyl groups and a phenyl group, each phenyl substituent optionally being substituted on the phenyl ring with one or more lower alkyl groups, include:

carbamoylalkyl groups wherein the alkyl moiety is a straight or branched C₁₋₆ alkyl group, optionally substituted with one or two members selected from the group consisting of the above-described straight and branched C₁₋₆ alkyl groups and the above-described phenyl groups optionally substituted on the phenyl ring with one to three straight and/or branched C₁₋₆ alkyl groups;

such as carbamoylmethyl, 2-carbamoylethyl, 1-carbamoylethyl, 3-carbamoylpropyl, 4-carbamoylbutyl, 5-carbamoylpentyl, 6-carbamoylhexyl, 1,1-dimethyl-2-carbamoylethyl, 2-methyl-3-carbamoylpropyl, 2-(N-methyl-N-phenylcarbamoyl)ethyl, N-(4-methylphenyl)carbamoylmethyl, 2-[N-methyl-N-(3-methylphenyl) carbamoyl]ethyl, N-(2-methylphenyl)carbamoylmethyl, 2-[N-ethyl-N-(3,4-dimethylphenyl)carbamoyl]ethyl, N-(2,4,6-trimethylphenyl) carbamoylmethyl, N,N-dimethylcarbamoylmethyl, N,N-diphenylcarbamoylmethyl, N-methyl-N-ethylcarbamoylmethyl, N-methylcarbamoylmethyl, and 2-(N-methylcarbamoyl)ethyl.

Examples of imidazolyl lower alkyl groups optionally substituted on the lower alkyl group with one or more members selected from the group consisting of a carbamoyl group and lower alkoxycarbonyl groups include:

imidazolylalkyl groups wherein the alkyl moiety is a straight or branched C₁₋₆ alkyl group, optionally substituted on the lower alkyl group with one or more members selected from the group consisting of a carbamoyl group and alkoxycarbonyl groups wherein the alkoxy moiety is a straight or branched C₁₋₆ alkoxy group;

such as, in addition to the above-described imidazolyl lower alkyl groups, 1-carbamoyl-2-[(1-, 2-, 4-, or 5-)imidazolyl]ethyl, 1-methoxycarbonyl-2-[(1-, 2-, 4-, or 5-)imidazolyl]ethyl, 1-carbamoyl-1-[(1-, 2-, 4-, or 5-)imidazolyl]methyl, 1-ethoxycarbonyl-1-[(1-, 2-, 4-, or 5-)imidazolyl]methyl, 1-carbamoyl-3-[(1-, 2-, 4-, or 5-)imidazolyl]propyl, 1-n-propoxycarbonyl-4-[(1-, 2-, 4-, or 5-)imidazolyl]butyl, 1-carbamoyl-5-[(1-, 2-, 4-, or 5-)imidazolyl]pentyl, and 1-tert-butoxycarbonyl-6-[(1-, 2-, 4-, or 5-)imidazolyl]hexyl.

Examples of amino-substituted lower alkyl groups optionally substituted on each amino group with one or more lower alkyl groups include:

amino-substituted straight and branched C₁₋₆ alkyl groups optionally substituted on the amino group with one or two straight and/or branched C₁₋₆alkyl groups;

such as aminomethyl, 2-aminoethyl, 1-aminoethyl, 3-aminopropyl, 4-aminobutyl, 5-aminopentyl, 6-aminohexyl, 1,1-dimethyl-2-aminoethyl, 2-methyl-3-aminopropyl, methylaminomethyl, 2-ethylaminoethyl, 3-n-propylaminopropyl, 3-isopropylaminopropyl, 4-n-butylaminobutyl, 5-n-pentylaminopentyl, 6-n-hexylaminohexyl, dimethylaminoethyl, 2-diisopropylaminopropyl, 3-diisopropylaminopropyl, (N-ethyl-N-n-propylamino)methyl, and 2-(N-methyl-N-n-hexylamino)methyl.

Examples of 2,3,4,5-tetrahydrofuryl groups optionally substituted on the 2,3,4,5-tetrahydrofuran ring with one or more oxo groups include:

2,3,4,5-tetrahydrofuryl groups optionally substituted on the 2,3,4,5-tetrahydrofuran ring with one or two oxo groups;

such as (2- or 3-)2,3,4,5-tetrahydrofuryl, 2-oxo-(3-, 4-, or 5-)2,3,4,5-tetrahydrofuryl, 3-oxo-(2-, 4-, or 5-) 2,3,4,5-tetrahydrofuryl, and 2,5-dioxo-(3- or 4-)2,3,4,5-tetrahydrofuryl.

Examples of pyrrolidinyl lower alkyl groups optionally substituted on the pyrrolidine ring with one or more lower alkyl groups include:

pyrrolidinylalkyl groups wherein the alkyl moiety is a straight or branched C₁₋₆ alkyl group, optionally substituted on the pyrrolidine ring with one to three above-described straight and/or branched C₁₋₆ alkyl groups;

such as [(1-, 2-, or 3-)pyrrolidinyl]methyl, 2-[(1-, 2-, or 3-)pyrrolidinyl]ethyl, 1-[(1-, 2-, or 3-) pyrrolidinyl]ethyl, 3-[(1-, 2-, or 3-)pyrrolidinyl]propyl, 4-[(1-, 2-, or 3-)pyrrolidinyl]butyl, 5-[(1-, 2-, or 3-) pyrrolidinyl]pentyl, 6-[(1-, 2-, or 3-)pyrrolidinyl]hexyl, 1,1-dimethyl-2-[(1-, 2-, or 3-)pyrrolidinyl]ethyl, 2-methyl-3-[(1-, 2-, or 3-)pyrrolidinyl]propyl, 1-ethyl-[(2- or 3-) pyrrolidinyl]methyl, 1-ethyl-[(2- or 3-)pyrrolidinyl]methyl, 2-methyl-[(1-, 3-, 4-, or 5-)pyrrolidinyl]methyl, 3-n-propyl-[(1-, 2-, 4-, or 5-)pyrrolidinyl]methyl, 1-n-butyl-[(2- or 3-) pyrrolidinyl]methyl, 2-n-pentyl-[(1-, 3-, 4-, or 5-)pyrrolidinyl]methyl, 1-n-hexyl-[(2- or 3-)pyrrolidinyl]methyl, 1,2-dimethyl-[(3-, 4-, or 5-) pyrrolidinyl]methyl, and 1,2,3-trimethyl-[(4- or 5-)pyrrolidinyl]methyl.

Examples of phenoxy lower alkanoyl groups include phenoxyalkanoyl groups wherein the alkanoyl moiety is a straight or branched C₂₋₆ alkanoyl group, such as 2-phenoxyacetyl, 3-phenoxypropionyl, 2-phenoxypropionyl, 4-phenoxybutyryl, 5-phenoxypentanoyl, 6-phenoxyhexanoyl, 2,2-dimethyl-3-phenoxypropionyl, and 2-methyl-3-phenoxypropionyl.

Examples of morpholino lower alkyl groups include morpholinoalkyl groups wherein the alkyl moiety is a straight or branched C₁₋₆ alkyl group, such as [(2-, 3-, or 4-) morpholino]methyl, 2-[(2-, 3-, or 4-)morpholino]ethyl, 1-[(2-, 3-, or 4-)morpholino]ethyl, 3-[(2-, 3-, or 4-) morpholino]propyl, 4-[(2-, 3-, or 4-)morpholino]butyl, 5-[(2-, 3-, or 4-)morpholino]pentyl, 6-[(2-, 3-, or 4-) morpholino]hexyl, 1,1-dimethyl-2-[(2-, 3-, or 4-)morpholino]ethyl, and 2-methyl-3-[(2-, 3-, or 4-)morpholino]propyl.

Examples of pyridyl lower alkanoyl groups include pyridylalkanoyl groups wherein the alkanoyl moiety is a straight or branched C₂₋₆ alkanoyl group, such as 2-[(2-, 3-, or 4-)pyridyl]acetyl, 3-[(2-, 3-, or 4-)pyridyl] propionyl, 2-[(2-, 3-, or 4-)pyridyl]propionyl, 4-[(2-, 3-, or 4-)pyridyl]butyryl, 5-[(2-, 3-, or 4-)pyridyl] pentanoyl, 6-[(2-, 3-, or 4-)pyridyl]hexanoyl, 2,2-dimethyl-3-[(2-, 3-, or 4-)pyridyl]propionyl, and 2-methyl-3-[(2-, 3-, or 4-)pyridyl]propionyl.

Examples of thienylcarbonyl groups include 2-thienylcarbonyl and 3-thienylcarbonyl.

Examples of thienyl lower alkanoyl groups include thienylalkanoyl groups wherein the alkanoyl moiety is a straight or branched C₂₋₆ alkanoyl group, such as 2-[(2- or 3-) thienyl]acetyl, 3-[(2- or 3-)thienyl]propionyl, 2-[(2- or 3-) thienyl]propionyl, 4-[(2- or 3-)thienyl]butyryl, 5-[(2- or 3-) thienyl]pentanoyl, 6-[(2- or 3-)thienyl]hexanoyl, 2,2-dimethyl-3-[(2- or 3-)thienyl]propionyl, and 2-methyl-3-[(2- or 3-)thienyl] propionyl.

Examples of cycloalkyl lower alkanoyl groups include C₃₋₈ cycloalkylalkanoyl groups wherein the alkanoyl moiety is a straight or branched C₂₋₆ alkanoyl group, such as 2-cyclopropylacetyl, 2-cyclohexylacetyl, 3-cyclopropylpropionyl, 2-cyclobutylpropionyl, 2-cyclopentylacetyl, 3-cyclopentylpropionyl, 4-cyclohexylbutyryl, 5-cycloheptylpentanoyl, 6-cyclooctylhexanoyl, 2,2-dimethyl-3-cyclohexylpropionyl, and 2-methyl-3-cyclopropylpropionyl.

Examples of isoxazolylcarbonyl groups optionally substituted on the isoxazole ring with one or more lower alkyl groups include isoxazoylcarbonyl groups optionally substituted on the isoxazole ring with one or two straight and/or branched C₁₋₆ alkyl groups, such as (3-, 4-, or 5-)isoxazolylcarbonyl, [3,5-dimethyl-4-isoxazolyl]carbonyl, [3-ethyl-(4- or 5-) isoxazolyl]carbonyl, [4-n-propyl-(3- or 5-)isoxazolyl]carbonyl, [5-n-butyl-(3- or 4-)isoxazolyl]carbonyl, [3-n-pentyl-(4- or 5-) isoxazolyl]carbonyl, and [4-n-hexyl-(3- or 5-)isoxazolyl]carbonyl.

Examples of pyrazylcarbonyl groups include 2-pyrazylcarbonyl.

Examples of piperidinylcarbonyl groups optionally substituted on the piperidine ring with one or more members selected from the group consisting of a benzoyl group and lower alkanoyl groups include:

piperidinylcarbonyl groups optionally substituted on the piperidine ring with one to three members selected from the group consisting of a benzoyl group and the above-described straight and branched C₁₋₆ alkanoyl groups;

such as (1-, 2-, 3-, or 4-)piperidinylcarbonyl, [1-acetyl-(2-, 3-, or 4-)piperidinyl]carbonyl, [1-benzoyl-(2-, 3-, or 4-)piperidinyl]carbonyl, [2-propionyl-(1-, 3-, 5-, or 6-)piperidinyl]carbonyl, [3-butyryl-(1-, 2-, 5-, or 6-)piperidinyl]carbonyl, [4-pentanoyl-(1-, 2-, or 3-)piperidinyl]carbonyl, [1-hexanoyl-(2-, 3-, or 4-) piperidinyl]carbonyl, [1-acetyl-4-benzoyl-(2-, 3-, 5-, or 6-) piperidinyl]carbonyl, and [1,2,4-triacetyl-(3-, 5-, or 6-) piperidinyl]carbonyl.

Examples of chromanylcarbonyl groups include 2-chromanylcarbonyl, 3-chromanylcarbonyl, 4-chromanylcarbonyl, 5-chromanylcarbonyl, 6-chromanylcarbonyl, 7-chromanylcarbonyl, and 8-chromanylcarbonyl.

Examples of isoindolinyl lower alkanoyl groups optionally substituted on the isoindoline ring with one or more oxo groups include:

isoindolinyl lower alkanoyl groups wherein the alkanoyl moiety is a straight or branched C₂₋₆ alkanoyl group, optionally substituted on the isoindoline ring with one or two oxo groups;

such as 2-[(1-, 2-, 4-, or 5-)isoindolinyl]acetyl, 3-[(1-, 2-, 4-, or 5-)isoindolinyl]propionyl, 2-[(1-, 2-, 4-, or 5-)isoindolinyl]propionyl, 4-[(1-, 2-, 4-, or 5-)isoindolinyl]butyryl, 5-[(1-, 2-, 4-, or 5-) isoindolinyl]pentanoyl, 6-[(1-, 2-, 4-, or 5-)isoindolinyl] hexanoyl, 2,2-dimethyl-3-[(1-, 2-, 4-, or 5-)isoindolinyl] propionyl, 2-methyl-3-[(1-, 2-, 4-, or 5-)isoindolinyl]propionyl, [1,3-dioxo-2-(2-, 4-, or 5-)isoindolinyl]acetyl, and [1-oxo-2-(2-, 3-, 4-, 5-, 6-, or 7-)isoindolinyl]acetyl.

Examples of thiazolidinyl lower alkanoyl groups optionally substituted on the thiazolidine ring with one or more members selected from the group consisting of an oxo group and a thioxo group include:

thiazolidinylalkanoyl groups wherein the alkanoyl moiety is a straight or branched C₂₋₆ alkanoyl group, optionally substituted on the thiazolidine ring with one or two members selected from the group consisting of an oxo group and a thioxo group;

such as 2-[(2-, 3-, 4-, or 5-)thiazolidinyl]acetyl, 3-[(2-, 3-, 4-, or 5-)thiazolidinyl]propionyl, 2-[(2-, 3-, 4-, or 5-)thiazolidinyl]propionyl, 4-[(2-, 3-, 4-, or 5-)thiazolidinyl]butyryl, 5-[(2-, 3-, 4-, or 5-) thiazolidinyl]pentanoyl, 6-[(2-, 3-, 4-, or 5-)thiazolidinyl] hexanoyl, 2,2-dimethyl-3-[(2-, 3-, 4-, or 5-)thiazolidinyl] propionyl, 2-methyl-3-[(2-, 3-, 4-, or 5-)thiazolidinyl]propionyl, [2-thioxo-4-oxo-2-(3- or 5-)thiazolidinyl]acetyl, [2-thioxo-2-(3-, 4-, or 5-)thiazolidinyl]acetyl, [2-oxo-2-(3-, 4-, or 5-) thiazolidinyl]acetyl, [2,4-dithioxo-2-(3- or 5-)thiazolidinyl]acetyl, and [2,4-dioxo-2-(3- or 5-)thiazolidinyl]acetyl.

Examples of piperidinyl lower alkanoyl groups include piperidinylalkanoyl groups wherein the alkanoyl moiety is a straight or branched C₂₋₆ alkanoyl group, such as 2-[(1-, 2-, 3-, or 4-)piperidinyl]acetyl, 3-[(1-, 2-, 3-, or 4-) piperidinyl]propionyl, 2-[(1-, 2-, 3-, or 4-)piperidinyl] propionyl, 4-[(1-, 2-, 3-, or 4-)piperidinyl]butyryl, 5-[(1-, 2-, 3- or 4-)piperidinyl]pentanoyl, 6-[(1-, 2-, 3-, or 4-) piperidinyl]hexanoyl, 2,2-dimethyl-3-[(1-, 2-, 3-, or 4-) piperidinyl]propionyl, and 2-methyl-3-[(1-, 2-, 3-, or 4-) piperidinyl]propionyl.

Examples of phenyl lower alkenylcarbonyl groups optionally substituted on the phenyl ring with one or more halogen atoms include:

phenylalkenylcarbonyl groups containing one to three double bonds wherein the alkenyl moiety is a straight or branched C₂₋₆ alkenyl group, optionally substituted on the phenyl ring with one to three halogen atoms;

such as styrylcarbonyl (trivial name: cinnamoyl group), 3-phenyl-2-propenylcarbonyl, 4-phenyl-2-butenylcarbonyl, 4-phenyl-3-butenylcarbonyl, 5-phenyl-4-pentenylcarbonyl, 5-phenyl-3-pentenylcarbonyl, 6-phenyl-5-hexenylcarbonyl, 6-phenyl-4-hexenylcarbonyl, 6-phenyl-3-hexenylcarbonyl, 4-phenyl-1,3-butadienylcarbonyl, 6-phenyl-1,3,5-hexatrienylcarbonyl, 2-chlorostyrylcarbonyl, 3-(4-bromophenyl)-2-propenylcarbonyl, 4-(3-fluorophenyl)-2-butenylcarbonyl, 4-(2,4-dichlorophenyl)-3-butenylcarbonyl, 5-(2,4,6-trifluorophenyl)-4-pentenylcarbonyl, 5-(4-iodophenyl)-3-pentenylcarbonyl, 6-(3-chlorophenyl)-5-hexenylcarbonyl, 6-(4-chlorophenyl)-4-hexenylcarbonyl, 6-(3,4-dichlorophenyl)-3-hexenylcarbonyl, 4-(3-chloro-4-fluorophenyl)-1,3-butadienylcarbonyl, and 6-(2,6-difluorophenyl)-1,3,5-hexatrienylcarbonyl.

Examples of phenyl lower alkenylcarbonyl groups optionally substituted on the phenyl ring with one or more lower alkylenedioxy groups include:

phenylalkenylcarbonyl groups containing one to three double bonds wherein the alkenyl moiety is a straight or branched C₂₋₆alkenyl group, optionally substituted on the phenyl ring with one or more of the above-described straight and branched C₁₋₄ alkylenedioxy groups;

such as 3,4-methylenedioxystyrylcarbonyl, 3-(2,3-ethylenedioxyphenyl)-2-propenylcarbonyl, 4-(3,4-trimethylenedioxyphenyl)-2-butenylcarbonyl, 4-(2,3-tetramethylenedioxyphenyl)-3-butenylcarbonyl, 5-(2,3-methylenedioxyphenyl)-4-pentenylcarbonyl, 5-(3,4-ethylenedioxyphenyl)-3-pentenylcarbonyl, 6-(2,3-trimethylenedioxyphenyl)-5-hexenylcarbonyl, 6-(3,4-tetramethylenedioxyphenyl)-4-hexenylcarbonyl, 6-(2,3-methylenedioxyphenyl)-3-hexenylcarbonyl, 4-(3,4-methylenedioxyphenyl)-1,3-butadienylcarbonyl, and 6-(2,3-methylenedioxyphenyl)-1,3,5-hexatrienylcarbonyl.

Examples of pyridyl lower alkenylcarbonyl groups include pyridylalkenylcarbonyl groups containing one to three double bonds wherein the alkenyl moiety is a straight or branched C₂₋₆ alkenyl group, such as 2-[(2-, 3-, or 4-)pyridyl] vinylcarbonyl, 3-[(2-, 3-, or 4-)pyridyl]-2-propenylcarbonyl, 4-[(2-, 3-, or 4-)pyridyl]-2-butenylcarbonyl, 4-[(2-, 3-, or 4-) pyridyl]-3-butenylcarbonyl, 5-[(2-, 3- or 4-)pyridyl]-4-pentenyl carbonyl, 5-[(2-, 3-, or 4-)pyridyl]-3-pentenylcarbonyl, 6-[(2-, 3-, or 4-)pyridyl]-5-hexenylcarbonyl, 6-[(2-, 3-, or 4-) pyridyl]-4-hexenylcarbonyl, 6-[(2-, 3-, or 4-)pyridyl]-3-hexenylcarbonyl, 4-phenyl-1,3-butadienylcarbonyl, and 6-[(2-, 3-, or 4-)pyridyl]-1,3,5-hexatrienylcarbonyl.

Examples of pyridylthio lower alkanoyl groups include pyridylthioalkanoyl groups wherein the alkanoyl moiety is a straight or branched C₂₋₆ alkanoyl group, such as 2-[(2-, 3-, or 4-)pyridylthio]acetyl, 3-[(2-, 3-, or 4-) pyridylthio]propionyl, 2-[(2-, 3-, or 4-)pyridylthio]propionyl, 4-[(2-, 3-, or 4-) pyridylthio]butyryl, 5-[(2-, 3-, or 4-) pyridylthio]pentanoyl, 6-[(2-, 3-, or 4-)pyridylthio]hexanoyl, 2,2-dimethyl-3-[(2-, 3-, or 4-)pyridylthio]propionyl, and 2-methyl-3-[(2-, 3-, or 4-)pyridylthio]propionyl.

Examples of indolylcarbonyl groups include 1-indolylcarbonyl, 2-indolylcarbonyl, 3-indolylcarbonyl, 4-indolylcarbonyl, 5-indolylcarbonyl, 6-indolylcarbonyl, and 7-indolylcarbonyl.

Examples of pyrrolylcarbonyl groups include 2-pyrrolylcarbonyl and 3-pyrrolylcarbonyl.

Examples of pyrrolidinylcarbonyl groups optionally substituted on the pyrrolidine ring with one or more oxo groups include pyrrolidinylcarbonyl groups optionally substituted on the pyrrolidine ring with one or two oxo groups, such as (1-, 2-, or 3-)pyrrolidinylcarbonyl, 2-oxo-(1-, 3-, 4-, or 5-) pyrrolidinylcarbonyl, 3-oxo-(1-, 2-, 4-, or 5-)pyrrolidinyl carbonyl, 2,5-dioxo-(1- or 3-)pyrrolidinylcarbonyl, and 2,3-dioxo-(1-, 4-, or 5-)pyrrolidinyl carbonyl.

Examples of benzofurylcarbonyl groups include 2-benzofurylcarbonyl, 3-benzofurylcarbonyl, 4-benzofurylcarbonyl, 5-benzofurylcarbonyl, 6-benzofurylcarbonyl, and 7-benzofurylcarbonyl.

Examples of indolyl lower alkanoyl groups include indolylalkanoyl groups wherein the alkanoyl moiety is a straight or branched C₂₋₆ alkanoyl group, such as 2-[(1-, 2-, 3-, 4-, 5-, 6-, or 7-)indolyl]acetyl, 3-[(1-, 2-, 3-, 4-, 5-, 6-, or 7-)indolyl]propionyl, 2-[(1-, 2-, 3-, 4-, 5-, 6-, or 7-)indolyl]propionyl, 4-[(1-, 2-, 3-, 4-, 5-, 6-, or 7-)indolyl]butyryl, 5-[(1-, 2-, 3-, 4-, 5-, 6-, or 7-)indolyl]pentanoyl, 6-[(1-, 2-, 3-, 4-, 5-, 6-, or 7-)indolyl]hexanoyl, 2,2-dimethyl-3-[(1-, 2-, 3-, 4-, 5-, 6-, or 7-)indolyl]propionyl, and 2-methyl-3-[(1-, 2-, 3-, 4-, 5-, 6-, or 7-)indolyl]propionyl.

Examples of benzothienylcarbonyl groups include 2-benzothienylcarbonyl, 3-benzothienylcarbonyl, 4-benzothienylcarbonyl, 5-benzothienylcarbonyl, 6-benzothienylcarbonyl, and 7-benzothienylcarbonyl.

Examples of phenyl lower alkanoyl groups optionally substituted on the phenyl ring with one or more halogen atoms include:

phenylalkanoyl groups wherein the alkanoyl moiety is a straight or branched C₂₋₆ alkanoyl group, optionally substituted on the phenyl ring with one to three halogen atoms;

such as 2-phenylacetyl, 3-phenylpropionyl, 2-phenylpropionyl, 4-phenylbutyryl, 5-phenylpentanoyl, 6-phenylhexanoyl, 2,2-dimethyl-3-phenylpropionyl, 2-methyl-3-phenylpropionyl, 2-(4-fluorophenyl)acetyl, 3-(2,5-difluorophenyl)propionyl, 2-(2,4-difluorophenyl)propionyl, 4-(3,4-difluorophenyl)butyryl, 5-(3,5-difluorophenyl)pentanoyl, 6-(2,6-difluorophenyl)hexanoyl, 2-(2-chlorophenyl)acetyl, 3-(3-chlorophenyl)propionyl, 2-(4-chlorophenyl)propionyl, 4-(2,3-dichlorophenyl)propionyl, 5-(2,4-dichlorophenyl)pentanoyl, 6-(2,5-dichlorophenyl)hexanoyl, 2-(3,4-dichlorophenyl)acetyl, 3-(2,6-dichlorophenyl)propionyl, 2-(3-fluorophenyl)propionyl, 4-(2-fluorophenyl)butyryl, 5-(3-bromophenyl)pentanoyl, 6-(4-iodophenyl)hexanoyl, 2-(2-bromophenyl)acetyl, 3-(4-bromophenyl) propionyl, 2-(3,5-dichlorophenyl)propionyl, 4-(2,4,6-trifluoro phenyl)butyryl, 5-(3,4-difluorophenyl)pentanoyl, 6-(2-iodophenyl) hexanoyl, 2-(3-iodophenyl)acetyl, 3-(4-iodophenyl)propionyl, 2-(2,3-dibromophenyl)propionyl, 4-(2,4-diiodophenyl)butyryl, and 2-(2,4,6-trichlorophenyl)acetyl.

Examples of phenylsulfonyl groups optionally substituted on the phenyl ring with one or more members selected from the group consisting of lower alkoxycarbonyl groups; a cyano group; a nitro group; amino groups optionally substituted with one or more lower alkanoyl groups; a hydroxy group; a carboxyl group; lower alkoxycarbonyl lower alkyl groups; halogen atoms; lower alkyl groups optionally substituted with one or more halogen atoms; and lower alkoxy groups optionally substituted with one or more halogen atoms include:

phenylsulfonyl groups optionally substituted on the phenyl ring with one to five members selected from the group consisting of the above-described lower alkoxycarbonyl groups wherein the alkoxy moiety is a straight or branched C₁₋₆ alkoxy group; a cyano group; a nitro group; the above-described amino groups optionally substituted with one or two straight and/or branched C₁₋₆ alkanoyl groups; a hydroxy group; a carboxyl group; the above-described alkoxycarbonylalkyl groups wherein the alkoxy moiety is a straight or branched C₁₋₆ alkoxy group and the alkyl moiety is a straight or branched C₁₋₆ alkyl group; halogen atoms; the above-described straight and branched C₁₋₆ alkyl groups optionally substituted with one to three halogen atoms; and the above-described straight and branched C₁₋₆ alkoxy groups optionally substituted with one to three halogen atoms;

such as phenylsulfonyl, 4-methoxyphenylsulfonyl, 3-methoxyphenylsulfonyl, 2-methoxyphenylsulfonyl, 2-trifluoromethoxyphenylsulfonyl, 3-trifluoromethoxyphenylsulfonyl, 4-trifluoromethoxyphenylsulfonyl, 3,4-dimethoxyphenylsulfonyl, 2,5-dimethoxyphenylsulfonyl, 2,4,6-trimethoxyphenylsulfonyl, 4-n-butoxyphenylsulfonyl, 2-methoxy-5-chlorophenylsulfonyl, 2-methoxy-5-methylphenylsulfonyl, 2-methoxy-4-methylphenylsulfonyl, 4-chlorophenylsulfonyl, 3-chlorophenylsulfonyl, 2-chlorophenylsulfonyl, 4-fluorophenylsulfonyl, 3-fluorophenylsulfonyl, 2-fluorophenylsulfonyl, 4-bromophenylsulfonyl, 3-bromophenylsulfonyl, 2-bromophenylsulfonyl, 2,6-dichlorophenylsulfonyl, 2,3-dichlorophenylsulfonyl, 2,5-dichlorophenylsulfonyl, 2,4-dichlorophenylsulfonyl, 3,4-dichlorophenylsulfonyl, 3,5-dichlorophenylsulfonyl, 2-chloro-4-fluorophenylsulfonyl, 2-bromo-5-chlorophenylsulfonyl, 2,5-difluorophenylsulfonyl, 2,4-difluorophenylsulfonyl, 2,6-difluorophenylsulfonyl, 3,4-difluorophenylsulfonyl, 2,4-dichloro-5-methylphenylsulfonyl, 2,4,5-trifluorophenylsulfonyl, 2,3,4,5,6-pentafluorophenylsulfonyl, 3-chloro-4-fluorophenylsulfonyl, 2-chloro-6-methylphenylsulfonyl, 2,4-dichloro-6-methylphenylsulfonyl, 2-methyl-3-chlorophenylsulfonyl, 2-methyl-3-chlorophenylsulfonyl, 4-methyl-3-chlorophenylsulfonyl, 2-methyl-5-fluorophenylsulfonyl, 2-methyl-4-bromophenylsulfonyl, 2-fluoro-4-bromophenylsulfonyl, 2,5-dimethyl-4-chlorophenylsulfonyl, 2-methylphenylsulfonyl, 3-methylphenylsulfonyl, 4-methylphenylsulfonyl, 2,5-dimethylphenylsulfonyl, 2,4,6-trimethylphenylsulfonyl, 2,3,6-trimethyl-4-methoxyphenylsulfonyl, 4-tert-butylphenylsulfonyl, 4-ethylphenylsulfonyl, 4-isopropylphenylsulfonyl, 2-trifluoromethylphenylsulfonyl, 3-trifluoromethylphenylsulfonyl, 4-trifluoromethylphenylsulfonyl, 2-methoxycarbonylphenylsulfonyl, 2-cyanophenylsulfonyl, 3-cyanophenylsulfonyl, 4-cyanophenylsulfonyl, 3-nitrophenylsulfonyl, 2-nitrophenylsulfonyl, 4-nitrophenylsulfonyl, 3-nitro-4-methylphenylsulfonyl, 3-nitro-6-methylphenylsulfonyl, 3-nitro-6-chlorophenylsulfonyl, 2-chloro-4-cyanophenylsulfonyl, 4-acetylaminophenylsulfonyl, 3-chloro-4-acetylaminophenylsulfonyl, 2-hydroxy-3,5-dichlorophenylsulfonyl, 2-hydroxyphenylsulfonyl, 3-hydroxyphenylsulfonyl, 4-hydroxyphenylsulfonyl, 2-nitro-4-methoxyphenylsulfonyl, 3-carboxyphenylsulfonyl, 4-carboxyphenylsulfonyl, 2-carboxyphenylsulfonyl, 4-(2-methoxycarbonylethyl)phenylsulfonyl, 3-carboxy-4-hydroxyphenylsulfonyl, 3-aminophenylsulfonyl, 2-aminophenylsulfonyl, and 4-aminophenylsulfonyl.

Examples of thienylsulfonyl groups optionally substituted on the thiophene ring with one or more members selected from the group consisting of halogen atoms and lower alkoxycarbonyl groups include:

thienylsulfonyl groups optionally substituted on the thiophene ring with one to three members selected from halogen atoms and the above-described alkoxycarbonyl groups wherein the alkoxy moiety is a straight or branched C₁₋₆ alkoxy group;

such as (2- or 3-)thienylsulfonyl, [2-chloro-(3-, 4-, or 5-)thienyl]sulfonyl, [2,3-dichloro-(4- or 5-) thienyl]sulfonyl, [2,5-dichloro-(3- or 4-)thienyl]sulfonyl, [2-bromo-(3-, 4-, or 5-)thienyl]sulfonyl, [2-fluoro-(3-, 4-, or 5-) thienyl]sulfonyl, (2,3,4-trichloro-5-thienyl)sulfonyl, [2-methoxycarbonyl-(3-, 4-, or 5-)thienyl]sulfonyl, [3-ethoxycarbonyl-(2-, 4-, or 5-)thienyl]sulfonyl, [3-n-propoxycarbonyl-(2-, 4-, or 5-)thienyl]sulfonyl, [2-tert-butoxycarbonyl-(3-, 4-, or 5-)thienyl]sulfonyl, [2-n-pentyloxycarbonyl-(3-, 4-, or 5-)thienyl]sulfonyl, [3-n-hexyloxycarbonyl-(2-, 4-, or 5-)thienyl]sulfonyl, [2,3-dimethoxycarbonyl-(4- or 5-)thienyl]sulfonyl, and [2-chloro-3-methoxycarbonyl-(4- or 5-)thienyl]sulfonyl.

Examples of quinolylsulfonyl groups include 2-quinolylsulfonyl, 3-quinolylsulfonyl, 4-quinolylsulfonyl, 5-quinolylsulfonyl, 6-quinolylsulfonyl, 7-quinolylsulfonyl, and 8-quinolylsulfonyl.

Examples of imidazolylsulfonyl groups optionally substituted on the imidazole ring with one or more lower alkyl groups include imidazolylsulfonyl groups optionally substituted on the imidazole ring with one to three above-described straight and branched C₁₋₆ alkyl groups, such as (1-, 2-, 4-, or 5-)imidazolylsulfonyl, [1-methyl-(2-, 4-, or 5-) imidazolyl]sulfonyl, [2-ethyl-(1-, 4-, or 5-)imidazolyl]sulfonyl, [1-isopropyl-(2-, 4-, or 5-)imidazolyl]sulfonyl, [4-n-butyl-(1-, 2-, or 5-)imidazolyl]sulfonyl, [5-n-pentyl-(1-, 2-, or 4-) imidazolyl]sulfonyl, [1-n-hexyl-(2-, 4-, or 5-)imidazolyl]sulfonyl, [1,2-dimethyl-(4- or 5-)imidazolyl]sulfonyl, and (1,2,4-trimethyl-5-imidazolyl)sulfonyl.

Examples of phenylsulfonyl groups optionally substituted on the phenyl ring with one or more lower alkylenedioxy groups include phenylsulfonyl groups optionally substituted on the phenyl ring with one or more one to three the above-described straight and branched C₁₋₄ alkylenedioxy groups, such as (3,4-ethylenedioxyphenyl)sulfonyl, (2,3-methylenedioxyphenyl)sulfonyl, (3,4-trimethylenedioxyphenyl)sulfonyl, and (2,3-tetramethylenedioxyphenyl)sulfonyl.

Examples of lower alkenylsulfonyl groups include straight and branched C₂₋₆ alkenylsulfonyl groups containing one to three double bonds, such as vinylsulfonyl, 1-propenylsulfonyl, 1-methyl-1-propenylsulfonyl, 2-methyl-1-propenylsulfonyl, 2-propenylsulfonyl, 2-butenylsulfonyl, 1-butenylsulfonyl, 3-butenylsulfonyl, 2-pentenylsulfonyl, 1-pentenylsulfonyl, 3-pentenylsulfonyl, 4-pentenylsulfonyl, 1,3-butadienylsulfonyl, 1,3-pentadienylsulfonyl, 2-pentene-4-ynylsulfonyl, 2-hexenylsulfonyl, 1-hexenylsulfonyl, 5-hexenylsulfonyl, 3-hexenylsulfonyl, 4-hexenylsulfonyl, 3,3-dimethyl-1-propenylsulfonyl, 2-ethyl-1-propenylsulfonyl, 1,3,5-hexatrienylsulfonyl, 1,3-hexadienylsulfonyl, and 1,4-hexadienylsulfonyl.

Examples of cycloalkyl-substituted lower alkylsulfonyl groups include C₃₋₈ cycloalkyl-substituted alkylsulfonyl groups wherein the alkyl moiety is a straight or branched C₁₋₆ alkyl group, such as cyclopropylmethylsulfonyl, cyclohexylmethylsulfonyl, 2-cyclopropylethylsulfonyl, 1-cyclobutylethylsulfonyl, cyclopentylmethylsulfonyl, 3-cyclopentylpropylsulfonyl, 4-cyclohexylbutylsulfonyl, 5-cycloheptylpentylsulfonyl, 6-cyclooctylhexylsulfonyl, 1,1-dimethyl-2-cyclohexylethylsulfonyl, and 2-methyl-3-cyclopropylpropylsulfonyl.

Examples of 3,4-dihydro-2H-1,4-benzoxazinylsulfonyl groups optionally substituted on the 3,4-dihydro-2H-1,4-benzoxazine ring with one or more lower alkyl groups include 3,4-dihydro-2H-1,4-benzoxazinylsulfonyl groups optionally substituted on the 3,4-dihydro-2H-1,4-benzoxazine ring with one to three above-described straight and/or branched C₁₋₆ alkyl groups, such as (2-, 3-, 4-, 5-, 6-, 7- or 8-)3,4-dihydro-2H-1,4-benzoxazinylsulfonyl, [4-methyl-(2-, 3-, 5-, 6-, 7- or 8-) 3,4-dihydro-2H-1,4-benzoxazinyl]sulfonyl, [5-ethyl-(2-, 3-, 4-, 6-, 7- or 8-)3,4-dihydro-2H-1,4-benzoxazinyl]sulfonyl, [6-n-propyl-(2-, 3-, 4-, 5-, 7- or 8-)3,4-dihydro-2H-1,4-benzoxazinyl]sulfonyl, [7-n-butyl-(2-, 3-, 5-, 6-, 7- or 8-) 3,4-dihydro-2H-1,4-benzoxazinyl]sulfonyl, [8-n-pentyl-(2-, 3-, 5-, 6-, 7- or 8-)3,4-dihydro-2H-1,4-benzoxazinyl]sulfonyl, [2-n-hexyl-(3-, 4-, 5-, 6-, 7- or 8-)3,4-dihydro-2H-1,4-benzoxazinyl]sulfonyl, [3-methyl-(2-, 4-, 5-, 6-, 7- or 8-) 3,4-dihydro-2H-1,4-benzoxazinyl]sulfonyl, [4,6-dimethyl-(2-, 3-, 5-, 7- or 8-)3,4-dihydro-2H-1,4-benzoxazinyl]sulfonyl, and [4,5,6-trimethyl-(2-, 3-, 7- or 8-)3,4-dihydro-2H-1,4-benzoxazinyl]sulfonyl.

Examples of pyrazolylsulfonyl groups optionally substituted on the pyrazole ring with one or more members selected from the group consisting of halogen atoms and lower alkyl groups include:

pyrazolylsulfonyl groups optionally substituted on the pyrazole ring with one to three members selected from the group consisting of halogen atoms and the above-described straight and branched C₁₋₆ alkyl groups;

such as (1-, 3-, 4-, or 5-)pyrazolylsulfonyl, (1,3-dimethyl-5-chloro-4-pyrazolyl)sulfonyl, [1-ethyl-(3-, 4-, or 5-) pyrazolyl]sulfonyl, [3-n-propyl-(1-, 4-, or 5-)pyrazolyl]sulfonyl, [4-n-butyl-(3-, 4-, or 5-)pyrazolyl]sulfonyl, [5-n-pentyl-(1-, 3-, or 4-)pyrazolyl]sulfonyl, [1-n-hexyl-(3-, 4-, or 5-) pyrazolyl]sulfonyl, [1,3-dimethyl-(4- or 5-)pyrazolyl]sulfonyl, (1,3,5-trimethyl-4-pyrazolyl)sulfonyl, [3-bromo-(1-, 4-, or 5-) pyrazolyl]sulfonyl, [4-fluoro-(1-, 3-, or 5-)pyrazolyl]sulfonyl, [5-iodo-(1-, 3-, or 4-)pyrazolyl]sulfonyl, [3,4-dichloro-(1- or 5-)pyrazolyl]sulfonyl, and (3,4,5-trichloro-4-pyrazolyl) sulfonyl.

Examples of isoxazolylsulfonyl groups optionally substituted on the isoxazole ring with one or more lower alkyl groups include isoxazolylsulfonyl groups optionally substituted on the isoxazole ring with one or two above-described straight and/or branched C₁₋₆alkyl groups, such as (3-, 4-, or 5-) isoxazolylsulfonyl, (3,5-dimethyl-4-isoxazolyl)sulfonyl, [3-methyl-(4- or 5-)isoxazolyl]sulfonyl, [3-ethyl-(4- or 5-) isoxazolyl]sulfonyl, [4-n-propyl-(3- or 5-)isoxazolyl]sulfonyl, [5-n-butyl-(3- or 4-)isoxazolyl]sulfonyl, [3-n-pentyl-(4- or 5-) isoxazolyl]sulfonyl, and [4-n-hexyl-(3- or 5-)isoxazolyl]sulfonyl.

Examples of thiazolylsulfonyl groups optionally substituted on the thiazole ring with one or more members selected from the group consisting of lower alkyl groups and an amino group, each amino substituent optionally being substituted with one or more lower alkanoyl groups, include:

thiazolylsulfonyl groups optionally substituted on the thiazole ring with one or two members selected from the group consisting of the above-described straight or branched C₁₋₆ alkyl groups and the above-described amino groups optionally substituted with one or two straight and/or branched C₁₋₆ alkanoyl groups;

such as (2-, 4-, or 5-)thiazolylsulfonyl, (2-acetylamino-4-methyl-5-thiazolyl)sulfonyl, [2-ethyl-(4- or 5-) thiazolyl]sulfonyl, [4-n-propyl-(2- or 5-)thiazolyl]sulfonyl, [5-n-butyl-(2- or 4-)thiazolyl]sulfonyl, [2-n-pentyl-(4- or 5-) thiazolyl]sulfonyl, [4-n-hexyl-(2- or 5-)thiazolyl]sulfonyl, (2,4-dimethyl-5-thiazolyl)sulfonyl, [2-amino-(4- or 5-) thiazolyl]sulfonyl, [2-formylamino-(4- or 5-)thiazolyl]sulfonyl, [4-n-propionylamino-(2- or 5-)thiazolyl]sulfonyl, [5-n-butyryl amino-(2- or 4-)thiazolyl]sulfonyl, [2-n-pentanoylamino-(4- or 5-)thiazolyl]sulfonyl, [4-n-hexanoylamino-(2- or 5-) thiazolyl]sulfonyl, (2,4-diacetyl-5-thiazolyl)sulfonyl, and [2-(N,N-diacetylamino)-(4- or 5-)thiazolyl]sulfonyl.

Examples of phenyl lower alkylsulfonyl groups include mono- and di-phenylalkyl groups wherein the alkyl moiety is a straight or branched C₁₋₆ alkyl group, such as benzylsulfonyl, 1-phenethylsulfonyl, 2-phenethylsulfonyl, 3-phenylpropylsulfonyl, 2-phenylpropylsulfonyl, 4-phenylbutylsulfonyl, 5-phenylpentylsulfonyl, 4-phenylpentylsulfonyl, 6-phenylhexylsulfonyl, 2-methyl-3-phenylpropylsulfonyl, 1,1-dimethyl-2-phenylethylsulfonyl, 1,1-dimethyl-1-phenylmethylsulfonyl, 1,1-diphenylmethylsulfonyl, 2,2-diphenylethylsulfonyl, 3,3-diphenylpropylsulfonyl, and 1,2-diphenylethylsulfonyl.

Examples of phenyl lower alkenylsulfonyl groups include:

phenylalkenylsulfonyl groups containing one to three double bonds wherein the alkenyl moiety is a straight or branched C₂₋₆ alkenyl group, optionally substituted on the phenyl ring with one to three halogen atoms;

such as styrylsulfonyl, 3-phenyl-2-propenylsulfonyl, 4-phenyl-2-butenylsulfonyl, 4-phenyl-3-butenylsulfonyl, 5-phenyl-4-pentenylsulfonyl, 5-phenyl-3-pentenylsulfonyl, 6-phenyl-5-hexenylsulfonyl, 6-phenyl-4-hexenylsulfonyl, 6-phenyl-3-hexenylsulfonyl, 4-phenyl-1,3-butadienylsulfonyl, 6-phenyl-1,3,5-hexatrienylsulfonyl, 2-chlorostyrylsulfonyl, 3-(4-bromophenyl)-2-propenylsulfonyl, 4-(3-fluorophenyl)-2-butenylsulfonyl, 4-(2,4-dichlorophenyl)-3-butenylsulfonyl, 5-(2,4,6-trifluorophenyl)-4-pentenylsulfonyl, 5-(4-iodophenyl)-3-pentenylsulfonyl, 6-(3-chlorophenyl)-5-hexenylsulfonyl, 6-(4-chlorophenyl)-4-hexenylsulfonyl, 6-(3,4-dichlorophenyl)-3-hexenylsulfonyl, 4-(3-chloro-4-fluorophenyl)-1,3-butadienylsulfonyl, and 6-(2,6-difluorophenyl)-1,3,5-hexatrienylsulfonyl.

Examples of naphthyloxycarbonyl groups include 1-naphthyloxycarbonyl and 2-naphthyloxycarbonyl.

Examples of lower alkynyloxycarbonyl groups include alkynyloxycarbonyl groups wherein the alkynyl moiety is a straight or branched C₂₋₆ alkynyl group, such as ethynyloxycarbonyl, 2-propynyloxycarbonyl, 2-butynyloxycarbonyl, 3-butynyloxycarbonyl, 1-methyl-2-propynyloxycarbonyl, 2-pentynyloxycarbonyl, and 2-hexynyloxycarbonyl.

Examples of lower alkenyloxycarbonyl groups include alkenyloxycarbonyl groups containing one to three double bonds wherein the alkenyl moiety is a straight or branched C₂₋₆ alkenyl group, such as vinyloxycarbonyl, 1-propenyloxycarbonyl, 1-methyl-1-propenyloxycarbonyl, 2-methyl-1-propenyloxycarbonyl, 2-propenyloxycarbonyl, 2-butenyloxycarbonyl, 1-butenyloxycarbonyl, 3-butenyloxycarbonyl, 2-pentenyloxycarbonyl, 1-pentenyloxycarbonyl, 3-pentenyloxycarbonyl, 4-pentenyloxycarbonyl, 1,3-butadienyloxycarbonyl, 1,3-pentadienyloxycarbonyl, 2-pentene-4-ynyloxycarbonyl, 2-hexenyloxycarbonyl, 1-hexenyloxycarbonyl, 5-hexenyloxycarbonyl, 3-hexenyloxycarbonyl, 4-hexenyloxycarbonyl, 3,3-dimethyl-1-propenyloxycarbonyl, 2-ethyl-1-propenyloxycarbonyl, 1,3,5-hexatrienyloxycarbonyl, 1,3-hexadienyloxycarbonyl, and 1,4-hexadienyloxycarbonyl.

Examples of phenyl lower alkoxy-substituted lower alkoxycarbonyl groups include phenylalkoxy-substituted alkoxycarbonyl groups wherein each of the two alkoxy moieties is a straight or branched C₁₋₆ alkoxy group, such as phenylmethoxymethoxycarbonyl, 2-(phenylmethoxy)ethoxycarbonyl, 1-(phenylmethoxy)ethoxycarbonyl, 3-(phenylmethoxy)propoxycarbonyl, 4-(phenylmethoxy)butoxycarbonyl, 5-(phenylmethoxy) pentyloxycarbonyl, 6-(phenylmethoxy)hexyloxycarbonyl, 1,1-dimethyl-2-(phenylmethoxy)ethoxycarbonyl, 2-methyl-3-(phenylmethoxy)propoxycarbonyl, 1-(2-phenylethoxy)ethoxycarbonyl, 2-(1-phenylethoxy)ethoxycarbonyl, 3-(3-phenylpropoxy)propoxy carbonyl, 4-(4-phenylbutoxy)butoxycarbonyl, 5-(5-phenylpentyloxy) pentyloxycarbonyl, 6-(6-phenylhexyloxy)hexyloxycarbonyl, (1,1-dimethyl-2-phenylethoxy)methoxycarbonyl, and 3-(2-methyl-3-phenylpropoxy)propoxycarbonyl.

Examples of cycloalkyloxycarbonyl groups optionally substituted on the cycloalkyl ring with one or more lower alkyl groups include:

cycloalkyloxycarbonyl groups wherein the cycloalkoxy moiety is a C₃₋₈ cycloalkoxy group, optionally substituted on the cycloalkyl ring with one to three above-described straight and branched C₁₋₆ alkyl groups;

such as cyclopropyloxycarbonyl, cyclobutyloxycarbonyl, cyclopentyloxycarbonyl, cyclohexyloxycarbonyl, cycloheptyloxycarbonyl, cyclooctyloxycarbonyl, 3-methyl-6-isopropylcyclohexyloxycarbonyl, 2-ethylcyclopropyloxycarbonyl, 2-n-propylcyclobutyloxycarbonyl, 3-n-butylcycloheptyloxycarbonyl, 3-n-pentylcyclooctyloxycarbonyl, 2-methylcyclopentyloxycarbonyl, and 2,3,6-trimethylcyclohexyloxycarbonyl.

Examples of isoxazolyl groups optionally substituted on the isoxazole ring with one or more lower alkyl groups include isoxazolyl groups optionally substituted on the isoxazole ring with one or two straight and/or branched C₁₋₆ alkyl groups, such as (3-, 4-, or 5-)isoxazolyl, 5-methyl-(3- or 4-) isoxazolyl, 3,5-dimethyl-4-isoxazolyl, 3-ethyl-(4- or 5-) isoxazolyl, 4-n-propyl-(3- or 5-)isoxazolyl, 5-n-butyl-(3- or 4-) isoxazolyl, 3-n-pentyl-(4- or 5-)isoxazolyl and 4-n-hexyl-(3- or 5-)isoxazolyl.

Examples of 5- to 7-membered saturated heterocyclic rings formed from R⁶ and R⁷ being linked together, together with the nitrogen atom to which they are bound, the heterocyclic ring optionally containing one or more additional heteroatoms, include:

5- to 7-membered saturated heterocyclic rings formed from R⁶ and R⁷ being linked together, together with the nitrogen atom to which they are bound, the heterocyclic group optionally containing one or more additional heteroatoms selected from oxygen, sulfur atom, and nitrogen atom;

such as pyrrolidine, piperazine, piperidine, morpholine, thiomorpholine, homopiperazine, homopiperidine, imidazolidine, thiazolidine, isothiazolidine, oxazolidine, isoxazolidine, isothiazolidine, and pyrazolidine.

Examples of phenyl groups optionally substituted on the phenyl ring with one or more members selected from the group consisting of halogen atoms; lower alkoxy groups optionally substituted with one or more halogen atoms; lower alkyl groups optionally substituted with one or more halogen atoms; a cyano group; and a hydroxy group include:

phenyl groups optionally substituted on the phenyl ring with one to three members selected from the group consisting of halogen atoms; the above-described straight and branched C₁₋₆ alkoxy groups optionally substituted with one to three halogen atoms; the above-described straight and branched C₁₋₆ alkyl groups optionally substituted with one to three halogen atoms; a cyano group; and a hydroxy group;

such as phenyl, 4-isopropylphenyl, 3-isopropylphenyl, 2-isopropylphenyl, 4-tert-butylphenyl, 4-methylphenyl, 3-methylphenyl, 2-methylphenyl, 2,3-dimethylphenyl, 2,4-dimethylphenyl, 3,5-dimethylphenyl, 2,4,6-trimethylphenyl, 4-methyl-3-methoxyphenyl, 4-trifluoromethylphenyl, 3-trifluoromethylphenyl, 2-trifluoromethylphenyl, 4-methyl-3-chlorophenyl, 4-chlorophenyl, 3-chlorophenyl, 2-chlorophenyl, 2-fluorophenyl, 3-fluorophenyl, 4-fluorophenyl, 3-bromophenyl, 3,4-dichlorophenyl, 3,5-dichlorophenyl, 3,4,5-trichlorophenyl, 2,4,6-trifluorophenyl, 3,5-difluorophenyl, 3-chloro-4-fluorophenyl, 2-chloro-5-fluorophenyl, 3-fluoro-4-methoxyphenyl, 3-chloro-4-methoxyphenyl, 3-chloro-4-hydroxyphenyl, 4-methoxyphenyl, 3-methoxyphenyl, 2-methoxyphenyl, 2,4-dimethoxyphenyl, 3,4-dimethoxyphenyl, 2,4,6-trimethoxyphenyl, 2-methoxy-5-chlorophenyl, 4-ethoxyphenyl, 4-trifluoromethoxyphenyl, 3-trifluoromethoxyphenyl, 2-trifluoromethoxyphenyl, 3-methoxy-5-trifluoromethyl phenyl, 2-cyanophenyl, 3-cyanophenyl, 4-cyanophenyl, 3-hydroxyphenyl, 2-hydroxyphenyl, and 4-hydroxyphenyl.

Examples of phenyl lower alkyl groups optionally substituted on the phenyl ring with one or more halogen atoms include:

mono- and di-phenylalkyl groups wherein the alyl moiety is a straight or branched C₁₋₆alkyl group, optionally substituted on each phenyl ring with one to three halogen atoms;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-diphenylmethyl, 2,2-diphenylethyl, 3,3-diphenylpropyl, 1,2-diphenylethyl, 4-chlorobenzyl, 2-chlorobenzyl, 3-chlorobenzyl, 2-fluorobenzyl, 3-fluorobenzyl, 4-fluorobenzyl, 2,3-dichlorobenzyl, and 2,4,6-trifluorobenzyl.

Examples of phenyl lower alkoxy groups optionally substituted on the phenyl ring with one or more halogen atoms include:

phenylalkoxy groups wherein the alkoxy moiety is a straight or branched C₁₋₆alkoxy group, optionally substituted on the phenyl ring with one to three halogen atoms;

such as benzyloxy, 2-phenylethoxy, 1-phenylethoxy, 3-phenylpropoxy, 4-phenylbutoxy, 5-phenylpentyloxy, 6-phenylhexyloxy, 1,1-dimethyl-2-phenylethoxy, 2-methyl-3-phenylpropoxy, 4-chlorobenzyloxy, 2-chlorobenzyloxy, 3-chlorobenzyloxy, 2-fluorobenzyloxy, 3-fluorobenzyloxy, 4-fluorobenzyloxy, 2,4-dibromobenzyloxy, and 2,4,6-trifluorobenzyloxy.

Examples of carbamoyl lower alkyl groups optionally substituted with one or more members selected from the group consisting of phenyl group and lower alkyl groups include:

carbamoylalkyl groups wherein the alkyl moiety is a straight or branched C₁₋₆ alkyl group, optionally substituted with one or two members selected from the group consisting of a phenyl group and the above-described straight and branched C₁₋₆ alkyl groups;

such as carbamoylmethyl, 2-carbamoylethyl, 1-carbamoylethyl, 3-carbamoylpropyl, 4-carbamoylbutyl, 5-carbamoylpentyl, 6-carbamoylhexyl, 1,1-dimethyl-2-carbamoylethyl, 2-methyl-3-carbamoylpropyl, 2-(N-methyl-N-phenylcarbamoyl)ethyl, N-phenylcarbamoylmethyl, 2-(N,N-dimethylcarbamoyl)ethyl, 3-(N-phenylcarbamoyl)propyl, 2-(N-ethyl-N-phenylcarbamoyl)ethyl, N,N-dimethylcarbamoylmethyl, N-methyl-N-ethylcarbamoylmethyl, N-methylcarbamoylmethyl, and 2-(N-methylcarbamoyl)ethyl.

Examples of phenyl lower alkylidene groups optionally substituted on the phenyl ring with one or more halogen atoms include:

phenylalkylidene groups wherein the alkylidene moiety is a straight or branched C₁₋₆ alkylidene group, optionally substituted on the phenyl ring with one to three halogen atoms;

such as phenylmethylidene, phenylethylidene, phenylpropylidene, phenylisopropylidene, phenylbutylidene, phenylpentylidene, phenylhexylidene, 2-chlorophenylmethylidene, 3-chlorophenylmethylidene, 4-chlorophenylmethylidene, 2-fluorophenylmethylidene, 3-fluorophenylmethylidene, 4-fluorophenylmethylidene, 2-bromophenylmethylidene, 3-bromophenylmethylidene, 4-bromophenylmethylidene, 2-iodophenylmethylidene, 2,3-dichlorophenylmethylidene, 2,4-difluorophenylmethylidene, 2,4,6-trichlorophenylmethylidene, 2,3,5-trifluorophenylmethylidene, and 2-fluoro-4-chlorophenylmethylidene.

Examples of phenyl lower alkoxycarbonyl groups include phenylalkoxycarbonyl groups wherein the alkoxy moiety is a straight or branched C₁₋₆ alkoxy group, such as benzyloxycarbonyl, 2-phenylethoxycarbonyl, 1-phenylethoxycarbonyl, 3-phenylpropoxy carbonyl, 4-phenylbutoxycarbonyl, 5-phenylpentyloxycarbonyl, 6-phenylhexyloxycarbonyl, 1,1-dimethyl-2-phenylethoxycarbonyl, and 2-methyl-3-phenylpropoxycarbonyl.

Examples of pyridyl groups optionally substituted on the pyridine ring with one or more members selected from the group consisting of a cyano group and lower alkyl groups include;

pyridyl groups optionally substituted on the pyridine ring with one to three members selected from the group consisting of a cyano group and the above-described straight and branched C₁₋₆ alkyl groups;

such as (2-, 3-, or 4-)pyridyl, 2-methyl-(3-, 4-, 5-, or 6-)pyridyl, 3-methyl-(2-, 4-, 5-, or 6-)pyridyl, 4-methyl-(2- or 3-)pyridyl, 2-cyano-(3-, 4-, 5-, or 6-)pyridyl, 3-cyano-(2-, 4-, 5-, or 6-)pyridyl, 4-cyano-(2- or 3-)pyridyl, 2,3-dimethyl-(4-, 5-, or 6-)pyridyl, 3,4,5-trimethyl-(2- or 6-) pyridyl, 2,4-dicyano-(3-, 5-, or 6-)pyridyl, 2,4,5-tricyano-(3- or 6-)pyridyl, and 2-methyl-4-cyano-(3-, 5-, or 6-)pyridyl.

Examples of 1,3-dioxolanyl lower alkyl groups include 1,3-dioxolanylalkyl groups wherein the alkyl moiety is a straight or branched C₁₋₆ alkyl group, such as [(2- or 4-)1,3-dioxolanyl]methyl, 2-[(2- or 4-)1,3-dioxolanyl]ethyl, 1-[(2- or 4-)1,3-dioxolanyl]ethyl, 3-[(2- or 4-)1,3-dioxolanyl] propyl, 4-[(2- or 4-)1,3-dioxolanyl]butyl, 1,1-dimethyl-2-[(2- or 4-)1,3-dioxolanyl]ethyl, 5-[(2- or 4-)1,3-dioxolanyl] pentyl, 6-[(2- or 4-)1,3-dioxolanyl]hexyl, 1-[(2- or 4-)1,3-dioxolanyl]isopropyl, and 2-methyl-3-[(1-, 2-, or 4-)1,3-dioxolanyl]propyl.

Examples of 5- to 8-membered saturated heterocyclic rings formed from R⁸ and R⁹ being linked together, together with the nitrogen atom to which they are bound, the heterocyclic ring optionally containing one or more additional heteroatoms, include:

5- to 8-membered saturated heterocyclic rings formed from R⁸ and R⁹ being linked together, together with the nitrogen atom to which they are bound, the heterocyclic ring optionally containing one or more additional heteroatoms selected from oxygen, nitrogen, and sulfur atom;

such as pyrrolidine, piperazine, piperidine, morpholine, thiomorpholine, imidazolidine, thiazolidine, isothiazolidine, oxazolidine, isoxazolidine, isothiazolidine, pyrazolidine, perhydroazepine, and perhydroazocine.

Examples of octahydropyrrolo[1,2-a]pyrazinyl groups optionally substituted on the pyrazine ring with one or more lower alkyl groups include octahydropyrrolo[1,2-a]pyrazinyl groups optionally substituted on the pyrazine ring with one to three straight and/or branched C₁₋₆alkyl groups.

Examples of 8-azabicyclo[3.2.1]octyl groups optionally substituted on the 8-azabicyclo[3.2.1]octyl group with one or more phenoxy groups, each phenoxy substituent optionally being substituted on the phenyl ring with one or more halogen atoms, include 8-azabicyclo[3.2.1]octyl groups optionally substituted on the 8-azabicyclo[3.2.1]octyl group with one to three phenoxy groups, each phenoxy substituent optionally being substituted on the phenyl ring with one to three halogen atoms.

Examples of 5- or 6-membered saturated heterocyclic rings formed from R¹¹ and R¹², or R¹³ and R¹⁴ being linked together, together with the nitrogen atom to which they are bound, the heterocyclic ring optionally containing one or more additional heteroatoms, include:

5- or 6-membered saturated heterocyclic rings formed from R¹¹ and R¹², or R¹³ and R¹⁴ being linked together, together with the nitrogen atom to which they are bound, the heterocyclic ring optionally containing one or more additional heteroatoms selected from oxygen, nitrogen, and sulfur atom;

such as pyrrolidine, piperazine, piperidine, morpholine, thiomorpholine, imidazolidine, thiazolidine, isothiazolidine, oxazolidine, isoxazolidine, isothiazolidine, and pyrazolidine.

Examples of phenyl groups optionally substituted on the phenyl ring with one or more members selected from the group consisting of lower alkyl groups optionally substituted with one or more halogen atoms; lower alkylthio groups; lower alkoxy groups optionally substituted with one or more halogen atoms; halogen atoms; a phenyl group; lower alkylamino groups; a cyano group; a phenoxy group; cycloalkyl groups; pyrrolidinyl groups optionally substituted with one or more oxo groups; 1,2,3,4-tetrahydroisoquinolylcarbonyl groups; 1,2,3,4-tetrahydroquinolylcarbonyl groups optionally substituted with one or more lower alkyl groups; 1,2,3,4-tetrahydroquinoxalinylcarbonyl groups optionally substituted with one or more lower alkyl groups; thiazolyl groups optionally substituted with one or more phenyl groups; a carbamoyl group;

phenyl lower alkoxy groups; lower alkylsulfonylamino groups; anilino groups optionally substituted with one or more halogen atoms; phenyl lower alkyl groups; and hydroxy-substituted lower alkyl groups include:

phenyl groups optionally substituted on the phenyl ring with one to three members selected from the group consisting of straight and branched C₁₋₆ alkyl groups optionally substituted with one to three halogen atoms; straight and branched C₁₋₆ alkylthio groups; straight and branched C₁₋₆ alkoxy groups optionally substituted with one to three halogen atoms; halogen atoms; a phenyl group; amino groups optionally substituted with one or two straight and/or branched C₁₋₆ alkyl groups; a cyano group; a phenoxy group; C₃₋₈ cycloalkyl groups; pyrrolidinyl groups optionally substituted with one or two oxo groups; 1,2,3,4-tetrahydroisoquinolylcarbonyl groups; 1,2,3,4-tetrahydroquinolylcarbonyl groups optionally substituted with one to three straight and/or branched C₁₋₆ alkyl groups; 1,2,3,4-tetrahydroquinoxalinylcarbonyl groups optionally substituted with one to three straight and/or branched C₁₋₆ alkyl groups; thiazolyl groups optionally substituted with one to three phenyl groups; a carbamoyl group; phenyl alkoxy groups wherein the alkoxy moiety is a straight or branched C₁₋₆ alkoxy group; straight and branched C₁₋₆ alkylsulfonylamino groups; anilino groups optionally substituted with one to three halogen atoms; phenyl alkyl groups wherein the alkyl moiety is a straight or branched C₁₋₆ alkyl group; and hydroxy-substituted alkyl groups wherein the alkyl moiety is a straight or branched C₁₋₆ alkyl group, substituted with one to three hydroxy groups;

such as (2-, 3-, or 4-)trifluoromethylphenyl, (2-, 3-, or 4-)methylthiophenyl, (2-, 3-, or 4-) trifluoromethoxyphenyl, (2-, 3-, or 4-)ethylphenyl, (2-, 3-, or 4-)propylphenyl, (2-, 3-, or 4-)butylphenyl, (2-, 3-, or 4-)pentylphenyl, (2-, 3-, or 4-)hexylphenyl, (2-, 3-, or 4-)isopropylphenyl, (2-, 3-, or 4-)chlorophenyl, (2-, 3-, or 4-)fluorophenyl, (2-, 3-, or 4-)phenylphenyl, (2-, 3-, or 4-)dimethylaminophenyl, (2-, 3-, or 4-)cyanophenyl, (2-, 3-, or 4-)phenyloxyphenyl, (3,4-, 2,3-, 2,6-, or 3,5-) dimethylphenyl, (3,4-, 2,3-, 2,6-, or 3,5-)difluorophenyl, 2-chloro-4-methylphenyl, (2-, 3-, or 4-)cyclohexylphenyl, (2-, 3-, or 4-)benzyloxyphenyl, (2-, 3-, or 4-) methylsulfonylaminophenyl, (2-, 3- or 4-)anilinophenyl, (3,4-, 2,3-, 2,6- or 3,5-)dimethoxyphenyl, 3-chloro-4-methoxyphenyl, 3-chloro-4-methylphenyl, 3-methoxy-5-trifluoromethylphenyl, 2-chloro-5-trifluoromethylphenyl, 2-chloro-6-cyanophenyl, 2-chloro-5-carbamoylphenyl, (2-, 3-, or 4-)phenylmethylphenyl, (2-, 3-, or 4-)pyrrolidinylphenyl, (2-, 3-, or 4-)[(1-, 2-, 3-, or 4-) (1,2,3,4-tetrahydroisoquinolylcarbonyl)]phenyl, (2-, 3-, or 4-) [(1-, 2-, 3-, or 4-)(6-methyl-1,2,3,4-tetrahydroquinolyl carbonyl)]phenyl, (2-, 3-, or 4-)(4-fluoroanilino)phenyl, (2-, 3- or 4-)[4-methyl-1-(1,2,3,4-tetrahydroquinoxalinyl)carbonyl]phenyl, and (2-, 3-, or 4-) [(4- or 5-) phenylthiazolyl-2-yl]phenyl.

Examples of phenyl lower alkyl groups optionally substituted on the phenyl ring with one or more members selected from the group consisting of lower alkyl groups optionally substituted with one or more halogen atoms; lower alkoxy groups optionally substituted with one or more halogen atoms; halogen atoms; and a phenyl group include:

phenyl alkyl groups wherein the alkyl moiety is a straight or branched C₁₋₆ alkyl group, optionally substituted on the phenyl ring with one to three members selected from the group consisting of straight and branched C₁₋₆ alkyl groups optionally substituted with one to three halogen atoms; straight and branched C₁₋₆ alkoxy groups optionally substituted with one to three halogen atoms; halogen atoms; and a phenyl group;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-diphenylmethyl, 2,2-diphenylethyl, 3,3-diphenylpropyl, 1,2-diphenylethyl, 4-chlorobenzyl, 2-chlorobenzyl, 3-chlorobenzyl, 3-fluorobenzyl, 4-fluorobenzyl, (2- or 4-)bromobenzyl, 2,3-dichlorobenzyl, 2,4-dichlorobenzyl, 3-chloro-4-fluorobenzyl, 2,4,6-trifluorobenzyl, 3-trifluoromethylbenzyl, 4-trifluoromethylbenzyl, 2-methylbenzyl, 3-methylbenzyl, 4-methylbenzyl, 4-tert-butylbenzyl, 2,4-dimethylbenzyl, 2,4,6-trimethylbenzyl, 2-phenylbenzyl, 3-phenylbenzyl, 4-phenylbenzyl, 2,4-diphenylbenzyl, 2,4,6-triphenylbenzyl, 2-trifluoromethoxybenzyl, 3-trifluoromethoxybenzyl, 4-trifluoromethoxybenzyl, 3-chloro-4-difluoromethoxybenzyl, 4-chloro-3-trifluoromethylbenzyl, 2-methoxybenzyl, 3-methoxybenzyl, 4-methoxybenzyl, 3,4-dimethoxybenzyl, 3,4,5-trimethoxybenzyl, 2-(4-methoxyphenyl)ethyl, 2-(2-methoxyphenyl)ethyl, and 2-(4-chlorophenyl)ethyl.

Examples of lower alkyl-substituted amino lower alkyl groups include:

aminoalkyl groups wherein the alkyl moiety is a straight or branched C₁₋₆ alkyl group, having on the amino group one or two straight and/or branched C₁₋₆ alkyl groups;

such as N-methylaminomethyl, N,N-diethylaminomethyl, N,N-di-n-propylaminoethyl, N,N-diisopropylaminoethyl, 3-(N,N-dimethylamino)propyl, 4-(N,N-dimethylamino)butyl, 5-(N,N-dimethylamino)pentyl, and 6-(N,N-dimethylamino)hexyl.

Examples of pyrazinyl lower alkyl groups optionally substituted on the pyrazine ring with one or more lower alkyl groups include:

pyrazinylalkyl groups wherein the alkyl moiety is a straight or branched C₁₋₆ alkyl group, optionally substituted on the pyrazine ring with one to three straight and/or branched C₁₋₆ alkyl groups;

such as (2- or 3-)pyrazinylmethyl, (1- or 2-)(2- or 3-pyrazinyl)ethyl, 3-(2- or 3-)pyrazinylpropyl, 4-(2- or 3-) pyrazinylbutyl, 5-(2- or 3-)pyrazinylpentyl, 6-(2- or 3-) pyrazinylhexyl, 2-methyl-5-pyrazinylmethyl, (1- or 2-)(2-methyl-5-pyrazinyl)ethyl, 3-(2-methyl-5-pyrazinyl)propyl, 4-(2-ethyl-5-pyrazinyl)butyl, 5-(2-ethyl-5-pyrazinyl)pentyl, and 6-(2-methyl-5-pyrazinyl)hexyl.

Examples of pyrazolyl lower alkyl groups optionally substituted on the pyrazoline ring with one or more lower alkyl groups include:

pyrazolylalkyl groups wherein the alkyl moiety is a straight or branched C₁₋₆ alkyl group, optionally substituted on the pyrazoline ring with one to three straight and/or branched C₁₋₆ alkyl groups;

such as (3-, 4-, or 5-)pyrazolylmethyl, (1- or 2-) (3-, 4-, or 5-)pyrazolylethyl, 3-(3-, 4-, or 5-)pyrazolylpropyl, 4-(3-, 4-, or 5-)pyrazolylbutyl, 5-(3-, 4-, or 5-)pyrazolylpentyl, 6-(3-, 4-, or 5-)pyrazolylhexyl, [1-methyl-(3-, 4-, or 5-) pyrazolyl]methyl, [1,5-dimethyl-(3- or 4-)pyrazolyl]methyl, and [1,5-dimethyl-(3- or 4-)pyrazolyl]ethyl.

Examples of piperidinyl groups optionally substituted on the piperidine ring with one or more members selected from the group consisting of lower alkyl groups; a benzoyl group; and phenyl lower alkyl groups optionally substituted on the phenyl ring with one or more members selected from halogen atoms and lower alkyl groups include:

piperidinyl groups optionally substituted on the piperidine ring with one to three members selected from the group consisting of straight and branched C₁₋₆ alkyl groups; a benzoyl group; and phenyl lower alkyl groups wherein the alkyl moiety is a straight or branched C₁₋₆ alkyl group, optionally substituted on the phenyl ring with one to three members selected from the group consisting of halogen atoms and straight and branched C₁₋₆ alkyl groups;

such as, N-methyl-(2-, 3-, or 4-)piperidinyl, N-ethyl-(2-, 3-, or 4-)piperidinyl, N-n-propyl-(2-, 3-, or 4-)piperidinyl, N-benzoyl-(2-, 3-, or 4-)piperidinyl, 1-benzyl-4-piperidinyl, 1-phenylethyl-4-piperidinyl, 1-(2-, 3-, or 4-)chlorophenylmethyl-4-piperidinyl, and 1-(2-, 3-, or 4-)methylphenylmethyl-4-piperidinyl, 1,2,3-trimethyl-(4-, 5-, or 6-)piperidinyl, 1-benzyl-3-methyl-(2-, 4-, 5-, or 6-)piperidinyl, and 1-benzoyl-2-benzyl-(3-, 4-, 5-, or 6-)piperidinyl.

Examples of 3,4-dihydrocarbostyril groups optionally substituted with one or more lower alkyl groups include 3,4-dihydrocarbostyril groups optionally substituted with one to three straight and/or branched C₁₋₆ alkyl groups, such as 3,4-dihydro-(5-, 6-, 7-, or 8-)carbostyril and (6-, 7-, or 8-)methyl-3,4-dihydro-5-carbostyril.

Examples of quinolyl groups optionally substituted with one or more lower alkyl groups include quinolyl groups optionally substituted with one to three straight and/or branched C₁₋₆ alkyl groups, such as (2-, 3-, 4-, 5-, 6-, 7- or 8-)quinolyl and 2-methyl-4-quinolyl.

Examples of carbazolyl groups optionally substituted with one or more lower alkyl groups include carbazolyl groups optionally substituted with one to three straight and branched C₁₋₆ alkyl groups, such as N-methyl-(2-, 3-, 4-, or 5-)carbazolyl and N-ethyl-(2-, 3-, 4-, or 5-)carbazolyl.

Examples of phenyl lower alkylcarbamoyl lower alkyl groups include phenylalkylcarbamoylalkyl groups wherein each of the two alkyl moieties is a straight or branched C₁₋₆ alkyl group, such as phenylmethylcarbamoylmethyl, (1- or 2-)phenylethyl carbamoylmethyl, (1- or 2-)phenylethylcarbamoylethyl, 3-(2-phenylethylcarbamoyl)propyl, 4-(2-phenylethylcarbamoyl)butyl, 5-(2-phenylethylcarbamoyl)pentyl, and 6-(2-phenylethylcarbamoyl) hexyl.

Examples of phenylcarbamoyl lower alkyl groups include phenylcarbamoylalkyl groups wherein the alkyl moiety is a straight or branched C₁₋₆ alkyl group, such as phenylcarbamoylmethyl, (1- or 2-)phenylcarbamoylethyl, 3-(phenylcarbamoyl)propyl, 4-(phenylcarbamoyl)butyl, 5-(phenylcarbamoyl)pentyl, and 6-(phenylcarbamoyl)hexyl.

Examples of anilino groups optionally substituted on the phenyl ring with one or more lower alkoxy groups, each lower alkoxy substituent optionally being substituted with one or more halogen atoms, include:

anilino groups optionally substituted on the phenyl ring with one to three straight and/or branched C₁₋₆ alkoxy groups, each alkoxy substituent optionally being substituted with one to three halogen atoms;

such as (2-, 3-, or 4-)chloromethoxyanilino, and (2-, 3-, or 4-)trifluoromethoxyanilino.

Examples of anilino groups substituted on the amino group with one or more lower alkyl groups and further substituted on the phenyl ring with one or more halogen atoms include:

anilino groups substituted on the amino group with one to three straight and/or branched C₁₋₆ alkyl groups and further substituted on the phenyl ring with one to three halogen atoms;

such as N-methyl-(2-, 3-, or 4-)chloroanilino, N-ethyl-(2-, 3-, or 4-)chloroanilino, N-methyl-(2-, 3-, or 4-) bromoanilino, N-methyl-(2-, 3-, or 4-)fluoroanilino, N-ethyl-(2-, 3-, or 4-)iodoanilino, and N-n-propyl-(2-, 3-, or 4-) chloroanilino.

Examples of 5- and 6-membered unsaturated heterocyclic rings formed from R⁸ and R⁹ being linked together, together with the nitrogen atom to which they are bound, include (2- or 3-) pyrroline, 1,2-dihydropyridine, 2,3-dihydropyridine, 1,2,3,4-tetrahydropyridine, and 1,2,5,6-tetrahydropyridine.

Examples of benzoyl groups optionally substituted on the phenyl ring with one or more members selected from the group consisting of lower alkyl groups optionally substituted with one or more halogen atoms; a phenyl group; halogen atoms; a cyano group; a phenoxy group; lower alkoxycarbonyl groups; pyrazolyl groups; and lower alkoxy groups optionally substituted with one or more halogen atoms include:

benzoyl groups optionally substituted on the phenyl ring with one to three members selected from the group consisting of the above-described straight and branched C₁₋₆ alkyl groups optionally substituted with one to three halogen atoms; a phenyl group; halogen atoms; a cyano group; a phenoxy group; the above-described straight and branched C₁₋₆ alkoxycarbonyl groups; pyrazolyl groups; and the above-described straight and branched C₁₋₆alkoxy groups optionally substituted with one to three halogen atoms;

such as benzoyl, 4-methylbenzoyl, 3-methylbenzoyl, 2-methylbenzoyl, 4-tert-butylbenzoyl, 2,4-dimethylbenzoyl, 2,4,6-trimethylbenzoyl, 3-trifluoromethylbenzoyl, 4-trifluoromethylbenzoyl, 2-trifluoromethylbenzoyl, 4-phenylbenzoyl, 4-chlorobenzoyl, 3-chlorobenzoyl, 2-chlorobenzoyl, 4-fluorobenzoyl, 3-fluorobenzoyl, 2-fluorobenzoyl, 3-bromobenzoyl, 2-bromobenzoyl, 4-bromobenzoyl, 3,4-dichlorobenzoyl, 2,3-dichlorobenzoyl, 2-chloro-4-fluorobenzoyl, 2-methoxy-5-chlorobenzoyl, 4-methoxybenzoyl, 3-methoxybenzoyl, 2-methoxybenzoyl, 3,4-dimethoxybenzoyl, 3,4,5-trimethoxybenzoyl, 3-trifluoromethoxybenzoyl, 4-trifluoromethoxybenzoyl, 2-trifluoromethoxybenzoyl, 3-cyanobenzoyl, 4-cyanobenzoyl, 2-cyanobenzoyl, 3-phenoxybenzoyl, 2-phenoxybenzoyl, 4-phenoxybenzoyl, 4-methoxycarbonylbenzoyl, 3-ethoxycarbonylbenzoyl, 2-tert-butoxycarbonylbenzoyl, and 4-(1-pyrazolyl)benzoyl.

Examples of alkanoyl groups include straight and branched C₁₋₁₀ alkanoyl groups, such as, in addition to the above-described lower alkanoyl groups, heptanoyl, octanoyl, nonanoyl, decanoyl, and 2-ethyl-hexanoyl.

Examples of phenyl lower alkanoyl groups optionally substituted on the phenyl ring with one or more members selected from the group consisting of halogen atoms and lower alkyl groups include:

phenylalkanoyl groups wherein the alkanoyl moiety is a straight or branched C₂₋₆ alkanoyl group, optionally substituted on the phenyl ring with one to three members selected from the group consisting of halogen atoms and straight and branched C₁₋₆ alkyl groups;

such as 2-phenylacetyl, 3-phenylpropionyl, 2-phenylpropionyl, 4-phenylbutyryl, 5-phenylpentanoyl, 6-phenylhexanoyl, 2,2-dimethyl-3-phenylpropionyl, 2-methyl-3-phenylpropionyl, 2-(4-fluorophenyl)acetyl, 3-(2,5-difluorophenyl)propionyl, 2-(2,4-difluorophenyl)propionyl, 4-(3,4-difluorophenyl)butyryl, 5-(3,5-difluorophenyl)pentanoyl, 6-(2,6-difluorophenyl)hexanoyl, 2-(2-chlorophenyl)acetyl, 3-(3-chlorophenyl)propionyl, 2-(4-chlorophenyl)propionyl, 4-(2,3-dichlorophenyl)propionyl, 5-(2,4-dichlorophenyl)pentanoyl, 6-(2,5-dichlorophenyl)hexanoyl, 2-(3,4-dichlorophenyl)acetyl, 3-(2,6-dichlorophenyl)propionyl, 2-(3-fluorophenyl)propionyl, 4-(2-fluorophenyl)butyryl, 5-(3-bromophenyl)pentanoyl, 6-(4-iodophenyl)hexanoyl, 2-(2-bromophenyl)acetyl, 3-(4-bromophenyl)propionyl, 2-(3,5-dichlorophenyl)propionyl, 4-(2,4,6-trifluorophenyl)butyryl, 5-(3,4-difluorophenyl)pentanoyl, 6-(2-iodophenyl)hexanoyl, 2-(3-iodophenyl)acetyl, 3-(4-iodophenyl)propionyl, 2-(2,3-dibromophenyl)propionyl, 4-(2,4-diiodophenyl)butyryl, 2-(2,4,6-trichlorophenyl)acetyl, 2-(4-methylphenyl)acetyl, 3-(2,5-dimethylphenyl)propionyl, 2-(2,4-diethylphenyl)propionyl, 4-(3,4-di-n-propylphenyl)butyryl, 2-(2-ethylphenyl)acetyl, 3-(3-n-propylphenyl)propionyl, 2-(4-tert-butylphenyl)propionyl, 2-(2,4,6-trimethylphenyl)acetyl, 2-(2,5-dichloro-4-methylphenyl)acetyl, 2-(3-methyl-4-chlorophenyl)acetyl, 4-(2-n-butylphenyl)butyryl, 5-(3-n-pentylphenyl)pentanoyl, and 6-(4-n-hexylphenyl)hexanoyl.

Examples of phenoxy lower alkanoyl groups optionally substituted on the phenyl ring with one or more halogen atoms include:

phenoxyalkanoyl groups wherein the alkanoyl moiety is a straight or branched C₂₋₆ alkanoyl group, optionally substituted on the phenyl ring with one to three halogen atoms;

such as, in addition to the above-described phenoxy lower alkanoyl groups, 2-(4-chlorophenoxy)acetyl, 2-(4-fluorophenoxy)acetyl, 3-(2,5-difluorophenoxy)propionyl, 2-(2,4-difluorophenoxy)propionyl, 4-(3,4-difluorophenoxy)butyryl, 5-(3,5-difluorophenoxy)pentanoyl, 6-(2,6-difluorophenoxy)hexanoyl, 2-(2-chlorophenoxy)acetyl, 3-(3-chlorophenoxy)propionyl, 2-(4-chlorophenoxy)propionyl, 4-(2,3-dichlorophenoxy)propionyl, 5-(2,4-dichlorophenoxy)pentanoyl, 6-(2,5-dichlorophenoxy)hexanoyl, 2-(3,4-dichlorophenoxy)acetyl, 3-(2,6-dichlorophenoxy)propionyl, 2-(3-fluorophenoxy)propionyl, 4-(2-fluorophenoxy)butyryl, 5-(3-bromophenoxy)pentanoyl, 6-(4-iodophenoxy)hexanoyl, 2-(2-bromophenoxy)acetyl, 3-(4-bromophenoxy)propionyl, 2-(3,5-dichlorophenoxy)propionyl, 4-(2,4,6-trifluorophenoxy)butyryl, 5-(3,4-difluorophenoxy)pentanoyl, 6-(2-iodophenoxy)hexanoyl, 2-(3-iodophenoxy)acetyl, 3-(4-iodophenoxy)propionyl, 2-(2,3-dibromophenoxy)propionyl, 4-(2,4-diiodophenoxy)butyryl, and 2-(2,4,6-trichlorophenoxy)acetyl.

Examples of phenyl lower alkenylcarbonyl groups include phenylalkenylcarbonyl groups containing one to three double bonds wherein the alkenyl moiety is a straight or branched C₂₋₆ alkenyl group, such as styrylcarbonyl (trivial name: cinnamoyl), 3-phenyl-2-propenylcarbonyl, 4-phenyl-2-butenylcarbonyl, 4-phenyl-3-butenylcarbonyl, 5-phenyl-4-pentenylcarbonyl, 5-phenyl-3-pentenylcarbonyl, 6-phenyl-5-hexenylcarbonyl, 6-phenyl-4-hexenylcarbonyl, 6-phenyl-3-hexenylcarbonyl, 4-phenyl-1,3-butadienylcarbonyl, and 6-phenyl-1,3,5-hexatrienylcarbonyl.

Examples of pyridylcarbonyl groups optionally substituted on the pyridine ring with one or more members selected from the group consisting of halogen atoms and lower alkyl groups, each lower alkyl substituent optionally being substituted with one or more halogen atoms, include:

pyridylcarbonyl groups optionally substituted on the pyridine ring with one to three members selected from the group consisting of halogen atoms and the above-described straight and branched C₁₋₆ alkyl groups optionally substituted with one to three halogen atoms;

such as (2-, 3-, or 4-)pyridylcarbonyl, 2-chloro-(3-, 4-, 5-, or 6-)pyridylcarbonyl, 2,6-dichloro-(3-, 4-, or 5-) pyridylcarbonyl, 2,3-dichloro-(4-, 5-, or 6-)pyridylcarbonyl, 2-trifluoromethyl-(3-, 4-, 5-, or 6-)pyridylcarbonyl, 2-bromo-(3-, 4-, 5-, or 6-)pyridylcarbonyl, 2,6-difluoro-(3-, 4-, or 5-) pyridylcarbonyl, 4-methyl-(2-, 3-, 5-, or 6-)pyridylcarbonyl, 3-chloro-(2-, 4-, 5-, or 6-)pyridylcarbonyl, 2,5-dibromo-(3-, 4-, or 5-)pyridylcarbonyl, 2-ethyl-4-chloro-(3-, 5-, or 6-) pyridylcarbonyl, 2,4,6-trifluoro-(3- or 5-)pyridylcarbonyl, 2,4-dimethyl-(3-, 5-, or 6-)pyridylcarbonyl, 2,4,6-trimethyl-(3- or 5-)pyridylcarbonyl, and 2-methyl-4-chloro-(3-, 5-, or 6-) pyridylcarbonyl.

Examples of piperidinylcarbonyl groups optionally substituted on the piperidine ring with one or more lower alkanoyl groups include piperidinylcarbonyl groups optionally substituted on the piperidine ring with one to three straight and/or branched C₁₋₆ alkanoyl groups, such as (2-, 3-, or 4-)piperidinylcarbonyl, 1-acetyl-(2-, 3-, or 4-) piperidinylcarbonyl, 1-n-propanoyl-(2-, 3-, or 4-) piperidinylcarbonyl, 1-isopropanoyl-(2-, 3-, or 4-) piperidinylcarbonyl, 1-n-butyryl-(2-, 3-, or 4-) piperidinylcarbonyl, 1-n-pentanoyl-(2-, 3-, or 4-) piperidinylcarbonyl, 1-n-hexanoyl-(2-, 3-, or 4-) piperidinylcarbonyl, 1,2-diacetyl-(3-, 4-, 5-, or 6-) piperidinylcarbonyl, 1,2,3-triacetyl-(4-, 5-, or 6-) piperidinylcarbonyl, 2-acetyl-(1-, 3-, 4-, 5-, or 6-) piperidinylcarbonyl, 3-propanoyl-(1-, 2-, 4-, 5-, or 6-) piperidinylcarbonyl, and 2-formyl-4-propanoyl-(1-, 3-, 5-, or 6-) piperidinylcarbonyl.

Examples of tetrahydropyranylcarbonyl groups include 2-tetrahydropyranylcarbonyl, 3-tetrahydropyranylcarbonyl, and 4-tetrahydropyranylcarbonyl.

Examples of benzothienylcarbonyl groups optionally substituted on the benzothiophene ring with one or more halogen atoms include benzothienylcarbonyl groups optionally substituted on the benzothiophene ring with one to three halogen atoms, such as (2-, 3-, 4-, 5-, 6-, or 7-)benzothienylcarbonyl, [3-chloro-(2-, 4-, 5-, 6-, or 7-)benzothienyl]carbonyl, [4-bromo-(2-, 3-, 5-, 6-, or 7-)benzothienyl]carbonyl, [5-fluoro-(2-, 3-, 4-, 6-, or 7-)benzothienyl]carbonyl, [6-iodo-(2-, 3-, 4-, 5-, or 7-)benzothienyl]carbonyl, [7-chloro-(2-, 3-, 4-, 5-, or 6-)benzothienyl]carbonyl, [2-chloro-(3-, 4-, 5-, 6-, or 7-)benzothienyl]carbonyl, [2,3-dichloro-(4-, 5-, 6-, or 7-)benzothienyl]carbonyl, and [3,4,6-trichloro-(2-, 5- or 7-)benzothienyl]carbonyl.

Examples of pyridyl lower alkyl groups optionally substituted on the pyridine ring with one or more members selected from the group consisting of halogen atoms and lower alkyl groups, each lower alkyl substituent optionally being substituted with one or more halogen atoms, include:

pyridylalkyl groups wherein the alkyl moiety is a straight or branched C₁₋₆alkyl group, optionally substituted on the pyridine ring with one to three members selected from the group consisting of halogen atoms and the above-described straight and branched C₁₋₆alkyl groups optionally substituted with one to three halogen atoms;

such as (2-, 3-, or 4-)pyridylmethyl, 2-[(2-, 3-, or 4-)pyridyl]ethyl, 1-[(2-, 3-, or 4-)pyridyl]ethyl, 3-[(2-, 3-, or 4-)pyridyl]propyl, 4-[(2-, 3-, or 4-)pyridyl]butyl, 1,1-dimethyl-2-[(2-, 3-, or 4-)pyridyl]ethyl, 5-[(2-, 3-, or 4-) pyridyl]pentyl, 6-[(2-, 3-, or 4-)pyridyl]hexyl, 1-[(2-, 3-, or 4-)pyridyl]isopropyl, 2-methyl-3-[(2-, 3-, or 4-) pyridyl]propyl, [2-chloro-(3-, 4-, 5-, or 6-)pyridyl]methyl, [2,3-dichloro-(4-, 5-, or 6-)pyridyl]methyl, [2-bromo-(3-, 4-, 5-, or 6-)pyridyl]methyl, [2,4,6-trifluoro-(3-, 5-, or 6-)pyridyl]methyl, [2-trifluoromethyl-(3-, 4-, 5-, or 6-)pyridyl]methyl, [2-methyl-(3-, 4-, 5-, or 6-) pyridyl]methyl, [2-ethyl-(3-, 4-, 5-, or 6-)pyridyl]methyl, 2-[2-n-propyl-(3-, 4-, 5-, or 6-)pyridyl]ethyl, 3-[2-n-butyl-(3-, 4-, 5-, or 6-)pyridyl]propyl, 4-[2-n-pentyl-(3-, 4-, 5-, or 6-)pyridyl]butyl, 5-[2-n-hexyl-(3-, 4-, 5-, or 6-)pyridyl]pentyl, 6-[2-isopropyl-(3-, 4-, 5-, or 6-)pyridyl]hexyl, [2-tert-butyl-(3-, 4-, 5-, or 6-)pyridyl]methyl, [2,4-dimethyl-(3-, 5-, or 6-) pyridyl]methyl, [2,4,6-trimethyl-(3- or 5-)pyridyl]methyl, [2,4-ditrifluoromethyl-(3-, 5-, or 6-)pyridyl]methyl, 2-(2,4-bistrifluoromethyl)-(3-, 5-, or 6-)pyridyl)ethyl, and 3-[2-methyl-6-chloro-(3-, 4-, or 5-)pyridyl]propyl.

Examples of thienyl lower alkyl groups optionally substituted on the thiophene ring with one or more halogen atoms include:

thienylalkyl groups wherein the alkyl moiety is a straight or branched C₁₋₆ alkyl group, optionally substituted on the thiophene ring with one to three halogen atoms;

such as [(2- or 3-)thienyl]methyl, 2-[(2- or 3-) thienyl]ethyl, 1-[(2- or 3-)thienyl]ethyl, 3-[(2- or 3-)thienyl] propyl, 4-[(2- or 3-)thienyl]butyl, 5-[(2- or 3-)thienyl]pentyl, 6-[(2- or 3-)thienyl]hexyl, 1,1-dimethyl-2-[(2- or 3-)thienyl]ethyl, 2-methyl-3-[(2- or 3-)thienyl]propyl, [2-chloro-(3-, 4-, or 5-)thienyl]methyl, [4-bromo-(2-, 3-, or 5-)thienyl]methyl, [5-fluoro-(2-, 3-, or 4-) thienyl]methyl, [3-iodo-(2-, 4-, or 5-)thienyl]methyl, [2,3-dichloro-(4- or 5-)thienyl]methyl, (2,4,5-trichloro-3-thienyl)methyl, 2-[2-fluoro-(3-, 4-, or 5-)thienyl]ethyl, 1-[4-iodo-(2-, 3-, or 5-)thienyl]ethyl, 3-[3-chloro-(2-, 4-, or 5-) thienyl]propyl, 4-[4,5-dichloro-(2- or 3-)thienyl]butyl, 5-(2,4,5-trichloro-3-thienyl) pentyl, and 6-[2-chloro-(3-, 4-, or 5-)thienyl]hexyl.

Examples of amino groups optionally substituted with one or more members selected from the group consisting of lower alkyl groups and lower alkanoyl groups include:

amino groups optionally substituted with one or two members selected from the group consisting of straight and branched C₁₋₆ alkyl groups and straight and branched C₁₋₆ alkanoyl groups;

such as amino, formylamino, acetylamino, propionylamino, butyrylamino, isobutyrylamino, pentanoylamino, tert-butylcarbonylamino, hexanoylamino, N,N-diacetylamino, N-acetyl-N-propionylamino, methylamino, ethylamino, n-propylamino, isopropylamino, n-butylamino, n-pentylamino, n-hexylamino, dimethylamino, 3-diethylamino, diisopropylamino, N-ethyl-N-n-propylamino, N-methyl-N-n-hexylamino, N-methyl-N-acetylamino, and N-ethyl-N-acetylamino.

Examples of phenyl lower alkyl groups optionally substituted on the phenyl ring with one or more members selected from the group consisting of lower alkoxy groups optionally substituted with one or more halogen atoms; a cyano group; lower alkyl groups optionally substituted with one or more halogen atoms; amino groups optionally substituted with one or more members selected from the group consisting of lower alkyl groups and lower alkanoyl groups; halogen atoms; lower alkoxycarbonyl groups; lower alkanoyloxy groups; lower alkylsulfonyl groups; lower alkylthio groups; and pyrrolidinyl groups include:

mono- and di-phenylalkyl groups wherein the alkyl moiety is a straight or branched C₁₋₆ alkyl group, optionally substituted on the phenyl ring with one to three members selected from the group consisting of the above-described straight and branched C₁₋₆ alkoxy groups optionally substituted with one to three halogen atoms; a cyano group; the above-described straight and branched C₁₋₆ alkyl groups optionally substituted with one to three halogen atoms; the above-described amino groups optionally substituted with one or two members selected from the group consisting of straight and branched C₁₋₆ alkyl groups and straight and branched C₁₋₆ alkanoyl groups; halogen atoms; the above-described straight and branched C₁₋₆ alkoxycarbonyl groups; the above-described straight and branched C₂₋₆ alkanoyloxy groups; the above-described straight and branched C₁₋₆ alkylsulfonyl groups; the above-described straight and branched C₁₋₆alkylthio groups; and pyrrolidinyl groups;

such as benzyl, 1-phenethyl, 2-phenethyl, 3-phenylpropyl, 2-phenylpropyl, 4-phenylbutyl, 5-phenylpentyl, 4-phenylpentyl, 6-phenylhexyl, 2-methyl-3-phenylpropyl, 1,1-dimethyl-2-phenylethyl, 1,1-diphenylmethyl, 2,2-diphenylethyl, 3,3-diphenylpropyl, 1,2-diphenylethyl, 4-chlorobenzyl, 2-chlorobenzyl, 3-chlorobenzyl, 3-fluorobenzyl, 4-fluorobenzyl, 2-fluorobenzyl, 4-bromobenzyl, 3-bromobenzyl, 2-bromobenzyl, 1-(2-chlorophenyl)ethyl, 2,3-dichlorobenzyl, 2,4,6-trifluorobenzyl, 2-trifluoromethylbenzyl, 3-trifluoromethylbenzyl, 4-trifluoromethylbenzyl, 2-methylbenzyl, 3-methylbenzyl, 4-methylbenzyl, 4-tert-butylbenzyl, 4-n-butylbenzyl, 2,4-dimethylbenzyl, 2,4,6-trimethylbenzyl, 2-phenylbenzyl, 4-phenylbenzyl, 2,4-diphenylbenzyl, 2,4,6-triphenylbenzyl, 2-trifluoromethoxybenzyl, 3-trifluoromethoxybenzyl, 4-trifluoromethoxybenzyl, 4-difluoromethoxybenzyl, 2-methoxybenzyl, 3-methoxybenzyl, 4-methoxybenzyl, 4-n-butoxybenzyl, 4-tert-butoxybenzyl, 1-(3-methoxyphenyl)ethyl, 1-(4-methoxyphenyl)ethyl, 1-(2-methoxyphenyl)ethyl, 3,4-dimethoxybenzyl, 3,4,5-trimethoxybenzyl, 4-methoxycarbonylbenzyl, 3-ethoxycarbonylbenzyl, 2-n-propoxycarbonylbenzyl, 2,4-dimethoxycarbonylbenzyl, 2,4,6-trimethoxycarbonylbenzyl, 1-(4-n-butoxyphenyl)ethyl, 4-tert-butoxycarbonylbenzyl, 4-methylthiobenzyl, 3-methylthiobenzyl, 2-methylthiobenzyl, 4-ethylthiobenzyl, 2,4-dimethylthiobenzyl, 2,4,6-trimethylthiobenzyl, 4-methylsulfonylbenzyl, 3-methylsulfonylbenzyl, 2-methylsulfonylbenzyl, 3,4-dimethylsulfonylbenzyl, 3,4,5-trimethylsulfonylbenzyl, 4-methoxy-3-chlorobenzyl, 4-(N-acetylamino)benzyl, 4-(N,N-diethylamino)benzyl, 4-(N,N-dimethylamino)benzyl, 4-(N-methylamino)benzyl, 3-aminobenzyl, 2-aminobenzyl, 4-aminobenzyl, 4-acetyloxybenzyl, 2,3-diaminobenzyl, 3,4,5-triaminobenzyl, 4-methyl-3-fluorobenzyl, 4-cyanobenzyl, 3-cyanobenzyl, 2-cyanobenzyl, 4-(1-pyrrolidinyl)benzyl, 4-methoxy-2-chlorobenzyl, and 3-chloro-5-methylbenzyl.

Examples of thiazolyl lower alkyl groups include thiazolylalkyl groups wherein the alkyl moiety is a straight or branched C₁₋₆alkyl group, such as [(2-, 4-, or 5-)thiazolyl]methyl, 2-[(2-, 4-, or 5-)thiazolyl]ethyl, 1-[(2-, 4-, or 5-)thiazolyl]ethyl, 3-[(2-, 4-, or 5-)thiazolyl]propyl, 4-[(2-, 4-, or 5-) thiazolyl]butyl, 5-[(2-, 4-, or 5-)thiazolyl]pentyl, 6-[(2-, 4-, or 5-)thiazolyl]hexyl, 1,1-dimethyl-2-[(2-, 4-, or 5-) thiazolyl]ethyl, and [2-methyl-3-[(2-, 4-, or 5-)thiazolyl]propyl.

Examples of imidazolyl lower alkyl groups optionally substituted on the imidazole ring with one or more lower alkyl groups include:

imidazolylalkyl groups wherein the alkyl moiety is a straight or branched C₁₋₆alkyl group, optionally substituted on the imidazole ring with one to three above-described straight and branched C₁₋₆alkyl groups;

such as [(1-, 2-, 4-, or 5-)imidazolyl]methyl, 2-[(1-, 2-, 4-, or 5-)imidazolyl]ethyl, 1-[(1-, 2-, 4-, or 5-) imidazolyl]ethyl, 3-[(1-, 2-, 4-, or 5-)imidazolyl]propyl, 4-[(1-, 2-, 4-, or 5-)imidazolyl]butyl, 1,1-dimethyl-2-[(1-, 2-, 4-, or 5-)imidazolyl]ethyl, 5-[(1-, 2-, 4-, or 5-) imidazolyl]pentyl, 6-[(1-, 2-, 4-, or 5-)imidazolyl]hexyl, 1-[(1-, 2-, 4-, or 5-)imidazolyl]isopropyl, 2-methyl-3-[(1-, 2-, 4-, or 5-)imidazolyl]propyl, [1-methyl-(2-, 4-, or 5-)imidazolyl]methyl, [1-ethyl-(2-, 4-, or 5-) imidazolyl]methyl, [1-n-propyl-(2-, 4-, or 5-)imidazolyl]methyl, [1-n-butyl-(2-, 4-, or 5-)imidazolyl]methyl, [1-n-pentyl-(2-, 4-, or 5-)imidazolyl]methyl, [1-n-hexyl-(2-, 4-, or 5-) imidazolyl]methyl, 2-[2-methyl-(1-, 4-, or 5-)imidazolyl]ethyl, 1-[1-ethyl-(2-, 4-, or 5-)imidazolyl]ethyl, 3-[1-ethyl-(2-, 4-, or 5-)imidazolyl]methyl, 4-[1-n-propyl-(2-, 4-, or 5-) imidazolyl]butyl, 5-[1-n-butyl-(2-, 4-, or 5-)imidazolyl]pentyl, 6-[1-n-pentyl-(2-, 4-, or 5-)imidazolyl]hexyl, [1,2-dimethyl-(4- or 5-)imidazolyl]methyl, and (1,2,4-trimethyl-5-imidazolyl)methyl.

Examples of pyrrolyl lower alkyl groups optionally substituted on the pyrrole ring with one or more lower alkyl groups include:

pyrrolylalkyl groups wherein the alkyl moiety is a straight or branched C₁₋₆ alkyl group, optionally substituted on the pyrrole ring with one to three above-described straight and branched C₁₋₆ alkyl groups;

such as [(1-, 2-, or 3-)pyrrolyl]methyl, 2-[(1-, 2-, or 3-)pyrrolyl]ethyl, 1-[(1-, 2-, or 3-)pyrrolyl]ethyl, 3-[(1-, 2-, or 3-)pyrrolyl]propyl, 4-[(1-, 2-, or 3-) pyrrolyl]butyl, 1,1-dimethyl-2-[(1-, 2-, or 3-)pyrrolyl]ethyl, 5-[(1-, 2-, or 3-)pyrrolyl]pentyl, 6-[(1-, 2-, or 3-)pyrrolyl]hexyl, 1-[(1-, 2-, or 3-)pyrrolyl]isopropyl, 2-methyl-3-[(1-, 2-, or 3-) pyrrolyl]propyl, [1-methyl-(2- or 3-)pyrrolyl]methyl, [1-ethyl-(2- or 3-)pyrrolyl]methyl, [1-n-propyl-(2- or 3-)pyrrolyl]methyl, [1-n-butyl-(2- or 3-)pyrrolyl]methyl, [1-n-pentyl-(2- or 3-) pyrrolyl]methyl, [1-n-hexyl-(2- or 3-)pyrrolyl]methyl, 2-[2-methyl-(1-, 3-, 4-, or 5-)pyrrolyl]ethyl, 1-[1-ethyl-(2- or 3-) pyrrolyl]ethyl, 3-[1-ethyl-(2- or 3-)pyrrolyl]methyl, 4-[1-n-propyl-(2- or 3-)pyrrolyl]butyl, 5-[1-n-butyl-(2- or 3-)pyrrolyl] pentyl, 6-[1-n-pentyl-(2- or 3-)pyrrolyl]hexyl, [1,2-dimethyl-(3-, 4-, or 5-)pyrrolyl]methyl, and [1,2,4-trimethyl-(3- or 5-) pyrrolyl]methyl.

Examples of lower alkylthio lower alkyl groups include alkylthioalkyl groups wherein each of the two alkyl moieties is a straight or branched C₁₋₆ alkyl group, such as methylthiomethyl, 2-methylthioethyl, 1-ethylthioethyl, 2-ethylthioethyl, 3-n-butylthiopropyl, 4-n-propylthiobutyl, 1,1-dimethyl-2-n-pentylthioethyl, 5-n-hexylthiopentyl, 6-methylthiohexyl, 1-ethylthioisopropyl, and 2-methyl-3-methylthiopropyl.

Examples of phenoxycarbonyl groups optionally substituted on the phenyl ring with one or more members selected from the group consisting of halogen atoms, lower alkyl groups, and lower alkoxy groups include:

phenoxycarbonyl groups optionally substituted on the phenyl ring with one to three members selected from the group consisting of halogen atoms, the above-described straight and branched C₁₋₆ alkyl groups, and the above-described straight and branched C₁₋₆ alkoxy groups;

such as phenoxycarbonyl, 4-chlorophenoxycarbonyl, 3-chlorophenoxycarbonyl, 2-chlorophenoxycarbonyl, 3,4-dichlorophenoxycarbonyl, 2,4,6-trichlorophenoxycarbonyl, 4-fluorophenoxycarbonyl, 3-fluorophenoxycarbonyl, 2-fluorophenoxycarbonyl, 2,4-difluorophenoxycarbonyl, 3,4,5-trifluorophenoxycarbonyl, 4-bromophenoxycarbonyl, 2-chloro-4-methoxyphenoxycarbonyl, 3-fluoro-5-methylphenoxycarbonyl, 4-methoxyphenoxycarbonyl, 3-methoxyphenoxycarbonyl, 2-methoxyphenoxycarbonyl, 3,4-dimethoxyphenoxycarbonyl, 2,4,5-trimethoxyphenoxycarbonyl, 4-methylphenoxycarbonyl, 3-methylphenoxycarbonyl, 2-methylphenoxycarbonyl, 2,5-dimethylphenoxycarbonyl, and 2,3,4-trimethylphenoxycarbonyl.

Examples of phenyl lower alkoxycarbonyl groups optionally substituted on the phenyl ring with one or more halogen atoms include:

phenylalkoxycarbonyl groups wherein the alkoxy moiety is a straight or branched C₁₋₆ alkoxy group, optionally substituted on the phenyl ring with one to three halogen atoms;

such as benzyloxycarbonyl, 2-phenylethoxycarbonyl, 1-phenylethoxycarbonyl, 3-phenylpropoxycarbonyl, 4-phenylbutoxycarbonyl, 5-phenylpentyloxycarbonyl, 6-phenylhexyloxycarbonyl, 1,1-dimethyl-2-phenylethoxycarbonyl, 2-methyl-3-phenylpropoxycarbonyl, 2-chlorobenzyloxycarbonyl, 3-chlorobenzyloxycarbonyl, 2-chlorobenzyloxycarbonyl, 3,4-dichlorobenzyloxycarbonyl, 2,4,6-trichlorobenzyloxycarbonyl, 4-fluorobenzyloxycarbonyl, 3-fluorobenzyloxycarbonyl, 2-fluorobenzyloxycarbonyl, 2,4-difluorobenzyloxycarbonyl, 3,4,5-trifluorobenzyloxycarbonyl, 4-bromobenzyloxycarbonyl, 4-nitrobenzyloxycarbonyl, and 3-nitrobenzyloxycarbonyl.

Examples of quinoxalinylcarbonyl groups include 2-quinoxalinylcarbonyl, 5-quinoxalinylcarbonyl, and 6-quinoxalinylcarbonyl.

Examples of phenyl lower alkanoyl groups include phenylalkanoyl groups wherein the alkanoyl moiety is a straight or branched C₂₋₆ alkanoyl group, such as 2-phenylacetyl, 3-phenylpropionyl, 2-phenylpropionyl, 4-phenylbutyryl, 5-phenylpentanoyl, 6-phenylhexanoyl, 2,2-dimethyl-2-phenylpropionyl, and 2-methyl-3-phenylpropionyl.

Among the carbostyril compounds represented by General Formula (1) or salts thereof, carbostyril compounds selected from the following compounds, or salts thereof are preferable:
5-[1-(biphenyl-4-ylmethyl)-8-methoxy-2-oxo-1,2,3,4-tetrahydroquinolin-5-ylmethyl]thiazolidine-2,4-dione,
5-[1-(4-chlorobenzyl)-8-methoxy-2-oxo-1,2,3,4-tetrahydroquinolin-5-ylmethyl]thiazolidine-2,4-dione,
5-[1-(4-bromobenzyl)-8-methoxy-2-oxo-1,2,3,4-tetrahydroquinolin-5-ylmethyl]thiazolidine-2,4-dione,
5-[1-(2-naphthylmethyl)-8-methoxy-2-oxo-1,2,3,4-tetrahydroquinolin-5-ylmethyl]thiazolidine-2,4-dione,
5-{1-[4-(heptyloxycarbonylamino)benzyl]-8-methoxy-2-oxo-1,2,3,4-tetrahydroquinolin-5-ylmethyl}thiazolidine-2,4-dione,
5-[1-(1-biphenyl-4-ylpiperidin-4-ylmethyl)-2-oxo-1,2,3,4-tetrahydroquinolin-5-ylmethyl]thiazolidine-2,4-dione,
5-{1-[1-(4-methylphenyl)piperidin-4-ylmethyl]-2-oxo-1,2,3,4-tetrahydroquinolin-5-ylmethyl}thiazolidine-2,4-dione,
5-{1-[4-(2-chlorobenzyloxycarbonylamino)benzyl]-8-methoxy-2-oxo-1,2,3,4-tetrahydroquinolin-5-ylmethyl}thiazolidine-2,4-dione,
1-(biphenyl-4-ylmethyl)-8-methoxy-5-(4-oxo-2-thioxothiazolidin-5-ylmethyl)-3,4-dihydro-1H-quinolin-2-one,
8-methoxy-1-methyl-5-(4-oxo-2-thioxothiazolidin-5-ylmethyl)-3,4-dihydro-1H-quinolin-2-one,
8-methoxy-1-(3-methylbutyl)-5-(4-oxo-2-thioxothiazolidin-5-ylmethyl)-3,4-dihydro-1H-quinolin-2-one,
1-propyl-8-methoxy-5-(4-oxo-2-thioxothiazolidin-5-ylmethyl)-3,4-dihydro-1H-quinolin-2-one,
1-isobutyl-8-methoxy-5-(4-oxo-2-thioxothiazolidin-5-ylmethyl)-3,4-dihydro-1H-quinolin-2-one,
8-methoxy-1-phenethyl-5-(4-oxo-2-thioxothiazolidin-5-ylmethyl)-3,4-dihydro-1H-quinolin-2-one, and
1-(4-phenylthiomethyl)benzyl-5-(4-oxo-2-thioxothiazolidin-5-ylmethyl)-3,4-dihydro-1H-quinolin-2-one.

The carbostyril compound of Formula (1) according to the present invention includes stereoisomers, optical isomers, hydrates, and like solvates thereof.

Among the compounds of the present invention, those having a basic group or groups can easily form salts with common pharmaceutically acceptable acids. Examples of such acids include hydrochloric acid, hydrobromic acid, nitric acid, sulfuric acid, phosphoric acid and other inorganic acid, methansulfonic acid, p-toluenesulfonic acid, acetic acid, citric acid, tartric acid, maleic acid, fumaric acid, malic acid, lactic acid and other organic acid, etc.

Among the compounds of the present invention, those having an acidic group or groups can easily form salts by reacting with pharmaceutically acceptable basic compounds. Examples of such basic compounds include sodium hydroxide, potassium hydroxide, calcium hydroxide, sodium carbonate, potassium carbonate, sodium hydrogencarbonate, potassium hydrogencarbonate, etc.

The following is an explanation of pharmaceutical preparations comprising the compound of the present invention as an active ingredient.

Such pharmaceutical preparations are obtained by formulating the compound of the present invention into usual pharmaceutical preparations, using usually employed diluents or excipients such as fillers, extenders, binders, wetting agents, disintegrants, surfactants, lubricants, etc.

The form of such pharmaceutical preparations can be selected from various forms according to the purpose of therapy. Typical examples include tablets, pills, powders, solutions, suspensions, emulsions, granules, capsules, suppositories, injections (solutions, suspensions, etc.) and the like.

To form tablets, any of various known carriers can be used, including, for example, lactose, white sugar, sodium chloride, glucose, urea, starch, calcium carbonate, kaolin, crystalline cellulose and other excipients; water, ethanol, propanol, simple syrup, glucose solutions, starch solutions, gelatin solutions, carboxymethylcellulose, shellac, methylcellulose, potassium phosphate, polyvinylpyrrolidone and other binders; dry starch, sodium alginate, agar powder, laminaran powder, sodium hydrogencarbonate, calcium carbonate, fatty acid esters of polyoxyethylenesorbitan, sodium laurylsulfate, stearic acid monoglyceride, starch, lactose and other disintegrants; white sugar, stearin, cacao butter, hydrogenated oils and other disintegration inhibitors; quaternary ammonium base, sodium lauryl sulfate and other absorption promoters; glycerin, starch and other wetting agents; starch, lactose, kaolin, bentonite, colloidal silicic acid and other adsorbents; purified talc, stearates, boric acid powder, polyethylene glycol and other lubricants; etc.

Such tablets may be coated with usual coating materials as required, to prepare, for example, sugar-coated tablets, gelatin-coated tablets, enteric-coated tablets, film-coated tablets, double- or multi-layered tablets, etc.

To form pills, any of various known carriers can be used, including, for example, glucose, lactose, starch, cacao butter, hydrogenated vegetable oils, kaolin, talc and other excipients; gum arabic powder, tragacanth powder, gelatin, ethanol and other binders; laminaran, agar and other disintegrants; etc.

To form suppositories, any of various known carriers can be used, including, for example, polyethylene glycol, cacao butter, higher alcohols, esters of higher alcohols, gelatin, semisynthetic glycerides, etc.

To form an injection, a solution, emulsion or suspension is sterilized and preferably made isotonic with blood. Any of various known widely used diluents can be employed to prepare the solution, emulsion or suspension. Examples of such diluents include water, ethanol, propylene glycol, ethoxylated isostearyl alcohol, polyoxylated isostearyl alcohol, fatty acid esters of polyoxyethylene sorbitan, etc. In this case, the pharmaceutical preparation may contain sodium chloride, glucose or glycerin in an amount sufficient to prepare an isotonic solution, and may contain usual solubilizers, buffers, analgesic agents, etc., and further, if necessary, coloring agents, preservatives, flavors, sweetening agents, etc., and/or other medicines.

The proportion of the compound of the present invention in the pharmaceutical preparation is not limited and can be suitably selected from a wide range. It is usually preferable that the pharmaceutical preparation contain the compound of the present invention in a proportion of 1 to 70 wt. %.

The route of administration of the pharmaceutical preparation according to the present invention is not limited, and the preparation is administered by a route suitable for the form of the preparation, patient's age and sex, conditions of the disease, and other conditions. For example, tablets, pills, solutions, suspensions, emulsions, granules and capsules are administered orally. Injections are intravenously administered singly or as mixed with usual injection transfusions such as glucose solutions, amino acid solutions or the like, or singly administered intramuscularly, intracutaneously, subcutaneously or intraperitoneally, as required. Suppositories are administered intrarectally.

The dosage of the pharmaceutical preparation is suitably selected according to the method of use, patient's age and sex, severity of the disease, and other conditions, and is usually about 0.001 to about 100 mg/kg body weight/day, and preferably 0.001 to 50 mg/kg body weight/day, in single or divided doses.

Since the dosage varies depending on various conditions, a dosage smaller than the above range may be sufficient or a dosage larger than the above range may be required.

The present invention, therefore, provides a pharmaceutical composition comprising the carbostyril compound as an active ingredient for the therapy and prophylaxis of various NF-κB-associated diseases and a method for the therapy and prophylaxis.

Diseases to be treated or prevented by the therapeutic/prophylactic composition of the invention are NF-κB-associated diseases, that is to say, diseases caused by the unwanted activation of genes under control of the transcriptional regulatory factor NF-κB. Examples of such diseases include ischemic diseases, inflammatory diseases, autoimmune diseases, cancer metastasis and invasion, and cachexia. Examples of ischemic diseases include ischemic diseases of organs (e.g. ischemic heart diseases such as myocardial infarction, acute heart failure, chronic heart failure, etc., ischemic brain diseases such as cerebral infarction, ischemic lung diseases such as pulmonary infarction), aggravation of symptoms after organ transplantation or organ surgery (e.g. aggravation of symptoms after heart transplantation, cardiac surgery, kidney transplantation, renal surgery, liver transplantation, hepatic surgery, bone marrow transplantation, skin grafting, corneal transplantation, and lung transplantation), reperfusion disorders, and post-PTCA restenosis. Examples of inflammatory diseases include various inflammatory diseases such as nephritis, hepatitis, arthritis, etc., acute renal failure, chronic renal failure, and arteriosclerosis. Examples of autoimmune diseases include but are not limited to rheumatism, multiple sclerosis, and Hashimoto's thyroiditis.

The pharmaceutical composition containing the carbostyril compound according to the present invention as an active ingredient is particularly suited for the therapy and prophylaxis of reperfusion disorders in ischemic diseases, aggravation of symptoms after organ transplantation or organ surgery, post-PTCA restenosis, cancer metastasis and invasion, and cachexia such as weight loss following the onset of a cancer.

The carbostyril compound (1) of the present invention or salts thereof have COX-2 inhibitory activity and are thus useful as COX-2 inhibitors. More specifically, the carbostyril compound (1) or salts thereof have COX-2 production-inhibitory activity and are thus useful as COX-2 production inhibitors.

Due to the COX-2 inhibitory activity, the carbostyril compound (1) or salts thereof exhibit radiosensitivity-enhancing effects in radiotherapy for cancer patients, and tumor-induced angiogenesis inhibitory activity. Therefore, the carbostyril compound (1) or salts thereof are useful as radiosensitivity enhancers for radiotherapy, or as tumor-induced angiogenesis inhibitors. Furthermore, due to the activity, the carbostyril compound (1) or salts thereof are useful as anticancer-effect reinforcers in radiotherapy or chemotherapy. As used herein, the term “chemotherapy” refers to a method of treating or preventing a tumor by using an antitumor agent. Examples of antitumor agents include those that can be used in the medical field, such as cisplatin, paclitaxel, anastrozole, calcium carbonate, capecitabine, carboplatin, Cell Pathways CP-461, docetaxel, doxorubicin, etoposide, fluoxymesterone, gemcitabine, goserelin, irinotecan, ketoconazole, letrozole, leucovorin, levamisole, megestrol, mitoxantrone, raloxifene, retinoic acid, tomoxifen, thiotepa, topotecan, toremifene, vinorelbine, vinblastine, vincristine, selenium (selenomethionine), sulindac sulfone, exemestane, eflornithine (DFMO), etc.

Examples of cancers to be treated or prevented include advanced malignancy, amyloidosis, neuroblastoma, meningioma, hemangiopericytoma, multiple brain metastases, glioblastoma multiforme, glioblastoma, brain stem glioma, poor prognosis malignant brain tumor, malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

Due to the COX-2 inhibitory activity, the carbostyril compound (1) or salts thereof are useful as therapeutic or preventive agents for COX-2-associated diseases and disorders, such as pain (chronic or acute pain), fever, inflammation, etc. COX-2-associated diseases and disorders are well known in the art to which the present invention pertains. Examples of COX-2-associated diseases and disorders include rheumatic fever; influenza- or other virus-infection-associated diseases such as cold; lumbar and cervical pain; headache; toothache; sprain and injury; myositis; sympathetically independent pain; synovitis; arthritis, including rheumatoid arthritis; degenerative joint diseases, including osteoarthrosis; gout and ankylosing spondylitis; tendinitis; skin-associated diseases such as psoriasis, eczema, burn, and dermatitis; injuries such as sports injuries; and disorders arising from surgical or dental treatment.

The carbostyril compound (1) or salts thereof are also useful as preventive or therapeutic agents for neurogenic pains. Neuropathic pain syndrome occurs after nerve injury. Even after the healing of the original injury, the resulting pain may continue for many months or years. Neuropathic pain may occur in a certain region of a peripheral nerve, a dorsal root, the spinal cord, or the brain. Neuropathic pain syndromes are originally classified according to the disease or disorder causing the syndrome. Examples of neuropathic pain syndromes include diabetic neuropathy; sciatica; nonspecific lumbar pain; multiple sclerosis pain; fibromyalgia; HIV-associated neuropathy; neuralgia such as neuralgia after herpes, or neuralgia of the trigeminal nerve; and pain caused by physical trauma, incision, cancer, poison, or acute inflammation.

Therefore, the carbostyril compound (1) or salts thereof are useful as anti-inflammatory agents, analgesics, etc., and can be administered separately or together with other COX-2 inhibitors, such as anti-inflammatory agents, analgesics, etc.

### BEST MODE FOR CARRYING OUT THE INVENTION

The following examples illustrate the invention in more detail.

### Example 1

### Effects of Test Compounds on COX-2 Gene Expression in Gastric Cancer Cell Line MKN-45

The human gastric cancer cell line MKN-45 obtained from JCRB (Japanese Collection of Research Bioresources) was cultured in Iscove's Modified Dulbecco's Medium (product of GIBCO) containing 10% fetal calf serum for 24 hours using a 6-well plate and a 5% CO₂ incubator (37° C., 5% CO₂). Test compound solutions prepared by diluting each test compound with DMSO (solvent) to a final concentration of 1 μM, and the same amount of DMSO as a control solution (containing no test compound) were added to the wells of the 6-well plate, and the cells were cultured in a CO₂ incubator (37° C., 5% CO₂) for 16 hours.

The cells were recovered, and Total RNA was prepared from the cells using Trizole reagent (product of Invitrogen) according to the standard method. Real-time PCR was performed using TaqManO Gene Expression Assays for human COX-2 (product of Applied Biosystems), and the amount of COX-2 mRNA in each test-compound-treated cell were determined.

The amount of COX-2 mRNA in test compound-treated cells were expressed as a percentage relative to that in the control cells set as 100% to determine the percent inhibition of COX-2 gene expression by each test compound. Each experiment was performed in triplicate, and the average of the three results was recorded.

The test compounds used in the experiments are as follows:

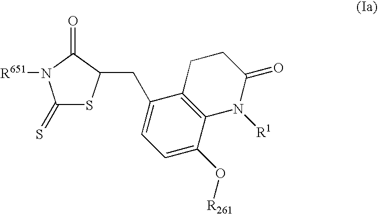

Test compound A: compound of formula (1a) wherein R¹ is methyl
Test compound B: compound of formula (1a) wherein R¹ is isopentyl
Test compound C: compound of formula (1a) wherein R¹ is 2-phenylethyl
   (in all the above test compounds, R₂₆₁ is methyl and R⁶⁵¹ is hydrogen).
   Test compounds A to C were prepared in the same manner as in Examples 960, 965, and 978 of WO2006/035954, respectively.

Table 1 shows the results.

TABLE 1

| Test compound | COX-2 gene expression inhibition (%) |
|---|---|
| Test compound A | >20 |
| Test compound B | >20 |
| Test compound C | >20 |

The real-time PCR was performed under the following conditions.

### Conditions of Real-Time PCR

a) Preparation of Standard Solutions for Calibration Curves of COX-2 mRNA and Beta-Actin mRNA

Standard solutions of COX-2 mRNA and beta-actin mRNA were prepared according to the ABI PRISM 7000 Sequence Detection System User Guide (Chapter 6 Analyzing Assay). More specifically, the amounts of COX-2mRNA and beta-actin mRNA in all the cell samples were preliminarily determined, and samples containing the highest levels of COX-2 mRNA and beta-actin mRNA among the solvent-control cell samples (samples with the lowest threshold cycle (Cₜ) value as determined based on the fluorescence detection using the ABI PRISM 7000 Sequence Detection System (product of Applied Biosystems)) were selected and used as standard solutions, respectively. Subsequently, dilution series of each standard solution were prepared to form a calibration curve of COX-2 mRNA and beta-actin mRNA levels. When inclusion of all the test samples in the calibration curve was found to be impossible, a sample with the lowest threshold cycle (Cₜ) value among the test-compound-treated cell samples was used as a standard solution to prepare a dilution series. In the dilution of the standard solutions, DEPC-treated water was used.

### b) PCR Reaction

PCR was performed according to the manufacturer's instructions for TaqMan® Gene Expression Assays (product of Applied Biosystems). More specifically, 12.5 μl of TaqMan® Universal PCR Master Mix, 1.25 μl of TaqMan® Gene Expression Assays for COX-2 mRNA, and 11.25 μl of cDNA solution, which was obtained in a similar manner to that of 3) of Example 3 described below, were added to each sample in a 96-well plate (“MicroAmp® Optical 96-well Reaction Plate”, product of Applied Biosystems), and stirred. A PCR reaction consisting of 50° C. for 2 minutes and 95° C. for 10 minutes, followed by 40 cycles consisting of 95° C. for 15 seconds and 60° C. for 1 minute was performed using a real-time PCR device (“ABI PRISM 7000 Sequence Detection System”, product of Applied Biosystems) with a data analysis software (“ABI PRISM 7000 SDS v1.1”, product of Applied Biosystems). The amount of each mRNA was determined from the calibration curve using the ABI PRISM 7000 Sequence Detection System.

c) Correction of COX-2 mRNA Amount

The amount of COX-2 mRNA was expressed relative to the amount of beta-actin mRNA using Excel 2000 (product of Microsoft Corporation), and used for data analysis.

### Example 2

### NF-kappaB Reporter Assay

(1) Preparation of pNF-kappaB-SEAP

pNF-kappaB-SEAP was obtained from Clonetech Laboratories Inc.

### (2) Culturing of MKN-45 Cells

Cryopreserved MKN-45 cells (product of JCRB (Japanese Collection of Research Bioresources)) were thawed and cultured in a standard medium in an incubator (37° C., 5% CO₂). Every three to five days, the cells were recovered by treatment with Trypsin-EDTA, then adequately diluted and subcultured. MKN-45 cells subcultured two or more times with a survival rate of 95% or more were used in each experiment.

### (3) Cell Seeding

The subcultured MKN-45 cells were harvested using Trypsin-EDTA solution, and the number of viable cells was counted. The cells were suspended in a standard medium to yield a 1.5×10⁵ cells/mL suspension. A 24-well plate was seeded with 500 μL/well of the cell suspension, and the cells were cultured in an incubator (37° C., 5% CO₂).

### (4) Transfection

The day after seeding, transfection was performed according to the following protocol.

(4.1) Preparation of Plasmid Solution 4 μL of 0.1 μg/μL pNFkappaB-SEAP was placed into each well and diluted with 50 μL of OPTI-MEM I (product of Invitrogen Japan K.K.) by mixing by inversion.

### (4.2) Preparation of Lipofectamine Dilution

1 μL of Lipofectamine 2000 reagent (product of Invitrogen) was placed into each well and diluted with 50 μL of OPTI-MEM I (product of Life Technologies Inc.) by mixing by inversion. The mixture was allowed to stand at room temperature for 5 to 10 minutes to prepare a Lipofectamine dilution.

### (4.3) Preparation of Transfection Mixture

The plasmid solution and the Lipofectamine dilution were mixed by inversion and allowed to stand at room temperature for 20 to 30 minutes to prepare a transfection mixture.

### (4.4) Addition to Cells

The culture medium was removed from the 24-well plate by suction. While care was taken not to dry the subcultured MKN-45 cells, the inner surface of the wells was washed with 1 mL/well of PBS. After the medium was replaced with 500 μL/well of OPTI-MEM I (product of Life Technologies Inc.), 100 μL/well of the transfection mixture was added, and the cells were cultured in a incubator (37° C., 5% CO₂). After about 5 hours of incubation, the medium was replaced with a standard medium, and the cells were further cultured in a CO₂ incubator (37° C., 5% CO₂) until the following day.

### (5) Addition of Test or Control Compound

On the following morning, the culture medium supernatant was removed by suction and replaced with 1 mL/well of the test compound solution or control solution diluted with a standard medium. The cells were cultured in a CO₂ incubator (37° C., 5% CO₂) for 1 hour. Each experiment was performed in triplicate.

### (6) Stimulus Addition

200 ng/mL IL-1β was added in an amount of 10 μL/well (final concentration: 2 ng/mL), and the cells were cultured in a CO₂ incubator (37° C., 5% CO₂) for 6 hours.

### (7) Reporter Assay

A reporter assay was performed using Reporter Assay Kit-SEAP (product of Toyobo Co.) according to the manufacturer's protocol.

More specifically, 500 μL of the culture broth was transferred into a tube and centrifuged at 15,000 rpm for 5 minutes at 4° C. The supernatant was transferred into a 96-well white plate. The positive control included in the Reporter Assay Kit-SEAP, and a standard medium as a blank control were each added in an amount of 20 μL/well to the 96-well white plate. Further, an inhibitor for inhibiting the AP activity of the MKN-45 cells was added in an amount of 20 μL/well. After sealing the plate, the contents were mixed well. The plate was placed into a CO₂ incubator (37° C., 5% CO₂) and allowed to stand for 30 minutes. Subsequently, the plate was allowed to stand on ice for 1 minute, followed by addition of 160 μL/well of Lumi-Phos Plus (a chemiluminescent substrate, product of Lumigen Inc.). The plate was sealed again and placed in a light-shielded state into a CO₂ incubator (37° C., 5% CO₂). After the plate was allowed to stand for 15 to 30 minutes, luminescence was measured for about 5 to about 10 seconds using a luminometer.

### (8) Results

The results of the reporter assay were expressed as Percent Control (or Inhibition), calculated according to the following formulae. The luminescence of each sample was calculated by subtracting the average of blank samples.

The results indicate that the carbostyril compound (I) or a salt thereof has an inhibiting activity against NF-κB activity.

$( Percent Control ) = \frac{\begin{matrix}( Luminescence of IL \text{-} 1 β \text{-} stimulated , \\ test compound \text{-} treated Cells )\end{matrix}}{\begin{matrix}( Luminescence of IL \text{-} 1 β \text{-} stimulated , \\ DMSO \text{-} treated Cells )\end{matrix}} \times 100$

| | | IL-1b(+) | |
|---|---|---|---|
| Test Compound *1 | | mean | SE |
| DMSO (0.05%) (control) | | 1.00 | 0.084 |
| 025 | 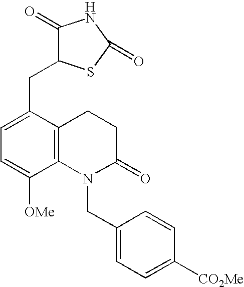 | 0.58 | 0.037 |
| 123 | 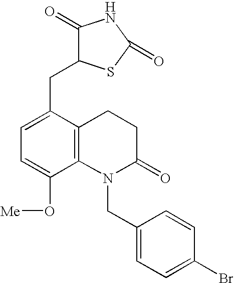 | 0.46 | 0.035 |
| 125 | 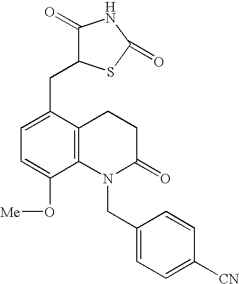 | 0.55 | 0.044 |
| 127 | 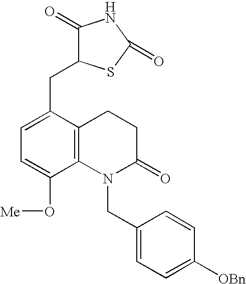 | 0.60 | 0.066 |
| 131 | 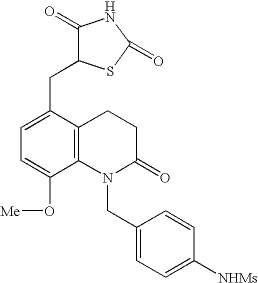 | 0.49 | 0.047 |
| 134 | 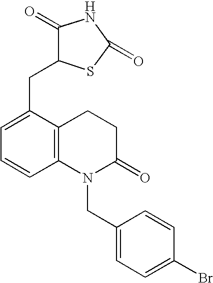 | 0.46 | 0.040 |
| 150 | 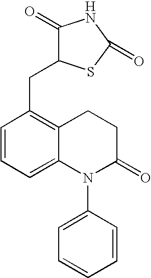 | 0.71 | 0.071 |
| 159 | 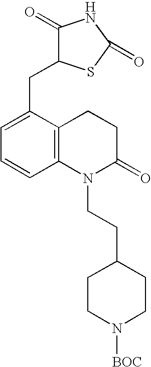 | 0.64 | 0.060 |
| 164 | 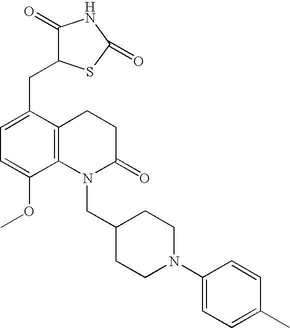 | 0.78 | 0.037 |
| 186 | 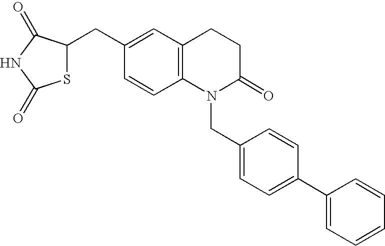 | 0.67 | 0.031 |
| 225 | 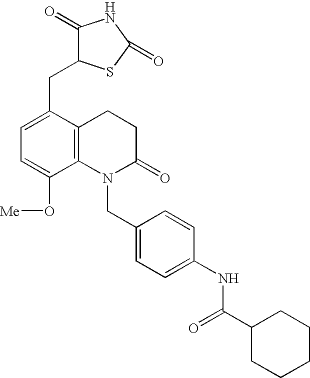 | 0.49 | 0.084 |
| 232 | 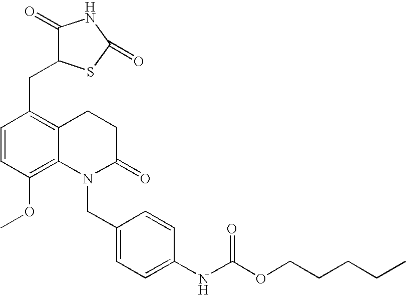 | 0.71 | 0.043 |
| 233 | 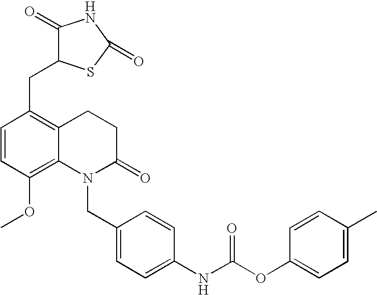 | 0.85 | 0.054 |
| 234 | 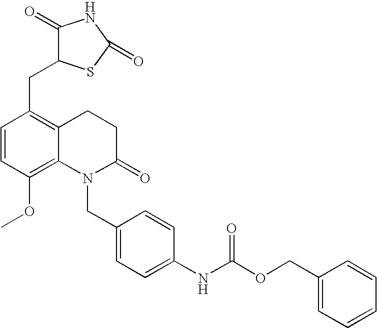 | 0.67 | 0.044 |
| 237 | 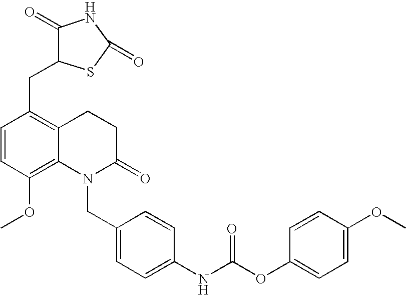 | 0.57 | 0.009 |
| 608 | 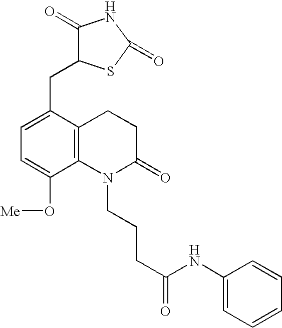 | 0.71 | 0.062 |
| 612 | 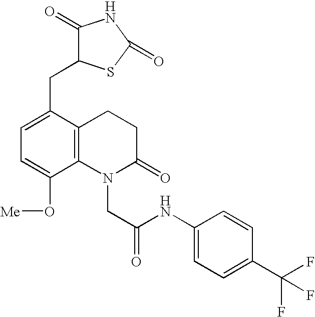 | 0.67 | 0.054 |
| 613 | 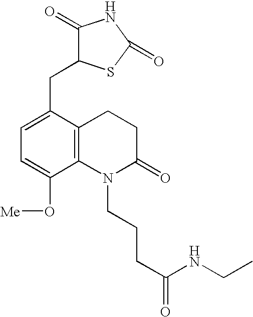 | 0.86 | 0.050 |
| 616 | 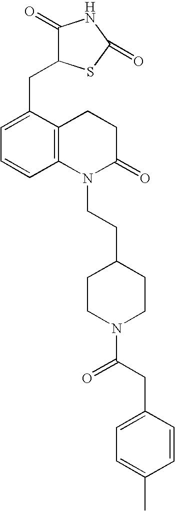 | 0.57 | 0.029 |
| 914 | 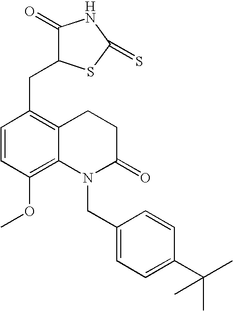 | 0.53 | 0.029 |
| 965 | 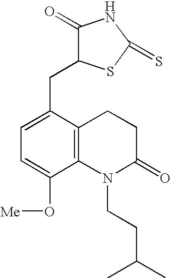 | 0.68 | 0.027 |
| 971 | 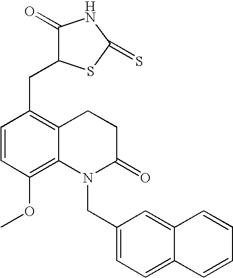 | 0.56 | 0.005 |
| 980 | 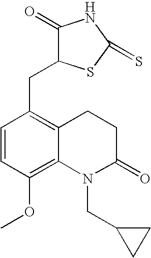 | 0.63 | 0.039 |
| 981 | 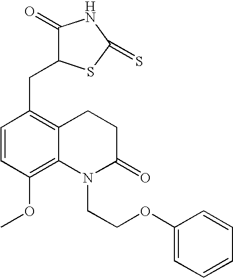 | 0.68 | 0.063 |
| 993 | 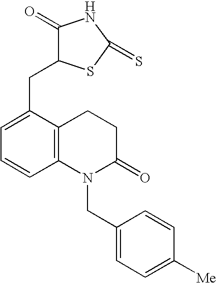 | 0.59 | 0.023 |
| 995 | 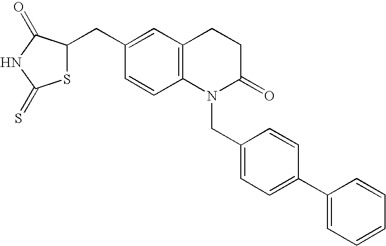 | 0.66 | 0.056 |
| 996 | 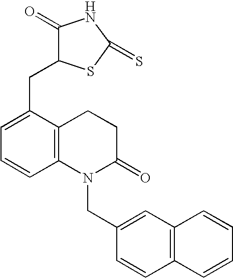 | 0.43 | 0.020 |

*1: Example No. of WO2006/035954

### Example 3

### 1) Extraction of RNA

RNA was extracted from the cells used in Example 2 according to the manufacturer's protocol for ABI Prism 6100 Nucleic Acid PrepStation (a nucleic acid isolation system, product of Applied Biosystems). More specifically, after removal of the culture supernatant and washing with 1 mL of PBS, 250 μL of Nucleic Acid Purification Lysis Solution (Part No. 4305895, Applied Biosystems) was added, and then agitated using a shaker for 2 to 3 minutes. The Lysis Solution containing the cell lysate was collected, and the total amount was transferred to the wells of RNA Purification Tray (Part No. 4305673, Applied Biosystems), which had been pre-wet with 40 μL of Nucleic Acid Purification Wash Solution I (Part No. 4305891, Applied Biosystems) beforehand. Aspiration (20% pressure) was performed for 2 minutes to adsorb the sample onto the RNA Purification Tray, followed by sequentially adding and aspirating (20% pressure, 2 min.) 500 μL of Wash Solution I, Nucleic Acid Purification (Part No. 4305891, Applied Biosystems), 400 μL of Wash Solution II, Nucleic Acid Purification (Part No. 4305890, product of Applied Biosystems), 400 μL of the Wash Solution II, and 300 μL of the Wash Solution II in that order. Aspiration (90% pressure) was performed for 5 minutes to remove the water content from the RNA Purification Trays. After addition of 150 μL of Elution Solution, Nucleic Acid Purification (Part No. 4305893, product of Applied Biosystems), aspiration (20% pressure) was performed for 2 minutes, while collecting the outflow. This was used as collected RNA solution.

### 2) Determination of the RNA Concentration

Absorbance of the collected RNA solution at 260 nm was measured using a microplate spectrophotometer (“SpectraMax Plus”, product of Molecular Devices) with an analysis software (“SOFTmaxPRO ver.3.1.2”, product of Molecular Devices), and the RNA concentration was determined from the absorbance. Likewise, absorbance at 280 nm was measured and the purity of the RNA was determined using the analysis software.

### 3) Preparation of cDNA

cDNA was synthesized according to the manufacturer's protocol for TaqMan® Reverse Transcription Reagents (Part No. N808-0234, product of Applied Biosystems). More specifically, about 0.5 μg of RNA, 2.5 μL of 10×RT buffer, 5.5 μL of MgCl₂ solution, 5 μL of dNTP mixture, 1.25 μL of random hexamer (primers), 0.5 μL of RNase inhibitor, and 0.63 μL of MultiScribe Reverse Transcriptase (product of Applied Biosystems) were added to each collected RNA sample in a tube, and DEPC-treated water was added to make a total volume of 25 μL and stirred. A reaction consisting of 25° C. for 10 minutes, 48° C. for 30 minutes, and 95° C. for 5 minutes was performed using a PCR thermal cycler (“TaKaRa PCR Thermal Cycler Dice”, product of Takara Bio Inc.) to prepare a cDNA solution.

### 4) Real-Time PCR

### PCR Reaction

PCR was performed according to the manufacturer's protocol for TaqMan® Gene Expression Assays (product of Applied Biosystems). More specifically, 5 μL of TaqMan® Fast Universal PCR Master Mix (product of Applied Biosystems), 0.5 μL of TaqMan® Gene Expression Assays for the target gene (gene-specific probe and primer sets, product of Applied Biosystems), and 4.5 μL of the above-obtained cDNA solution were added to each sample and stirred. A PCR reaction consisting of heating at 95° C. for 20 seconds, followed by 40 cycles consisting of heating at 95° C. for 3 seconds and annealing and extension at 60° C. for 30 seconds was performed using a real-time PCR device (“Applied Biosystems 7500 Real-Time PCR System”) with SDS v1.4 Software (product of Applied Biosystems). Threshold cycle (Ct) values were calculated using the software.

The Assay IDs of the TaqMan® Gene Expression Assays for the target genes used are as follows:

Hs00153133_m1 for COX2

Hs00174128_m1 for TNFα

Hs00174103_m1 for IL-8

Hs99999902_m1 for ACTB

### 5) Method of Determining Relative mRNA Expression Levels

The expression level of each gene was determined according to the following formula using the level of ACTB as a reference standard.

(Relative mRNA expression level)=2̂−{(Ct value of the target gene)−(Ct value of ACTB)}

The relative mRNA expression levels achieved by various pharmaceuticals were determined by setting the relative mRNA expression level in IL-1β-stimulated, DMSO-treated MKN-45 cells as 100%.

The results indicate that the carbostyril compound (I) or a salt thereof has an inhibiting activity against COX-2, TNF-α, and/or IL-8, particularly an inhibiting activity against production of COX-2, TNF-α, and/or IL-8, more particularly an inhibiting activity against gene expression of COX-2, TNF-α, and/or IL-8.

| | | | Mean | | | SD | | |
|---|---|---|---|---|---|---|---|---|
| No. | Compounds*1 | IL-1 | COX2 | TNFa | IL-8 | COX2 | TNFa | IL-8 |
| | DMSO (0.05%) | + | 100.0% | 100.0% | 100.0% | 4.0% | 11.5% | 16.7% |
| | 025 | + | 38.3% | 53.0% | 59.9% | 4.8% | 10.9% | 1.7% |
| | 123 | + | 28.6% | 28.5% | 48.3% | 2.7% | 7.6% | 16.6% |
| | 125 | + | 29.2% | 52.5% | 45.4% | 3.3% | 2.3% | 3.7% |
| | 127 | + | 31.0% | 49.8% | 44.8% | 1.3% | 10.5% | 8.0% |
| | 131 | + | 80.9% | 71.3% | 77.9% | 3.0% | 10.9% | 9.7% |
| | 134 | + | 33.1% | 36.6% | 55.4% | 5.9% | 2.6% | 2.4% |
| | 150 | + | 53.7% | 53.9% | 52.0% | 2.8% | 18.8% | 0.9% |
| | 159 | + | 27.7% | 33.5% | 34.0% | 3.6% | 4.5% | 1.2% |
| | 164 | + | 21.7% | 30.0% | 24.8% | 2.8% | 9.1% | 3.1% |
| | 186 | + | 29.7% | 70.8% | 67.7% | 11.8% | 18.0% | 3.7% |
| | 225 | + | 32.9% | 44.6% | 50.0% | 3.3% | 10.7% | 9.7% |
| | 232 | + | 29.9% | 28.9% | 33.8% | 5.7% | 10.1% | 5.7% |
| | 233 | + | 24.9% | 28.9% | 30.4% | 1.8% | 10.7% | 0.8% |
| | 234 | + | 24.0% | 32.4% | 28.3% | 2.2% | 15.6% | 1.8% |
| | 237 | + | 25.9% | 26.2% | 25.9% | 5.4% | 18.6% | 4.9% |
| | 608 | + | 42.4% | 30.8% | 36.5% | 4.0% | 7.6% | 2.9% |
| | 612 | + | 33.4% | 31.1% | 41.8% | 6.8% | 6.9% | 3.7% |
| | 613 | + | 112.4% | 94.0% | 91.5% | 7.4% | 17.9% | 6.5% |
| | 616 | + | 26.3% | 31.4% | 35.3% | 20% | 8.3% | 2.8% |
| | 914 | + | 32.9% | 66.7% | 61.9% | 5.1% | 27.1% | 5.1% |
| | 965 | + | 37.6% | 63.0% | 61.2% | 4.3% | 13.7% | 1.5% |
| | 971 | + | 30.8% | 63.4% | 55.9% | 6.5% | 22.1% | 4.5% |
| | 980 | + | 53.8% | 98.0% | 79.4% | 8.2% | 8.9% | 6.3% |
| | 981 | + | 37.8% | 75.7% | 70.4% | 5.0% | 17.1% | 9.1% |
| | 993 | + | 27.6% | 46.8% | 45.6% | 3.8% | 21.5% | 8.7% |
| | 995 | + | 28.1% | 29.3% | 38.3% | 2.5% | 7.2% | 2.4% |
| | 996 | + | 31.3% | 41.3% | 50.1% | 4.5% | 11.4% | 0.1% |

*1Example No. of WO2006/035954

1. A preventive or therapeutic agent for an NF-κB-associated disease comprising as an active ingredient a carbostyril compound represented by General Formula (1) 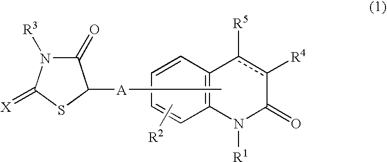
or a salt thereof,
wherein A is a direct bond, a lower alkylene group, or a lower alkylidene group;
X is an oxygen atom or a sulfur atom;
the bond between the 3 and 4 positions of the carbostyril skeleton is a single bond or a double bond;
R⁴ and R⁵ each represent a hydrogen atom, with the proviso that when the bond between the 3 and 4 positions of the carbostyril skeleton is a double bond, R⁴ and R⁵ instead may be linked together in the form of a —CH═CH—CH═CH— group;
R¹ is one of the following (1-1) to (1-30):
(1-1) a hydrogen atom,
(1-2) a lower alkyl group,
(1-3) a phenyl lower alkyl group optionally substituted on the phenyl ring with one or more members selected from the group consisting of a phenyl group, lower alkyl groups, lower alkoxy groups, halogen atoms, —(B)ₗNR⁶R⁷ groups, a nitro group, a carboxy group, lower alkoxycarbonyl groups, a cyano group, phenyl lower alkoxy groups, a phenoxy group, a piperidinyl lower alkoxycarbonyl groups, amino lower alkoxycarbonyl groups optionally substituted with one or more cycloalkyl groups, 2-imidazolinylcarbonyl groups optionally substituted on the 2-imidazoline ring with one or more lower alkylthio groups, 3-pyrrolinylcarbonyl groups optionally substituted on the 3-pyrroline ring with one or more lower alkyl groups, thiazolidinylcarbonyl groups optionally substituted on the thiazolidine ring with a phenyl group, 3-azabicyclo[3.2.2]nonylcarbonyl groups, piperidinyl lower alkyl groups, anilino lower alkyl groups optionally substituted on the amino group with one or more lower alkyl groups, phenylthio lower alkyl groups, indolinyl lower alkyl groups, and piperidinylcarbonyl groups optionally substituted on the piperidine ring with one or more lower alkyl groups,
(1-4) a cycloalkyl lower alkyl group,
(1-5) a phenoxy lower alkyl group,
(1-6) a naphthyl lower alkyl group,
(1-7) a lower alkoxy lower alkyl group,
(1-8) a carboxy lower alkyl group,
(1-9) a lower alkoxycarbonyl lower alkyl group,
(1-10) a pyridyl lower alkyl group optionally substituted on the pyridine ring with one or more members selected from the group consisting of halogen atoms; piperidinyl groups; a morpholino group; piperazinyl groups optionally substituted on the piperazine ring with one or more members selected from the group consisting of a phenyl group and lower alkyl group; thienyl groups; a phenyl group; pyridyl groups; piperidinyl lower alkyl groups; phenylthio lower alkyl groups; biphenyl groups; lower alkyl groups optionally substituted with one or more halogen atoms; pyridylamino groups; pyridylcarbonylamino groups; lower alkoxy groups; anilino lower alkyl groups optionally substituted on the amino group with one or more lower alkyl groups; and anilino groups optionally substituted on the amino group with one or more lower alkyl groups,
(1-11) a cyano lower alkyl group,
(1-12) an -A₁-CONR⁸R⁹ group,
(1-13) a group of the following formula 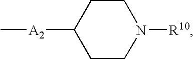
(1-14) a phenyl group,
(1-15) a quinolyl lower alkyl group,
(1-16) a lower alkoxy lower alkoxy-substituted lower alkyl group,
(1-17) a hydroxy-substituted lower alkyl group,
(1-18) a thiazolyl lower alkyl group optionally substituted on the thiazole ring with one or more members selected from the group consisting of halogen atoms, a phenyl group, thienyl groups, and pyridyl groups,
(1-19) a lower alkyl group optionally substituted with one or more halogen atoms,
(1-20) a lower alkylsilyloxy lower alkyl group,
(1-21) a phenoxy lower alkyl group optionally substituted on the phenyl ring with one or more members selected from the group consisting of lower alkyl groups optionally substituted with one or more halogen atoms; lower alkoxy groups; halogen atoms; lower alkenyl groups; cycloalkyl groups; a nitro group; and a phenyl group,
(1-22) a phenylthio lower alkyl group optionally substituted on the phenyl ring with one or more halogen atoms,
(1-23) a piperidinyl lower alkyl groups optionally substituted on the piperidine ring with one or more members selected from the group consisting of phenyl lower alkyl groups and a phenyl group,
(1-24) a piperazinyl lower alkyl group optionally substituted on the piperazine ring with one or more phenyl groups,
(1-25) a 1,2,3,4-tetrahydroisoquinolyl lower alkyl group,
(1-26) a naphthyloxy lower alkyl group,
(1-27) a benzothiazolyloxy lower alkyl group optionally substituted on the benzothiazole ring with one or more alkyl groups,
(1-28) a lower alkyl group substituted with one or more members selected from the group consisting of quinolyloxy groups and isoquinolyloxy groups,
(1-29) a pyridyloxy lower alkyl group optionally substituted on the pyridine ring with one or more lower alkyl groups,
(1-30) a lower alkenyl;
R² is one of the following (2-1) to (2-33):
(2-1) a hydrogen atom,
(2-2) a lower alkoxy group,
(2-3) a lower alkyl group,
(2-4) a carboxy lower alkoxy group,
(2-5) a lower alkoxycarbonyl lower alkoxy group,
(2-6) a hydroxy group,
(2-7) a phenyl lower alkoxy group optionally substituted on the phenyl ring with one or more members selected from the group consisting of halogen atoms; lower alkyl groups optionally substituted with one or more halogen atoms; lower alkylthio groups optionally substituted with one or more halogen atoms; lower alkoxy groups; a nitro group; lower alkylsulfonyl groups; lower alkoxycarbonyl groups; phenyl lower alkenyl groups; lower alkanoyloxy groups; and 1,2,3-thiadiazolyl groups,
(2-8) a piperidinyl lower alkoxy group optionally substituted on the piperidine ring with one or more lower alkyl groups,
(2-9) an amino-substituted lower alkoxy group optionally substituted with one or more lower alkyl groups,
(2-10) a lower alkenyloxy group,
(2-11) a pyridyl lower alkoxy group optionally substituted on the pyridine ring with one or more lower alkyl groups, each lower alkyl substituent optionally being substituted with one or more halogen atoms,
(2-12) a lower alkynyloxy group,
(2-13) a phenyl lower alkynyloxy group,
(2-14) a phenyl lower alkenyloxy group,
(2-15) a furyl lower alkoxy group optionally substituted on the furan ring with one or more lower alkoxycarbonyl groups,
(2-16) a tetrazolyl lower alkoxy group optionally substituted on the tetrazole ring with one member selected from the group consisting of a phenyl group, phenyl lower alkyl groups, and cycloalkyl lower alkyl groups,
(2-17) a 1,2,4-oxadiazolyl lower alkoxy group optionally substituted on the 1,2,4-oxadiazole ring with a phenyl group, the phenyl substituent optionally being substituted on the phenyl ring with one or more lower alkyl groups,
(2-18) an isoxazolyl lower alkoxy group optionally substituted on the isoxazole ring with one or more lower alkyl groups,
(2-19) a 1,3,4-oxadiazolyl lower alkoxy group optionally substituted on the 1,3,4-oxadiazole ring with a phenyl group, the phenyl substituent optionally being substituted on the phenyl ring with one or more lower alkyl groups,
(2-20) a lower alkanoyl lower alkoxy group,
(2-21) a thiazolyl lower alkoxy group optionally substituted on the thiazole ring with one or more members selected from the group consisting of lower alkyl groups and a phenyl group, each phenyl substituent optionally being substituted on the phenyl ring with one or more halogen atoms,
(2-22) a piperidinyloxy group optionally substituted on the piperidine ring with one or more benzoyl groups, each benzoyl substituent optionally being substituted on the phenyl ring with one or more halogen atoms,
(2-23) a thienyl lower alkoxy group,
(2-24) a phenylthio lower alkoxy group,
(2-25) a carbamoyl-substituted lower alkoxy group optionally substituted with one or more lower alkyl groups,
(2-26) a benzoyl lower alkoxy group,
(2-27) a pyridylcarbonyl lower alkoxy group,
(2-28) an imidazolyl lower alkoxy group optionally substituted on the imidazole ring with one or more phenyl lower alkyl groups,
(2-29) a phenoxy lower alkoxy group,
(2-30) a phenyl lower alkoxy-substituted lower alkoxy group,
(2-31) a 2,3-dihydro-1H-indenyloxy group,
(2-32) an isoindolinyl lower alkoxy group optionally substituted on the isoindoline ring with one or more oxo groups,
(2-33) a phenyl group;
R³ is one of the following (3-1) to (3-19):
(3-1) a hydrogen atom,
(3-2) a lower alkyl group,
(3-3) a hydroxy-substituted lower alkyl group,
(3-4) a cycloalkyl lower alkyl group,
(3-5) a carboxy lower alkyl group,
(3-6) a lower alkoxycarbonyl lower alkyl group,
(3-7) a phenyl lower alkyl group optionally substituted on the phenyl ring with one or more members selected from the group consisting of halogen atoms; lower alkyl groups optionally substituted with one or more halogen atoms; lower alkoxy groups optionally substituted with one or more halogen atoms; a phenyl group; lower alkoxycarbonyl groups; a phenoxy group; lower alkylthio groups; lower alkylsulfonyl groups; phenyl lower alkoxy groups; and amino groups optionally substituted with one or more lower alkanoyl groups,
(3-8) a naphthyl lower alkyl group,
(3-9) a furyl lower alkyl group optionally substituted on the furan ring with one or more lower alkoxycarbonyl groups,
(3-10) a thiazolyl lower alkyl group optionally substituted on the thiazole ring with one or more members selected from the group consisting of lower alkyl groups and a phenyl group, each phenyl substituent optionally being substituted on the phenyl ring with one or more optionally halogen-substituted lower alkyl groups,
(3-11) a tetrazolyl lower alkyl group optionally substituted on the tetrazole ring with one or more lower alkyl groups,
(3-12) a benzothienyl lower alkyl group optionally substituted on the benzothiophene ring with one or more halogen atoms,
(3-13) a lower alkynyl group,
(3-14) a lower alkenyl group,
(3-15) a phenyl lower alkenyl group,
(3-16) a benzoimidazolyl lower alkyl group,
(3-17) a pyridyl lower alkyl group,
(3-18) an imidazolyl lower alkyl group optionally substituted on the imidazole ring with one or more phenyl lower alkyl groups,
(3-19) a quinolyl lower alkyl group;
B is a carbonyl group or an —NHCO— group;
l is 0 or 1;
R⁶ and R⁷ each independently represent one of the following (4-1) to (4-79):
(4-1) a hydrogen atom,
(4-2) a lower alkyl group,
(4-3) a lower alkanoyl group,
(4-4) a lower alkylsulfonyl group optionally substituted with one or more halogen atoms,
(4-5) an alkoxycarbonyl group optionally substituted with one or more halogen atoms,
(4-6) a hydroxy-substituted lower alkyl group,
(4-7) a pyridylcarbonyl group optionally substituted on the pyridine ring with one or more members selected from the group consisting of pyrrolyl groups and halogen atoms,
(4-8) a pyridyl group optionally substituted on the pyridine ring with one or more members selected from the group consisting of lower alkyl groups and lower alkoxy groups,
(4-9) a pyridyl lower alkyl group,
(4-10) a phenyl group optionally substituted on the phenyl ring with one or more members selected from the group consisting of halogen atoms; lower alkyl groups optionally substituted with one or more halogen atoms; a phenoxy group; lower alkoxy groups optionally substituted with one or more halogen atoms; lower alkylthio groups; lower alkylsulfonyl groups; amino groups optionally substituted with one or more members selected from the group consisting of lower alkyl groups and lower alkanoyl groups; pyrrolidinyl groups optionally substituted on the pyrrolidine ring with one or more oxo groups; piperidinyl groups optionally substituted on the piperidine ring with one or more lower alkyl groups; lower alkenyl groups; an aminosulfonyl group; a hydroxy group; carbamoyl groups optionally substituted with one or more lower alkyl groups; phenyl lower alkoxy groups; and a cyano group,
(4-11) a cycloalkyl group optionally substituted on the cycloalkyl ring with one or more lower alkyl groups,
(4-12) a benzoyl group optionally substituted on the phenyl ring with one or more members selected from the group consisting of halogen atoms; a phenoxy group; a phenyl group; lower alkyl groups optionally substituted with one or more halogen atoms; lower alkoxy groups; lower alkanoyl groups; a nitro group; a cyano group; amino groups optionally substituted with one or more members selected from the group consisting of a phenyl group and lower alkyl groups; pyrrolidinyl groups optionally substituted on the pyrrolidine ring with one or more oxo groups; pyrrolyl groups; pyrazolyl groups; 1,2,4-triazolyl groups; and imidazolyl groups,
(4-13) a benzoyl group substituted on the phenyl ring with one or more lower alkylenedioxy groups,
(4-14) a cycloalkylcarbonyl group,
(4-15) a furylcarbonyl group,
(4-16) a naphthylcarbonyl group,
(4-17) a phenoxycarbonyl group optionally substituted on the phenyl ring with one or more members selected from the group consisting of lower alkoxy groups, lower alkyl groups, halogen atoms, and a nitro group,
(4-18) a phenyl lower alkoxycarbonyl group optionally substituted on the phenyl ring with one or more members selected from the group consisting of halogen atoms and a nitro group,
(4-19) a piperidinyl group optionally substituted on the piperidine ring with one or more members selected from the group consisting of lower alkyl groups; lower alkanoyl groups; benzoyl groups optionally substituted on the phenyl ring with one or more halogen atoms; and phenyl groups optionally substituted on the phenyl ring with one or more halogen atoms,
(4-20) a tetrahydropyranyl lower alkyl group,
(4-21) a cycloalkyl lower alkyl group,
(4-22) a lower alkenyl group,
(4-23) a phenyl lower alkyl group optionally substituted on the alkyl group with one or more lower alkoxycarbonyl groups; and optionally substituted on the phenyl ring with one or more members selected from the group consisting of halogen atoms, lower alkyl groups optionally substituted with one or more halogen atoms, lower alkoxy groups optionally substituted with one or more halogen atoms, and a hydroxy group,
(4-24) a lower alkylenedioxy-substituted phenyl lower alkyl group,
(4-25) a furyl lower alkyl group,
(4-26) a carbamoyl lower alkyl group optionally substituted with one or more members selected from lower alkyl groups and a phenyl group, each phenyl substituent optionally being substituted on the phenyl ring with one or more lower alkyl groups,
(4-27) a lower alkoxy lower alkyl group,
(4-28) an imidazolyl lower alkyl group optionally substituted on the lower alkyl group with one or more members selected from the group consisting of a carbamoyl group and lower alkoxycarbonyl groups,
(4-29) an amino-substituted lower alkyl group optionally substituted with one or more lower alkyl groups,
(4-30) a 2,3,4,5-tetrahydrofuryl group optionally substituted on the 2,3,4,5-tetrahydrofuran ring with one or more oxo groups,
(4-31) a lower alkoxycarbonyl lower alkyl group,
(4-32) a pyrrolidinyl lower alkyl group optionally substituted on the pyrrolidine ring with one or more lower alkyl groups,
(4-33) a phenoxy lower alkanoyl group,
(4-34) a morpholino lower alkyl group,
(4-35) a indolyl group,
(4-36) a thiazolyl group,
(4-37) a 1,2,4-triazolyl group,
(4-38) a pyridyl lower alkanoyl group,
(4-39) a thienylcarbonyl group,
(4-40) a thienyl lower alkanoyl group,
(4-41) a cycloalkyl lower alkanoyl group,
(4-42) an isoxazolylcarbonyl group optionally substituted on the isoxazole ring with one or more lower alkyl groups,
(4-43) a pyrazylcarbonyl group,
(4-44) a piperidinylcarbonyl group optionally substituted on the piperidine ring with one or more members selected from a benzoyl group and lower alkanoyl groups,
(4-45) a chromanylcarbonyl group,
(4-46) an isoindolinyl lower alkanoyl group optionally substituted on the isoindoline ring with one or more oxo groups,
(4-47) a thiazolidinyl lower alkanoyl group optionally substituted on the thiazolidine ring with one or more members selected from an oxo group and a thioxo group,
(4-48) a piperidinyl lower alkanoyl group,
(4-49) a phenyl lower alkenylcarbonyl group optionally substituted on the phenyl ring with one or more halogen atoms,
(4-50) a phenyl lower alkenylcarbonyl group substituted on the phenyl ring with one or more alkylenedioxy groups,
(4-51) a pyridyl lower alkenyl carbonyl group,
(4-52) a pyridylthio lower alkanoyl group,
(4-53) an indolylcarbonyl group,
(4-54) a pyrrolylcarbonyl group,
(4-55) a pyrrolidinylcarbonyl group optionally substituted on the pyrrolidine ring with one or more oxo groups,
(4-56) a benzofurylcarbonyl group,
(4-57) an indolyl lower alkanoyl group,
(4-58) a benzothienylcarbonyl group,
(4-59) a phenyl lower alkanoyl group optionally substituted on the phenyl ring with one or more halogen atoms,
(4-60) a phenylsulfonyl group optionally substituted on the phenyl ring with one or more members selected from the group consisting of lower alkoxycarbonyl groups; a cyano group; a nitro group; amino groups optionally substituted with one or more alkanoyl groups; a hydroxy group; a carboxyl group; lower alkoxycarbonyl lower alkyl groups; halogen atoms; lower alkyl groups optionally substituted with one or more halogen atoms; and lower alkoxy groups optionally substituted with one or more halogen atoms,
(4-61) a thienylsulfonyl group optionally substituted on the thiophene ring with one or more members selected from the group consisting of halogen atoms and lower alkoxycarbonyl groups,
(4-62) a quinolylsulfonyl group,
(4-63) an imidazolylsulfonyl group optionally substituted on the imidazole ring with one or more lower alkyl groups,
(4-64) a phenylsulfonyl group optionally substituted on the phenyl ring with one or more lower alkylenedioxy groups,
(4-65) a lower alkenylsulfonyl group,
(4-66) a cycloalkyl lower alkylsulfonyl group,
(4-67) a 3,4-dihydro-2H-1,4-benzoxazinylsulfonyl group optionally substituted on the 3,4-dihydro-2H-1,4-benzoxazine ring with one or more lower alkyl groups,
(4-68) a pyrazolylsulfonyl group optionally substituted on the pyrazole ring with one or more members selected from halogen atoms and lower alkyl groups,
(4-69) an isoxazolylsulfonyl group optionally substituted on the isoxazole ring with one or more lower alkyl groups,
(4-70) a thiazolylsulfonyl group optionally substituted on the thiazole ring with one or more members selected from the group consisting of lower alkyl groups and an amino group, each amino substituent optionally being substituted with one or more alkanoyl groups,
(4-71) a phenyl lower alkylsulfonyl group,
(4-72) a phenyl lower alkenylsulfonyl group,
(4-73) a naphthyloxycarbonyl group,
(4-74) a lower alkynyloxycarbonyl group,
(4-75) a lower alkenyloxycarbonyl group,
(4-76) a phenyl lower alkoxy-substituted lower alkoxycarbonyl group,
(4-77) a cycloalkyloxycarbonyl group optionally substituted on the cycloalkyl ring with one or more lower alkyl groups,
(4-78) a tetrazolyl group,
(4-79) an isoxazolyl group optionally substituted on the isoxazole ring with one or more lower alkyl groups; or instead,
R⁶ and R⁷ may be linked together to form, together with the nitrogen atom to which they are bound, a 1,2,3,4-tetrahydroisoquinolyl group, an isoindolinyl group, or a 5- to 7-membered saturated heterocyclic group, the heterocyclic group optionally containing one or more additional heteroatoms and optionally being substituted with one to three members from the following (5-1) to (5-28):
(5-1) lower alkyl groups,
(5-2) lower alkoxy groups,
(5-3) an oxo group,
(5-4) a hydroxy group,
(5-5) pyridyl lower alkyl groups,
(5-6) phenyl groups optionally substituted on the phenyl ring with one or more members selected from the group consisting of halogen atoms; lower alkoxy groups optionally substituted with one or more halogen atoms; lower alkyl groups optionally substituted with one or more halogen atoms; a cyano group; and a hydroxy group,
(5-7) lower alkylenedioxy-substituted phenyl lower alkyl groups,
(5-8) phenyl lower alkyl groups optionally substituted on the phenyl ring with one or more halogen atoms,
(5-9) pyrimidyl groups,
(5-10) pyrazyl groups,
(5-11) cycloalkyl groups,
(5-12) phenyl lower alkoxy groups optionally substituted on the phenyl ring with one or more halogen atoms,
(5-13) benzoyl groups optionally substituted on the phenyl ring with one or more halogen atoms,
(5-14) benzoyl groups substituted on the phenyl ring with one or more lower alkylenedioxy groups,
(5-15) carbamoyl lower alkyl groups optionally substituted with one or more members selected from the group consisting of a phenyl group and lower alkyl groups,
(5-16) benzoxazolyl groups,
(5-17) lower alkoxycarbonyl groups,
(5-18) a carbamoyl group,
(5-19) phenyl lower alkylidene groups optionally substituted on the phenyl ring with one or more halogen atoms,
(5-20) phenyl lower alkoxycarbonyl groups,
(5-21) pyridyl groups optionally substituted on the pyridine ring with one or more members selected from the group consisting of a cyano group and lower alkyl groups,
(5-22) furyl lower alkyl groups,
(5-23) tetrahydropyranyl groups,
(5-24) imidazolyl lower alkyl groups,
(5-25) naphthyl groups,
(5-26) 2,3-dihydro-1H-indenyl groups,
(5-27) 1,3-dioxolanyl lower alkyl groups,
(5-28) -(A₃)ₘNR¹¹R¹² groups;
A₁ is a lower alkylene group;
R⁸ and R⁹ each independently represent one of the following (6-1) to (6-25):
(6-1) a hydrogen atom,
(6-2) a lower alkyl group,
(6-3) a phenyl group optionally substituted on the phenyl ring with one or more members selected from the group consisting of lower alkyl groups optionally substituted with one or more halogen atoms; lower alkylthio groups; lower alkoxy groups optionally substituted with one or more halogen atoms; halogen atoms; a phenyl group; lower alkylamino groups; a cyano group; a phenoxy group; cycloalkyl groups; pyrrolidinyl groups optionally substituted with one or more oxo groups; 1,2,3,4-tetrahydroisoquinolylcarbonyl groups; 1,2,3,4-tetrahydroquinolylcarbonyl groups optionally substituted with one or more lower alkyl groups; 1,2,3,4-tetrahydroquinoxalinylcarbonyl groups optionally substituted with one or more lower alkyl groups; thiazolyl groups optionally substituted with one or more phenyl groups; a carbamoyl group; phenyl lower alkoxy groups; lower alkylsulfonylamino groups; anilino groups optionally substituted with one or more halogen atoms; phenyl lower alkyl groups; and hydroxy-substituted lower alkyl groups,
(6-4) a cycloalkyl group,
(6-5) a cycloakyl lower alkyl group,
(6-6) a carbamoyl lower alkyl group,
(6-7) a phenyl lower alkyl group optionally substituted on the phenyl ring with one or more members selected from the group consisting of lower alkyl groups optionally substituted with one or more halogen atoms; lower alkoxy groups optionally substituted with one or more halogen atoms; halogen atoms; and a phenyl group,
(6-8) lower alkyl-substituted amino lower alkyl group,
(6-9) a naphthyl group,
(6-10) a naphthyl lower alkyl group,
(6-11) a tetrahydronaphthyl lower alkyl group,
(6-12) a fluorenyl group,
(6-13) a pyridyl group,
(6-14) a pyridyl lower alkyl group,
(6-15) a pyrimidinyl group,
(6-16) a pyrazinyl lower alkyl group optionally substituted on the pyrazine ring with one or more lower alkyl groups,
(6-17) a thiazolyl group,
(6-18) a pyrazolyl lower alkyl group optionally substituted on the pyrazole ring with one or more lower alkyl groups,
(6-19) a thienyl lower alkyl group
(6-20) a piperidinyl group optionally substituted on the piperidine ring with one or more members selected from the group consisting of lower alkyl groups; a benzoyl group; and phenyl lower alkyl groups optionally substituted on the phenyl ring with one or more members selected from the group consisting of halogen atoms and lower alkyl groups,
(6-21) an indolyl group,
(6-22) an indazolyl group,
(6-23) a 3,4-dihydrocarbostyril optionally substituted with one or more lower alkyl groups,
(6-24) a quinolyl group optionally substituted with one or more lower alkyl groups,
(6-25) a carbazolyl group optionally substituted with one or more lower alkyl groups; or
R⁸ and R⁹ may be linked together to form, together with the nitrogen atom to which they are bound, a 5- to 8-membered saturated heterocyclic group optionally containing one or more additional heteroatoms and optionally substituted on the heterocyclic ring with one or more members selected from the group consisting of the following (6-28-1) to (6-28-24):
(6-28-1) lower alkyl groups,
(6-28-2) phenyl lower alkyl groups optionally substituted on the phenyl ring with one or more members selected from halogen atoms and lower alkoxy groups optionally substituted with one or more halogen atoms,
(6-28-3) naphthyl lower alkyl groups,
(6-28-4) phenyl lower alkylcarbamoyl lower alkyl groups,
(6-28-5) phenylcarbamoyl lower alkyl groups,
(6-28-6) phenyl lower alkoxycarbonyl groups,
(6-28-7) phenoxy lower alkyl groups optionally substituted on the phenyl ring with one or more members selected from the group consisting of halogen atoms and lower alkyl groups optionally substituted with one or more halogen atoms,
(6-28-8) biphenyl groups,
(6-28-9) phenyl groups optionally substituted on the phenyl ring with one or more halogen atoms,
(6-28-10) 2,3-dihydroindenyl groups optionally substituted with one or more halogen atoms,
(6-28-11) benzothiazolyl groups optionally substituted with one or more halogen atoms,
(6-28-12) pyridyl groups optionally substituted with one or more halogen atoms,
(6-28-13) benzothienyl groups,
(6-28-14) benzoisothiazolyl groups,
(6-28-15) thienopyridyl groups,
(6-28-16) a carbamoyl group,
(6-28-17) phenyl lower alkoxy groups optionally substituted on the phenyl ring with one or more halogen atoms,
(6-28-18) phenoxy groups optionally substituted with one or more halogen atoms,
(6-28-19) benzoyl groups optionally substituted on the phenyl ring with one or more members selected from halogen atoms and lower alkoxy groups,
(6-28-20) anilino groups optionally substituted on the phenyl ring with one or more lower alkyl groups, each lower alkyl substituent optionally being substituted with one or more halogen atoms,
(6-28-21) anilino groups substituted on the amino group with one or more lower alkyl groups, and optionally further substituted on the phenyl ring with one or more halogen atoms,
(6-28-22) benzofuryl groups,
(6-28-23) naphthyl groups,
(6-28-24) an oxo group; or
R⁸ and R⁹ may be linked together to form, together with the nitrogen atom to which they are bound, a 5- or 6-membered unsaturated heterocyclic group, the unsaturated heterocyclic group optionally being substituted on the heterocyclic ring with one or more members selected from the group consisting of the following (6-29-1) to (6-29-3):
(6-29-1) phenyl groups optionally substituted with one or more halogen atoms,
(6-29-2) 2,3-dihydroindenyl groups,
(6-29-3) benzothienyl groups; or instead,
R⁸ and R⁹ may be linked together to form, together with the nitrogen atom to which they are bound, a 1,2,3,4-tetrahydroquinolyl group; a 1,2,3,4-tetrahydroisoquinolyl group, a 1,3-dihydroisoindolyl group; an octahydropyrrolo[1,2-a]pyrazinyl group optionally substituted on the pyrazine ring with one or more lower alkyl groups; or an 8-azabicyclo[3.2.1]octyl group optionally substituted on the 8-azabicyclo[3.2.1]octyl group with one or more phenoxy groups, each phenoxy substituent optionally being substituted on the phenyl ring with one or more halogen atoms;
A₂ is a lower alkylene group;
R¹⁰ is one of the following (7-1) to (7-44):
(7-1) a hydrogen atom,
(7-2) a lower alkyl group,
(7-3) an alkoxycarbonyl group optionally substituted with one or more halogen atoms,
(7-4) a benzoyl group optionally substituted on the phenyl ring with one or more members selected from the group consisting of lower alkyl groups optionally substituted with one or more halogen atoms; a phenyl group; halogen atoms; a cyano group; a phenoxy group; lower alkoxycarbonyl groups; pyrazolyl groups; and lower alkoxy groups optionally substituted with one or more halogen atoms,
(7-5) an alkanoyl group,
(7-6) a phenyl lower alkanoyl group optionally substituted on the phenyl ring with one or more members selected from the group consisting of halogen atoms and lower alkyl groups,
(7-7) a cycloalkyl lower alkanoyl group,
(7-8) a phenyl group optionally substituted on the phenyl ring with one or more lower alkyl groups,
(7-9) a phenoxy lower alkanoyl group optionally substituted on the phenyl ring with one or more halogen atoms,
(7-10) a phenyl lower alkenylcarbonyl group,
(7-11) a pyridylcarbonyl group optionally substituted on the pyridine ring with one or more members selected from the group consisting of halogen atoms and lower alkyl groups, each lower alkyl substituent optionally being substituted with one or more halogen atoms,
(7-12) a furylcarbonyl group,
(7-13) a thienylcarbonyl group,
(7-14) a piperidinylcarbonyl group optionally substituted on the piperidine ring with one or more lower alkanoyl groups,
(7-15) a pyrrolidinylcarbonyl group optionally substituted on the pyrrolidine ring with one or more oxo groups,
(7-16) a tetrahydropyranylcarbonyl group,
(7-17) a naphthylcarbonyl group,
(7-18) an indolylcarbonyl group,
(7-19) a benzofurylcarbonyl group,
(7-20) a benzothienylcarbonyl group optionally substituted on the benzothiophene ring with one or more halogen atoms,
(7-21) a furyl lower alkyl group,
(7-22) a pyridyl lower alkyl group optionally substituted on the pyridine ring with one or more members selected from the group consisting of halogen atoms and lower alkyl groups, each lower alkyl substituent optionally being substituted with one or more halogen atoms,
(7-23) a thienyl lower alkyl group optionally substituted on the thiophene ring with one or more halogen atoms,
(7-24) a phenyl lower alkyl group optionally substituted on the phenyl ring with one or more members selected from the group consisting of lower alkoxy groups optionally substituted with one or more halogen atoms; a cyano group; lower alkyl groups optionally substituted with one or more halogen atoms; amino groups optionally substituted with one or more members selected from the group consisting of lower alkyl groups and lower alkanoyl groups; halogen atoms; lower alkoxycarbonyl groups; lower alkanoyloxy groups; lower alkylsulfonyl groups; lower alkylthio groups; and pyrrolidinyl groups,
(7-25) a thiazolyl lower alkyl group,
(7-26) an imidazolyl lower alkyl group optionally substituted on the imidazole ring with one or more lower alkyl groups,
(7-27) a pyrrolyl lower alkyl group optionally substituted on the pyrrole ring with one or more lower alkyl groups,
(7-28) a cycloalkyl lower alkyl group,
(7-29) a lower alkylthio lower alkyl group,
(7-30) a phenoxycarbonyl group optionally substituted on the phenyl ring with one or more members selected from the group consisting of halogen atoms, lower alkyl groups, and lower alkoxy groups,
(7-31) a phenyl lower alkoxycarbonyl group optionally substituted on the phenyl ring with one or more halogen atoms,
(7-32) a naphthyloxycarbonyl group,
(7-33) a lower alkynyloxycarbonyl group,
(7-34) a cycloalkylcarbonyl group,
(7-35) a quinoxalinylcarbonyl group,
(7-36) a —CO—NR¹³R¹⁴ group,
(7-37) a piperidinyl group optionally substituted on the piperidine ring with one or more lower alkyl groups,
(7-38) a cycloalkyl group,
(7-39) a tetrahydropyranyl group,
(7-40) a lower alkoxy lower alkyl group,
(7-41) a tetrahydro-2H-thiopyranyl group,
(7-42) a naphthyl group,
(7-43) a biphenyl group,
(7-44) a lower alkylsilyl lower alkoxycarbonyl group;
A³ is a lower alkylene group;
m is 0 or 1;
R¹¹ and R¹² each independently represent one of the following (8-1) to (8-5):
(8-1) a hydrogen atom,
(8-2) a lower alkyl group,
(8-3) a lower alkanoyl group,
(8-4) a phenyl lower alkanoyl group,
(8-5) a phenyl group optionally substituted on the phenyl ring with one or more halogen atoms; or instead,
R¹¹ and R¹² may be linked together to form, together with the nitrogen atom to which they are bound, a 5- or 6-membered saturated heterocyclic group which optionally contains one or more additional heteroatoms, the heterocyclic group optionally being substituted with one to three members selected from the group consisting of the following (9-1) and (9-2):
(9-1) lower alkyl groups,
(9-2) a phenyl group; and
R¹³ and R¹⁴ each independently represent one of the following (10-1) to (10-3):
(10-1) a hydrogen atom,
(10-2) a lower alkyl group,
(10-3) a phenyl group, or instead
R¹³ and R¹⁴ may be linked together to form, together with the nitrogen atom to which they are bound, a 5- or 6-membered saturated heterocyclic group which optionally contains one or more additional heteroatoms.

2. The agent according to claim 1 wherein the NF-κB-associated disease is selected from the group consisting of reperfusion disorders in ischemic diseases, aggravation of the prognosis of organ transplantation or organ surgery, post-PTCA restenosis, cancer metastasis and invasion, and cachexia.

3. The agent according to claim 1, which is for regulating transcription of cytokines and/or adhesion factors.

4. The agent according to claim 3, which is for inhibiting activity of COX-2, TNF-α, and/or IL-8.

5. A preventive or therapeutic agent for a COX-2-associated disease or disorder comprising the carbostyril compound of claim 1 or a salt thereof as an active ingredient.

6. An anticancer-effect reinforcer for use in radiotherapy or chemotherapy comprising the carbostyril compound of claim 1 or a salt thereof as an active ingredient.

7. A radiosensitivity enhancer for use in radiotherapy comprising the carbostyril compound of claim 1 or a salt thereof as an active ingredient.

8. A tumor-induced angiogenesis inhibitor comprising the carbostyril compound of claim 1 or a salt thereof as an active ingredient.

9. A method for reinforcing anticancer effects in radiotherapy or chemotherapy, comprising administering an effective amount of the carbostyril compound of claim 1 or a salt thereof to a patient.

10. Use of the carbostyril compound of claim 1 or a salt thereof to produce an anticancer-effect reinforcer for use in radiotherapy or chemotherapy.

11. The agent according to claim 1, wherein the carbostyril compound is represented by General Formula (1) in which R⁴ and R⁵ each represent a hydrogen atom.

12. The agent according to claim 11, wherein the carbostyril compound is represented by General Formula (1) in which a group of the formula 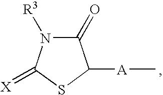
in which R³, A and X are as defined in claim 1 above, is bound to the 3, 4, 5, 6, 7 or 8 position of the carbostyril skeleton.

13. The agent according to claim 12, wherein the carbostyril compound is represented by General Formula (1) in which the bond between the 3 and 4 positions of the carbostyril skeleton is a single bond, and the group of the formula, 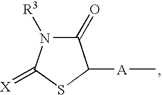
in which R³, A and X are as defined in claim 1 above, is bound to the 5 or 6 position of the carbostyril skeleton.

14. The agent according to claim 12 or 13, wherein the carbostyril compound is represented by General Formula (1) in which A is a lower alkylene group or a lower alkylidene group.

15. The agent according to claim 14, wherein the carbostyril compound is represented by General Formula (1) in which R¹ is one of (1-2), (1-3), (1-4), (1-6), (1-10), (1-12), (1-13), (1-18) and (1-21) as defined in claim 1 above.

16. The agent according to claim 15, wherein the carbostyril compound is represented by General Formula (1) in which the group of the formula 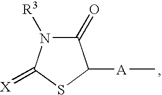
in which R³, A and X are as defined in claim 1 above, is bound to the 5 position of the carbostyril skeleton.

17. The agent according to claim 16, wherein the carbostyril compound is represented by General Formula (1) in which R¹ is a phenyl lower alkyl group optionally substituted on the phenyl ring with one or more members selected from the group consisting of a phenyl ring, halogen atoms, —(B)ₗNR⁶R⁷ groups wherein B, l, R⁶ and R⁷ are as defined in claim 1, lower alkoxycarbonyl groups, and phenyl lower alkoxy groups.

18. The agent according to claim 17, wherein the carbostyril compound is represented by General Formula (1) in which A is a lower alkylene group, R² is a hydrogen atom or a lower alkoxy group, R³ is a hydrogen atom, and X is an oxygen atom or a sulfur atom.

19. The agent according to claim 16, wherein the carbostyril compound is represented by General Formula (1) in which A is a lower alkylene group, R¹ is a lower alkyl group, R² is a hydrogen atom or a lower alkoxy group, R³ is a hydrogen atom, and X is an oxygen atom or a sulfur atom.

20. The agent according to claim 16, wherein the carbostyril compound is represented by General Formula (1) in which A is a lower alkylene group, R¹ is a naphthyl lower alkyl group, R² is a hydrogen atom or a lower alkoxy group, R³ is a hydrogen atom, and X is an oxygen atom or a sulfur atom.

21. The agent according to claim 16, wherein the carbostyril compound is represented by General Formula (1) in which A is a lower alkylene group, R¹ is a group of the formula 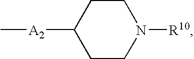
in which R¹⁰ and A₂ are as defined in claim 1 above, R² is a hydrogen atom or a lower alkoxy group, R³ is a hydrogen atom, and X is an oxygen atom or a sulfur atom.

22. The agent according to claim 12, wherein the carbostyril compound is represented by General Formula (1) in which the bond between the 3 and 4 positions of the carbostyril skeleton is a double bond, and a group of the formula 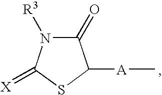
in which R³, A and X are as defined in claim 1 above, is bound to the 3, 4 or 5 position of the carbostyril skeleton.

23. The agent according to claim 22, wherein the carbostyril compound is represented by General Formula (1) in which R¹ is one of (1-2) and (1-3) as defined in claim 1.

24. The agent according to claim 23, wherein the carbostyril compound is represented by General Formula (1) in which A is a lower alkylene group or a lower alkylidene group, and R² is a hydrogen atom or a lower alkoxy group.

25. The agent according to claim 1, wherein the carbostyril compound is represented by General Formula (1) in which the bond between the 3 and 4 positions of the carbostyril skeleton is a double bond, and R⁴ and R⁵ are linked together in the form of a —CH═CH—CH═CH— group.

26. The agent according to claim 25, wherein the carbostyril compound is represented by General Formula (1) in which a group of the formula 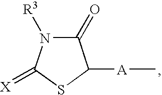
in which R³, A and X are as defined in claim 1 above, is bound to the 7 position of the carbostyril skeleton.

27. The agent according to claim 26, wherein the carbostyril compound is represented by General Formula (1) in which R¹ is one of (1-2) and (1-3) as defined in claim 1 above.

28. The agent according to claim 27, wherein the carbostyril compound is represented by General Formula (1) in which A is a lower alkylene group or a lower alkylidene group, R² and R³ are both hydrogen atoms, and X is an oxygen atom or a sulfur atom.

29. The agent according to claim 1, wherein the carbostyril compound is represented by General Formula (1) in which A is a direct bond.

30. The agent according to claim 1, wherein the carbostyril compound is represented by General Formula (1) in which A is a lower alkylene group.

31. The agent according to claim 1, wherein the carbostyril compound is represented by General Formula (1) in which A is a lower alkylidene group.

32. The agent according to any one of claims 29 to 31, wherein the carbostyril compound is represented by General Formula (1) in which the bond between the 3 and 4 positions of the carbostyril skeleton is a single bond or a double bond, and R⁴ and R⁵ each represent a hydrogen atom.

33. The agent according to any one of claims 29 to 31, wherein the carbostyril compound is represented by General Formula (1) in which the bond between the 3 and 4 positions of the carbostyril skeleton is a double bond, and R⁴ and R⁵ are linked together in the form of a —CH═CH—CH═CH— group.

34. The agent according to claim 1, wherein the carbostyril compound is selected from the group consisting of the following compounds:
5-[1-(biphenyl-4-ylmethyl)-8-methoxy-2-oxo-1,2,3,4-tetrahydroquinolin-5-ylmethyl]thiazolidine-2,4-dione,
5-[1-(4-chlorobenzyl)-8-methoxy-2-oxo-1,2,3,4-tetrahydroquinolin-5-ylmethyl]thiazolidine-2,4-dione,
5-[1-(4-bromobenzyl)-8-methoxy-2-oxo-1,2,3,4-tetrahydroquinolin-5-ylmethyl]thiazolidine-2,4-dione,
5-[1-(2-naphthylmethyl)-8-methoxy-2-oxo-1,2,3,4-tetrahydroquinolin-5-ylmethyl]thiazolidine-2,4-dione,
5-{1-[4-(heptyloxycarbonylamino)benzyl]-8-methoxy-2-oxo-1,2,3,4-tetrahydroquinolin-5-ylmethyl}thiazolidine-2,4-dione,
5-[1-(1-biphenyl-4-ylpiperidin-4-ylmethyl)-2-oxo-1,2,3,4-tetrahydroquinolin-5-ylmethyl]thiazolidine-2,4-dione,
5-{1-[1-(4-methylphenyl)piperidin-4-ylmethyl]-2-oxo-1,2,3,4-tetrahydroquinolin-5-ylmethyl}thiazolidine-2,4-dione,
5-{1-[4-(2-chlorobenzyloxycarbonylamino)benzyl]-8-methoxy-2-oxo-1,2,3,4-tetrahydroquinolin-5-ylmethyl}thiazolidine-2,4-dione,
1-(biphenyl-4-ylmethyl)-8-methoxy-5-(4-oxo-2-thioxothiazolidin-5-ylmethyl)-3,4-dihydro-1H-quinolin-2-one,
8-methoxy-1-methyl-5-(4-oxo-2-thioxothiazolidin-5-ylmethyl)-3,4-dihydro-1H-quinolin-2-one,
8-methoxy-1-(3-methylbutyl)-5-(4-oxo-2-thioxothiazolidin-5-ylmethyl)-3,4-dihydro-1H-quinolin-2-one,
1-propyl-8-methoxy-5-(4-oxo-2-thioxothiazolidin-5-ylmethyl)-3,4-dihydro-1H-quinolin-2-one,
1-isobutyl-8-methoxy-5-(4-oxo-2-thioxothiazolidin-5-ylmethyl)-3,4-dihydro-1H-quinolin-2-one,
8-methoxy-1-phenethyl-5-(4-oxo-2-thioxothiazolidin-5-ylmethyl)-3,4-dihydro-1H-quinolin-2-one, and
1-(4-phenylthiomethyl)benzyl-5-(4-oxo-2-thioxothiazolidin-5-ylmethyl)-3,4-dihydro-1H-quinolin-2-one; or a salt thereof.
